(19) United States
(12) Patent Application Publication
(10) Pub. No.: US 2026/0250655 A1
(43) Pub. Date: Aug. 27, 2026

(54) HELICASE-CYTIDINE DEAMINASE COMPLEXES AND METHODS OF USE

(71) Applicant: ILLUMINA, INC., San Diego, CA (US)

(72) Inventors: Gaetano Speciale, San Diego, CA (US); Stephen Gross, San Diego, CA (US); Dewei Joel Toh, Singapore (SG); Leslie Yee Ming Beh, Singapore (SG)

(21) Appl. No.: 18/879,424

(22) PCT Filed: Sep. 29, 2023
(86) PCT No.: PCT/IB2023/059798

§ 371 Date: Dec. 27, 2024

Related U.S. Application Data
(60) Provisional application No. 63/412,221, filed on Sep. 30, 2022.
(60) Provisional application No. 63/416,143, filed on Oct. 14, 2022.

(51) Int. Cl. C12N 9/78; C12N 9/14; C12N 15/10

(52) U.S. Cl. CPC C12N 9/78; C12N 9/14; C12N 15/1093; C12Y 305/04005; C12Y 306/04012

(57) ABSTRACT

Protein complexes including a cytidine deaminase and a helicase. In some embodiments, the cytidine deaminase is an altered cytidine deaminase. In some embodiments, the protein complex converts 5 methylcytosine to thymine. Kits, compositions, and methods of use for the protein complexes including a cytidine deaminase and a helicase are also described.

### CROSS-REFERENCE TO RELATED APPLICATIONS

This application claims the benefit of U.S. Provisional Application Ser. No. 63/412,221, filed on Sep. 30, 2022, and U.S. Provisional Application Ser. No. 63/416,143, filed on Oct. 14, 2022, each of which is incorporated by reference herein in its entirety.

### SEQUENCE LISTING

This application contains a Sequence Listing electronically submitted via EFS-Web to the United States Patent and Trademark Office as an XML file entitled “0531_002462W001_SL.xml” having a size of 429 kilobytes and created on Sep. 25, 2023. The information contained in the Sequence Listing is incorporated by reference herein.

### FIELD

Embodiments of the present disclosure relate to preparing nucleic acids for sequencing or other applications. In particular, embodiments of the proteins, methods, compositions, and kits provided herein relate to mapping of methylation status by using sequencing libraries and other methods.

### BACKGROUND

Modified DNA cytosines, including 5-methylcytosine (5mC) and 5-hydroxymethyl cytosine (5hmC), are a well-studied epigenetic modification that play fundamental roles in human development and disease. Its genome-wide distribution of differs between tissue types and between healthy and diseased states. In recent years, 5mC has also gained prominence as a tool for clinical diagnostics: its distribution in cell-free DNA (cfDNA)—obtained from a liquid biopsy—can be used for the tissue-specific prediction of early-stage cancer or monitoring of cancer recurrence or remission after treatment. As a result, there has been an intense focus on developing methods for mapping modified DNA cytosines at single base resolution, with minimal loss of sample DNA quantity, quality, and complexity. Current methods for mapping modified DNA cytosines, however, exhibit limitations including (i) degradation of sample DNA due to prolonged chemical treatment at non-neutral pH and high temperatures, (ii) loss of sample DNA complexity due to conversion of unmethylated DNA bases to uracil, resulting in low complexity genome mapping, (iii) multi-step conversion, requiring both enzymes and chemical treatment, and (iv) for antibody-based 5mC detection, resolution of detection is limited to ~150 bp, precluding the identification of its exact location in the genome.

5-hydroxymethylcytosine (5hmC) is an oxidized derivative of the widely studied epigenetic modification 5-methylcytosine (5mC). Increasing evidence supports the biological importance of 5hmC in diverse developmental processes in mammals, such as neurogenesis. As such, there is widespread interest in determining the localization of 5hmC in DNA from healthy and diseased patients. The majority of methods for mapping 5-hydroxymethylcytosine (5hmC) require bisulfite treatment, which results in significant DNA loss and damage. Recent methods for mapping 5hmC have been developed, such as oxBS-seq, TAB-seq, and ACE-seq, but some include bisulfite treatment, and all involve multiple steps using different enzymes.

### SUMMARY OF THE APPLICATION

The present disclosure provides proteins, methods, compositions, and kits for determining the methylation status of DNA and RNA using a cytidine deaminase and a helicase in close physical proximity. Unlike current methods for mapping methylation status of cytosine (C) nucleotides, the present disclosure presents one-step, fully enzymatic methods using an altered cytidine deaminases that selectively act on certain modified cytosines of target nucleic acids and converts them to thymidine (T) or modified thymidine analogs. The altered cytidine deaminases described herein circumvent the limitations of currently available methods for mapping methylated cytosine nucleotides because (i) they are active at near-neutral pH and physiological temperature, (ii) unmethylated cytosines are reacted at a decreased rate so sample DNA complexity is preserved, (iii) conversion of methylated C to T is a single-step enzymatic reaction, (iv) the process results in detection of methylated C at single base resolution, and (v) the process maintains DNA complexity, simplifying analysis by next-generation sequencing. The altered and wild-type (WT) cytidine deaminases described herein typically have greater catalytic activity on single-stranded nucleic acids. The use of a helicase and a cytidine deaminase in close physical proximity during treatment of a nucleic acid is expected to demonstrate improved catalytic activity on double-stranded nucleic acids.

The present disclosure also provides proteins, methods, compositions, and kits for mapping 5-hydroxymethylcytosine (5hmC) nucleotides present in DNA and RNA. Current methods for mapping methylation status of 5hmC nucleotides include a step of modifying or blocking 5hmC nucleotides. For instance, the ACE-seq method (Schutsky et al., Nature biotechnology, 10.1038/nbt.4204. 8 Oct. 2018, doi:10.1038/nbt.4204) blocks 5hmC by conversion to 5ghmC using the enzyme β-glucosyltransferase (βGT). Unlike current methods for mapping methylation status of 5hmC nucleotides, the methods presented herein do not require modifying or blocking 5hmC nucleotides. Instead, the present disclosure presents one-step, fully enzymatic methods using altered cytidine deaminases that selectively act on certain modified cytosines of target nucleic acids and converts them to uracil (U) or thymidine (T) but do not act on 5hmC, 5-formylcytosine (5fC), or 5-carboxycytosine (5-caC). The altered cytidine deaminases described herein circumvent the limitations of currently available methods for mapping 5hmC nucleotides because (i) harsh chemical treatments that cause significant loss of DNA and RNA are not required, (ii) the conversion is a single-step enzymatic reaction, and (iii) the process results in detection of 5hmC at single base resolution.

The present disclosure includes altered cytidine deaminases, including a protein complex of an altered cytidine deaminase and a second protein, such as a helicase. In one embodiment, an altered cytidine deaminase includes amino acid substitution mutations at positions functionally equivalent to (Tyr/Phe)130 and Tyr132 in a wild-type APOBEC3A protein. In another embodiment, an altered cytidine deaminase includes an amino acid substitution mutation at a position functionally equivalent to (Tyr/Phe)130 in a wild-type APOBEC3A protein, where the substitution mutation is (Tyr/Phe)130Trp. The (Tyr/Phe)130 of an altered cytidine deaminase can be Tyr130, and the wild-type APOBEC3A protein is SEQ ID NO:3. The present disclosure also includes a polynucleotide encoding an altered cytidine deaminase.

The present disclosure also provides compositions that include the altered cytidine deaminase described herein, including a protein complex of a cytidine deaminase and a second protein, such as a helicase. In one embodiment, a composition can further include at least one of (i) a sample including DNA including at least one modified cytosine, where the modified cytosine is 5-methyl cytosine (5mC), 5-hydroxymethyl cytosine (5hmC), 5-formyl cytosine (5fC), 5-carboxy cytosine (5caC), or a combination thereof; or (ii) a buffer, such as one having a pH that is lower than 7; or (iii) combinations thereof.

Also provided by the present disclosure are methods for using a cytidine deaminase described herein, including a protein complex of an altered cytidine deaminase and a second protein, such as a helicase. In one embodiment, a method includes providing a sample of DNA suspected of including single-stranded DNA including at least one 5-methyl cytosine (5mC), at least one 5-hydroxymethyl cytosine (5hmC), at least one 5-formyl cytosine (5fC), at least one 5-carboxy cytosine (5CaC), or a combination thereof; contacting the single-stranded DNA with an altered cytidine deaminase under conditions suitable for (i) conversion of 5-methylcytosine (5mC) to thymidine (T) by deamination at a greater rate than conversion of cytosine (C) to uracil (U) by deamination, to result in converted single-stranded DNA, or (ii) conversion of C to U by deamination and 5mC to T by deamination at a greater rate than conversion of 5-hydroxymethyl cytosine (5hmC) to 5-hydroxymethyl uracil (5hmU) by deamination; and processing the converted single-stranded DNA to produce a sequencing library.

In another embodiment, a method includes providing a sample of DNA suspected of including double-stranded DNA including at least one 5-methyl cytosine (5mC), at least one 5-hydroxymethyl cytosine (5hmC), at least one 5-formyl cytosine (5fC), at least one 5-carboxy cytosine (5caC), or a combination thereof; processing the double-stranded DNA to produce a sequencing library; denaturing the sequencing library to result in a single-stranded DNA; contacting the single-stranded DNA with an altered cytidine deaminase under conditions suitable for (i) conversion of 5-methylcytosine (5mC) to thymidine (T) by deamination at a greater rate than conversion of cytosine (C) to uracil (U) by deamination, or (ii) conversion of C to U by deamination and 5mC to T by deamination at a greater rate than conversion of 5-hydroxymethyl cytosine (5hmC) to 5-hydroxymethyl uracil (5hmU) by deamination, to result in converted single-stranded DNA; and converting the converted single-stranded DNA to a converted double-stranded DNA sequencing library.

In one embodiment, a method can include detecting the location of a modified cytosine in a target nucleic acid. The method can include (a) contacting target nucleic acids suspected of including at least one modified cytosine with the altered cytidine deaminase of claim 1 or 2 to produce converted nucleic acids including at least one converted cytosine; and (b) detecting the at least one converted cytosine in the converted nucleic acids of (a). The detecting can include sequencing the converted nucleic acids or hybridizing nucleic acid probes to the converted nucleic acids.

In embodiments where the detecting includes sequencing the converted nucleic acids, the method can further include (c) comparing the sequence of the converted nucleic acids with an untreated reference sequence to determine which cytosines in the target nucleic acids are modified.

In embodiments where the detecting includes hybridizing the converted nucleic acids to nucleic acid probes, then the method can further include the nucleic acid probes can be present on an analyte array, and the method can further include sequencing the hybridized converted nucleic acids. In another embodiment the method can further include amplifying the converted nucleic acid, where the nucleic acid probes include two primers for amplification of a predetermined sequence, where the primers anneal to regions of converted nucleic acids including at least one converted cytosine with a greater affinity than to the regions of converted nucleic acids where at least one cytosine is not a converted cytosine, and where the presence of an amplified product is indicative of a modified cytosine in the target nucleic acid. In another embodiment the method can further include cleaving a single stranded DNA (ssDNA) reporter substrate by a CRISPR-based system, where the ssDNA reporter substrate includes a fluorophore and a quencher, where the presence of fluorescence is indicative of a modified cytosine in the target nucleic acid. In another embodiment the converted nucleic acids can be present in a fixed cell, where the nucleic acid probes include a fluorescent labeled probe, and where the nucleic acid probes anneal to a predetermined sequence of converted nucleic acids including at least one converted cytosine with a greater affinity than to the regions of converted nucleic acids where at least one cytosine is not a converted cytosine, where the presence of cell-associated fluorescence is indicative of a modified cytosine in the target nucleic acid.

In one embodiment of detecting the location of a modified cytosine in a target nucleic acid, the target nucleic acids can be obtained from a subject, and the detecting can include obtaining a pattern of cytosine modification in the converted nucleic acids. In some embodiments, the method can further include comparing the pattern of cytosine modification in the converted nucleic acids with the pattern of cytosine modification in a reference nucleic acid. For instance, the subject can be one that has or is at risk of having a disease or condition, and the reference nucleic acid can be from a normal subject. In one embodiment, a pattern of cytosine modification is linked in-cis to a coding region that is correlated with a disease or condition. For instance, the pattern of cytosine modification is linked in-cis to a coding region, where the coding region in the reference nucleic acid is transcriptionally active or transcriptionally inactive. The comparing can further include determining if the pattern of cytosine modification of the converted nucleic acid indicates the coding region is transcriptionally active or transcriptionally inactive in the subject. The transcription of the coding region can be correlated with a disease or condition.

In one aspect, this disclosure describes protein complexes including a cytidine deaminase and a helicase. The cytidine deaminase may be a wild-type cytidine deaminase, or it may be an altered cytidine deaminase. In some embodiments, the altered cytidine deaminase may have an amino acid substitution mutation at a position functionally equivalent to (Tyr/Phe)130 in a wild-type APOBEC3A protein.

In another aspect, this disclosure describes a composition including a protein complex including a cytidine deaminase and a helicase, and a DNA sample including double-stranded DNA including at least one 5mC.

In another aspect, this disclosure describes a method including providing a sample of DNA suspected of including dsDNA including at least one 5mC, contacting the sample with a protein complex descried herein under conditions suitable for conversion of 5mC to T to result in converted dsDNA, wherein the 5mC is converted to T, and processing the converted dsDNA.

In another aspect, this disclosure describes a protein complex including a means for converting a 5mC to a T, and a means for directionally separating double-stranded nucleic acids.

Terms used herein will be understood to take on their ordinary meaning in the relevant art unless specified otherwise. Several terms used herein and their meanings are set forth below.

As used herein, the terms “organism” and “subject” are used interchangeably and refer to microbes (e.g., prokaryotic or eukaryotic) animals and plants. An example of an animal is a mammal, such as a human.

As used herein, the term “target nucleic acid,” is intended as a semantic identifier for the nucleic acid in the context of a method, composition, or kit set forth herein, and does not necessarily limit the structure or function of the nucleic acid beyond what is otherwise explicitly indicated. Reference to a nucleic acid such as a target nucleic acid includes both single-stranded and double-stranded nucleic acids, and both DNA and RNA, unless indicated otherwise. The term library refers to the collection of target nucleic acids containing known common sequences, such as a universal sequence or adapter, at their 3′ and 5′ ends.

As used herein, the term “adapter” and its derivatives, e.g., universal adapter, refers generally to any linear oligonucleotide which can be attached to a target nucleic acid. An adapter can be single-stranded or double-stranded DNA, or can include both double-stranded and single-stranded regions. An adapter can include a universal sequence that is substantially identical, or substantially complementary, to at least a portion of a primer, for example a universal primer; an index (also referred to herein as a barcode or tag) to assist with downstream error correction, identification, or sequencing; and/or a unique molecular identifier. In some embodiments, the adapter is substantially non-complementary to the 3′ end or the 5′ end of any target sequence present in the sample. In some embodiments, suitable adapter lengths are in the range of about 6-100 nucleotides, about 12-60 nucleotides, or about 15-50 nucleotides in length. For instance, the terms “adaptor” and “adapter” are used interchangeably.

As used herein, the term “universal,” when used to describe a nucleotide sequence, refers to a region of sequence that is common to two or more nucleic acid molecules where the molecules also have regions of sequence that differ from each other. A universal sequence that is present in different members of a collection of nucleic acids can be used as, for instance, a “landing pad” in a subsequent step to anneal a nucleotide sequence that can be used as a primer for addition of another nucleotide sequence, such as an index, to a target nucleic acid. A universal sequence that is present in different members of a collection of nucleic acids can allow capture of multiple different nucleic acids using a population of universal capture nucleic acids, e.g., capture oligonucleotides that are complementary to a portion of the universal sequence, e.g., a universal capture sequence. Non-limiting examples of universal capture sequences include sequences that are identical to or complementary to P5 and P7 primers. Similarly, a universal sequence present in different members of a collection of molecules can allow the replication (e.g., sequencing) or amplification of multiple different nucleic acids using a population of universal primers that are complementary to a portion of the universal sequence, e.g., a universal anchor sequence. In one embodiment universal anchor sequences are used as a site to which a universal primer (e.g., a sequencing primer for read 1 or read 2) anneals for sequencing. A capture oligonucleotide or a universal primer therefore includes a sequence that can hybridize specifically to a universal sequence.

The terms “P5” and “P7” may be used when referring to a universal capture sequence or a capture oligonucleotide. The terms “P5′” (P5 prime) and “P7′” (P7 prime) refer to the complement of P5 and P7, respectively. It will be understood that any suitable universal capture sequence or a capture oligonucleotide can be used in the methods presented herein, and that the use of P5 and P7 are exemplary embodiments only. Uses of capture oligonucleotides such as P5 and P7 or their complements on flow cells are known in the art, as exemplified by the disclosures of WO 2007/010251, WO 2006/064199, WO 2005/065814, WO 2015/106941, WO 1998/044151, and WO 2000/018957, which are incorporated by reference as to P5 and P7 and their uses. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein.

As used herein, the term “primer” and its derivatives refer generally to any nucleic acid that can hybridize to a target sequence of interest. Typically, the primer functions as a substrate onto which nucleotides can be polymerized by a polymerase or to which a polynucleotide can be ligated; in some embodiments, however, the primer can become incorporated into the synthesized nucleic acid strand and provide a site to which another primer can hybridize to prime synthesis of a new strand that is complementary to the synthesized nucleic acid molecule. In some embodiments, the primer can be used for hybridization to a predetermined sequence, for instance a predetermined sequence that includes one or more nucleotides that identify the location of a modified cytosine. In one embodiment, a “primer” includes a sequence present in a guide RNA used with a CRISPR-based system to hybridize to a predetermined sequence. The primer can include any combination of nucleotides or analogs thereof. In some embodiments, the primer is a single-stranded oligonucleotide or polynucleotide.

The terms “polynucleotide,” “oligonucleotide,” and “nucleic acid” are used interchangeably herein to refer to a polymeric form of nucleotides of any length, and may include ribonucleotides, deoxyribonucleotides, analogs thereof, or mixtures thereof. The terms should be understood to include, as equivalents, analogs of either DNA, RNA, cDNA, or antibody-oligo conjugates made from nucleotide analogs and to be applicable to single stranded (such as sense or antisense) and double stranded polynucleotides. The term as used herein also encompasses cDNA, that is complementary or copy DNA produced from a RNA template, for example by the action of reverse transcriptase.

As used herein, an “index” (also referred to as an “index region,” “index adaptor,” “tag,” or a “barcode”) refers to a unique nucleic acid tag that can be used to identify a sample or source of the nucleic acid material, or a compartment in which a target nucleic acid was present. The index can be present in solution, on a solid-support, or attached to or associated with a solid-support and released in solution or compartment. When nucleic acid samples are derived from multiple sources, the nucleic acids in each nucleic acid sample can be tagged with different nucleic acid tags such that the source of the sample can be identified. Any suitable index or set of indexes can be used, as known in the art and as exemplified by the disclosures of U.S. Pat. No. 8,053,192, PCT Publication No. WO 05/068656, and U.S. Pat. Publication No. 2013/0274117. In some embodiments, an index can include a six-base Index 1 (i7) sequence, an eight-base Index 1 (i7) sequence, an eight-base Index 2 (i5e) sequence, a ten-base Index 1 (i7) sequence, or a ten-base Index 2 (i5) sequence from Illumina, Inc. (San Diego, CA).

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), ligation extension, or ligation chain reaction. An amplicon can be a nucleic acid molecule having a single copy of a particular nucleotide sequence (e.g. a PCR product) or multiple copies of the nucleotide sequence (e.g. a concatemeric product of RCA). A first amplicon of a target nucleic acid is typically a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

As used herein, “amplify”, “amplifying” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. Amplification is typically the exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR).

As used herein, the term “polymerase chain reaction” (“PCR”) refers to the method of Mullis U.S. Pat. Nos. 4,683,195 and 4,683,202, which describe a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as PCR. Because the desired amplified segments of the polynucleotide of interest become the predominant nucleic acid sequences (in terms of concentration) in the mixture, they are said to be “PCR amplified”. In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

As used herein, “amplification conditions” and its derivatives, generally refers to conditions suitable for amplifying one or more nucleic acid sequences. In some embodiments, the amplification conditions can include isothermal conditions or alternatively can include thermocycling conditions, or a combination of isothermal and thermocycling conditions. In some embodiments, the conditions suitable for amplifying one or more nucleic acid sequences include polymerase chain reaction (PCR) conditions. Typically, the amplification conditions refer to a reaction mixture that is sufficient to amplify nucleic acids such as one or more target sequences flanked by a universal sequence, or target specific primers, or to amplify an amplified target sequence flanked by one or more adapters. Generally, the amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates (dNTPs) to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. Typically, but not necessarily, amplification conditions can include thermocycling; in some embodiments, amplification conditions include a plurality of cycles where the steps of annealing, extending and separating are repeated. Typically, the amplification conditions include cations such as Mg²⁺ or Mn²⁺ and can also include various modifiers of ionic strength.

As defined herein “multiplex amplification” refers to selective and non-random amplification of two or more target sequences within a sample using at least one target-specific primer. In some embodiments, multiplex amplification is performed such that some or all of the target sequences are amplified within a single reaction vessel. The “plexy” or “plex” of a given multiplex amplification refers generally to the number of different target-specific sequences that are amplified during that single multiplex amplification. In some embodiments, the plexy can be about 12-plex, 24-plex, 48-plex, 96-plex, 192-plex, 384-plex, 768-plex, 1536-plex, 3072-plex, 6144-plex or higher. It is also possible to detect the amplified target sequences by several different methodologies (e.g., gel electrophoresis followed by densitometry, quantitation with a bioanalyzer or quantitative PCR, hybridization with a labeled probe; incorporation of biotinylated primers followed by avidin-enzyme conjugate detection; incorporation of ³²P-labeled deoxynucleotide triphosphates into the amplified target sequence).

As used herein, the term “amplification site” refers to a site in or on an array where one or more amplicons can be generated. An amplification site can be further configured to contain, hold or attach at least one amplicon that is generated at the site.

As used herein, the terms “array,” “analyte array,” and “microarray” are used interchangeably and refer to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, droplets, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “compartment” is intended to mean an area or volume that separates or isolates something from other things. Exemplary compartments include, but are not limited to, vials, tubes, wells, droplets, boluses, beads, vessels, surface features, flow cell, or areas or volumes separated by physical forces such as fluid flow, magnetism, electrical current or the like. In one embodiment, a compartment is a well of a multi-well plate, such as a 96- or 384-well plate. As used herein, a droplet may include a hydrogel bead, which is a bead for encapsulating one or more nuclei or cell, and includes a hydrogel composition. In some embodiments, the droplet is a homogeneous droplet of hydrogel material or is a hollow droplet having a polymer hydrogel shell. Whether homogenous or hollow, a droplet may be capable of encapsulating one or more nuclei or cells. In some embodiments, the droplet is a surfactant stabilized droplet. In some embodiments, a single cell or Nuclei is present per compartment. In some embodiments, two or more cells or Nuclei are present per compartment. In some embodiments, each compartment contains a compartment-specific index. In some embodiments, the index is in solution or attached or associated with a solid-phase in each compartment.

The term “flow cell” as used herein refers to a chamber comprising a solid surface across which one or more fluid reagents can be flowed. Examples of flow cells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082.

As used herein, the term “clonal population” refers to a population of nucleic acids that is homogeneous with respect to a particular nucleotide sequence. The homogenous sequence is typically at least 10 nucleotides long, but can be even longer including for example, at least 50, 100, 250, 500 or 1000 nucleotides long. A clonal population can be derived from a single target nucleic acid or template nucleic acid. Typically, all of the nucleic acids in a clonal population will have the same nucleotide sequence. It will be understood that a small number of mutations (e.g., due to amplification artifacts) can occur in a clonal population without departing from clonality.

As used herein, a “pattern of cytosine modification,” also referred to as a “methylation profile,” refers to the pattern with which both methylation and unmethylation of cysteines is distributed in the genome of a cell or an organism. A “pattern” is inclusive of both modified cytosines and non-modified cytosines. The pattern can be defined in several distribution dimensions: by organ, by tissue, by status of disease or pathological condition (e.g., cancer, neurophysiological), by genome segment (e.g., chromosome or genetic coordinates on a chromosome), by gene, by CpG island, a group of cytosines, or by the site of a modified cytosine. A pattern of cytosine modification can have a known correlation with a disease or pathological condition, or correlation of a pattern of cytosine modification with a disease or pathological condition can be identified using methods described herein. A pattern of cytosine modification can be present at a specific locus (e.g., location) in a genome, and that specific location can be a single modified cytosine or a set of modified cytosines, e.g., a CpG island. A pattern of cytosine modification can be identified by using a predetermined sequence, e.g., a method of using an altered cytidine deaminase can be designed and practiced with the intent of determining a pattern of cytosine modification, for instance, the methylation status of one of more specific cytosines, the methylation status of one or more specific cytosines present at a specific location of a genome, or the combination thereof.

As used herein, the term “each,” when used in reference to a collection of items, is intended to identify an individual item in the collection but does not necessarily refer to every item in the collection unless the context clearly dictates otherwise.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

Unless otherwise specified, “a,” “an,” “the,” and “at least one” are used interchangeably and mean one or more than one.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have,” “has,” “having,” “include,” “includes,” “including,” “comprise,” “comprises,” “comprising” or the like are used in their open ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Conditions that are “suitable” for an event to occur, such as converting 5 methylcytosine to thymidine by deamination, or “suitable” conditions are conditions that do not prevent such events from occurring. Thus, these conditions permit, enhance, facilitate, and/or are conducive to the event.

As used herein, “providing” in the context of a protein, sample of DNA or RNA, or composition means making the protein, sample of DNA or RNA, or composition, purchasing the protein, sample of DNA or RNA, or composition, or otherwise obtaining the protein, sample of DNA or RNA, or composition.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

While polynucleotide sequences encoding an altered cytidine deaminase, helicase, or fusion protein are described herein as DNA sequences, it is understood that the complements, reverse sequences, and reverse complements of the DNA sequences can be easily determined by the skilled person. It is also understood that the sequences described herein as DNA sequences can be converted from a DNA sequence to an RNA sequence by replacing each thymidine nucleotide with a uracil nucleotide.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 4.5, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1A-C shows the deamination scheme of APOBEC3A. Cytosine (C), 5-methylcytosine (5mC), and 5-hydroxymethylcytosine (5hmC) nucleobases in single-stranded DNA are well characterized substrates of APOBEC3A. FIG. 1A shows conversion of C to uracil (U) by APOBEC3A. FIG. 1B shows conversion of 5mC to thymidine (T) by APOBEC3A. FIG. 1C shows conversion of 5hmC to 5-hydroxymethyl uracil (5hmU) by APOBEC3. “ξ” denotes the connection of the nucleobases to a DNA molecule.

FIG. 1D-F shows the result of treating a DNA sample with a wild-type APOBEC3A enzyme (FIG. 1D) or an example of one-step detection of 5mC using an altered cytidine deaminase described herein (FIG. 1E). The top strand of FIG. 1D-F shows C, 5mC, and/or 5hmC bases, and the bottom strand of FIG. 1C-D underlines the changed bases. In FIG. 1D, 5mC nucleobases are marked with CH₃, 5hmC nucleobases are marked with CH₂—OH, 5-hydroxymethyl uracil nucleobases are designated with small case “u” and uracil nucleobases are designated with capital “U.”

FIG. 2 shows cytosine and examples of modified cytosine nucleobases in DNA. The ““denotes the connection of the nucleobases to a DNA molecule.

FIG. 3 is a schematic showing alignment of cytidine deaminase amino acid sequences using the Clustal O algorithm. An “*” (asterisk) indicates positions which have a single, fully conserved residue between all cytidine deaminases. A”:” (colon) indicates conservation between groups of strongly similar properties as below—roughly equivalent to scoring >0.5 in the Gonnet PAM 250 matrix. A “.” (period) indicates conservation between groups of weakly similar properties as below—roughly equivalent to scoring=<0.5 and >0 in the Gonnet PAM 250 matrix. The amino acids marked with “\hat{ }” show the ZDD motif SEQ ID NO: 12 (e.g., above amino acids 70 to 106 of sp|P31941|1-199). The amino acids marked with “\hat{ }” and “#” show the ZDD motif SEQ ID NO:13 (e.g., above amino acids 70 to 153 of sp|P31941|1-199). sp|P31941|1-199 is a human APOBEC3A, SEQ ID NO:3; XP_045219544.1 is an APOBEC3A from Macaca fascicularis, SEQ ID NO:19; AER45717.1 is an APOBEC3A from Pongo pygmaeus, SEQ ID NO:20; XP_003264816.1 is an APOBEC3A from Nomascus leucogenys, SEQ ID NO:21; PNI48846.1 is an APOBEC3A from Pan troglodytes, SEQ ID NO:22; and AD085886.1 is an APOBEC3A from Gorilla gorilla, SEQ ID NO:23.

FIG. 4 shows a schematic of restriction enzyme (SwaI)-based assay for deamination by cytidine deaminase. X is H in the context of C, and X is methyl in the context of 5mC, and X is hydroxymethyl in the context of 5hmC. “Matched oligos” refers to complete complementarity between the two oligonucleotides; “mismatched oligos” refers to incomplete complementarity between the two oligonucleotides. A mismatch results in cleavage of the double stranded oligonucleotides by SwaI.

FIG. 5A-B show a positive control experiment of SwaI-based assay using synthesized oligonucleotides. FIG. 5A shows sequences of synthesized oligonucleotides. oLB1609, SEQ ID NO:24; oLB1610, SEQ ID NO:25; oLB1611, SEQ ID NO:26; oLB1612, SEQ ID NO:27; oLB1679, SEQ ID NO:28; oJT1910, and oJT1911. FIG. 5B shows visualization of results of SwaI digestion. “Matched” refers to complete complementarity between the two oligonucleotides; “mismatched” refers to incomplete complementarity between the two oligonucleotides. A mismatch results in cleavage of the double stranded oligonucleotides by SwaI.

FIG. 6 shows a positive control deamination experiment using a commercially available APOBEC3A enzyme.

FIG. 7A-B shows an SDS-PAGE panel of APOBEC3A(Y130X) proteins. FIG. 7A shows SDS-PAGE analysis of purified APOBEC3A(Y130X) mutant proteins. FIG. 7B shows SDS-PAGE analysis of purified APOBEC3A(Y130A_Y132H) mutant proteins.

FIG. 8 shows results of APOBEC3A(Y130X) deamination end-point assay panel using SwaI assay readout.

FIG. 9 shows a bar graph representation of APOBEC3A(Y130X) deaminase activity.

FIG. 10 shows deaminase activity of wild type (NEB APOBEC) and mutant APOBEC variants on C, 5mC, and 5hmC substrates. Percent deamination values were determined from a SwaI restriction enzyme assay and quantified as in FIG. 4. C deamination activity was measured in two independent experiments corresponding to the left and right panels.

FIG. 11A-F shows APOBEC3A(Y130A) time course C and 5mC deamination reaction. FIG. 11A-C shows the reaction at 37° C., and FIG. 11D-F shows the reaction at 22° C. FIG. 11A and FIG. 11D) Y130A deamination SwaI-based assay at 37° C. and 22° C., respectively, visualized by 15% Urea-PAGE with FAM filter. FIG. 11B and FIG. 11E) Graphical representation of Y130A time course C and 5mC deamination reaction at 37° C. and 22° C., respectively. FIG. 11C and FIG. 11F) Table depicting percentage deamination at 37° C. and 22° C., respectively, at various time points.

FIG. 12 shows preliminary Michaelis-Menten kinetics of Y130A on C and 5mC oligonucleotide substrates.

FIG. 13 shows DNA oligonucleotide substrates for evaluating deaminase activity of a double mutant cytidine deaminase. Set(A), SEQ ID NO:29; Set(B), SEQ ID NO:30, 31, 32, and 36, respectively.

FIG. 14 shows percent deamination at each NCN motif in the DNA oligo substrate (A) after incubation (37° C., 6 hour reaction) with APOBEC3A mutants. This metric is calculated as the percentage of C>T (cytosine to thymidine) mutations at each position as determined by DNA sequencing.

FIG. 15 shows percent deamination at each NCN motif in the DNA oligo substrate (A) after incubation (37° C., 1 hour reaction) with APOBEC3A mutants. This metric is calculated as the percentage of C>T mutations at each position.

FIG. 16 shows percent deamination at each NCpGN motif in DNA oligo substrate (B) after incubation with APOBEC3A mutants. Four different DNA oligos were mixed together as a substrate for APOBEC3A deamination. Percent deamination is calculated as the percentage of C>T mutations in each NCpGN motif Methylated and unmethylated forms of each NCpGN motif are assayed (32 sites in total).

FIG. 17A-B shows the time course of APOBEC3A(Y130W) deamination against C, 5mC, and 5hmC-containing substrates. The SwaI restriction enzyme assay was performed to measure deaminase activity of APOBEC3A(Y130W). Percent deamination is calculated as the ratio of the cut band intensity to the (uncut+cut) band intensity. FIG. 17A, gel images and FIG. 17B, quantification of band intensities.

FIG. 18A-B shows comparison of APOBEC3A deaminase activity against C, 5mC, and oxidized derivatives. The SwaI restriction enzyme assay was performed with a 90 min reaction time to measure deaminase activities of APOBEC3A wild type and Y130W mutant enzymes. A no-protein control was included to account for potential degradation of oligonucleotide substrates and account for non-specific activity of SwaI during the course of the assay. Percent deamination was calculated as the ratio of the cut band intensity to the (uncut+cut) band intensity. FIG. 18A, gel images and FIG. 18B, quantification of band intensities, subtracted from the corresponding no-protein control lanes.

FIG. 19 shows a method for 5hmC detection using deaminase-based sequencing.

FIG. 20A-B shows comparison of different reaction conditions and the resulting methylation reporting on pUC19 (CG methylated) and Lambda (fully unmethylated) with the altered cytidine deaminase having Y130A and Y132H. FIG. 20A is the methylation level of methylated pUC19 and unmethylated lambda DNA using the altered cytidine deaminase under different concentrations. FIG. 20B is the methylation level of methylated pUC19 and unmethylated lambda DNA using the altered cytidine deaminase under different buffers.

FIG. 21 shows impact of RNAse A on deamination of methylated pUC19 and unmethylated lambda as determined by sequencing.

FIG. 22A-B shows activity of APOBEC Y130A-Y132H on 5hmC. (FIG. 22A) Strategy for construction of control oligo; (FIG. 22B) Observed methylation level on control oligo substrate. mC, 5mC; hmC, 5hmC.

FIG. 23 shows analysis of regional methylation in CpG islands indicate that deaminase-based assay produces the expected methylation profile. mC-deaminase-seq, APOBEC3A Y130A-Y132H.

FIG. 24 shows performance of SNV and indel calling with and without methylation conversion with APOBEC3A Y130A-Y132H. mC-deaminase-seq, APOBEC3A Y130A-Y132H.

FIG. 25A-F shows visualization of DMRs identified by EM-Seq™ and the altered cytidine deaminase assay in ZNF154 gene. Representation of methylation level across this region is shown in (FIG. 25A) HCC2218-Normal, EM-Seq™ conversion, (FIG. 25B) HCC2218-Tumor, EM-Seq™ conversion, (FIG. 25C) HCC2218-Normal, altered cytidine deaminase assay, (FIG. 25D) HCC2218-Tumor, altered cytidine deaminase assay, (FIG. 25E) differentially methylated regions (DMRs) called between Tumor/Normal samples using EM-Seq™ data, and (FIG. 25F) differentially methylated regions (DMRs) called between Tumor/Normal sample s using altered cytidine deaminase data.

FIG. 26 shows recall and precision of DMRs in HCC1187 Tumor/Normal and HCC2218 Tumor/Normal paired genomes. DMRs from each workflow were compared to DMRs called by EM-Seq™, which was used as the truth set. BiSulfite identifies a majority of the DMRs that EM-Seq™ identifies. Additionally, the mC-selective deamination protocols described here in Method A, Method B, and Method C are able to identify most of the DMRs identified by EM-Seq™ mC-deaminase-seq, APOBEC3A Y130A-Y132H.

FIG. 27A-B shows methylation levels detected in promoter regions using Method A and EM-Seq™. (FIG. 27A) Methylation level at H3K36me3 regions, which are expected to be hypermethylated, (FIG. 27B) Methylation levels at H3K27ac regions, which are expected to be hypomethylated. Dotted traces, methylation levels detected in promoter regions using Method A; solid traces, methylation levels detected in promoter regions using EM-Seq™.

FIG. 28 shows tumor signal for 0% spike-in of tumor DNA into normal DNA vs a 10% spike-in of tumor DNA into normal DNA. Methylation level of HCC2218 normal DNA at individual CpG sites within the PanSeer cancer panel were assessed to create a baseline. Methylation level at individual CpG sites were then assessed in separate replicates of HCC2218 normal DNA and a 10% spike-in of HCC2218 tumor DNA into HCC2218 normal DNA. Tumor signal indicates the fraction of CpGs that have a significantly different methylation level compared to the background.

FIG. 29 shows a diagram depicting the benefits of 5mC>T conversion for enrichment.

FIG. 30A-C shows different workflows for enrichment of methyl-converted libraries. (FIG. 30A) Hybridization after conversion and amplification, in which specialized probe designs shown in FIG. 29 are typically employed. (FIG. 30B) Illumina Methyl Capture EPIC Workflow where standard probe designs are employed prior to bisulfite conversion, requiring higher DNA inputs. (FIG. 30C) Workflow for data presented for use with an altered cytidine deaminase (mC-deaminase) with enrichment.

FIG. 31A-B shows enrichment performance of altered cytidine deaminase (mC-deaminase-seq) libraries. (FIG. 31A) Read enrichment performance of altered cytidine deaminase libraries as compared to libraries with no methylation conversion. (FIG. 31B) Correlation of regional methylation levels in CpG islands measured in the unenriched (WGS) sample as compared to the enriched sample.

FIG. 32 shows a schematic of qPCR-based detection of 5mC in a genomic locus of interest. Selective 5mC deamination by APOBEC3A(Y130A/Y132H) results in a DNA template that is fully complementary to qPCR primers, and can therefore be amplified. No deamination by APOBEC3A(Y130A/Y132H) will be observed in unmethylated substrates, resulting in qPCR primers being mismatched to the target site, therefore compromising amplification. “Selective 5mC->T deamination” refers to the result of incubating the ssDNA substrate with the altered cytidine deaminase APOBEC3A Y130A/Y132H, and the underlined thymidine nucleotides are the result of selective deamination of 5mC. “Non-selective 5mC->T and C->U deamination” refers to the result of incubating the ssDNA substrate with a wild type APOBEC3A, and the uracil and underlined thymidine nucleotides are the result of non-selective deamination by wild type APOBEC3A.

FIG. 33 demonstrates qPCR assay using purified APOBEC proteins show decrease in Cq value after treatment of methylated ssDNA substrate with Y130A_Y132H. NEB APOBEC, APOBEC protein from New England Biolabs; Y130A, Y130A/Y132H, Y130A/E72A, and Y130A/Y132/E72A are the substitution mutations present in four APOBEC3A proteins. The E72A mutation abrogates APOBEC activity and is used as a negative control to show observed Cq value differences are due to the mutant APOBEC enzymes.

FIG. 34 shows CRISPR-Cas12 mediated detection of 5mC. Altered cytidine deaminase-mediated conversion of 5mC to T restores full complementarity of the Cas12 guide RNA for its DNA target, allowing the Cas12-guide RNA protein complex to engage the converted substrate. This activates the collateral cleavage activity of Cas12, resulting in cleavage of the reporter ssDNA containing a fluorophore and quencher. Liberation of the fluorophore leads to increased fluorescence, which is measured in a standard fluorimeter. F, fluorophore; q, quencher.

FIG. 35A-C shows incubation of substrate DNA with APOBEC3A(Y130A/Y132H) results in high 5mC deamination and low but detectable C deamination. The ssDNA oligo substrate detailed in FIG. 32 was treated with the enzyme and subsequently analyzed by Illumina sequencing. The % methylation at each C or 5mC site in the oligo is calculated as the % conversion to T. In this experiment, varying concentrations of APOBEC3A(Y130A/Y132H) were tested at different reaction temperatures and times. An elevated level of C deamination was observed at 25 C, with increased enzyme concentration and reaction time. 1 h, 3 h, and 6 h refer to the hours of incubation time; 0.75 uM, 1.5 uM, and 4 uM refer to the micromolar amounts of the enzyme used in each reaction; and 25 C, 30 C, and 37 C refer to temperature of the reaction. In each histogram the 17 bars on the left are C deamination, and the 16 bars on the right are 5mC deamination. The nucleotide triplets on the X-axis of the histograms for each micromolar amount of enzyme are the following: ACT, ACT, CCT, TCT, ACA, GCA, CCA, GCT, GCC, ACG, ACG, ACT, TCG, CCT, TCG, GCA, ACG, TCA, TCC, ACG, ACA, ACG, GCC, ACG, TCG, CCA, GCA, GCG, TCG, GCC, ACA, GCG, TCA.

FIG. 36 shows detection of 5mC in fixed cell or tissue preparations using FISH. Fixed biological samples are permeabilized and denatured to render DNA accessible to an altered cytidine deaminase. Enzymatic deamination selectively converts 5mC to T. Methylation events are detected using fluorescent probes that are specific to the converted DNA sequence.

FIG. 37A to 37O show amino acid sequences of SEQ ID NOs: 16, 17, 37-67, 92-101, and 105-108.

FIG. 38 shows a schematic representation of one embodiment consistent with the present disclosure. dsDNA is being unwound by a helicase fused to a cytidine deaminase, which is acting on 5mC. After treatment with the helicase fused to a cytidine deaminase, the 5mC has been converted into thymidine. This results in a T-G mismatch.

Schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION OF EXEMPLARY EMBODIMENTS

Described herein is a one-step, enzymatic method for mapping modified cytosines, such as 5mC and 5hmC, at single base resolution using a cytidine deaminase in combination with a helicase. The working examples provided herein describe cytidine deaminases, including altered cytidine deaminases, based on APOBEC3A. It is expected that other APOBEC proteins, either as unmodified, wild-type proteins or modified as described herein, can be used. As used herein, the term “cytidine deaminase” refers to either a wild-type cytidine deaminase or a cytidine deaminase that includes any mutations described herein. The prophetic examples provided herein describe helicases used in combination with altered APOBEC proteins and protein complexes, such as fusion proteins, including helicases and altered APOBEC proteins. As used herein, the term “protein complex” refers to a grouping of more than one protein. The proteins of a protein complex may be attached, or they may be unattached. A protein complex may include a fusion protein consisting of more than one protein fused together using a peptide linker. A protein complex may include a protein covalently attached to another protein using a linkage other than a peptide linker. A protein complex may include two or more proteins that are not attached, but are combined for a common use.

Described herein is a one-step, enzymatic method for mapping modified cytosines, such as 5mC and 5hmC, at single base resolution using an altered cytidine deaminase. The working examples provided herein describe altered cytidine deaminases based on APOBEC3A, and it is expected that other APOBEC proteins, modified as described herein, can be used.

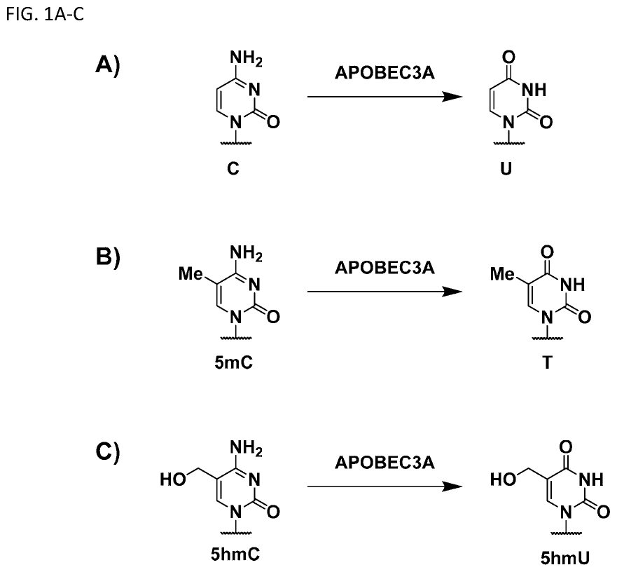

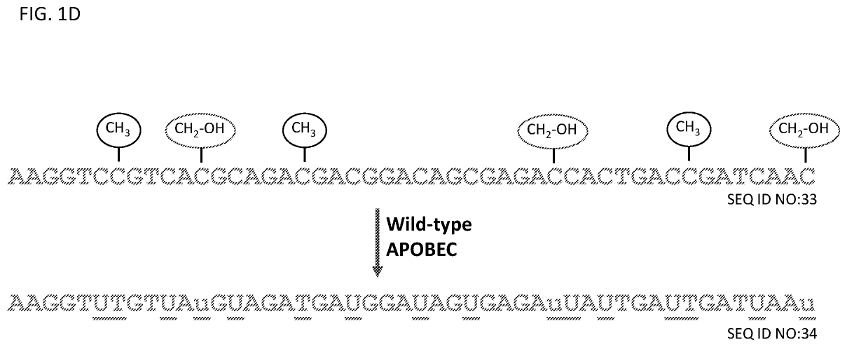

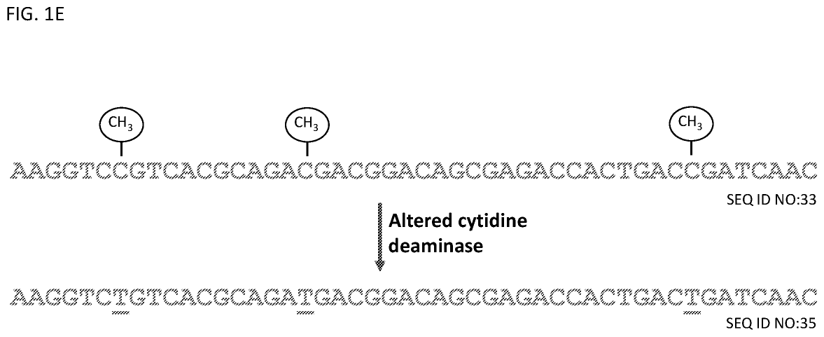

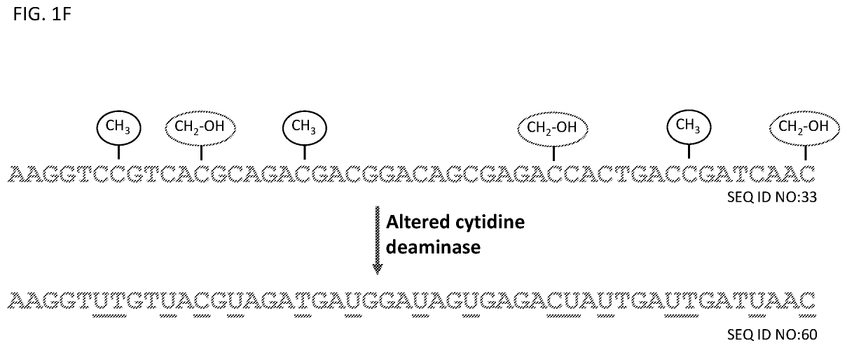

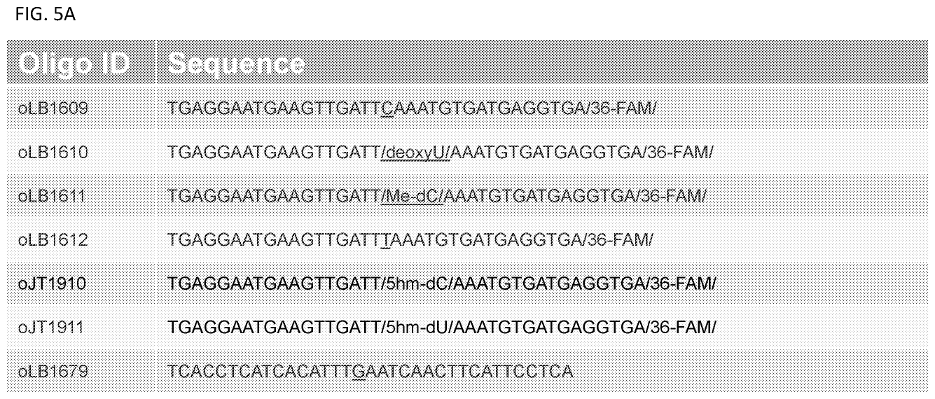

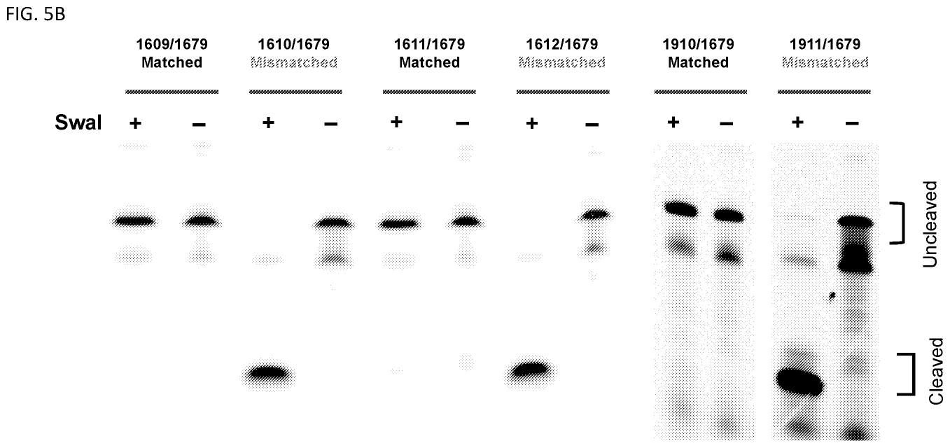

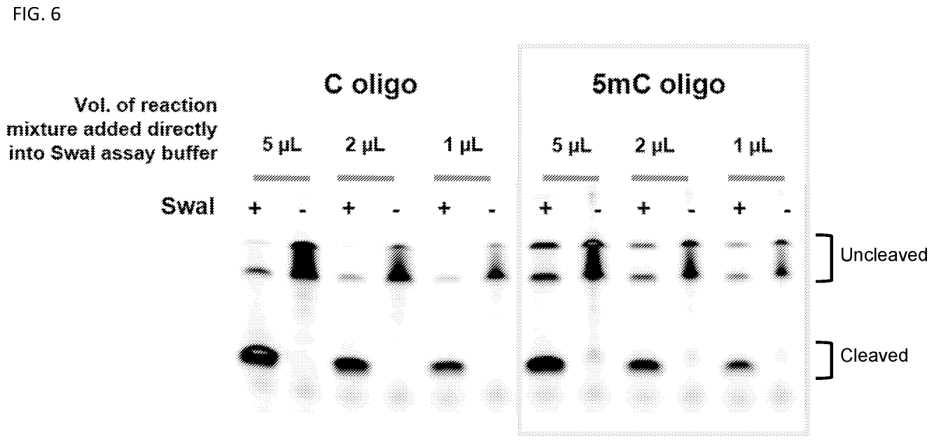

Wild type APOBEC3A deaminates cytosine (C), 5-methylcytosine (5mC), and 4-hydroxymethylcytosine (5hmC) efficiently in single-stranded DNA (FIG. 1A-C). Treatment of DNA, such as genomic DNA, with wild type APOBEC3A results in the conversion of C to uracil (U), 5mC to thymidine (T), and 5hmC to 5-hydroxymethyl uracil. These conversions reduce the complexity of the DNA to three bases for sequencing (FIG. 1D). Point mutations in human APOBEC3A proteins were produced in previous analyses and the ability of the mutant APOBEC3A proteins to convert cytosine to uracil was determined. Modification of the tyrosine residue at position 130 to alanine (Y130A) consistently resulted in an APOBEC protein with no activity (see FIG. 6c of Bulliard et al., 2011, J Virol., 85(4):1765-1776, and FIG. 5a of Shi et al., 2017, Nat Struct Mol Biol., 24(2):131-139). Proceeding contrary to Bulliard and Shi, the inventors made the surprising and unexpected discovery that certain mutations at position 130 of APOBEC3A alter the enzyme's rate of deamination on 5mC compared to C substrates.

As described herein, the cognate tyrosine (Y) at position 130 was individually mutated to all possible canonical amino acid substitutions, including A, C, D, E, F, G, H, I, K, L, M, N, P, Q, R, S, T, V, and W, and evaluated for activity on C, 5mC, and 5hmC substrates. For example, an APOBEC3A mutant containing a tyrosine to alanine point mutation in position 130 (Y130A) was found to preferentially deaminate 5mC instead of C (5mC was converted to T at a greater rate than C was converted to U), and an APOBEC3A mutant containing a tyrosine to leucine point mutation in position 130 (Y130L) was found to preferentially deaminate C instead of 5mC (C was converted to U at a greater rate than 5mC was converted to T). The deamination of 5mC to T leads to C to T mutations which can be identified by standard sequencing methods. As a result, in one embodiment, the treatment of DNA with a protein complex of the present disclosure preferentially converts 5mC to thymidine (FIG. 1E). Analysis of the sample DNA after treatment with the altered cytidine deaminase described herein, for example, by sequencing of the sample DNA, and optional comparison to a reference (e.g., reference sequence) permits easy identification of C to T point mutations, and these point mutations are inferred as 5mC positions. Wild-type APOBEC proteins and other cytidine deaminases typically act on single-stranded (ss) DNA. For some workflows, treatment of double-stranded (ds) DNA is preferred. Thus, to modify cytidine deaminases to act on dsDNA, the inventors explore coupling a cytidine deaminase to a protein capable of separating the two strands of a double-stranded nucleic acid, such as dsDNA. Helicases are a class of such proteins. The inventors explore different methods of attaching or complexing the two enzymes and investigate many different helicases to determine which are compatible with recombinant expression. The protein complexes are additionally assayed for compatibility with existing sequencing workflows.

Sequencing of the sample DNA after treatment with a protein complex described herein and optional comparison to a reference sequence permits easy identification of C to T point mutations, and these point mutations are inferred to be 5mC positions. The protein complexes described herein enable one-step modification of dsDNA samples for facile identification of 5mC.

In another example, an APOBEC3A mutant containing a tyrosine to tryptophan point mutation in position 130 (Y130W) maintained the ability to deaminate C and 5mC to U and T, respectively, but lost the ability to deaminate 5hmC, 5fC, and 5caC. When the sequence of the nucleic acid exposed to this APOBEC3A mutant is determined, C and 5mC are deaminated to U and T, respectively, and are read out as T by a sequencer. On the other hand, 5hmC is not deaminated by the APOBEC3A mutant and is read out as a C. (FIG. 1F). 5fC and 5caC are also not deaminated by this APOBEC3A mutant, and therefore cannot be distinguished from 5hmC. However, the abundance of 5fC and 5caC is orders of magnitude lower than 5hmC in human genomic DNA, approaching the detection limit of mass spectrometers used for such measurements (Ito et al., 2011, Science 333, 1300-1303 (2011); Wagner et al., 2015, Angew. Chem. Int. Edn Engl. 54, 12511-12514 (2015); Bachman et al., 2015, Nat. Chem. Biol. 11, 555-557 (2015)). Therefore, signals from 5fC and 5caC should be insignificant compared to 5hmC.

### Cytidine Deaminases

Provided herein are protein complexes including cytidine deaminases, fusion proteins including cytidine deaminases, compositions including a cytidine deaminase, methods of using a cytidine deaminase, and kits that include an cytidine deaminase. Each of these protein complexes may be compatible with wild-type cytidine deaminases or with altered cytidine deaminases. Cytidine deaminases include apolipoprotein B mRNA editing enzymes, catalytic polypeptide-like (APOBEC) and activation induced cytidine deaminase (AID). Wild-type APOBEC and AID cytidine deaminases have the activity of deaminating cytidine (C) of DNA and/or RNA to form uridine (U). One type of altered cytidine deaminase preferentially deaminates 5mC instead of C (i.e., converts 5mC to T at a greater rate than converting C to U) and is referred to herein as having “cytosine-defective deaminase activity” or “5mC-enhanced” or “5mC-selecting” deaminase activity. A second type of altered cytidine deaminase preferentially deaminates C instead of 5mC (i.e., converts C to U at a greater rate than converting 5mC to T) and is referred to herein as having “5mC-defective deaminase activity.” A third type of altered cytidine deaminase preferentially deaminates C and 5mC to U and T, respectively, and has significantly reduced deamination of 5hmC, 5fC, and 5caC. The third type is referred to herein as having “5hmC-defective deaminase activity.” Unless the context indicates otherwise, reference to an altered cytidine deaminase includes altered cytidine deaminases having cytosine-defective deaminase activity, altered cytidine deaminases having 5mC-defective deaminase activity, and altered cytidine deaminases having 5hmC-defective deaminase activity. Unless the context indicates otherwise, reference to a “cytidine deaminase” includes both wild-type cytidine deaminases, altered cytidine deaminases, and wild-type cytidine deaminases and altered cytidine deaminases structurally similar to a reference cytidine deaminase. Structural similarity is described herein.

An “altered cytidine deaminase,” “mutant cytidine deaminase,” “modified cytidine deaminase,” or “recombinant cytidine deaminase” of the present disclosure is one that includes one or more of the substitution mutations described herein. Typically, the one or more substitution mutations provide the unexpected property of an altered deamination profile, e.g., alters its ability to preferentially deaminate one form of cytosine over another.

Whether a protein has cytidine deaminase activity may be determined by in vitro assays. On example of an in vitro assay is based on digestion with the restriction enzyme SwaI (see Example 1). A protein that can deaminate 5mC to thymidine has cytidine deaminase activity.

An altered cytidine deaminase that preferentially deaminates 5mC instead of C (i.e., has cytosine-defective deaminase activity) can have a catalytic efficiency that is at least 10-fold, at least 50-fold, or at least 100-fold higher on 5mC than C substrates. In one embodiment, an altered cytidine deaminase that preferentially deaminates 5mC instead of C can have a catalytic efficiency that is no greater than 1500-fold higher on 5mC than C substrates. An altered cytidine deaminase that preferentially deaminates C instead of 5mC (i.e., has 5mC-defective deaminase activity) can have a catalytic efficiency that is at least 10-fold, at least 50-fold, or at least 100-fold higher on C than 5mC substrates. In one embodiment, an altered cytidine deaminase that preferentially deaminates C instead of 5mC can have a catalytic efficiency that is no greater than 1500-fold higher on C than 5mC substrates.

When compared to a wild type cytidine deaminase, an altered cytidine deaminase that deaminates C and 5mC to U and T, respectively, and has significantly reduced deamination of 5hmC (i.e., has 5hmC-defective deaminase activity), the deamination of 5hmC by an altered cytidine deaminase disclosed herein is reduced by at least 80%, at least 90%, or at least 99% compared to the wild type cytidine deaminase. In one embodiment, the deamination of 5hmC by an altered cytidine deaminase disclosed herein is undetectable using an assay such as the SwaI-based assay described herein.

In certain embodiments, an altered cytidine deaminase of the present disclosure is based on a wild-type cytidine deaminase, e.g., a member of the APOBEC protein family. An altered cytidine deaminase of the present disclosure that is “based on” a member of the APOBEC protein family means the altered cytidine deaminase is an APOBEC protein that includes one or more of the substitution mutations described herein as compared to a reference APOBEC sequence. A cytidine deaminase of the present disclosure that is “based on” a member of the APOBEC protein family can also include conserved and/or non-conserved mutations as described herein.

The APOBEC protein family includes, but is not limited to, subfamilies AID, APOBEC1, APOBEC2, APOBEC3 (including 3A, 3B, 3C, 3D, 3F, 3G, 3H), and APOBEC4. A wild-type cytidine deaminase can be a member of the APOBEC family of proteins or have structural similarity to a member of the APOBEC family of proteins. An altered cytidine deaminase of the present disclosure can be based on a member of the AID subfamily, the APOBEC1 subfamily, the APOBEC2 subfamily, the APOBEC3 subfamily (e.g., the 3A subfamily, the 3B subfamily, the 3C subfamily, the 3D subfamily, the 3F subfamily, the 3G subfamily, or the 3H subfamily), or the APOBEC4 subfamily. A wild-type cytidine deaminase can be a member of the APOBEC protein family from a vertebrate, such as a mammal. Examples of mammals include, but are not limited to, rodents, primates, rabbit, bovine (e.g., cow), porcine (e.g., pig), and equine (e.g., horse). An example of a primate is a human, or a chimpanzee.

The APOBEC protein family is a member of the large cytidine deaminase superfamily that contains a canonical zinc-dependent deaminase (ZDD) signature motif embedded within a core cytidine deaminase fold. This fold includes a five-stranded mixed beta (b)-sheet surrounded by six alpha (a)-helices with the order a1-b1-b2-a2-b3-a3-b4-a4-b5-a5-a6 (Salter et al., Trends Biochem Sci. 2016 41(7):578-594. doi:10.1016/j.tibs.2016.05.001; Salter et al., Trends Biochem. Sci. 2018, 43(8):606-622 doi.org/10.1016/j.tibs.2018.04.013). Each cytidine deaminase domain core structure of APOBEC proteins contains a highly conserved spatial arrangement of the catalytic center residues of a zinc-binding motif H-[P/A/V]-E-X_{[23-28]}-P-C-X_{[2-4]}-C(SEQ ID NO: 12) (referred to herein as the ZDD motif, where X is any amino acid, and the subscript range of numbers after X refers to the number of amino acids) (Salter et al., Trends Biochem Sci. 2016 41(7):578-594. doi:10.1016/j.tibs.2016.05.001). Without intending to be limited by theory, the H and two C residues coordinate a Zn atom, and the E residue polarizes a water molecule near the Zn-atom for catalysis (Chen et al., 2021, Viruses, 13:497, doi.org/10.3390/v13030497).

Some members of the APOBEC protein family, e.g., the AID subfamily, the APOBEC1 subfamily, the APOBEC2 subfamily, the APOBEC3A subfamily, the APOBEC3C subfamily, the APOBEC3H subfamily, and the APOBEC4 subfamily, include one copy of the ZDD motif. Other members of the APOBEC protein family, e.g., the APOBEC3B subfamily, the APOBEC3D subfamily, the APOBEC3F subfamily, and the APOBEC3G subfamily, include two copies of the ZDD motif, but often only the C-terminal copy is active (Salter et al., Trends Biochem Sci. 2016 41(7):578-594. doi:10.1016/j.tibs.2016.05.001). Thus, a cytidine deaminase disclosed herein includes one or two ZDD motifs. In one embodiment, a wild-type cytidine deaminase that is a member of the APOBEC3A subfamily or an altered cytidine deaminase based on a member of the APOBEC3A subfamily includes the following ZDD motif: HXEX₂₄SW(S/T)PCX_{[2-4]}CX₆FX₈LX₅R(L/I)YX_{[8-11]}LX₂LX_{[10]}M (SEQ ID NO: 13) (where X is any amino acid, and the subscript number or range of numbers after X refers to the number of amino acids) (Salter et al., Trends Biochem Sci. 2016 41(7):578-594. doi:10.1016/j.tibs.2016.05.001).

In one embodiment, a wild-type cytidine deaminase that is a member of the following subfamilies or an altered cytidine deaminase based on a member of the following subfamilies APOBEC3A, APOBEC3B, APOBEC3C, APOBEC3D, APOBEC3F, and APOBEC3G, can include one or more highly conserved sites that are part of the active site and within the ZDD motif SEQ ID NO:12. The sites include tryptophan at position 98 and serine or threonine at position 99 (Kouno et al., 2017, Nat. Comm, 8:15024, DOI: 10.1038/ncomms15024).

In addition to the ZDD motif, a member of the APOBEC protein family also includes other highly conserved residues that are part of the active site but not present as part of the ZDD motif SEQ ID NO: 12. A member of the APOBEC3A subfamily, APOBEC3B subfamily, APOBEC3C subfamily, APOBEC3D subfamily, APOBEC3F subfamily, and APOBEC3G subfamily typically includes one or more of the following highly conserved sites that are part of the active site: arginine at position 28; histidine, asparagine, or arginine at position 29; serine or threonine, preferably threonine, at position 31; asparagine or aspartic acid at position 57; tyrosine or phenylalanine at position 130; asparagine or tyrosine at position 131; asparagine, tyrosine, or phenylalanine, preferably tyrosine, at position 132; and arginine or lysine at position 189 (Kouno et al., 2017, Nat. Comm, 8:15024, DOI: 10.1038/ncomms15024).

An altered cytidine deaminase of the present disclosure includes a substitution mutation at one or more residues when compared to a wild-type cytidine deaminase. A substitution mutation can be at the same position, or a functionally equivalent position compared to the wild-type cytidine deaminase. Wild-type cytidine deaminases and functionally equivalent positions are described in detail herein. The skilled person will readily appreciate that an altered cytidine deaminase described herein is not naturally occurring.

A wild-type cytidine deaminase can be a member of the APOBEC protein family. Essentially any known member of the APOBEC protein family can be a wild-type cytidine deaminase. The skilled person can easily identify members of each of the subfamilies by using a publicly available database such as the Protein database available at the National Center for Biotechnology Information (ncbi.nlm.nih.gov/protein) and searching for APOBEC1, APOBEC2, APOBEC3A, APOBEC3B, APOBEC3C, APOBEC3D, APOBEC3F, APOBEC3G, APOBEC3H, APOBEC4, or, when identifying members of the AID (Activation-induced cytidine deaminase) family. A wild-type cytidine deaminase has the activity of binding single-stranded DNA (ssDNA) and deaminating a cytosine present on the ssDNA to convert it to uracil. In one embodiment, a wild-type cytidine deaminase has the activity of binding single-stranded RNA (ssRNA) and deaminating a cytosine present on the ssRNA to convert it to uracil. Methods for determining whether a protein binds ssDNA or ssRNA and deaminates a cytosine present are known to the skilled person.

In one embodiment, a cytidine deaminase has an amino acid sequence that is based on a reference sequence which is a member of the APOBEC protein family, such as a wild-type APOBEC protein. The altered cytidine deaminase may includes a ZDD motif H-[P/A/V]-E-X_{[23-28]}-P-C-X_{[2-4]}-C(SEQ ID NO: 12), and at least one substitution mutation disclosed herein. Optionally, an altered cytidine deaminase includes other active site residues disclosed herein. Non-limiting examples of reference cytidine deaminase proteins are shown in the following table.

TABLE 1
Examples of members of the APOBEC protein subfamilies.

| APOBEC | |
|---|---|
| protein family | Non-limiting examples |
| AID | UniProt: Q9GZX7 (SEQ ID NO: 1); UniProt: G3QLD2 |
| | (SEQ ID NO: 37); UniProt Q9WVE0 (SEQ ID NO: 38) |
| APOBEC1 | UniProt: P41238 (SEQ ID NO: 2); NCBI XP_030856728.1 |
| | (SEQ ID NO: 39); UniProt P51908 (SEQ ID NO: 40) |
| APOBEC2 | UniProt: Q9Y235 (SEQ ID NO: 4); UniProt G3SGN8 |
| | (SEQ ID NO: 41); UniProt Q9WV35 (SEQ ID NO: 42) |
| APOBEC3A | UniProt: P31941(SEQ ID NO: 3); |
| | GenBank: XP_045219544.1 (SEQ ID NO: 19) |
| | GenBank: AER45717.1 (SEQ ID NO: 20); GenBank: |
| | XP_003264816.1 (SEQ ID NO: 21); GenBank: PNI48846.1 |
| | (SEQ ID NO: 22); GenBank: ADO85886.1 (SEQ ID NO: 23) |
| APOBEC3B | UniProt: Q9UH17 (SEQ ID NO: 5); UniProt G3QV16 |
| | (SEQ ID NO: 43); UniProt F6M3K5 (SEQ ID NO: 44) |
| APOBEC3C | UniProt: Q9NRW3 (SEQ ID NO: 6); UniProt Q694B5 |
| | (SEQ ID NO: 45); UniProt B0LW74 (SEQ ID NO: 46) |
| APOBEC3D | UniProt: Q96AK3 (SEQ ID NO: 7); NCBI NP_001332895.1 |
| | (SEQ ID NO: 47); NCBI NP_001332931.1 (SEQ ID NO: 48) |
| APOBEC3F | UniProt: Q8IUX4 (SEQ ID NO: 8); UniProt G3RD21 |
| | (SEQ ID NO: 49); UniProt Q1G0Z6 (SEQ ID NO: 50) |
| APOBEC3G | UniProt: Q9HC16 (SEQ ID NO: 9); UniProt Q694C1 |
| | (SEQ ID NO: 51); UniProt U5NDB3 (SEQ ID NO: 52) |
| APOBEC3H | UniProt: Q6NTF7 (SEQ ID NO: 10); UniProt B7T0U7 |
| | (SEQ ID NO: 53); UniProt Q19Q52 (SEQ ID NO: 54) |
| APOBEC4 | UniProt: Q8WW27(SEQ ID NO: 11); NCBI XP_004028087.1 |
| | (SEQ ID NO: 55); UniProt Q497M3 (SEQ ID NO: 56) |

UniProt, database of protein sequence and functional information, available at UniProt.org; GenBank, collection of nucleotide sequences and their protein translations, available at ncbi.nlm.nih.gov/protein/.

In one embodiment, a cytidine deaminase has an amino acid sequence that is based on a reference sequence which is a member of the APOBEC3A subfamily and includes a ZDD motif HXEX₂₄SW(S/T)PCX_{[2-4]}CX₆FX₈LX₅R(L/I)YX_{[8-11]}LX₂LX_{[10]}M (SEQ ID NO: 13) (where X is any amino acid, and the subscript number or range of numbers after X refers to the number of amino acids) and at least one substitution mutation disclosed herein. In one embodiment, the substitution mutation is a substitution mutation at the underlined tyrosine, such as a substitution mutation to alanine (A). Optionally, the altered cytidine deaminase includes other active site residues disclosed herein. In one embodiment, the substitution mutation is a substitution mutation at the underlined tyrosine (Y), such as a substitution mutation to alanine (A) or to tryptophan (W).

In one embodiment, the amino acid sequence of a cytidine deaminase includes the amino acids of a member of the APOBEC3A subfamily: X_{[16-26]}-GRXXTXLCYXV-X₁₅-GXXXN-X₁₂-HAEXXF-X₁₄-YXXTWXXSWSPC-X_{[2-4]}-CA-X₅-FL-X₇-LXIXXXR(L/I)Y-X₈-GLXXLXXXG-X₅-M-X₄-FXXCWXXFV-X₆-FXPW-X₁₃-LXXI-X_{[2-6]} (SEQ ID NO:14), (where X is any amino acid, and the subscript number or range of numbers after X refers to the number of amino acids present) and an altered cytidine deaminase further includes at least one substitution mutation disclosed herein. In one embodiment, the substitution mutation is a substitution mutation at the underlined tyrosine (Y), such as a substitution mutation to alanine (A) or to tryptophan (W).

In one embodiment, the amino acid sequence of a cytidine deaminase includes a subset of the amino acids of a member of the APOBEC3A subfamily: X₂₆-GRXXTXLCYXV-X₁₅-G-X₁₆-HAEXXF-X₁₄-YXXTWXXSWSPC-X₄-CA-X₅-FL-X₇-LXIFXXR(L/I)Y-X₈-GLXXLXXXG-X₅-M-X₄-FXXCWXXFV-X₆-FXPW-X₁₃-LXXI-X₆ (SEQ ID NO:15), (where X is any amino acid, and the subscript number or range of numbers after X refers to the number of amino acids), or a subset thereof, and at least one substitution mutation disclosed herein. In one embodiment, the substitution mutation is a substitution mutation at the underlined tyrosine (Y), such as a substitution mutation to alanine (A) or to tryptophan (W).

A substitution mutation can be at the same position or a functionally equivalent position compared to a reference cytidine deaminase. By “functionally equivalent” it is meant that the altered cytidine deaminase has the amino acid substitution at the amino acid position in a reference cytidine deaminase that has the same functional role in both the reference cytidine deaminase and the altered cytidine deaminase.

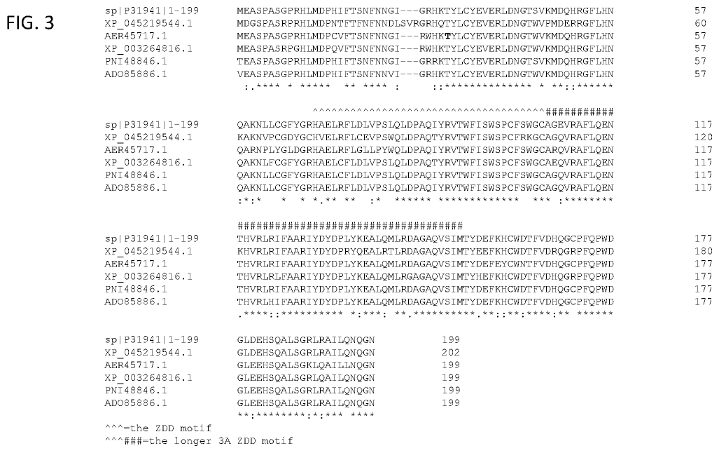

In general, functionally equivalent substitution mutations in two or more different cytidine deaminases occur at homologous amino acid positions in the amino acid sequences of the cytidine deaminases. Hence, use herein of the term “functionally equivalent” also encompasses mutations that are “positionally equivalent” or “homologous” to a given mutation, regardless of whether or not the particular function of the mutated amino acid is known. It is possible to identify the locations of functionally equivalent and positionally equivalent amino acid residues in the amino acid sequences of two or more different cytidine deaminases on the basis of sequence alignment and/or molecular modelling. An example of a sequence alignment to identify positionally equivalent and/or functionally equivalent residues is set forth in FIG. 3. For example, the residues in the members of the APOBEC3A subfamily in FIG. 3 that are vertically aligned are considered positionally equivalent as well as functionally equivalent to the corresponding residue in the human APOBEC3A amino acid sequence. Thus, for example, as shown in FIG. 3, the tyrosine at residue 130 of the APOBEC3A proteins of Homo sapiens, Pongo pygmaeus, Nomascus leucogenys, Pan troglodytes, and Gorilla gorilla and the tyrosine at residue 133 of the APOBEC3A protein from Macaca fascicularis are functionally equivalent and positionally equivalent. The skilled person can easily identify functionally equivalent residues in cytidine deaminases.

In one embodiment, a cytidine deaminase, such as an altered cytidine deaminase, has an amino acid sequence that is structurally similar to a wild-type cytidine deaminase disclosed herein. In one embodiment, a wild-type cytidine deaminase is one that includes the amino acid sequence of a sequence listed in Table 1, SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:14, or SEQ ID NO: 15.

As used herein, an altered cytidine deaminase, such as an altered cytidine deaminase, may be “structurally similar” to a wild-type cytidine deaminase if the amino acid sequence of the altered cytidine deaminase possesses a specified amount of sequence similarity and/or sequence identity compared to the wild-type cytidine deaminase.

Structural similarity of two amino acid sequences can be determined by aligning the residues of the two sequences (for example, a candidate cytidine deaminase and a reference, or wild-type, cytidine deaminase described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate cytidine deaminase is the cytidine deaminase being compared to the reference cytidine deaminase. A candidate cytidine deaminase that has structural similarity with a reference cytidine deaminase and cytidine deaminase activity is an altered cytidine deaminase.

Unless modified as otherwise described herein, a pair-wise comparison analysis of amino acid sequences can be conducted, for instance, by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Current Protocols in Molecular Biology, Ausubel et al., eds., Current Protocols, a joint venture between Greene Publishing Associates, Inc. and John Wiley & Sons, Inc., supplemented through 2004). One example of an algorithm that is suitable for determining structural similarity is the BLAST® algorithm, which is described in Altschul et al., J. Mol. Biol. 215:403-410 (1990). The BLAST® algorithm can be used to calculate percent sequence identity and percent sequence similarity between two sequences. Software for performing BLAST® analyses is publicly available through the National Center for Biotechnology Information.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. Thus, in one embodiment the amino acid sequence of a cytidine deaminase protein having sequence similarity to a reference sequence may include conservative substitutions of amino acids present in that reference sequence.

A conservative substitution for an amino acid in a protein may be selected from other members of the class to which the amino acid belongs. For example, it is well-known in the art of protein biochemistry that an amino acid belonging to a grouping of amino acids having a particular size or characteristic (such as charge, hydrophobicity, or hydrophilicity) can be substituted for another amino acid without altering the activity of a protein, particularly in regions of the protein that are not directly associated with biological activity. For example, amino acids having a non-polar side chain include alanine, glycine, isoleucine, leucine, methionine, phenylalanine, proline, tryptophan, and valine; amino acids having a hydrophobic side chain include glycine, alanine, valine, leucine, isoleucine, proline, phenylalanine, methionine, and tryptophan; amino acids having a polar side chain include arginine, asparagine, aspartic acid, glutamine, glutamic acid, histidine, lysine, serine, cysteine, tyrosine, and threonine; and amino acids having an uncharged side chain include glycine, serine, cysteine, asparagine, glutamine, tyrosine, and threonine.

Thus, as used herein, reference to a cytidine deaminase as described herein, such as reference to the amino acid sequence of one or more SEQ ID NOs described herein can include a protein with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference cytidine deaminase. Examples of altered cytidine deaminases having similarity with a reference amino acid sequence includes those having, for instance, at least 80%, at least 85%, at least 90%, or at least 95% similarity with SEQ ID NO:16 and having an alanine at amino acid 130. Other examples of altered cytidine deaminases having similarity with a reference amino acid sequence includes those having, for instance, at least 80%, at least 85%, at least 90%, or at least 95% similarity or identity with SEQ ID NO:17 and having an alanine at amino acid 130 and a histidine at amino acid 132.

Alternatively, as used herein, reference to a cytidine deaminase as described herein, such as reference to the amino acid sequence of one or more SEQ ID NOs described herein can include a protein with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference cytidine deaminase. Examples of altered cytidine deaminases having identity with a reference amino acid sequence includes those having, for instance, at least 80%, at least 85%, at least 90%, or at least 95% similarity with SEQ ID NO:16 and having an alanine (A) at amino acid 130. Other examples of altered cytidine deaminases having identity with a reference amino acid sequence includes those having, for instance, at least 80%, at least 85%, at least 90%, or at least 95% similarity or identity with SEQ ID NO:17 and having an alanine (A) at amino acid 130 and a histidine (H) at amino acid 132.

### Substitution Mutations

An altered cytidine deaminase of the present disclosure includes a substitution mutation at a position functionally equivalent to tyrosine at position 130 (Y130) in a member of the APOBEC3A subfamily (for instance, SEQ ID NO:3). Accordingly, an alignment can be produced using a member of the APOBEC3A subfamily (for instance, SEQ ID NO:3) and another candidate cytidine deaminase from the APOBEC3A subfamily or a different APOBEC subfamily. In one embodiment, the candidate is selected from APOPEC subfamilies APOBEC1 or AID. An example of an algorithm that can be used to produce an alignment is Clustal O. In some APOBEC family proteins, the wild-type residue at a position functionally equivalent to Y130 is phenylalanine (F).

In another embodiment, an altered cytidine deaminase of the present disclosure includes a substitution mutation at a position functionally equivalent to the tyrosine (Y) of ZDD motif HXEX₂₄SW(S/T)PCX_{[2-4]}CX₆FX₈LX₅R(L/I)YX_{[8-11]}LX₂LX_{[10]}M (SEQ ID NO:13) in a member of the APOBEC family, such as a member of the APOBEC3A subfamily. The underlined tyrosine (Y) of SEQ ID NO: 13 is the position functionally equivalent to the tyrosine amino acid 130 of the APOBEC3A protein SEQ ID NO: 3.

In one embodiment, the substitution mutation at a position functionally equivalent to Y130 increases cytidine deaminase activity and preferentially acts on 5mC compared to cytosine (i.e., has cytosine-defective deaminase activity). The substitution mutation can be a mutation to alanine (A), glycine phenylalanine, histidine, glutamine, methionine, asparagine, lysine, valine, aspartic acid, glutamic acid, serine, cysteine, proline, or threonine. For example, the altered cytidine deaminase can comprise SEQ ID NO:102, wherein X is selected from A, G, F, H, Q, M, N, K, V, D, E, S, C, P or T (and is not Y), or can comprise SEQ ID NO: 103, wherein Z is selected from A, G, F, H, Q, M, N, K, V, D, E, S, C, P or T (and is not Y), preferably, in one embodiment, X or Z is A or L. In an exemplary aspect of this embodiment, the substitution mutation at a position functionally equivalent to Y130 is a mutation to alanine. Specific examples of altered cytidine deaminases having increased activity and preferentially acts on 5mC compared to cytosine include SEQ ID NO: 16.

An altered cytidine deaminase of the present disclosure optionally includes a second substitution mutation at a position two, three, four, or five amino acids on the C-terminal side of the Y130 position, or functionally equivalent to the Y130 position. In one embodiment, the second mutation is a tyrosine, tryptophan, cysteine, histidine, or phenylalanine at a position two, three, four, or five amino acids on the C-terminal side of the Y130 position, or functionally equivalent to the Y130 position. In one embodiment, the second mutation is at a position functionally equivalent to tyrosine at position 132 (Y132) in a member of the APOBEC3A subfamily (for instance, SEQ ID NO:3). An APOBEC protein, such as an APOBEC3A protein, containing substitution mutations at both the first site, at a position functionally equivalent to Y130, and the second site, at a position two, three, four, or five amino acids on the C-terminal side of the Y130 position, increases the preferential activity to act on 5mC compared to the same APOBEC protein, such as an APOBEC3A protein, containing one substitution mutation at Y130. In one embodiment, the substitution mutation at the second position is an amino acid having a positively charged side chain and selected from arginine, histidine, lysine, or a polar side chain selected from glutamine. In one embodiment, the substitution mutation at the second position is histidine, such as Y132 to histidine. The double mutant containing both first and second mutations can be any substitution mutation at a position functionally equivalent to Y130 described herein and any second substitution mutation at a position two, three, four, or five amino acids on the C-terminal side of the Y130 position described herein, in any combination. For example, the altered cytidine deaminase can be SEQ ID NO: 3, 15, or 16 and have a substitution at Y130 and Y132, or the position functionally equivalent to Y130 and Y132 as described herein. One example of an altered cytidine deaminase is SEQ ID NO: 104 comprising Y130X and Y132Z, where X is selected from (A), (L), or (W) (preferably (A)), and Z is selected from (R), (H), (L), or (Q), preferably (H). This encompasses examples including, but not limited to, for example Y130A and Y132R, Y130A and Y132H, Y130A and Y132L, Y130A and Y132Q, Y130L and Y132R, Y130L and Y132H, Y130L and Y132L, Y130L and Y132Q, Y130W and Y132R, Y130W and Y132H, Y130W and Y132L, Y130W and Y130Q, or any suitable combinations therein. In one embodiment, the double mutant includes substitution mutations Y130A and Y132H. Specific examples of altered cytidine deaminases having both substitution mutations and preferentially acting on 5mC compared to the APOBEC protein having just the single substitution mutation at cytosine include SEQ ID NO:17 or a sequence having at least 90%, at least 95%, at least 98%, at least 99% sequence identity to SEQ ID NO:17 and comprising Y130A and Y132H. In another embodiment, the 5mC-selective deaminase further comprises a single substitution at a position functionally equivalent to 133 in a wild-type APOBEC3A, such as SEQ ID NO:3, (e.g., D133, preferably D133W).

The person of ordinary skill in the art can confirm the 5mC or 5hmC preferential deaminase activity of the arginine, glutamine, histidine and lysine substitution mutations at the second position in the double mutants described above. For example, double mutants constructed, to create an altered cytidine deaminase having a first substitution mutation at a position functionally equivalent to Y130 and a second arginine, glutamine, histidine or lysine substitution mutation at the tyrosine position two amino acids on the C-terminal side of the Y130 position, and then evaluated for deamination of C residues in one assay and deamination of 5mC residues in a second assay. Using an assay such as the SwaI-based assay described herein, the ratio of 5mC deamination and C deamination was compared to identify those double mutants that preferentially deaminate 5mC compared to C. One of ordinary skill in the art could similarly test double mutants having a tyrosine at a position three, four or five positions C-terminal to the position functionally equivalent to Y130 and confirm that a substitution mutation at that position to arginine, glutamine, histidine or lysine, in combination with a mutation at the position functionally equivalent to Y130 (such as Y130A), as double mutants that preferentially deaminate 5mC compared to C. Some embodiments presented herein relate to substitution mutations that result in 5mC-defective deaminase activity (i.e., converts C to U at a greater rate than converting 5mC to T). In one embodiment, the substitution mutation at a position functionally equivalent to Y130 increases cytidine deaminase activity and preferentially acts on cytosine compared to 5mC and is a mutation to an amino acid having a non-polar side chain or a hydrophobic side chain, such as leucine (L) or tryptophan (W). In an exemplary aspect of this embodiment, the substitution mutation at a position functionally equivalent to Y130 is a mutation to leucine. Other examples of mutations that result in increased preferential deamination activity on cytosine compared to 5mC include a single mutant with Y132P, and double mutants with a substitution mutation at Y130V and Y132H, or Y130W and Y132H. Specific examples of altered cytidine deaminases having increased cytidine deaminase activity and preferentially acts on cytosine compared to 5mC include SEQ ID NO:18 or a sequence having at least 90%, at least 95%, at least 98%, at least 99% sequence identity to SEQ ID NO:18 and comprising Y130L.

In one embodiment, the substitution mutation is at a position functionally equivalent to Y130 that results in 5hmC-defective deaminase activity (i.e., preferentially deaminates C and 5mC to U and T, respectively, and has significantly reduced deamination of 5hmC). In an exemplary aspect of this embodiment, the substitution mutation at a position functionally equivalent to Y130 is a mutation to an amino acid having a non-polar side chain or a hydrophobic side chain, such as tryptophan (W). Specific examples of altered cytidine deaminases having the ability to deaminate C and 5mC to U and T, respectively, but reduced ability to deaminate 5hmC, preferably no detectable ability to deaminate 5hmC include SEQ ID NO:59 or a sequence having at least 90%, at least 95%, at least 98%, at least 99% sequence identity to SEQ ID NO:59 and comprising Y130W.

An altered cytidine deaminase described herein can include additional mutations. Typically, additional mutations do not unduly alter the activity of the altered cytidine deaminase. One or more additional mutations can be a conservative mutation.

An altered cytidine deaminase described herein can also include additional protein domains. For example, an altered cytidine deaminase may include a domain for detection, such as a fluorescent protein, an affinity tag such as a His-tag, or a conjugation domain such as SpyTag for attachment to another protein.

An altered cytidine deaminase described herein can be a truncated protein. A truncated protein is a fragment of an altered cytidine deaminase of the present disclosure that retains the ability to deaminate 5mC to thymidine. A truncated altered cytidine deaminase can include a deletion of 1 to 13 amino acids on the N-terminal end of the protein, a deletion of 1 to 3 amino acids on the C-terminal end of the protein, or a combination thereof.

An altered cytidine deaminase of the present application may be an altered cytidine deaminase described in International Patent Application No. PCT/US2023/017846, entitled “ALTERED CYTIDINE DEAMINASES AND METHODS OF USE,” filed on Apr. 7, 2023, and the altered deaminases described therein are hereby incorporated by reference in their entirety. Other altered cytosine deaminases can be used in the practice of this invention, including, but not limited to, for example, the hyperactive altered forms of APOBEC, for instance, those described in U.S. Pat. No. 10,961,525 entitled “Hyperactive AID/APOBEC and HMC Dominant TET enzymes”, the content of the altered APOBECs incorporated herein by reference in their entirety (for example, but not limited to chimeric deaminases comprising a first domain of APOBEC 4B (A3B) and the catalytic domain of APOBECC3A (A3A), A3B protein having amino acid mutations at D196H, T197I, delta(206-210), Ins(206)GIG, R212H, Q213K, W228S, I230K, M235R, C239H, E241K, E342K, Y343H, Y350D, R351H, and E363D). Further, other altered APOBEC proteins are described in U.S. Pat. No. 9,896,726 entitled “Methods and compositions for discrimination between cytosine and modifications thereof, and for methylome analysis”, the altered cytosine deaminases incorporated by reference herein. The listing of wild-type and altered cytosine deaminases that can be used in the present disclosure herein is not exhaustive and it is contemplated others may be used and contemplated with this disclosure.

### Helicases

Provided herein are compositions including a helicase and a cytidine deaminase, such as an altered cytidine deaminase described herein, and methods of using a helicase in combination with a cytidine deaminase, such as an altered cytidine deaminase described herein. Wild-type helicases directionally separate double-stranded nucleic acids, commonly during replication of double-stranded DNA. During DNA replication, each separated DNA strand is amplified using a polymerase to prepare two daughter strands from a single double-stranded parent template. There are also many varieties of helicases that separate RNA and are implicated in RNA metabolism.

While some passive helicases exist, most typically require energy to break the hydrogen bonds between base pairs. This energy is usually provided by ATP hydrolysis (Johnson D S, Bai L, Smith B Y, Patel S S, Wang M D. Single-molecule studies reveal dynamics of DNA unwinding by the ring-shaped T7 helicase. Cell. 2007 Jun. 29; 129(7):1299-309. doi:10.1016/j.cell.2007.04.038. PMID: 17604719; PMCID: PMC2699903.). Helicases can move along a segment of double-stranded DNA, leaving behind a window of opened single-stranded DNA. Helicases can move in a 3′ to 5′ direction, or in a 5′ to 3′ direction. Helicase movement can lead with the N-terminus or the C-terminus of the protein. Some helicases are localized to the negative strand of a nucleic acid, while others are localized to the positive strand. Each of these properties may be considered when selecting a helicase to use for a given function or in a given context.

Helicases are regarded as vital to all life forms, and have been identified in eukaryotes, prokaryotes, archaea, and viruses. There are six superfamilies of helicases (SF1-SF6), defined by rough structure and shared sequence motifs (Singleton, Martin R et al. “Structure and mechanism of helicases and nucleic acid translocases.” Annual review of biochemistry vol. 76 (2007): 23-50. doi:10.1146/annurev.biochem.76.052305.115300). Helicases belonging to SF3-SF6 form a ring around a nucleic acid as they process along the strands. Helicases frequently form multimers, such as homo-hexamers. In other instances, helicases are active as monomers or dimers. Other numbers of subunits are possible and are considered, particularly for the modified proteins of this disclosure. While it is typically thought that, in nature, multimeric helicases exist as homo-tetramers or homodimers, formation of hetero-tetramers and heterodimers is possible.

The helicases of this disclosure may be mutated to change their function. For example, mutations may be made to a protein sequence to result in a helicase with faster or slower processive movement, depending on the desired activity of the resultant enzyme.

In embodiments where a helicase having more than one monomer is used, any suitable number of monomers may be altered, such as two, three, four, five, six, seven, or eight monomers. In embodiments, the alteration may be present on each monomer. Alternately, a modification may be present on only a subset of the monomers, resulting in a hetero-multimeric helicase. In particular, when a helicase is modified to include a large structural change, such as in attachment of a cytidine deaminase, the multimeric helicase assembly may have improved structural stability when only a portion of the monomers include the structural change. For example, it may be desirable to form a mutant DnaB helicase assembly wherein two or three of the six monomers are fused to a cytidine deaminase. In embodiments wherein a dimeric helicase is used, one or both of the monomers may be altered.

Assembly of helicase monomers is typically stochastic. Providing a predetermined ratio of modified to unmodified monomers in a given helicase multimer may influence the number of modified monomers per multimer. The ratio of modified to unmodified monomers may be controlled in any suitable method. In embodiments wherein the helicase is expressed as a nucleic acid construct, a different expression cassette may be included for each of the unaltered and altered monomers. Expression cassette components such as promoters, polyadenylation signals, and post-transcriptional elements may be selected to achieve a desired level of expression. Each expression cassette may be provided on a single construct, such as a single plasmid. Alternatively, separate constructs with each nucleic acid expression cassette can be provided in a predetermined ratio to control the level of each protein produced. The ratio of modified to unmodified monomers can alternatively or additionally be controlled using protein modifications. Suitable protein modifications include, but are not limited to, degradation tags such as ubiquitin, and localization tags such as nuclear localization signals.

In certain embodiments, a helicase is attached to a cytidine deaminase by an amino acid linker. Amino acid linkers are described herein. A nucleic acid construct for recombinant expression of a helicase as a fusion protein with a cytidine deaminase may be prepared. From such a construct, the fusion protein may be recombinantly expressed in a cell and purified from culture media or cell lysate. Standard methods of recombinant protein overexpression may be suitable for preparation of the fusion protein.

In embodiments, it may be desirable to provide to a cell a nucleic acid construct encoding a fusion protein of a cytidine deaminase and a helicase in combination with a nucleic acid construct encoding an unmodified helicase. The cell may then be incubated in conditions suitably for recombinant expression of both constructs simultaneously, and the resultant protein may be purified from cell lysate or culture media. In this manner, a hetero-multimeric helicase including a combination of modified and unmodified monomers may be obtained.

In one or more embodiments, the helicase includes an ATP-dependent helicase. In one or more embodiments, the helicase does not include an ATP-dependent helicase.

In the following amino acid motifs, the character “X” is used to represent any amino acid. The character “h” is used to represent any hydrophobic amino acid, typically either valine, leucine, isoleucine, phenylalanine, methionine, or tryptophan. The character “y” is used to represent any hydrophilic amino acid, typically either serine, threonine, histidine, aspartate, glutamine, glutamate, lysine, arginine, or asparagine.

In one or more embodiments, the amino acid sequence of a helicase includes a subset of the amino acids of a member of the helicase Superfamily 1, including Motif I:hhXGXAGyGKS (SEQ ID NO:70), Motif Ia:XXhXXyy (SEQ ID NO:71), Motif II: hhhDEXy (SEQ ID NO:72), Motif III: hhhhGDXyQ (SEQ ID NO:73), Motif IV: xxhXyyXR (SEQ ID NO:74), Motif V: XXThXXXQGhyhyyV (SEQ ID NO:75), Motif VI: VAhTRXyy (SEQ ID NO:76), or a combination thereof. (Singleton, Martin R et al. “Structure and mechanism of helicases and nucleic acid translocases.” Annual review of biochemistry vol. 76 (2007): 23-50., Hall, M C, and S W Matson. “Helicase motifs: the engine that powers DNA unwinding.” Molecular microbiology vol. 34, 5 (1999): 867-77.)

In one or more embodiments, the amino acid sequence of a helicase includes a subset of the amino acid of a member of the helicase Superfamily 2, including Motif I:hhXXXyGXGKT (SEQ ID NO:77), Motif Ia: XhhhXPyy (SEQ ID NO:78), Motif II: hhhDEXH (SEQ ID NO:79), Motif III: hXhSAThhh (SEQ ID NO:80), Motif IV: hhFXXyXy (SEQ ID NO:81), Motif V: hXXTXXXXXGhyhXyh (SEQ ID NO:82), Motif VI: QXXGRXXR (SEQ ID NO:83), or a combination thereof. (Singleton, Martin R et al. “Structure and mechanism of helicases and nucleic acid translocases.” Annual review of biochemistry vol. 76 (2007): 23-50., Hall, M C, and S W Matson. “Helicase motifs: the engine that powers DNA unwinding.” Molecular microbiology vol. 34, 5 (1999): 867-77.)

In one or more embodiments, the amino acid sequence of a helicase includes a subset of the amino acids of a member of the helicase Superfamily 3, including Motif A: hhhXGPXGTGKS (SEQ ID NO:84), Motif B: hhXhDD (SEQ ID NO:85), Motif C: hhhTTN (SEQ ID NO:86), or a combination thereof. (Singleton, Martin R et al. “Structure and mechanism of helicases and nucleic acid translocases.” Annual review of biochemistry vol. 76 (2007): 23-50., Hall, M C, and S W Matson. “Helicase motifs: the engine that powers DNA unwinding.” Molecular microbiology vol. 34, 5 (1999): 867-77.)

In one or more embodiments, the amino acid sequence of a helicase includes a subset of the amino acid of the helicase Family 4, including Motif 1: XhXhhXARXXhGKT (SEQ ID NO:87), Motif 1a: VLXhSLEM (SEQ ID NO:88), Motif 2, hIhhDYL (SEQ ID NO:89), Motif 3, IXXIXXyLKAhAXyLXPhXXhXQ (SEQ ID NO:90), Motif 4, PXXXDLRXSGXIXQXADXIh (SEQ ID NO:91), or a combination thereof. (Singleton, Martin R et al. “Structure and mechanism of helicases and nucleic acid translocases.” Annual review of biochemistry vol. 76 (2007): 23-50., Hall, M C, and S W Matson. “Helicase motifs: the engine that powers DNA unwinding.” Molecular microbiology vol. 34, 5 (1999): 867-77.)

In one or more embodiments, the amino acid sequence of a helicase includes a Walker motif, including a Walker A motif: GXXXXGKTS (SEQ ID NO:68) and/or a Walker B motif: RKXXXGXXXLhhhDE (SEQ ID NO:69).

In one or more embodiments, the helicase includes a RecQ family helicase. RecQ family helicases are a subfamily of helicase Superfamily 2 and are typically identified by a conserved RecQ motif Examples of suitable RecQ family helicases include RecQ from E. coli (UniProt:P15043, SEQ ID NO:92), BLM helicase from H. sapiens (UniProt:B7ZKN7, SEQ ID NO:57), RecQ from B. subtilis (UniProt:034748, SEQ ID NO:93), WRN helicase from H. sapiens (UniProt:W14191, SEQ ID NO:94), Sgsl helicase from S. cerevisiae (UniProt:P35187, SEQ ID NO:95), and DDM1 helicase from A. thaliana (UniProt:Q9XFH4, SEQ ID NO: 96).

In one or more embodiments, the helicase may be of bacterial or archaeal origin. In one or more embodiments, the helicase is RadA from S. pneumoniae (UniProt:Q8DRPO, SEQ ID NO:97), MCM from M. thernoautotrophicum (UniprotO27798, SEQ ID NO:98), or the MCM4,6,7 complex from S. pombe (UniProt:P29458, P49731, 075001, SEQ ID NOs:99-101).

In one or more embodiments, the helicase is a mammalian helicase. Examples of suitable mammalian helicase include BLM DNA helicase from H. sapiens (UniProt:B7ZKN7, SEQ ID NO:57), DnaB helicase from H. sapiens (UniProt:Q8NG08, SEQ ID NO:58), DnaB helicase from P. paniscus (UniProt:AOA2R8ZJK1, SEQ ID NO:59), DnaB helicase from P. abelii (UniProt:AOA2R8ZJK1, SEQ ID NO:60), DnaB helicase from P. troglodytes (UniProt:AOA2J8KTT1, SEQ ID NO:105), DNA helicase B from G. gorilla (UniProt:G3RJF6, SEQ ID NO:62), DNA helicase B from M. fascicularis (UniProt:A1A7N9CCZ5, SEQ ID NO:63), and DNA helicase B from P. tephrosceles (UniProt:AOA8C9LSO1, SEQ ID NO:64).

In some embodiments, the helicase is or has structural similarity to a reference helicase. Examples of a reference helicase include, but are not limited to, the helicase proteins of SEQ NO:57-62. As used herein, a helicase may be “structurally similar” to a reference helicase if the amino acid sequence of the helicase possesses a specified amount of sequence similarity and/or sequence identity compared to the reference helicase. Structural similarity of two amino acid sequences can be determined as described herein. A candidate helicase that has structural similarity with a reference helicase and the helicase activity described herein is a helicase that is expected to be useful in the disclosed methods. In one embodiment the amino acid sequence of a helicase protein having sequence similarity to a reference sequence may include conservative substitutions of amino acids present in that reference sequence. Conservative substitutions are described herein.

Thus, as used herein, reference to a helicase as described herein, such as reference to the amino acid sequence of one or more SEQ ID NOs of a cytidine deaminase described herein, such as SEQ ID NOs:57-62, can include a protein with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference UDG.

Alternatively, as used herein, reference to a helicase as described herein, such as reference to the amino acid sequence of one or more SEQ ID NOs described herein, such as SEQ ID NOs:57-62, can include a protein with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference UDG.

### Methods of Helicase-Cytidine Deaminase Attachment

In certain embodiments, it may be desirable for a helicase and a cytidine deaminase to be in close physical proximity during treatment of a nucleic acid. As described herein, the cytidine deaminases, including altered cytidine deaminases, of this disclosure can deaminate 5mC. The catalytic activity of these proteins may be significantly higher on single-stranded nucleic acids than double-stranded nucleic acids. In some instances, it may be desirable for the cytidine deaminases of this disclosure to demonstrate improved catalytic activity on double-stranded nucleic acids. To achieve this, a protein complex comprising a cytidine deaminase, such as an altered cytidine deaminase, and an enzyme that can separate the two strands of a double-stranded nucleic acid, such as a helicase, may be used. In some embodiments, the protein complex comprises a cytidine deaminase attached to the enzyme that can separate the two strands of a double-stranded nucleic acid, such as a helicase. When the cytidine deaminase is spatially near an enzyme such as a helicase, the cytidine deaminase may act on the strands separated by the helicase. In some embodiments, a region of separated single strands may re-form a double-stranded structure after a protein complex such as a helicase-cytidine deaminase fusion has moved away.

In some embodiments, a cytidine deaminase may be attached to a helicase to form a helicase-cytidine deaminase complex. In some of these embodiments, treatment of a dsDNA substrate with a helicase-cytidine deaminase complex may exhibit higher levels of 5mC deamination than a comparable treatment wherein the helicase and cytidine deaminase are not attached.

In some embodiments, a helicase and a cytidine deaminase may be recombinantly expressed. In some of these embodiments, the helicase and the cytidine deaminase are each expressed as separate recombinant proteins and prepared as a helicase-cytidine deaminase protein complex. In some other embodiments, the helicase and the cytidine deaminase may be expressed as a fusion protein.

In some embodiments, the helicase-cytidine deaminase complex may be a fusion protein. In some embodiments, the cytidine deaminase and the helicase may be attached N-terminus to N-terminus, N-terminus to C-terminus, or C-terminus to C-terminus. The helicase and the cytidine deaminase may be attached in any suitable order. For example, the helicase may be attached to the C-terminus of the cytidine deaminase.

In some embodiments, the helicase may be attached to a non-terminal region of the cytidine deaminase. As used herein, attachment of a protein to a “non-terminal region” of another protein describes attachment of a protein at any residue other than the N-terminal residue and the C-terminal residue. Alternately, the cytidine deaminase may be attached to a non-terminal region of the helicase. Suitable loops may be identified by their high b-factor. In some embodiments, the helicase may be attached to a loop functionally equivalent to the 60-67 loop in APOBEC3A. In some embodiments, the helicase may be attached to a loop functionally equivalent to the 40-45 loop in APOBEC3A.

In embodiments wherein the helicase-cytidine deaminase complex is a fusion protein, the fusion protein may include any additional components. For example, the fusion protein may include a purification tag, such as a His-tag, a FLAG-tag, or a Myc-tag. The fusion protein may be further modified to include a reactive handle such as a free cystine. In embodiments, the fusion protein may include a fluorescent protein or another detectable marker.

In embodiments wherein the helicase-cytidine deaminase complex is a fusion protein, they may be connected by any suitable linker. The physical proximity of the helicase to the cytidine deaminase may play a role in the efficiency of 5mC deamination. For example, when the cytidine deaminase and the helicase are far apart, the cytidine deaminase may not access the ssDNA window opened by the helicase with a high frequency, leading to a lower rate of 5mC deamination. Conversely, if the helicase and cytidine deaminase are attached with no linker or a short linker, both may not be sterically capable of acting on the same dsDNA substrate. It should be understood by one of ordinary skill in the art that selection of a linker for a bifunctional fusion protein involves considerable inventive effort. In some embodiments, it may be preferable to have a structured linker, such as an alpha helical linker. In some other embodiments, it may be preferable to have an unstructured linker, such as a glycine-serine linker. The linker may have any suitable length, for example, the linker may include at least two amino acids, at least four amino acids, at least six amino acids, at least ten amino acids, at least 12 amino acids, at least 16 amino acids, at least 20 amino acids, at least 30 amino acids, or at least 40 amino acids. The linker may be at most 100 amino acids, at most 80 amino acids, at most 60 amino acids, at most 40 amino acids, at most 20 amino acids, or at most ten amino acids.

In some embodiments, the helicase-cytidine deaminase complex is covalently attached. In some of these embodiments, each protein may be provided with a modification that enables attachment. For example, one protein may be provided attached to an avidin moiety, such as streptavidin or neutravidin, and the other protein may be provided attached to a biotin moiety.

Attachment moieties may be attached to each protein by any suitable method. The helicase and/or the cytidine deaminase may be recombinantly expressed as a fusion to a peptide attachment moiety. In some embodiments, a cytidine deaminase and a helicase may be obtained as independent proteins and further modified to include a peptide attachment moiety. For instance, each protein may be separately recombinantly expressed, or they may be co-expressed and co-purified. Each protein may be purchased. Each protein may be purified from a natural source, such as tissue lysate. A peptide attachment moiety may include any additional protein or peptide that is usually capable of forming a covalent bond with a partner. Other attachment systems may be suitable, including systems that do not form a covalent bond, but form a strong bond with a dissociation constant of less than, for example, 10 nM. Examples of suitable attachment pairs include SpyTag/SpyCatcher, SnoopTag/SnoopCatcher, biotin/avidin, and leucine zippers.

Alternately or additionally, the helicase and/or the cytidine deaminase may be recombinantly expressed or provided as a fusion to a reactive handle. A reactive handle may be further reacted to enable attachment of the helicase to the cytidine deaminase.

In embodiments wherein a reactive handle is used, the reactive handle may be attached to any suitable portion of the cytidine deaminase and/or the helicase. The reactive handle may be attached to the N-terminus, the C-terminus, or a non-terminal region of the cytidine deaminase. The reactive handle may be attached to N-terminus, the C-terminus, or a non-terminal region of the helicase.

### Additional Considerations

In nature, helicases often work in concert with additional enzymes when acting on dsDNA (Seo, Yeon-Soo, and Young-Hoon Kang. “The Human Replicative Helicase, the CMG Complex, as a Target for Anti-cancer Therapy.” Frontiers in molecular biosciences vol. 5 26. 29 Mar. 2018, doi:10.3389/fmolb.2018.00026). In some embodiments, it may be advantageous to include additional enzymes in the compositions and methods described herein to improve helicase activity. Examples of enzymes that may be used include, but are not limited to, topoisomerases. Topoisomerases that may be used include DNA topoisomerase 1 from H. sapiens (UniProt:P11387, SEQ ID NO:65) and DNA topoisomerase 2-α from H. sapiens (UniProt:P11388, SEQ ID NO:66).

In embodiments wherein an ATP-dependent helicase is used, it may be advantageous to include a high concentration of ATP. In some embodiments, the helicase may require additional cofactors for use. Any suitable cofactors may be used to improve activity of the helicase.

### Polynucleotides Encoding Cytidine Deaminases and Helicases

Cytidine deaminases, helicases, and fusion proteins described herein also may be identified in terms of the polynucleotide that encodes the protein. Thus, this disclosure provides polynucleotides that encode a cytidine deaminase, helicase, or fusion protein described herein. Alternately, this disclosure provides polynucleotides that hybridize, under standard hybridization conditions, to a polynucleotide that encodes a cytidine deaminase, helicase, or fusion protein described herein, and the complements of such polynucleotide sequences.

A polynucleotide as described herein can include any polynucleotide that encodes a cytidine deaminase, such as an altered cytidine deaminase, helicase, fusion protein of the present disclosure or a combination thereof. Thus, the nucleotide sequence of the polynucleotide may be deduced from the amino acid sequence that is to be encoded by the polynucleotide. The proteins of this disclosure can be encoded by multiple codons, and certain translation systems (e.g., prokaryotic or eukaryotic cells) often exhibit codon bias, e.g., different organisms often prefer one of the several synonymous codons that encode the same amino acid. As such, polynucleotides presented herein are optionally “codon optimized,” meaning that the polynucleotides are synthesized to include codons that are preferred by the particular translation system being employed to express the protein. For example, when it is desirable to express the protein in a bacterial cell (or even a particular strain of bacteria), the polynucleotide can be synthesized to include codons most frequently found in the genome of that bacterial cell, for efficient expression of the altered cytidine deaminase. A similar strategy can be employed when it is desirable to express the altered cytidine deaminase in a eukaryotic cell, e.g., the nucleic acid can include codons preferred by that eukaryotic cell.

A polynucleotide described herein may also, advantageously, be included in a suitable expression vector to express the altered cytidine deaminase encoded therefrom in a suitable host. Incorporation of cloned DNA into a suitable expression vector for subsequent transformation of a host cell and subsequent selection of the transformed cells is well known to those skilled in the art as provided in Sambrook et al. (1989), Molecular cloning: A Laboratory Manual, Cold Spring Harbor Laboratory. Suitable host cells include, but are not limited to, E. coli and S. cerevisiae.

Such an expression vector includes a vector having a polynucleotide described herein operably linked to heterologous regulatory sequences, such as promoter regions, that are capable of effecting expression of said DNA fragments. The term “operably linked” refers to a juxtaposition wherein the components described are in a relationship permitting them to function in their intended manner. Such vectors may be transformed into a suitable host cell to provide for the expression of an altered cytidine deaminase.

The nucleic acid molecule may encode a mature protein or a protein having a pro-sequence, including that encoding a leader sequence on the preprotein which is then cleaved by the host cell to form a mature protein. The vectors may be, for example, plasmid, virus or phage vectors provided with an origin of replication, and optionally a promoter for the expression of said nucleotide and optionally a regulator of the promoter. The vectors may contain one or more selectable markers, such as, for example, an antibiotic resistance gene.

Regulatory elements required for expression include promoter sequences to bind RNA polymerase and to direct an appropriate level of transcription initiation and also translation initiation sequences for ribosome binding. For example, a bacterial expression vector may include a promoter such as the lac promoter and for translation initiation the Shine-Dalgarno sequence and the start codon AUG. Similarly, a eukaryotic expression vector may include a heterologous or homologous promoter for RNA polymerase II, a downstream polyadenylation signal, the start codon AUG, and a termination codon for detachment of the ribosome. Such vectors may be obtained commercially or be assembled from the sequences described by methods well known in the art.

Transcription of DNA encoding an altered cytidine deaminase may be optimized by including an enhancer sequence in the vector. Enhancers are cis-acting elements of DNA that act on a promoter to increase the level of transcription. Vectors will also generally include origins of replication in addition to the selectable markers.

### Making and Isolating Cytidine Deaminases, Helicases, and Fusion Proteins

Generally, polynucleotides encoding a cytidine deaminase, helicase, or fusion protein as presented herein can be made by cloning, recombination, in vitro synthesis, in vitro amplification and/or other available methods. A variety of recombinant methods can be used for expressing an expression vector that encodes a cytidine deaminase, helicase, or fusion protein presented herein. Methods for making recombinant polynucleotides, expression, and isolation of expressed products are well known and described in the art.

Polynucleotides encoding wild type cytidine deaminases can be obtained from a source and subjected to mutagenesis to introduce the substitution mutation described herein. In general, any available mutagenesis procedure can be used for making a cytidine deaminase, particularly an altered cytidine deaminase, described herein. Procedures that can be used include, but are not limited to: site-directed point mutagenesis, in vitro or in vivo homologous recombination, oligonucleotide-directed mutagenesis, mutagenesis by total gene synthesis, and many others known to persons skilled in the art.

Polynucleotides encoding wild type helicases can be obtained from a source and subjected to mutagenesis to introduce the substitution mutations described herein. In general, any mutagenesis procedure can be used for making a helicase described herein. Procedures that can be used include, but are not limited to: site-directed point mutagenesis, in vitro or in vivo homologous recombination, oligonucleotide-directed mutagenesis, mutagenesis by total gene synthesis, and many others known to persons skilled in the art.

Polynucleotides encoding the fusions proteins described herein can be prepared using any available molecular cloning techniques. Preferably, a polynucleotide encoding a fusion of a helicase and a cytidine deaminase may be prepared using a scarless or semi-scarless gene assembly technique. Examples of suitable techniques include, but are not limited to, Golden Gate assembly, Gibson assembly, and restriction enzyme ligation. Template polynucleotides encoding a helicase and/or a cytidine deaminase may be obtained from a source as described herein.

Polynucleotides encoding additional protein components desired for preparation of a cytidine deaminase or helicase may be obtained from a source and subjected to mutagenesis or cloning to prepare the altered proteins described herein. Examples of additional protein components include linkers, affinity tags, protein conjugation tags, and additional enzymes.

Additional useful references for mutation, recombinant, and in vitro nucleic acid manipulation methods (including cloning, expression, PCR, and the like) include Berger and Kimmel, Guide to Molecular Cloning Techniques, Methods in Enzymology volume 152 Academic Press, Inc., San Diego, Calif (Berger); Kaufman et al. (2003) Handbook of Molecular and Cellular Methods in Biology and Medicine Second Edition Ceske (ed) CRC Press (Kaufman); The Nucleic Acid Protocols Handbook Ralph Rapley (ed) (2000) Cold Spring Harbor, Humana Press Inc (Rapley); Chen et al. (ed) PCR Cloning Protocols, Second Edition (Methods in Molecular Biology, volume 192) Humana Press; and in Viljoen et al. (2005) Molecular Diagnostic PCR Handbook Springer, ISBN 1402034032.

In addition, many kits are commercially available for the purification of plasmids or other relevant nucleic acids from cells. An isolated polynucleotide can be further manipulated to produce other polynucleotides, used to transfect or transform cells, incorporated into related vectors and introduced into cells for expression, and/or the like. Typical cloning vectors contain transcription and translation terminators, transcription and translation initiation sequences, and promoters useful for regulation of the expression of the particular target nucleic acid. The vectors optionally include generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both.

Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); and Atlas and Parks (eds) The Handbook of Microbiological Media (1993) CRC Press, Boca Raton, Fla. Construction of vectors containing a nucleic acid encoding a cytidine deaminase described herein employs standard ligation techniques known in the art. See, e.g., Sambrook et al, Molecular Cloning: A Laboratory Manual., Cold Spring Harbor Laboratory Press (1989) or Ausubel, R. M., ed. Current Protocols in Molecular Biology (1994).

A variety of protein isolation and detection methods are known and can be used to isolate a cytidine deaminase, helicase, or fusion protein e.g., from recombinant cultures of cells expressing the recombinant proteins presented herein. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

A cytidine deaminase, helicase, or fusion protein can be prepared as an isolated protein or polynucleotide. An “isolated” protein or polynucleotide is one that has been removed from a cell. For instance, an isolated protein is a polypeptide that has been removed from the cytoplasm or from the membrane of a cell, and many of the proteins, nucleic acids, and other cellular material of its natural environment are no longer present. Proteins that are produced outside of a cell, e.g., through chemical or recombinant means, are considered to be isolated by definition, as they were never present in a cell.

### Methods of Use

The cytidine deaminases, helicases, and fusion proteins provided by the present disclosure can be easily integrated into essentially any application for identifying modified cytosines. For instance, altered cytidine deaminases, helicases, and fusion proteins can be integrated into applications that include sequencing library preparation. Examples of sequencing library preparation include, but are not limited to, whole genome, accessible (e.g., ATAC), conformational state (e.g., HiC), and reduced representation bisulfite sequencing (RRBS). It can be particularly useful in essentially any application using low input DNA or RNA such as, but not limited to, single cell combinatorial indexing (sci) methods like sci-WGS-seq, sci-MET-seq, and sci-ATAC-seq, sci-RNA-seq, and cell free DNA-based methods. Specific applications include, but are not limited to, identifying one or more patterns of cytosine modification such as determining methylation on CpG islands (Example 15) and reduced representation bisulfite sequencing (RRBS); variant calling, including SNV/indel, copy number variation (CNV), short tandem repeats (STR), and structural variants (SV) (Example 16); detecting differentially methylated regions (DMRs) (Example 17); measuring methylation at promoters (Example 18); and detecting tumor DNA (Example 19).

The altered cytidine deaminases provided by the present disclosure can be easily integrated into essentially any application that includes locus-specific methylation profiling. Typical locus-specific detection of epigenetic methylated cytosines, such as 5mC, require the use of 5mC-specific antibodies, or multi-step chemical or chemoenzymatic transformations that lead to deamination of C or 5mC to U/T to enable differentiation of the two C-isoforms. When combined with various in vitro detection methodologies, these approaches can be strong approaches to detect 5mC at defined loci. However, these methods can be confounded by antibody cross-reactivity and stability, or the toxicity and complex workflows required by chemical and chemoenzymatic approaches. Use of an altered cytidine deaminase described herein in a single enzymatic deamination protocol permits selective conversion of 5mC to T that is compatible with a number of in vitro diagnostic modalities, resulting in locus-specific detection of 5mC.

Instead of using destructive methods for identifying methylated cytosines, integrating the cytidine deaminases, helicases, and fusion proteins provided by the present disclosure into methods for identifying modified cytosines, such as sequencing library production and locus-specific methylation profiling, results in the more efficient enzyme-catalyzed conversion of modified cytosines during generation of target nucleic acids, thereby permitting more efficient detection of modified cytosines, particularly from double-stranded nucleic acids. The altered cytidine deaminases, helicases, and fusion proteins of the present disclosure also provide better sequencing data and better retention of genetic information, which is demonstrated by high variant calling performance (see Example 16). Furthermore, as an enzymatic method for conversion, the use of the altered cytidine deaminases enables high coverage uniformity and low sample damage, which results in lower nucleic acid input requirements. A multitude of sequencing library methods are known to a skilled person that can be used in the construction of whole-genome or targeted libraries.

In general, methods for using a cytidine deaminase of the present disclosure include contacting target nucleic acids, e.g., DNA or RNA, with the enzyme, under conditions suitable for conversion of modified cytidines, such as 5mC, to thymidine, or for conversion of unmodified cytidine to uracil. Because amplification of DNA does not preserve the modification status of cytidine (e.g., the methylation status of 5mC and 5hmC is not retained), use of a cytidine deaminase typically occurs before amplification of target DNA. Target nucleic acids can be contacted with cytidine deaminase at essentially any time in a method before an amplification, provided the DNA is double-stranded. For instance, target nucleic acids can be contacted with cytidine deaminase while the nucleic acids are inside of a fixed or unfixed cell or nucleus, after isolation of genomic or cell free DNA or mRNA, before or after fragmentation, or before or after tagmentation. The skilled person will recognize that target nucleic acids can be contacted with a cytidine deaminase after addition of a universal sequence and/or an adapter, provided the universal sequence and/or an adapter is not added by amplification.

A method for using an altered cytidine deaminase can include the optional step of comparison of the treated target nucleic acid with an untreated nucleic acid or comparison of the treated target nucleic acid with a nucleic acid treated with a wild type cytidine deaminase. For instance, in embodiments where the treated nucleic acid is sequenced, the sequence can be compared to a reference sequence thereby permitting easy identification of point mutations and inference of modified cytosines. Thus, in embodiments where an altered cytidine deaminase having cytosine-defective deaminase activity (i.e., converts 5mC to T at a greater rate than converting C to U) is used, C to T point mutations can be easily identified, and these point mutations are inferred as 5mC positions. In embodiments where an altered cytidine deaminase having 5hmC-defective deaminase activity (i.e., preferentially deaminates C and 5mC to U and T, respectively, and has significantly reduced deamination of 5hmC) is used, the absence of C to T point mutations can be easily identified, and the absence point mutations are inferred as 5hmC positions. In embodiments where the treated nucleic acid is not sequenced, the nucleic acid can be treated with an altered cytidine deaminase and compared to the nucleic acid that is untreated, i.e., not contacted with an altered cytidine deaminase. Here the read-out typically depends on the assay method, for instance when an amplification is used (e.g., Example 21) the relative amounts of amplification can be easily identified and the presence or absence of a 5mC or a pattern of cytosine modification at a predetermined sequence inferred.

Reaction conditions suitable for conversion of modified cytosine, such as 5mC, to thymidine by a cytidine deaminase described herein include, but are not limited to, a substrate of target nucleic acid that is double-stranded (ds) DNA or RNA suspected of including at least one modified cytosine, pH, temperature of the reaction, time of the reaction, and concentration of the cytidine deaminase and/or ds DNA or RNA substrate.

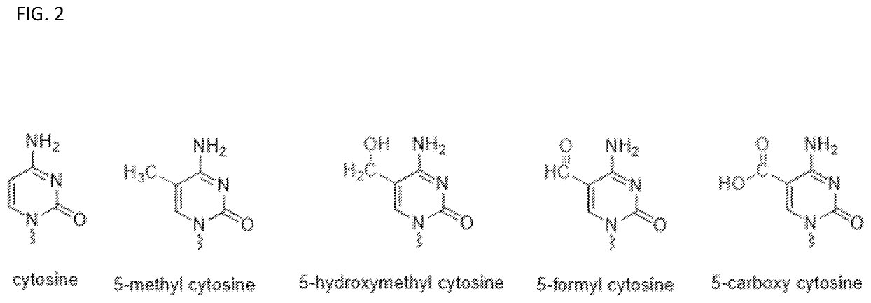

Target nucleic acids useful in the methods of the present disclosure are described herein. A modified cytosine present on a substrate double-stranded (ds) DNA or RNA includes, but is not limited to, 5-methyl cytosine (5mC), 5-hydroxymethyl cytosine (5hmC), 5-formyl cytosine (5fC), and 5-carboxy cytosine (5CaC) (FIG. 2). In one embodiment, the modified cytosine is 5-methyl cytosine. Methods that use double-stranded target DNA for generating a sequencing library can be modified to include treatment with a cytosine deaminase described herein. In some embodiments, dsDNA that is used in a tagmentation reaction or for adapter attachment can treated with a cytidine deaminase.

In some embodiments, target nucleic acids useful in the methods of the present disclosure may include additional motifs. For some helicases of interest, certain motifs may preferably improve recruitment of the helicase to a target nucleic acid. These types of motifs may be referred to herein as “helicase recruitment motifs.” Examples of helicase recruitment motifs that may be included in a target dsDNA sequence include Y-shaped adapter structures, partial duplexes, ssDNA bubbles, forks, blunt duplexes, three-strand junctions, and others. In some embodiments, an adapter may include more than one motif to recruit a helicase. Either the 3′ end or the 5′ end of a target nucleic acid may include a motif. In some embodiments, both the 3′ and the 5′ end of a target nucleic acid include a motif.

In some embodiments, a cytidine deaminase as presented herein can be used to differentiate between 5-methyl cytosine (5mC) and 5-hydroxymethyl cytosine (5hmC). In such an embodiment, a sample of DNA suspected of including single-stranded DNA comprising at least one 5-methyl cytosine (5mC) or 5-hydroxymethyl cytosine (5hmC) is modified to prevent a cytidine deaminase from converting 5hmC to thymidine. Methods for blocking deaminase activity are known in the art, and any one of a number of methods can be used to protect 5hmC from deaminase activity. As one example, target DNA can be treated to modify 5hmC but not 5mC such that 5hmC is an unsuitable substrate for cytidine deaminase activity. In a specific example, a glucosyltransferase enzyme can be used to glucosylate 5hmC but not 5mC. Glucosyltransferase enzymes are known to those of skill in the art, and include, for example, β-glucosyltransferase (OGT). By way of example, the enzyme T4 β-glucosyltransferase is commercially available (OGT, NEB) and can be used for modification of 5hmC. Methods for using a βGT to glucosylate 5hmC are known in the art, and can be used in conjunction with the use of cytidine deaminase enzymes as presented here. For example, a sample of DNA can be treated with a βGT to glucosylate 5hmC in the sample DNA prior to treating the DNA with the cytidine deaminase enzyme. By treating the sample DNA with a βGT, 5hmC is protected from the deaminase activity of the cytidine deaminase. Thus, 5mC will be detected in downstream readout, such as sequencing, PCR, array, and the like, as a thymidine. In contrast, any protected 5hmC sites will be detected as cytosine in the same readout. Enzymes, buffers, and conditions for performing glucosylation of 5hmC are known in the art, as exemplified by the methods disclosed in Schutsky et al., Nature biotechnology, 10.1038/nbt.4204. 8 Oct. 2018, doi:10.1038/nbt.4204. A specific example of modification of 5hmC to protect it from cytidine deaminase activity is provided in Examples 9, 10, and 11. In some embodiments, a cytidine deaminase as presented herein can be used in in vitro diagnostic (IVD) approaches for profiling methylation in a locus-specific manner. Current methods for methylation biomarker detection typically include digestion of genomic DNA with methylation-sensitive enzymes and then quantitative PCR (qPCR) at a locus of interest to quantify the extent of restriction enzyme digestion, and therefore the percent methylation at that site. This is followed by mismatch-sensitive qPCR of bisulfite-treated DNA, where 5mC is read out as a lack of 5mC>T conversion. These methods, however, have drawbacks. The recognition site of the methyl-sensitive restriction enzyme must be present in the methylated region of the target locus. Bisulfite treatment requires a large quantity of starting DNA and results in conversion to a low complexity genome (unmethylated cytosines—which represent the majority of cytosines in the genome—are converted to U and read as T). This reduced complexity of the genomic template constrains the design of qPCR primers that hybridize specifically to the locus of interest. During bisulfite conversion, DNA is intrinsically damaged or lost, which can hinder downstream analysis. DNA damage decreases coverage uniformity of the genome, which can lead to bias coverage. Furthermore, incomplete bisulfite conversion has the potential to adversely affect results, since it can exaggerate DNA methylation levels (Sam et al., PLoS One. 2018; 13(6); Ehrich et al., Nucleic Acids Res. Oxford University Press; 2007; 35: e29).

5mC to T conversion by the altered cytidine deaminases described herein obviates the need for restriction enzymes or bisulfite treatment, and preserves DNA complexity. The resulting modifications of one or more cytosines can be detected using established in vitro diagnostic (IVD) approaches for profiling methylation in a locus-specific manner. Examples of approaches include detection of 5mC loci via amplification, e.g., quantitative PCT (qPCR) (Example 21), detection of 5mC loci using a CRISPR-based system, e.g., CRISPR-Cas12 (Example 22), spatial detection of 5mC using molecular cytogenic methods, e.g., fluorescence in situ hybridization (FISH) (Example 23), and array-based detection of 5mC (Example 24). In one embodiment, in vitro diagnostic (IVD) approaches for profiling methylation in a locus-specific manner use one or more primers to anneal to a predetermined sequence that may include one or more modified cytosines. After treatment of target nucleic acids with an altered cytidine deaminase, the modified cytosines present in the target nucleic acids are converted as described herein (e.g., 5mC is converted to T), and primers can be easily designed to anneal with higher affinity to a predetermined sequence when it includes nucleotides resulting from the deaminase treatment (e.g., a T nucleotide where a 5mC was present prior to treatment). For example, as described in Example 21, primers used for an amplification bind with greater affinity to a nucleic acid that includes T nucleotides where 5mC nucleotides were present prior to treatment. The annealing of a primer to a predetermined sequence that includes the expected 5mC to T conversion(s) allows one to infer the location of a modified cytosine in the untreated target nucleic acid. A primer that binds with greater affinity to a nucleic acid that includes T nucleotides where 5mC nucleotides were present prior to treatment can include at least 1, at least 2, at least 3, at least 4 or at least 5 nucleotides that will base-pair with a nucleotide that results from conversion of 5mC to T, i.e., an adenine (A), and when amplification is used, then a second primer for the reverse strand that has a T instead of guanine (G).

In some embodiments, target nucleic acids obtained from a subject can be treated with an altered cytidine deaminase to result in converted nucleic acids, and a pattern of cytosine modification can be identified in the converted nucleic acids. The pattern of cytosine modification can optionally be compared with the pattern of cytosine modification in a reference nucleic acid. In embodiments where a pattern of cytosine modification correlates with a disease or condition, the method can be used in diagnostic or prognostic applications. For instance, the subject can have or be at risk of having a disease or condition, and the reference nucleic acid can be from a normal subject, e.g., a subject that does not have and is not at risk for the disease or condition. The pattern of cytosine modification can be associated with a disease or condition (e.g., the target nucleic acid can be a predetermined sequence), and identification in the subject of a pattern of cytosine modification associated with a disease or condition can indicate the subject has or is at risk of having the disease or condition. For instance, a pattern of cytosine modification can be linked in-cis to a coding region that is correlated with a disease or condition and identification of that pattern, or absence of that pattern, in the subject can be used for diagnosis or prognosis. In one embodiment, the coding region can be one that is transcriptionally active or transcriptionally inactive in a reference nucleic acid. The comparison of the converted nucleic acid to the reference nucleic acid can include determining if the pattern of cytosine modification of the converted nucleic acid indicates the coding region is transcriptionally active or transcriptionally inactive in the subject. When that coding region is associated with a disease or condition, the status of transcriptional activity can be used for diagnosis or prognosis.

Comparison of a pattern of cytosine modification in a subject can also be used in identifying changes in a pattern of cytosine modification in a subject over time. For instance, a subject can have a disease or conditions and is undergoing treatment, or a subject had a disease or condition and is cured (e.g., the subject was treated and no signs of the disease or condition are present) or in remission (e.g., the subject was treated and signs of the disease or condition are reduced). Target nucleic acids from the subject at different times, e.g., before treatment started, during treatment, after treatment is stopped, can be compared and a pattern of cytosine modification of a sequence, e.g., a predetermined sequence compared and used to determine the progress of a treatment or the status of the disease or condition in the subject.

In some embodiments where detection of 5mC nucleotides uses amplification, the use of a polymerase that disfavors uracil can aid in reducing the amplification of treated target nucleic acids that include spurious C to U conversion that may result from use of an altered cytidine deaminase. B-family polymerases are known to exhibit “uracil read-ahead” function which causes stalling of the polymerase at uracil residues (Greagg et al., 1999, PNAS USA; 96(16):9045-50). Examples of B-family polymerases that disfavor uracil include archaeal B-family polymerases from Pyrococcus furiosus (Pfu), Thermococcus kodakarensis (KOD), Thermococcus litoralis (Tli/Vent), Pyrococcus woesei (Pwo), and Thermococcus fumicolans (Tfu). Other examples of uracil-disfavoring polymerases include Phusion™, Q5®, and Kapa HiFi™. In other embodiments where amplification of nucleic acids containing uracil nucleotides is desired, the use of a uracil tolerant polymerase can be used. Examples of uracil-tolerant polymerases include PhusionU™, Q5U®, KapaU™, Taq, and Dpo4.

Wild-type cytidine deaminases typically function at near-neutral pH, e.g., pH 7. Altered cytidine deaminases described herein can have increased activity at below neutral pH. In some embodiments, the pH of a reaction that includes an altered cytidine deaminase described herein can be no greater than pH 6.9, no greater than pH 6.7, no greater than pH 6.5, no greater than pH 6.3, no greater than pH 6.1, no greater than pH 6.0, no greater than pH 5.9, no greater than pH 5.7, no greater than pH 5.5, no greater than pH 5.3, no greater than pH 5.2, or no greater than pH 5.1. In some embodiments, the pH of a reaction that includes an altered cytidine deaminase described herein can be at least pH 5.1, at least pH 5.3, at least pH 5.5, at least pH 5.7, at least pH 5.9, at least pH 6.1, at least pH 6.3, at least pH 6.5, at least pH 6.7, or at least pH 6.9. In some embodiments, the pH of a reaction that includes an altered cytidine deaminase described herein can be no greater than pH 7.5, no greater than pH 7.3, or no greater than pH 7.1. Examples of ranges of pH in a reaction include at least 5.1 to no greater than 6.9, at least 5.1 to no greater than 6.5, at least 5.1 to no greater than 6.3, or at least 5.1 to no greater than 6.1. The activity of an altered cytidine deaminase to deaminate a 5mC oligonucleotide substrate can be an increased catalytic activity that is at least 10-fold greater, at least 50-fold greater, or at least 100-fold greater when comparing activity (see Example 3).

It is expected that a cytidine deaminase, including an altered cytidine deaminase, can function in essentially any buffer. Examples of useful buffers include, but are not limited to: a citrate buffer, such as the citrate buffer available from Thermo Fisher Scientific (Cat. No. #005000); sodium acetate buffer, Bis Tris-Propane HCl; and Tris-HCl Tris. Examples of other buffers include, but are not limited to, Bicine, DIPSO, glycylglycine, HEPES, imidazole, malonate, MES, MOPS, PB, phosphate, PIPES, SPG, succinate, TAPS, TAPSO, trincine. In some embodiments a reducing agent such as dithiothreitol (DTT) can be present. In some embodiments a divalent cation is not included

A deamination reaction can occur at a temperature of 25° C. to 37° C., such as 37° C. Some cytidine deaminases described herein preferentially deaminate a modified cytosine to thymine at a faster rate than deamination of cytosine to uracil. Thus, in some embodiments the time of reaction can be used to maximize the difference of deamination of modified cytosine versus deamination of cytosine. In one embodiment, the reaction can proceed for at least 15 minutes, at least 30 minutes, at least 45 minutes, at least 60 minutes, at least 90 minutes, at least 120 minutes, or at least 150 minutes, and for no greater than 15 minutes, no greater than 30 minutes, no greater than 45 minutes, no greater than 60 minutes, no greater than 90 minutes, no greater than 120 minutes, no greater than 150 minutes, or no greater than 180 minutes.

In one embodiment, a deamination reaction can include a cytidine deaminase at a concentration from at least 0.5 micromolar (μM) to no greater than 5 μM. For instance, the concentration of the enzyme can be at least 0.5, at least 1 μM, at least 2 μM, at least 3 μM, at least 4 μM, or 5 μM, and/or no greater than 5 μM, no greater than 4 μM, no greater than 3 μM, no greater than 2 μM, no greater than 1 μM, or 0.5 μM. In one embodiment, a deamination reaction can include nucleic acids at a concentration of at least 1 picomolar (pM) to no greater than 2 μM. For instance, the concentration of nucleic acids can be at least 1 pM, at least 3 pM, at least 6 pM, at least 10 pM, at least 100 pM, at least 1 nanomolar (nM), at least 40 nM, at least 400 nM, at least 500 nM, at least, 600 nM, at least 700 nM, at least 800 nM, at least 900 nM, or 1 μM, and/or no greater than 1 μM, no greater than 900 nM, no greater than 800 nM, no greater than 700 nM, no greater than 600 nM, no greater than 500 nM, no greater than 400 nM, no greater than 40 nM, no greater than 1 nM, no greater than 100 nM, no greater than 6 nM, or no greater than 3 nM.

In one embodiment, a deamination reaction can include an RNAse. RNase A has been implicated in increasing activity of cytidine deaminases (Bransteitter et al., Proceedings of the National Academy of Sciences of the United States of America 100, no. 7 (2003): 4102-7. doi.org/10.1073/pnas.0730835100). When activity of an altered cytidine deaminase of the present disclosure was determined in the presence of RNAse A the opposite was observed. When RNAse A was included in the reaction, an altered cytidine deaminase having cytosine-defective deaminase activity (i.e., converts 5mC to T at a greater rate than converting C to U) had reduced activity, and the reduced activity was more pronounced for off-target cytosine deamination. Thus, RNAse A resulted in greater selectivity for deamination of 5mC compared to C. An RNAse A can be included in a deamination reaction at a concentration from at least 1 microgram/milliliter (ug/ml) to no greater than 20 μM. For instance, the concentration of RNAse A can be at least 1 ug/ml, at least 2 ug/ml, at least 3 ug/ml, at least 4 ug/ml, 5 ug/ml, 6 ug/ml, 7 ug/ml, 8 ug/ml, or 9 ug/ml, and/or no greater than 50 ug/ml, no greater than 40 ug/ml, no greater than 30 ug/ml, no greater than 20 ug/ml, no greater than 19 ug/ml, no greater than 18 ug/ml, no greater than 17 ug/ml, no greater than 16 ug/ml, no greater than 15 ug/ml, no greater than 14 ug/ml, no greater than 13 ug/ml, no greater than 12 ug/ml, or no greater than 11 ug/ml. In one embodiment, the concentration of RNAse A is from 2 ug/ml to 13 ug/ml, or from 5 ug/ml to 10 ug/ml.

### Target Nucleic Acids

The target nucleic acids contacted with a protein complex and used in the methods, compositions, and kits provided herein may be essentially any nucleic acid of known or unknown sequence. Sequencing may result in determination of the sequence of the whole or a part of the target molecule. In one embodiment, target nucleic acids can be processed into templates suitable for amplification by the placement of universal amplification sequences, e.g., sequences present in a universal adaptor, at the ends of each target fragment.

Target nucleic acids are typically derived from primary nucleic acids present in a sample, such as a biological sample. The primary nucleic acids may originate as DNA or RNA. DNA primary nucleic acids may originate in double-stranded DNA (dsDNA) form (e.g., genomic DNA, genomic DNA fragments, cell-free DNA, and the like) from a sample or may originate in single-stranded form from a sample. RNA primary nucleic acids may be mRNA or non-coding RNA, e.g., microRNA or small interfering RNA. The precise sequence of the polynucleotide molecules from a primary nucleic acid sample is generally not material to the disclosure and may be known or unknown.

Target nucleic acids of this disclosure typically adopt a double helix arrangement. Target nucleic acids can be formed as a double-stranded double helix of B-DNA. In some embodiments, a single strand of nucleic acid may form a double helix. Alternate arrangements of nucleic acids are also possible, including A-DNA, and Z-DNA. In embodiments, the nucleic acid of interest may be circular, such as plasmid DNA. The nucleic acid of interest may have a complex tertiary structure, such as that found in telomeres. The tertiary structure of a nucleic acid may impact which helicases are suitable to unwind the nucleic acid.

The primary nucleic acid molecules may represent the entire genetic complement of an organism, e.g., genomic DNA molecules which include both intron and exon sequences, as well as non-coding regulatory sequences such as promoter and enhancer sequences. The primary nucleic acid molecules may represent the entire genetic complement of specific cells of an organism, e.g., from tumor cells, where the genomic DNA molecules which include both intron and exon sequences, as well as non-coding regulatory sequences such as promoter and enhancer sequences. In one embodiment, particular subsets of genomic DNA can be used, such as, for example, particular chromosomes, DNA associated with open chromatin, DNA associated with closed chromatin, or one or more specific sequences such as a region of a specific gene (e.g., targeted sequencing). In one embodiment, the primary nucleic acid molecules may represent a particular subset of DNA, e.g., DNA having a specific sequence that anneals with a primer such as one used for targeted sequencing or target enrichment. In one embodiment, a particular subset of DNA can be used, such as cell-free DNA, which can include DNA of the subject including DNA from normal cells, DNA from diseased cells such as tumor cells, and/or DNA from fetal cells.

The primary nucleic acid molecules may represent the entire transcriptome of cells of an organism, e.g., mRNA molecules. The primary nucleic acid molecules may represent the entire transcriptome of specific cells of an organism, e.g., from tumor cells or for instance the cells of a tissue. In one embodiment, the primary nucleic acid molecules may represent a particular subset of mRNA, e.g., mRNA having a specific sequence that anneals with a primer such as one used for targeted sequencing or target enrichment.

A sample, such as a biological sample, can include nucleic acid molecules obtained from biopsies, tumors, scrapings, swabs, blood, mucus, urine, stool, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include cultured cells. In some embodiments, the sample can include nucleic acid molecules obtained from an animal such as a human or mammalian source. In another embodiment, the sample can include nucleic acid molecules obtained from a non-mammalian source such as a plant, bacteria, virus, or fungus. In some embodiments, the source of the nucleic acid molecules may be an archived or extinct sample or species.

Additional non-limiting examples of sources of biological samples can include whole organisms as well as a sample obtained from a patient. The biological sample can be obtained from any biological fluid or tissue and can be in a variety of forms, including fluid, e.g., liquid or gas, tissue, solid tissue, and preserved forms such as dried, frozen, and fixed forms. The sample may be of any biological tissue, cells or fluid. Such samples include, but are not limited to, sputum, blood, serum, plasma, blood cells (e.g., white cells), ascitic fluid, urine, saliva, tears, sputum, vaginal fluid (discharge), washings obtained during a medical procedure (e.g., pelvic or other washings obtained during biopsy, endoscopy or surgery), tissue, nipple aspirate, core or fine needle biopsy samples, cell-containing body fluids, peritoneal fluid, and pleural fluid, or cells therefrom, and free floating nucleic acids such as cell-free circulating DNA. Biological samples may also include sections of tissues such as frozen or fixed sections taken for histological purposes or micro-dissected cells or extracellular parts thereof. In some embodiments, the sample can be a blood sample, such as, for example, a whole blood sample. In another example, the sample is an unprocessed dried blood spot (DBS) sample. In yet another example, the sample is a formalin-fixed paraffin-embedded (FFPE) sample. In yet another example, the sample is a saliva sample. In yet another example, the sample is a dried saliva spot (DSS) sample.

Exemplary biological samples from which target nucleic acids can be derived include, for example, those from a eukaryote, for instance a mammal, such as a rodent, mouse, rat, rabbit, guinea pig, ungulate, horse, sheep, pig, goat, cow, cat, dog, primate, human or non-human primate; a plant, such as Arabidopsis thaliana, corn, sorghum, oat, wheat, rice, canola, or soybean; an algae, such as Chlamydomonas reinhardtii; a nematode such as Caenorhabditis elegans; an insect, such as Drosophila melanogaster, mosquito, fruit fly, honey bee or spider; a fish, such as zebrafish; a reptile; an amphibian, such as a frog or Xenopus laevis; a Dictyostelium discoideum; a fungi, such as Pneumocystis carinii, Takifugu rubripes, yeast, Saccharamoyces cerevisiae, or Schizosaccharomyces pombe; or a protozoan such as Plasmodium falciparum. Target nucleic acids can also be derived from a prokaryote such as a bacterium, Escherichia coli, Staphylococcus or Mycoplasma pneumoniae; an archaeon; a virus such as Hepatitis C virus or human immunodeficiency virus; or a viroid. Target nucleic acids can be derived from a homogeneous culture or population of organisms described herein or alternatively from a collection of several different organisms, for example, in a community or ecosystem.

In some embodiments, a biological sample includes tissue that is processed to obtain the desired primary nucleic acids. In some embodiments, cells are used obtain the desired primary nucleic acids. In some embodiments, nuclei are used to obtain the desired primary nucleic acids. The method can further include dissociating cells, and/or isolating nuclei from cells. Methods for isolating cells and nuclei from tissue are available (WO 2019/236599).

In some embodiments, nucleic acids present in tissue, in cells, or in isolated nuclei can be processed depending on the desired read-out. For instance, nucleic acids can be fixed during processing, and useful fixation methods are available (WO 2019/236599). Fixation can be useful to preserve a sample or maintain contiguity of analytes from a sample, a cell, or a nucleus. Fixation methods preserve and stabilize tissue, cell, and nucleus morphology and architecture, inactivate proteolytic enzymes, strengthen samples, cells, and nuclei so they can withstand further processing and staining, and protect against contamination. Examples of methods where fixation can be useful include, but are not limited to, whole genome sequencing of isolated nuclei and chromosome conformation capture methods such as H-C. Common methods of fixation include perfusion, immersion, freezing, and drying (Srinivasan et al., Am J Pathol. 2002 December; 161(6): 1961-1971.doi: 10.1016/S0002-9440(10)64472-0). In some embodiments such as whole genome sequencing, isolated nuclei can be processed to dissociate nucleosomes from DNA while leaving the nuclei intact, and methods for generating nucleosome-free nuclei are available (WO 2018/018008).

In some embodiments, primary nucleic acids in bulk, e.g., from a plurality of cells, can be used to produce a sequencing library as described herein. In other embodiments, individual cells or nuclei can be used as sources of primary nucleic acids to obtain sequence information from single cells and nuclei. Many different single cell library preparation methods are known in the art, including, but not limited to, Drop-seq, Seq-well, and single cell combinatorial indexing (“sci-”) methods. Companies providing single cell products and related technologies include, but are not limited to, Illumina™, 10X Genomics™, Takara Biosciences™, BD Biosciences™, Bio-Rad Laboratories™, 1Cellblo™, Isoplexis™, CellSee™, Nanoselect™, and Dolomite Bio™. Sci-seq is a methodological framework that employs split-pool barcoding to uniquely label the nucleic acid contents of large numbers of single cells or nuclei. Typically, the number of nuclei or cells can be at least two. The upper limit is dependent on the practical limitations of equipment (e.g., multi-well plates, number of indexes) used in other steps of the methods as described herein. The number of nuclei or cells that can be used is not intended to be limiting and can number in the billions.

The target nucleic acids used in the methods and compositions of the present disclosure can be derived by fragmentation. Random fragmentation refers to the fragmentation of a polynucleotide molecule from a primary nucleic acid sample in a non-ordered fashion by enzymatic, chemical, or mechanical methods. Such fragmentation methods are known in the art and use standard methods (Sambrook and Russell, Molecular Cloning, A Laboratory Manual, third edition). Moreover, random fragmentation is designed to produce fragments irrespective of the sequence identity or position of nucleotides comprising and/or surrounding the break. In one embodiment, the random fragmentation is by mechanical means such as nebulization or sonication to produce fragments of about 50 base pairs in length to about 1500 base pairs in length, still more particularly 50-700 base pairs in length, yet more particularly 50-400 base pairs in length. Most particularly, the method is used to generate smaller fragments of from 50-150 base pairs in length

Fragmentation of polynucleotide molecules by mechanical means (nebulization, sonication, and Hydroshear, for example) results in fragments with a heterogeneous mix of blunt and 3′- and 5′-overhanging ends. It is therefore desirable to repair the fragment ends using methods or kits (such as the Lucigen™ DNA terminator End Repair Kit) known in the art to generate ends that are optimal for insertion, for example, into blunt sites of cloning vectors. In a particular embodiment, the fragment ends of the population of nucleic acids are blunt ended. More particularly, the fragment ends are blunt ended and phosphorylated. The phosphate moiety can be introduced via enzymatic treatment, for example, using polynucleotide kinase.

In a particular embodiment, the target fragment sequences are prepared with single overhanging nucleotides by, for example, activity of certain types of DNA polymerase such as Taq polymerase or Klenow exo-minus polymerase which has a non-template-dependent terminal transferase activity that adds a single deoxynucleotide, for example, deoxyadenosine (A) to the 3′ ends of a DNA molecule, for example, a PCR product. Such enzymes can be used to add a single nucleotide ‘A’ to the blunt ended 3′ terminus of each strand of the double-stranded target fragments. Thus, an ‘A’ could be added to the 3′ terminus of each end repaired strand of the double-stranded target fragments by reaction with Taq or Klenow exo-minus polymerase, while the universal adapter polynucleotide construct could be a T-construct with a compatible ‘T’ overhang present on the 3′ terminus of each region of double-stranded nucleic acid of the universal adapter. This end modification also prevents self-ligation of both vector and target such that there is a bias towards formation of target nucleic acids having a universal adapter at each end.

In one embodiment, fragmentation can be accomplished using a process often referred to as tagmentation. Tagmentation uses a transposome complex and combines into a single step fragmentation and ligation to add universal adapters (WO 2016/130704). A transposome complex is a transposase bound to a transposase recognition site and can insert the transposase recognition site into a target nucleic acid in a process sometimes termed “tagmentation.” In some such insertion events, one strand of the transposase recognition site may be transferred into the target nucleic acid. Such a strand is referred to as a “transferred strand.” In one embodiment, a transposome complex includes a dimeric transposase having two subunits, and two non-contiguous transposon sequences. In another embodiment, a transposase includes a dimeric transposase having two subunits, and a contiguous transposon sequence.

Some embodiments can include the use of a hyperactive Tn5 transposase and a Tn5-type transposase recognition site (Goryshin and Reznikoff, J. Biol. Chem., 273:7367 (1998)), or MuA transposase and a Mu transposase recognition site comprising R1 and R2 end sequences (Mizuuchi, K., Cell, 35: 785, 1983; Savilahti, H, et al., EMBO J., 14: 4893, 1995). Tn5 Mosaic End (ME) sequences can also be used by a skilled artisan.

Examples of transposon sequences useful with the methods and compositions described herein are provided in U.S. Patent Application Pub. No. 2012/0208705, U.S. Patent Application Pub. No. 2012/0208724 and Int. Patent Application Pub. No. WO 2012/061832. In some embodiments, a transposon sequence includes a first transposase recognition site and a second transposase recognition site.

Some transposome complexes useful herein include a transposase having two transposon sequences. In some such embodiments, the two transposon sequences are not linked to one another, in other words, the transposon sequences are non-contiguous with one another. Examples of such transposomes are known in the art (see, for instance, U.S. Patent Application Pub. No. 2010/0120098).

In one embodiment, tagmentation is used to produce target nucleic acids that include different universal sequences at each end. This can be accomplished by using two types of transposome complexes, where each transposome complex includes a different nucleotide sequence that is part of the transferred strand.

In some embodiments, tagmentation results in the formation of a 5′ overhang on one or both ends. This overhang is a region of ssDNA, which may not be a suitable helicase substrate. In some embodiments, the tagmented population of target nucleic acids may be treated with a polymerase and dNTPs to fill in the overhang, resulting in a fully double-stranded nucleic acid. In some of these embodiments, the resulting nucleic acid may have a single adenosine overhang at the 3′ end. Optionally, this A overhang may be removed.

A population of target nucleic acids can have an average strand length that is desired or appropriate for a particular application of the methods, compositions, or kits set forth herein. For example, the average strand length can be less than about 100,000 nucleotides, 50,000 nucleotides, 10,000 nucleotides, 5,000 nucleotides, 1,000 nucleotides, 500 nucleotides, 100 nucleotides, or 50 nucleotides. Alternatively or additionally, the average strand length can be greater than about 10 nucleotides, 50 nucleotides, 100 nucleotides, 500 nucleotides, 1,000 nucleotides, 5,000 nucleotides, 10,000 nucleotides, 50,000 nucleotides, or 100,000 nucleotides. The average strand length for a population of target nucleic acids can be in a range between a maximum and minimum value set forth herein. It will be understood that amplicons generated at an amplification site (or otherwise made or used herein) can have an average strand length that is in a range between an upper and lower limit selected from those exemplified above.

In some cases, a population of target nucleic acids can be produced under conditions or otherwise configured to have a maximum length for its members. For example, the maximum length for the members that are used in one or more steps of a method set forth herein or that are present in a particular composition can be less than 100,000 nucleotides, less than 50,000 nucleotides, less than 10,000 nucleotides, less than 5,000 nucleotides, less than 1,000 nucleotides, less than 500 nucleotides, less than 100 nucleotides, or less than 50 nucleotides. Alternatively or additionally, a population of target nucleic acids can be produced under conditions or otherwise configured to have a minimum length for its members. For example, the minimum length for the members that are used in one or more steps of a method set forth herein or that are present in a particular composition can be more than 10 nucleotides, more than 50 nucleotides, more than 100 nucleotides, more than 500 nucleotides, more than 1,000 nucleotides, more than 5,000 nucleotides, more than 10,000 nucleotides, more than 50,000 nucleotides, or more than 100,000 nucleotides. The maximum and minimum strand length for target nucleic acids in a population can be in a range between a maximum and minimum value set forth above. It will be understood that amplicons generated at an amplification site (or otherwise made or used herein) can have maximum and/or minimum strand lengths in a range between the upper and lower limits exemplified above.

In some embodiments, a sample can be enriched for sequences of interest, e.g., a predetermined sequence. For example, a subset of genes or regions of the genome are isolated and sequenced, or a subset of genes or regions of the genome are interrogated by other methods, such as a locus-specific in vitro diagnostic method. A predetermined sequence can be, for instance, one that can have a pattern of cytosine modification.

In some embodiments, target enrichment works by capturing genomic regions of interest by hybridization to target-specific probes that can be used to physically separate target DNA that has hybridized to bait probes from all other DNA in solution, which are then washed away. For example, some methods of enrichment use biotinylated probes, which are then isolated by magnetic pulldown with streptavidin-coated magnetic particles. In another example, some methods of enrichment use analyte arrays, also known as microarrays, that allow for the hybridization of predetermined sequences.

Enrichment can occur, for example, prior to treatment with cytidine deaminase. In such embodiments, enriching a nucleic acid of interest, or a fragment thereof, such as enriching DNA in a sample, may include any suitable enrichment techniques. In some embodiments, enrichment of DNA may include enrichment through molecular inversion probes, in solution capture, pulldown probes, bait sets, standard PCR, multiplex PCR, hybrid capture, endonuclease digestion, DNase I hypersensitivity, and selective circularization. Enrichment can be achieved through negative selection of nucleic acids by eliminating undesired material. This sort of enrichment includes ‘footprinting’ techniques or ‘subtractive’ hybrid capture. During the former, the target sample is safe from nuclease activity through the protection of protein or by single and double stranded arrangements. During the latter, nucleic acids that bind ‘bait’ probes are eliminated.

In some embodiments, enriching can comprise amplification using target-specific primers. In some embodiments, amplification is performed subsequent to another form of enrichment. Typically, however, in embodiments where amplification is used for enrichment, the amplification step occurs after treatment with deaminase, to preserve methylation status of the target DNA. In some such embodiments, amplification can include PCR amplification or genome-wide amplification.

In some embodiments, enrichment can occur after treatment with a cytidine deaminase. Typically, methods used to identity methylated cytosines result in the loss of DNA complexity due to conversion of unmethylated DNA bases to uracil, resulting in a low complexity genome and limits the use of sequences that specifically hybridize to a predetermined sequence. Accordingly, typical methods for identifying methylated cytosines are more difficult to use in methods that include enrichment, such as hybrid-enrichment sequencing and amplicon-based targeted sequencing, after conversion of methylated cytosines. In contrast, because of (i) the 5mC to T conversion by altered cytidine deaminases and (ii) only a small percentage of cytosines are methylated and expected to be converted by an altered cytidine deaminase. Examples of enrichment-based methods that can be used after treatment of a target nucleic acid with an altered cytidine deaminase include but are not limited to analyte arrays, use of primers for selective amplification, CRISPR-Cas systems, and molecular cytogenic techniques such as FISH. Examples of arrays include, for instance, methylation arrays for interrogation of selected methylation sites across a genome (e.g., the Infinium MethylationEPIC BeadChip, Illumina).

### Attachment of Universal Adapters

In some embodiments, a target nucleic acid used in a method, composition, or kit described herein can include a universal adapter attached to each end. A target nucleic acid having a universal adapter at each end can be referred to as a “modified target nucleic acid.” Methods for attaching a universal adapter to each end of a target nucleic acid used in a method described herein are known to the person skilled in the art. The attachment can be through tagmentation using transposase complexes (WO 2016/130704), or through standard library preparation techniques using ligation (U.S. Pat. Pub. No. 2018/0305753). Attachment of a universal adapter to the ends of a target nucleic acid can occur before or after treatment of the target nucleic acid with a cytidine deaminase.

In some embodiments, adapters compatible with the methods and compositions described herein may include additional regions not generally included in sequencing adapters. For some helicases of interest, certain motifs may preferably improve recruitment of the helicase to a target nucleic acid. Examples of motifs that may be included in an adapter sequence include Y-shaped adapter structures, partial duplexes, ssDNA bubbles, forks, blunt duplexes, three-strand junctions, and others. In some embodiments, an adapter may include more than one motif to recruit a helicase. Either the 3′ end or the 5′ end may include a motif. In some embodiments, both the 3′ and the 5′ end include a motif.

In one embodiment, double-stranded target nucleic acids from a sample, e.g., a fragmented sample that has been contacted with a cytidine deaminase and converted from single-stranded to double-stranded nucleic acids, are treated by first ligating identical universal adaptor molecules to the 5′ and 3′ ends of the double-stranded target nucleic acids. In one embodiment, the universal adapters are “matched” adapters or Y adapters because the two strands of the adaptors are formed by annealing complementary polynucleotide strands. In one embodiment, the universal adapters used in the method of the disclosure are referred to as “mismatched” adaptors because the adaptors include a region of sequence mismatch, i.e., they are not formed by annealing fully complementary polynucleotide strands. The general features of mismatched adaptors are further described in Gormley et al., U.S. Pat. No. 7,741,463, and Bignell et al., U.S. Pat. No. 8,053,192). The universal adaptor typically includes universal capture binding sequences that aid in immobilizing the target nucleic acids on an array for subsequent sequencing, and universal primer binding sites useful for the sequencing. In another embodiment, a sample is contacted with a protein complex, such as an cytidine deaminase and a helicase or a fusion protein as described herein. Double-stranded target nucleic acids from the sample are then subjected to tagmentation with a transposome complex that inserts a universal adapter, or sequences that can be used to add a universal adapter, into the target double-stranded nucleic acids.

A universal adapter can optionally include at least one index. An index can be used as a marker characteristic of the source of particular target nucleic acids on a flow cell(U.S. Pat. No. 8,053,192). Generally, the index is a synthetic sequence of nucleotides that is part of the universal adapter which is added to the target nucleic acids as part of the library preparation step. Accordingly, an index is a nucleic acid sequence which is attached to each of the target molecules of a particular sample, the presence of which is indicative of, or is used to identify, the sample or source from which the target molecules were isolated.

Preferably an index may be up to 20 nucleotides in length, more preferably 1-10 nucleotides, and most preferably 4-6 nucleotides in length. A four nucleotide index gives a possibility of multiplexing 256 samples on the same array, a six base index enables 4096 samples to be processed on the same array.

The precise nucleotide sequence of the universal adapters is generally not material to the disclosure and may be selected by the user such that the desired sequence elements are ultimately included in the common sequences of the plurality of different modified target nucleic acids, for example, to provide for the universal capture binding sequences for immobilizing the target nucleic acids on an array for subsequent sequencing, and binding sites for particular sets of universal amplification primers and/or sequencing primers. Additional sequence elements may be included, for example, to provide binding sites for sequencing primers which will ultimately be used in sequencing of target nucleic acids in the library, sequencing of an index, or products derived from amplification of the target nucleic acids in the library, for example on a solid support.

In order to prepare a library of deaminase-treated DNA for analysis using a sequencing platform, it may be useful to make additional modifications to the target DNA, either prior to or after treatment with cytidine deaminase. In some embodiments, single-stranded deaminase-treated DNA is prepared for sequencing using a single-stranded library preparation method, as is known in the art. Such methods include, but are not limited to, template switching based second strand synthesis, adapters containing a single-stranded splint overhang, and the like. Reagents for performing single-stranded library preparation methods are commercially available. Examples include xGen ssDNA & Low-Input DNA Library Prep Kit (Integrated DNA Technologies catalog number 10009859), previously sold as Accel-NGS (Swift Biosciences), NGS Single Stranded DNA Library Prep Kit (BioDynami catalog number 30082). Another example includes single-reaction single-stranded library (SRSLY) as set forth in Troll et al., BMC Genomics 20, 1023 (2019).

In some embodiments, library preparation modifications are made to double-stranded target DNA prior to treatment with an altered cytidine deaminase. Methods for library preparation of double-stranded DNA template are known in the art, and include Y-adaptor ligation, transposome-based tagmentation, and the like. It will be appreciated by those of skill in the art that methods of double-strand library preparation often include one or more amplification steps using for example, PCR. In such methods, the amplification step may be deferred until after cytidine deaminase treatment, to preserve the methylation status of the template strand. For example, in Y-adapter ligation methods, the Y-adapters can be ligated to the double-stranded template, after which the adapter-ligated template DNA is denatured and treated with cytidine deaminase as described elsewhere herein. Following treatment with cytidine deaminase, the resulting treated single-strand DNA molecules can be amplified using PCR, bridge amplification, and other methods as are commonly known in the art.

### Preparation of Immobilized Samples for Sequencing

The library of modified target nucleic acids, e.g., target nucleic acids having universal adapters at each end, can be prepared for sequencing. Methods for attaching modified target nucleic acids to a substrate are known in the art. In one embodiment, modified fragments are enriched using a plurality of capture oligonucleotides having specificity for the modified fragments, and the capture oligonucleotides can be immobilized on a surface of a solid substrate such as a flow cell or a bead. For instance, capture oligonucleotides can include a first member of a universal binding pair, and where a second member of the binding pair is immobilized on a surface of a solid substrate. Likewise, methods for amplifying immobilized target nucleic acids include, but are not limited to, bridge amplification and exclusion amplification (also referred to as kinetic exclusion amplification (KEA). Methods for immobilizing and amplifying prior to sequencing are described in, for instance, Bignell et al. (U.S. Pat. No. 8,053,192), Gunderson et al. (WO2016/130704), Shen et al. (U.S. Pat. No. 8,895,249), and Pipenburg et al. (U.S. Pat. No. 9,309,502).

A pooled sample can be immobilized in preparation for sequencing. Sequencing can be performed as an array of single molecules or can be amplified prior to sequencing. The amplification can be carried out using one or more immobilized primers. The immobilized primer(s) can be, for instance, a lawn on a planar surface, or on a pool of beads. The pool of beads can be isolated into an emulsion with a single bead in each “compartment” of the emulsion. At a concentration of only one template per “compartment,” only a single template is amplified on each bead.

The term “solid-phase amplification” as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers is/are immobilized on the solid support. Solid phase PCR covers systems such as emulsions, where one primer is anchored to a bead and the other is in free solution, and colony formation in solid phase gel matrices wherein one primer is anchored to the surface, and one is in free solution.

In some embodiments, the solid support comprises a patterned surface. A “patterned surface” refers to an arrangement of different regions in or on an exposed layer of a solid support. For example, one or more of the regions can be features where one or more amplification primers are present. The features can be separated by interstitial regions where amplification primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. Exemplary patterned surfaces that can be used in the methods and compositions set forth herein are described in U.S. Pat. Nos. 8,778,848, 8,778,849 and 9,079,148, and U.S. Pat. Appl. Pub. No. 2014/0243224.

In some embodiments, the solid support includes an array of wells or depressions in a surface. This may be fabricated as is generally known in the art using a variety of techniques, including, but not limited to, photolithography, stamping techniques, molding techniques and micro-etching techniques. As will be appreciated by those of skill in the art, the technique used will depend on the composition and shape of the array substrate.

The features in a patterned surface can be wells in an array of wells (e.g. microwells or nanowells) on glass, silicon, plastic or other suitable solid supports with patterned, covalently-linked gel such as poly(N-(5-azidoacetamidylpentyl)acrylamide-co-acrylamide) (PAZAM, see, for example, US Pub. No. 2013/184796, WO 2016/066586, and WO 2015/002813). The process creates gel pads used for sequencing that can be stable over sequencing runs with a large number of cycles. The covalent linking of the polymer to the wells is helpful for maintaining the gel in the structured features throughout the lifetime of the structured substrate during a variety of uses. However, in many embodiments the gel need not be covalently linked to the wells. For example, in some conditions silane free acrylamide (SFA, see, for example, U.S. Pat. No. 8,563,477) which is not covalently attached to any part of the structured substrate, can be used as the gel material.

In particular embodiments, a structured substrate can be made by patterning a solid support material with wells (e.g. microwells or nanowells), coating the patterned support with a gel material (e.g. PAZAM, SFA, or chemically modified variants thereof, such as the azidolyzed version of SFA (azido-SFA)) and polishing the gel coated support, for example via chemical or mechanical polishing, thereby retaining gel in the wells but removing or inactivating substantially all of the gel from the interstitial regions on the surface of the structured substrate between the wells. Primer nucleic acids can be attached to gel material. A solution of modified target nucleic acids can then be contacted with the polished substrate such that individual modified target nucleic acids will seed individual wells via interactions with primers attached to the gel material; however, the target nucleic acids will not occupy the interstitial regions due to absence or inactivity of the gel material. Amplification of the modified target nucleic acids will be confined to the wells since absence or inactivity of gel in the interstitial regions prevents outward migration of the growing nucleic acid colony. The process can be conveniently manufactured, being scalable and utilizing conventional micro- or nanofabrication methods.

Although the disclosure encompasses “solid-phase” amplification methods in which only one amplification primer is immobilized (the other primer usually being present in free solution), in one embodiment the solid support is provided with both the forward and the reverse primers immobilized. In practice, there will be a plurality of identical forward primers and/or a plurality of identical reverse primers immobilized on the solid support, since the amplification process requires an excess of primers to sustain amplification. References herein to forward and reverse primers are to be interpreted accordingly as encompassing a plurality of such primers unless the context indicates otherwise.

As will be appreciated by the skilled reader, any given amplification reaction requires at least one type of forward primer and at least one type of reverse primer specific for the template to be amplified. However, in certain embodiments the forward and reverse primers may include template-specific portions of identical sequence, and may have entirely identical nucleotide sequence and structure (including any non-nucleotide modifications). In other words, it is possible to carry out solid-phase amplification using only one type of primer, and such single-primer methods are encompassed within the scope of the disclosure. Other embodiments may use forward and reverse primers which contain identical template-specific sequences but which differ in some other structural features. For example, one type of primer may contain a non-nucleotide modification which is not present in the other.

Primers for solid-phase amplification are preferably immobilized by single point covalent attachment to the solid support at or near the 5′ end of the primer, leaving the template-specific portion of the primer free to anneal to its cognate template and the 3′ hydroxyl group free for primer extension. Any suitable covalent attachment means known in the art may be used for this purpose. The chosen attachment chemistry will depend on the nature of the solid support, and any derivatization or functionalization applied to it. The primer itself may include a moiety, which may be a non-nucleotide chemical modification, to facilitate attachment. In a particular embodiment, the primer may include a sulphur-containing nucleophile, such as phosphorothioate or thiophosphate, at the 5′ end. In the case of solid-supported polyacrylamide hydrogels, this nucleophile will bind to a bromoacetamide group present in the hydrogel. A more particular means of attaching primers and templates to a solid support is via 5′ phosphorothioate attachment to a hydrogel comprised of polymerized acrylamide and N-(5-bromoacetamidylpentyl) acrylamide (BRAPA), as described in Int. Pub. No. WO 05/065814.

Certain embodiments of the disclosure may make use of solid supports that include an inert substrate or matrix (e.g. glass slides, polymer beads, etc.) which has been “functionalized,” for example by application of a layer or coating of an intermediate material including reactive groups which permit covalent attachment to biomolecules, such as polynucleotides. Examples of such supports include, but are not limited to, polyacrylamide hydrogels supported on an inert substrate such as glass. In such embodiments, the biomolecules (e.g. polynucleotides) may be directly covalently attached to the intermediate material (e.g. the hydrogel), but the intermediate material may itself be non-covalently attached to the substrate or matrix (e.g. the glass substrate). The term “covalent attachment to a solid support” is to be interpreted accordingly as encompassing this type of arrangement.

The pooled samples may be amplified on beads wherein each bead contains a forward and reverse amplification primer. In one embodiment, a library of modified target nucleic acids is used to prepare clustered arrays of nucleic acid colonies, analogous to those described in U.S. Pub. No. 2005/0100900, U.S. Pat. No. 7,115,400, WO 00/18957 and WO 98/44151 by solid-phase amplification and more particularly solid phase isothermal amplification. The terms “cluster” and “colony” are used interchangeably herein to refer to a discrete site on a solid support including a plurality of identical immobilized nucleic acid strands and a plurality of identical immobilized complementary nucleic acid strands. The term “clustered array” refers to an array formed from such clusters or colonies. In this context, the term “array” is not to be understood as requiring an ordered arrangement of clusters.

The term “solid phase” or “surface” is used to mean either a planar array wherein primers are attached to a flat surface, for example, glass, silica or plastic microscope slides or similar flow cell devices; beads, wherein either one or two primers are attached to the beads and the beads are amplified; or an array of beads on a surface after the beads have been amplified.

Clustered arrays can be prepared using either a process of thermocycling, as described in WO 98/44151, or a process whereby the temperature is maintained as a constant, and the cycles of extension and denaturing are performed using changes of reagents. Such isothermal amplification methods are described in patent application numbers WO 02/46456 and U.S. Pub. No. 2008/0009420.

It will be appreciated that any of the amplification methodologies described herein or generally known in the art may be used with universal or target-specific primers to amplify immobilized DNA fragments. Suitable methods for amplification include, but are not limited to, the polymerase chain reaction (PCR), strand displacement amplification (SDA), transcription mediated amplification (TMA) and nucleic acid sequence-based amplification (NASBA), as described in U.S. Pat. No. 8,003,354. The above amplification methods may be employed to amplify one or more nucleic acids of interest. For example, PCR, including multiplex PCR, SDA, TMA, NASBA and the like may be utilized to amplify immobilized DNA fragments. In some embodiments, primers directed specifically to the polynucleotide of interest are included in the amplification reaction.

Other suitable methods for amplification of polynucleotides may include oligonucleotide extension and ligation, rolling circle amplification (RCA) (Lizardi et al., Nat. Genet. 19:225-232 (1998)) and oligonucleotide ligation assay (OLA) (See generally U.S. Pat. Nos. 7,582,420, 5,185,243, 5,679,524 and 5,573,907; EP 0 320 308 B1; EP 0 336 731 B1; EP 0 439 182 B1; WO 90/01069; WO 89/12696; and WO 89/09835) technologies. It will be appreciated that these amplification methodologies may be designed to amplify immobilized DNA fragments. For example, in some embodiments, the amplification method may include ligation probe amplification or oligonucleotide ligation assay (OLA) reactions that contain primers directed specifically to the nucleic acid of interest. In some embodiments, the amplification method may include a primer extension-ligation reaction that contains primers directed specifically to the nucleic acid of interest. As a non-limiting example of primer extension and ligation primers that may be specifically designed to amplify a nucleic acid of interest, the amplification may include primers used for the Golden Gate assay (Illumina, Inc., San Diego, CA) as exemplified by U.S. Pat. Nos. 7,582,420 and 7,611,869.

DNA nanoballs can also be used in combination with methods, systems, compositions and kits as described herein. Methods for creating and using DNA nanoballs for genomic sequencing can be found at, for example, US patents and publications U.S. Pat. No. 7,910,354, 2009/0264299, 2009/0011943, 2009/0005252, 2009/0155781, 2009/0118488 and as described in, for example, Drmanac et al. (2010, Science 327(5961): 78-81). Briefly, following production of modified target nucleic acids, the modified target nucleic acids are circularized and amplified by rolling circle amplification (Lizardi et al., 1998. Nat. Genet. 19:225-232; US 2007/0099208 A1). The extended concatemeric structure of the amplicons promotes coiling creates compact DNA nanoballs. The DNA nanoballs can be captured on substrates, preferably to create an ordered or patterned array such that distance between each nanoball is maintained thereby allowing sequencing of the separate DNA nanoballs. In some embodiments such as those used by Complete Genomics (Mountain View, Calif), consecutive rounds of adapter addition, amplification, and digestion are carried out prior to circularization to produce head to tail constructs having several target nucleic acids separated by adapter sequences.

Exemplary isothermal amplification methods that may be used in a method of the present disclosure include, but are not limited to, Multiple Displacement Amplification (MDA) as exemplified by, for example Dean et al., Proc. Natl. Acad. Sci. USA 99:5261-66 (2002) or isothermal strand displacement nucleic acid amplification exemplified by, for example U.S. Pat. No. 6,214,587. Other non-PCR-based methods that may be used in the present disclosure include, for example, strand displacement amplification (SDA) which is described in, for example Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyper-branched strand displacement amplification which is described in, for example Lage et al., Genome Res. 13:294-307 (2003). Isothermal amplification methods may be used with, for instance, the strand-displacing Phi 29 polymerase or Bst DNA polymerase large fragment, 5′->3′ exo-for random primer amplification of genomic DNA. The use of these polymerases takes advantage of their high processivity and strand displacing activity. High processivity allows the polymerases to produce fragments that are 10-20 kb in length. As set forth above, smaller fragments may be produced under isothermal conditions using polymerases having low processivity and strand-displacing activity such as Klenow polymerase. Additional description of amplification reactions, conditions and components are set forth in detail in the disclosure of U.S. Pat. No. 7,670,810.

In some embodiments, isothermal amplification can be performed using kinetic exclusion amplification (KEA), also referred to as exclusion amplification (ExAmp). A nucleic acid library of the present disclosure can be made using a method that includes a step of reacting an amplification reagent to produce a plurality of amplification sites that each includes a substantially clonal population of amplicons from an individual target nucleic acid that has seeded the site. In some embodiments, the amplification reaction proceeds until a sufficient number of amplicons are generated to fill the capacity of the respective amplification site. Filling an already seeded site to capacity in this way inhibits target nucleic acids from landing and amplifying at the site thereby producing a clonal population of amplicons at the site. In some embodiments, apparent clonality can be achieved even if an amplification site is not filled to capacity prior to a second target nucleic acid arriving at the site. Under some conditions, amplification of a first target nucleic acid can proceed to a point that a sufficient number of copies are made to effectively outcompete or overwhelm production of copies from a second target nucleic acid that is transported to the site. For example, in an embodiment that uses a bridge amplification process on a circular feature that is smaller than 500 nm in diameter, it has been determined that after 14 cycles of exponential amplification for a first target nucleic acid, contamination from a second target nucleic acid at the same site will produce an insufficient number of contaminating amplicons to adversely impact sequencing-by-synthesis analysis on an Illumina sequencing platform.

In some embodiments, amplification sites in an array can be, but need not be, entirely clonal. Rather, for some applications, an individual amplification site can be predominantly populated with amplicons from a first modified target nucleic acid and can also have a low level of contaminating amplicons from a second modified target nucleic acid. An array can have one or more amplification sites that have a low level of contaminating amplicons so long as the level of contamination does not have an unacceptable impact on a subsequent use of the array. For example, when the array is to be used in a detection application, an acceptable level of contamination would be a level that does not impact signal to noise or resolution of the detection technique in an unacceptable way. Accordingly, apparent clonality will generally be relevant to a particular use or application of an array made by the methods set forth herein. Exemplary levels of contamination that can be acceptable at an individual amplification site for particular applications include, but are not limited to, at most 0.1%, 0.5%, 1%, 5%, 10% or 25% contaminating amplicons. An array can include one or more amplification sites having these exemplary levels of contaminating amplicons. For example, up to 5%, 10%, 25%, 50%, 75%, or even 100% of the amplification sites in an array can have some contaminating amplicons. It will be understood that in an array or other collection of sites, at least 50%, 75%, 80%, 85%, 90%, 95% or 99% or more of the sites can be clonal or apparently clonal.

In some embodiments, kinetic exclusion can occur when a process occurs at a sufficiently rapid rate to effectively exclude another event or process from occurring. Take, for example, the making of a nucleic acid array where sites of the array are randomly seeded with modified target nucleic acids from a solution and copies of the modified target nucleic acids are generated in an amplification process to fill each of the seeded sites to capacity. In accordance with the kinetic exclusion methods of the present disclosure, the seeding and amplification processes can proceed simultaneously under conditions where the amplification rate exceeds the seeding rate. As such, the relatively rapid rate at which copies are made at a site that has been seeded by a first target nucleic acid will effectively exclude a second nucleic acid from seeding the site for amplification. Kinetic exclusion amplification methods can be performed as described in detail in the disclosure of U.S. Pat. Appl. Pub. No. 2013/0338042.

Kinetic exclusion can exploit a relatively slow rate for initiating amplification (e.g. a slow rate of making a first copy of a modified target nucleic acids) vs. a relatively rapid rate for making subsequent copies of the modified target nucleic acids (or of the first copy of the modified target nucleic acids). In the example of the previous paragraph, kinetic exclusion occurs due to the relatively slow rate of modified target nucleic acids seeding (e.g. relatively slow diffusion or transport) vs. the relatively rapid rate at which amplification occurs to fill the site with copies of the modified target nucleic acid seed. In another exemplary embodiment, kinetic exclusion can occur due to a delay in the formation of a first copy of a modified target nucleic acid that has seeded a site (e.g. delayed or slow activation) vs. the relatively rapid rate at which subsequent copies are made to fill the site. In this example, an individual site may have been seeded with several different modified target nucleic acids (e.g. several modified target nucleic acids can be present at each site prior to amplification). However, first copy formation for any given modified target nucleic acid can be activated randomly such that the average rate of first copy formation is relatively slow compared to the rate at which subsequent copies are generated. In this case, although an individual site may have been seeded with several different modified target nucleic acids, kinetic exclusion will allow only one of those to be amplified. More specifically, once a first modified target nucleic acid has been activated for amplification, the site will rapidly fill to capacity with its copies, thereby preventing copies of a second modified target nucleic acid from being made at the site.

In one embodiment, the method is carried out to simultaneously (i) transport modified target nucleic acids to amplification sites at an average transport rate, and (ii) amplify the modified target nucleic acids that are at the amplification sites at an average amplification rate, wherein the average amplification rate exceeds the average transport rate (U.S. Pat. No. 9,169,513). Accordingly, kinetic exclusion can be achieved in such embodiments by using a relatively slow rate of transport. For example, a sufficiently low concentration of modified target nucleic acids can be selected to achieve a desired average transport rate, with lower concentrations resulting in slower average rates of transport. Alternatively or additionally, a high viscosity solution and/or presence of molecular crowding reagents in the solution can be used to reduce transport rates. Examples of useful molecular crowding reagents include, but are not limited to, polyethylene glycol (PEG), ficoll, dextran, or polyvinyl alcohol. Exemplary molecular crowding reagents and formulations are set forth in U.S. Pat. No. 7,399,590, which is incorporated herein by reference. Another factor that can be adjusted to achieve a desired transport rate is the average size of the target nucleic acids.

An amplification reagent can include further components that facilitate amplicon formation, and in some cases increase the rate of amplicon formation. An example is a recombinase. A recombinase can facilitate amplicon formation by allowing repeated invasion/extension. More specifically, a recombinase can facilitate invasion of a modified target nucleic acid by the polymerase and extension of a primer by the polymerase using the modified target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g. via heating or chemical denaturation) is not required. As such, recombinase-facilitated amplification can be carried out isothermally. It is generally desirable to include ATP, or other nucleotides (or in some cases non-hydrolysable analogs thereof) in a recombinase-facilitated amplification reagent to facilitate amplification. A mixture of recombinase and single-stranded binding (SSB) protein is particularly useful as SSB can further facilitate amplification. Exemplary formulations for recombinase-facilitated amplification include those sold commercially as TwistAmp kits by TwistDx (Cambridge, UK). Useful components of recombinase-facilitated amplification reagent and reaction conditions are set forth in U.S. Pat. Nos. 5,223,414 and 7,399,590.

Another example of a component that can be included in an amplification reagent to facilitate amplicon formation and in some cases to increase the rate of amplicon formation is a helicase. A helicase can facilitate amplicon formation by allowing a chain reaction of amplicon formation. The process can occur more rapidly than standard PCR since a denaturation cycle (e.g. via heating or chemical denaturation) is not required. As such, helicase-facilitated amplification can be carried out isothermally. A mixture of helicase and single-stranded binding (SSB) protein is particularly useful as SSB can further facilitate amplification. Exemplary formulations for helicase-facilitated amplification include those sold commercially as IsoAmp kits from Biohelix (Beverly, MA). Further, examples of useful formulations that include a helicase protein are described in U.S. Pat. Nos. 7,399,590 and 7,829,284. It should be noted that multiple helicases may be used for multiple functions in the methods of this disclosure. For example, a first helicase may be provided as a fusion protein with an altered APOBEC3A to convert modified cytosine to thymidine, and a second helicase may be provided later to facilitate amplicon formation.

Yet another example of a component that can be included in an amplification reagent to facilitate amplicon formation and in some cases increase the rate of amplicon formation is an origin binding protein. The origin of replication for a gene may be bound by an origin recognition complex (ORC) which includes a number of origin binding proteins. Inclusion of an origin binding protein in an amplicon formation reaction may improve recruitment of additional enzymes required for or useful in amplification.

### Methods of Sequencing

Following attachment of modified target nucleic acids to a surface, the sequence of the immobilized and amplified modified target nucleic acids is determined. Sequencing can be carried out using any suitable sequencing technique, and methods for determining the sequence of immobilized and amplified modified target nucleic acids, including strand re-synthesis, are known in the art and are described in, for instance, Bignell et al. (U.S. Pat. No. 8,053,192), Gunderson et al. (WO2016/130704), Shen et al. (U.S. Pat. No. 8,895,249), and Pipenburg et al. (U.S. Pat. No. 9,309,502).

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a modified target nucleic acid can be an automated process. Preferred embodiments include sequencing-by-synthesis (“SBS”) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

In one embodiment, a nucleotide monomer includes locked nucleic acids (LNAs) or bridged nucleic acids (BNAs). The use of LNAs or BNAs in a nucleotide monomer increases hybridization strength between a nucleotide monomer and a sequencing primer sequence present on an immobilized modified target nucleic acid.

SBS techniques can use nucleotide monomers that have a terminator moiety or those that lack any terminator moieties. Methods using nucleotide monomers lacking terminators include, for example, pyrosequencing and sequencing using γ-phosphate-labeled nucleotides, as set forth in further detail herein. In methods using nucleotide monomers lacking terminators, the number of nucleotides added in each cycle is generally variable and dependent upon the template sequence and the mode of nucleotide delivery. For SBS techniques that use nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can use nucleotide monomers that have a labeled moiety or those that lack a labeled moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments where two or more different nucleotides are present in a sequencing reagent, the different nucleotides can be distinguishable from each other, or alternatively the two or more different labels can be indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320). In pyrosequencing, released PPi can be detected by being immediately converted to adenosine triphosphate (ATP) by ATP sulfurase, and the level of ATP generated can be detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g. A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026. This approach is being commercialized by Solexa (now Illumina Inc.), and is also described in WO 91/06678 and WO 07/123,744. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

In some reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth herein.

In particular embodiments some or all of the nucleotide monomers can include reversible terminators. In such embodiments, reversible terminators/cleavable fluorophores can include fluorophores linked to the ribose moiety via a 3′ ester linkage (Metzker, Genome Res. 15:1767-1776 (2005)). Other approaches have separated the terminator chemistry from the cleavage of the fluorescent label (Ruparel et al., Proc Natl Acad Sci USA 102: 5932-7 (2005)). Ruparel et al. described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluorophore and effectively reverses the termination. Examples of modified nucleotides are also described in U.S. Pat. Nos. 7,427,673, and 7,057,026.

Additional exemplary SBS systems and methods which can be used with the methods and systems described herein are described in U.S. Pat. App. Pub. Nos. 2007/0166705, 2006/0188901, 2006/0240439, 2006/0281109, 2012/0270305, and 2013/0260372, U.S. Pat. No. 7,057,026, PCT Pub. No. WO 05/065814, U.S. Pat. App. Pub. No. 2005/0100900, and PCT Pub. Nos. WO 06/064199 and WO 07/010,251.

Some embodiments can use detection of four different nucleotides using fewer than four different labels. For example, SBS can be performed using methods and systems described in the incorporated materials of U.S. Pat. App. Pub. No. 2013/0079232. As a first example, a pair of nucleotide types can be detected at the same wavelength, but distinguished based on a difference in intensity for one member of the pair compared to the other, or based on a change to one member of the pair (e.g., via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected In no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in U.S. Pub. No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can use sequencing by ligation techniques. Such techniques use DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597.

Some embodiments can use nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”, Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003)). In such embodiments, the modified target nucleic acid passes through a nanopore. The nanopore can be a synthetic pore or biological membrane protein, such as α-hemolysin. As the modified target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008)). Data obtained from nanopore sequencing can be stored, processed and analyzed as set forth herein. In particular, the data can be treated as an image in accordance with the exemplary treatment of optical images and other images that is set forth herein.

Some embodiments can use methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414, or nucleotide incorporations can be detected with zero-mode waveguides as described, for example, in U.S. Pat. No. 7,315,019, and using fluorescent nucleotide analogs and engineered polymerases as described, for example, in U.S. Pat. No. 7,405,281 and U.S. Pub. No. 2008/0108082. The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008)). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in U.S. Pub. Nos. 2009/0026082; 2009/0127589; 2010/0137143; and 2010/0282617. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different modified target nucleic acids are manipulated simultaneously. In particular embodiments, different modified target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the modified target nucleic acids can be in an array format. In an array format, the modified target nucleic acids can be typically bound to a surface in a spatially distinguishable manner. The modified target nucleic acids can be bound by direct covalent attachment, attachment to a bead or other particle or binding to a polymerase or other molecule that is attached to the surface. The array can include a single copy of a modified target nucleic acid at each site (also referred to as a feature) or multiple copies having the same sequence can be present at each site or feature. Multiple copies can be produced by amplification methods such as, bridge amplification or emulsion PCR as described in further detail herein.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of cm², in parallel. Accordingly, the present disclosure provides integrated systems capable of preparing and detecting nucleic acids using techniques known in the art such as those exemplified herein. Thus, an integrated system of the present disclosure can include fluidic components capable of delivering amplification reagents and/or sequencing reagents to one or more immobilized modified target nucleic acids, the system including components such as pumps, valves, reservoirs, fluidic lines and the like. A flow cell can be configured and/or used in an integrated system for detection of target nucleic acids. Exemplary flow cells are described, for example, in U.S. Pat. Nos. 8,241,573 and 8,951,781. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Pat. No. 8,951,781.

While the embodiments presented herein are generally described using a sequencing platform (such as a sequencing by synthesis platform) as a readout, one of ordinary skill in the art will recognize that nucleic acids modified by the protein complexes presented herein can also be detected using any other suitable readout methodology. For example, the location and identity of modified cytosines can be assessed using a microarray. Any of a variety of analyte arrays (also referred to as “microarrays”) known in the art can be used in a method or system set forth herein. A typical array contains analytes, each having an individual probe or a population of probes. In the latter case, the population of probes at each analyte is typically homogenous having a single species of probe. For example, in the case of a nucleic acid array, each analyte can have multiple nucleic acid molecules each having a common sequence. However, in some implementations the populations at each analyte of an array can be heterogeneous. Similarly, protein arrays can have analytes with a single protein or a population of proteins typically, but not always, having the same amino acid sequence. The probes can be attached to the surface of an array for example, via covalent linkage of the probes to the surface or via non-covalent interaction(s) of the probes with the surface. In some implementations, probes, such as nucleic acid molecules, can be attached to a surface via a gel layer as described, for example, in U.S. patent application Ser. No. 13/784,368 and US Pat. App. Pub. No. 2011/0059865 A1.

Example arrays include, without limitation, a BeadChip™ Array available from Illumina, Inc.™ (San Diego, Calif.) or others such as those where probes are attached to beads that are present on a surface (e.g. beads in wells on a surface) such as those described in U.S. Pat. Nos. 6,266,459; 6,355,431; 6,770,441; 6,859,570; or 7,622,294; or PCT Publication No. WO 00/63437. Further examples of commercially available microarrays that can be used include, for example, an Affymatrix™ Genechip™ microarray or other microarray synthesized in accordance with techniques sometimes referred to as VLSIPS™ (Very Large Scale Immobilized Polymer Synthesis) technologies. A spotted microarray can also be used in a method or system according to some implementations of the present disclosure. An example spotted microarray is a CodeLink™ Array available from Amersham Biosciences. Another microarray that is useful is one that is manufactured using inkjet printing methods such as SurePrint™ Technology available from Agilent Technologies.

In some embodiments, a protein complex as presented herein can be used to convert 5-methyl cytosine (5mC) to thymidine (T) by deamination as described herein, such as by providing a sample of DNA suspected of including double-stranded DNA including at least one 5-methyl cytosine (5mC), at least one 5-hydroxymethyl cytosine (5hmC), at least one 5-formyl cytosine (5fC), at least one 5-carboxy cytosine (5CaC), or a combination thereof; contacting the DNA with the protein complex under conditions suitable for conversion of 5 methylcytosine (5mC) to thymidine (T) by deamination at a greater rate than conversion of cytosine (C) to uracil (U) by deamination, to result in converted double-stranded DNA, wherein 5mC, 5hmC, 5fC, and/or 5CaC are converted to T.

Depending on the desired result, the sample may be treated with the protein complex before or during preparation of a sequencing library. In some embodiments, the sample is contacted with the protein complex before tagmentation. It may be desirable to tagment and add adapters to the sample prior to treatment with the protein complexes described herein. As the protein complexes described herein are active on double-stranded nucleic acids, the sample may be treated prior to steps which separate the two strands of a sample, such as bridge amplification. It may be desirable to keep the two strands of a sample proximally close during preparation of a sample for sequencing.

In some embodiments, the sample is contacted with the protein complex after tagmentaiton. In some embodiments, the sample is contacted with the protein complex after tagmentation and after treatment with a polymerase to produce a dsDNA sample. As described above, a helicase, such as some helicases consistent with those described herein, may be more active on a double-stranded nucleic acid. For this reason, it may be desirable to convert the sample to a fully double-stranded form prior to treatment with the protein complexes described herein.

In a specific embodiment, a cytidine deaminase of the present disclosure can be used to detect 5hmC as described herein, such as by providing a sample of DNA suspected of including single-stranded DNA that has at least one 5-hydroxymethyl cytosine (5hmC); contacting the DNA with the altered cytidine deaminase under conditions suitable for conversion of unmodified cytosine to uracil and 5mC to thymidine and no detectable conversion of 5hmC to 5hmU.

The converted double-stranded DNA can then be processed as needed to facilitate hybridization to a microarray. For example, the converted DNA can be amplified. Any one of a number of amplification methods as are known in the art can be performed. For example, whole-genome amplification or amplification using universal primers that hybridize to a common region in the converted DNA, such as an adaptor sequence, can be used. Additionally or alternatively, the converted DNA can be fragmented. Fragmentation can be performed prior to or following amplification, or in the absence of amplification. Any one of a number of fragmentation methods as are known in the art can be performed. As one example, fragmentation can be performed using an enzymatic process, such as a restriction endonuclease or other enzyme capable of cleaving the converted DNA. As another example, fragmentation can be performed using mechanical means, such as shearing using, for example a sonication device such as those supplied by Covaris. The fragmented converted DNA can then be precipitated and/or resuspended in a buffer suitable for hybridization to a microarray. Following hybridization, the methylation state of regions of interest, such as a specific CpG locus or loci, can be interrogated at specific locations on the microarray. Methods of preparing converted DNA for microarray analysis are known in the art. One example of such methods is described in the Methylation Protocol Guide for the Infinium HD Assay from Illumina (San Diego, CA). Whereas such a protocol guide may describe use of a microarray designed for interrogation of bisulfite-converted DNA, it will be understood that array features, specifically probe sequences, can be specifically designed for DNA that has not been bisulfite converted. As an example, a commercially available microarray such as the Infinium MethylationEPIC BeadChip (Illumina) is specifically designed to hybridize with DNA fragments with reduced complexity, as found in bisulfite converted DNA, where most if not all cytosines are converted to thymidine. Thus, for example, the same CpG sites can be interrogated in non-bisulfite-converted DNA by using a microarray including probes designed to hybridize to the same regions of native, non-bisulfite-converted DNA. One of skill in the art could readily obtain such a microarray. In one embodiment, a custom array could be designed using the manifest for an array such as the Infinium MethylationEPIC BeadChip, by using the “Forward Sequence” to identify a probe sequence including native DNA sequence that covers a similar or identical sequence region for the allele-specific probe sequences, which are designed to hybridize to DNA sequences where most or all cytosines have been converted to thymidine. Using such an array designed to hybridize to native (non-bisulfite-converted) DNA sequences, the methodologies and analysis methods described in the Methylation Protocol Guide for the Infinium HD Assay from Illumina (San Diego, CA) could be followed to identify methylated CpG sites in the sample DNA.

### Compositions

The present disclosure also provides compositions that include a protein complex. The protein complex typically includes a cytidine deaminase and helicase, or a helicase-cytidine deaminase complex as described herein. The composition can include one or more additional other components in addition to the cytidine deaminase and helicase. For example, the other component can include a double-stranded DNA or RNA substrate that includes, or is suspected of including, at least one modified cytosine. Typically, the modified cytosine is a 5-methyl cytosine. the modified cytosine may also be, a 5-hydroxymethyl cytosine, a 5-formyl cytosine (5fC), a 5-carboxy cytosine (5CaC), or a combination thereof. In another example, a double-stranded DNA or RNA substrate can be one including one or more known modified cytosine, e.g., a double-stranded DNA or RNA substrate that can be used as a control to measure conversion efficiency. In another example, the other component can include a buffer having a pH that is described herein. In another example, the other components can include a buffer described herein, such as a citrate buffer, a sodium acetate buffer, or a Bis-Tris buffer. In another example, the other component can include a reductant, including but not limited to, DTT and/or TCEP, as well as zinc and zinc salts.

A composition can include a polynucleotide encoding a cytidine deaminase described herein. The polynucleotide can be present in a vector, such as a plasmid or virus vector. A vector that includes the polynucleotide can be present in a host cell, such as E. coli.

### Kits

The present disclosure also provides kits for determining the methylation status of DNA or RNA. A kit includes at least one cytidine deaminase and helicase or a protein complex as described herein and one or more other components in a suitable packaging material in an amount sufficient for at least one reaction. Examples of other components include a positive control polynucleotide, such as a double-stranded DNA including one or more known modified cytosines for use in measuring conversion efficiency, or a negative control polynucleotide, such as a double-stranded DNA including unmodified cytosines. Another component can be a glucosyltransferase, such as T4-beta glucosyltransferase. Optionally, other reagents such as buffers and solutions needed to use the cytidine deaminase and nucleotide solution are also included. Instructions for use of the packaged components are also typically included.

As used herein, the phrase “packaging material” refers to one or more physical structures used to house the contents of the kit. The packaging material is constructed by known methods, preferably to provide a sterile, contaminant-free environment. The packaging material has a label which indicates that the components can be used for determining the methylation status of DNA or RNA. In addition, the packaging material contains instructions indicating how the materials within the kit are employed to practice a reaction with a cytidine deaminase. As used herein, the term “package” refers to a solid matrix or material such as glass, plastic, paper, foil, and the like, capable of holding within fixed limits the polypeptides. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

The invention is defined in the claims. However, below there is provided a non-exhaustive listing of non-limiting exemplary aspects. Any one or more of the features of these aspects may be combined with any one or more features of another example, embodiment, or aspect described herein.

### EXEMPLARY ASPECTS

Aspect 1 is a protein complex comprising a cytidine deaminase and a helicase.

Aspect 2 is the protein complex of aspect 1, wherein the cytidine deaminase and the helicase are attached.

Aspect 3 is the protein complex of aspect 1 or 2, wherein the cytidine deaminase and the helicase comprise a fusion protein.

Aspect 4 is the protein complex of any preceding aspect, wherein the fusion protein comprises a linker.

Aspect 5 is the protein complex of any preceding aspect, wherein the cytidine deaminase and the helicase are biochemically conjugated.

Aspect 6 is the protein complex of any preceding aspect, wherein the cytidine deaminase is an altered cytidine deaminase comprising an amino acid substitution mutation at a position functionally equivalent to (Tyr/Phe)130 in a wild-type APOBEC3A protein.

Aspect 7 is the protein complex of any preceding aspect, wherein the (Tyr/Phe)130 is Tyr130, and the wild-type APOBEC3A protein is SEQ ID NO:3.

Aspect 8 is the protein complex of any preceding aspect, wherein the substitution mutation at the position functionally equivalent to Tyr130 comprises a mutation to Ala, Val, or Trp.

Aspect 9 is the protein complex of any preceding aspect, wherein the altered cytidine deaminase converts 5-methyl cytosine (5mC) to thymidine (T) by deamination at a greater rate than conversion of cytosine (C) to uracil (U) by deamination.

Aspect 10 is the protein complex of any preceding aspect, wherein the rate is at least 100-fold greater.

Aspect 11 is the protein complex of any preceding aspect, wherein the altered cytidine deaminase is member of the AID subfamily, the APOBEC1 subfamily, the APOBEC2 subfamily, the APOBEC3A subfamily, the APOBEC3B subfamily, the APOBEC3C subfamily, the APOBEC3D subfamily, the APOBEC3F subfamily, the APOBEC3G subfamily, the APOBEC3G subfamily, the APOBEC3H subfamily, or the APOBEC4 subfamily.

Aspect 12 is the protein complex of any preceding aspect, wherein the altered cytidine deaminase comprises a ZDD motif H-[P/A/V]-E-X[23-28]-P-C-X[2-4]-C(SEQ ID NO: 12).

Aspect 13 is the protein complex of any preceding aspect, wherein the altered cytidine deaminase is a member of the APOBEC3A subfamily and comprises a ZDD motif HXEX24SW(S/T)PCX[2-4]CX6FX8LX5R(L/I)YX[8-11]LX2LX[10]M (SEQ ID NO:13).

Aspect 14 is the protein complex of any preceding aspect, wherein the helicase unwinds double-stranded DNA (dsDNA).

Aspect 15 is the protein complex of any preceding aspect, wherein the helicase is a member of helicase Superfamily 1, Superfamily 2, Superfamily 3, or Family 4.

Aspect 16 is the protein complex of any preceding aspect, wherein the helicase comprises Walker A motif (SEQ ID NO:68), a Walker B motif (SEQ ID NO:69), or both.

Aspect 17 is the protein complex of any preceding aspect, wherein the helicase in a member of RecQ family.

Aspect 18 is the protein complex of any preceding aspect, wherein the helicase comprises a RecQ helicase from Escherichia coli (SEQ ID NO:92).

Aspect 19 is the protein complex of any preceding aspect, wherein the protein complex converts 5mC to T by deamination in dsDNA at a greater rate than a comparable protein complex without a helicase.

Aspect 20 is the protein complex of any preceding aspect, wherein the attached protein converts 5mC to T by deamination in dsDNA at a greater rate than a comparable protein complex that does not comprise a fusion protein.

Aspect 21 is a composition comprising: the protein complex of any one of aspects 1-20; and a DNA sample comprising single-stranded DNA comprising at least one 5mC.

Aspect 22 is the composition of any preceding aspect, wherein the sample comprises genomic DNA.

Aspect 23 is the composition of any preceding aspect, wherein the genomic DNA is from a single cell or is a mixture from a plurality of cells.

Aspect 24 is a composition comprising: a protein complex comprising: a cytidine deaminase, and a helicase; and a sample comprising double-stranded DNA comprising at least one 5mC.

Aspect 25 is the composition of any preceding aspect, wherein the cytidine deaminase and the helicase are attached.

Aspect 26 is the composition of any preceding aspect, wherein the cytidine deaminase and the helicase comprise a fusion protein.

Aspect 27 is the composition of any preceding aspect, wherein the fusion protein comprises a linker.

Aspect 28 is the composition of any preceding aspect, wherein the cytidine deaminase and the helicase are chemically conjugated.

Aspect 29 is the composition of any preceding aspect, wherein the sample comprises a helicase recruitment motif.

Aspect 30 is the composition of any preceding aspect, wherein the helicase recruitment motif comprises a Y-shaped adapter structure, a partial duplex, a single-stranded DNA (ssDNA) bubble, a fork, a blunt duplex, a three-strand junction, or a combination thereof.

Aspect 31 is the composition of any preceding aspect, wherein the sample comprises genomic DNA, cell free DNA.

Aspect 32 is the composition of any preceding aspect, wherein the genomic DNA is from a single cell or is a mixture from a plurality of cells.

Aspect 33 is the composition of any preceding aspect, wherein the cytidine deaminase comprises an altered cytidine deaminase comprising an amino acid substitution mutation at a position functionally equivalent to (Tyr/Phe)130 in a wild-type APOBEC3A protein.

Aspect 34 is the composition of any preceding aspect, wherein the wherein the (Tyr/Phe)130 is Tyr130, and the wild-type APOBEC3A protein is SEQ ID NO:3.

Aspect 35 is the composition of any preceding aspect, wherein the substitution mutation at the position functionally equivalent to Tyr130 comprises a mutation to Ala, Val, or Trp.

Aspect 36 is the composition of any preceding aspect, wherein the altered cytidine deaminase converts 5-methyl cytosine (5mC) to thymidine (T) by deamination at a greater rate than conversion of cytosine (C) to uracil (U) by deamination.

Aspect 37 is the composition of any preceding aspect, wherein the rate is at least 100-fold greater.

Aspect 38 is the composition of any preceding aspect, wherein the altered cytidine deaminase is a member of the AID subfamily, the APOBEC1 subfamily, the APOBEC2 subfamily, the APOBEC3A subfamily, the APOBEC3B subfamily, the APOBEC3C subfamily, the APOBEC3D subfamily, the APOBEC3F subfamily, the APOBEC3G subfamily, the APOBEC3G subfamily, the APOBEC3H subfamily, or the APOBEC4 subfamily.

Aspect 39 is the composition of any preceding aspect, wherein the altered cytidine deaminase comprises a ZDD motif H-[P/A/V]-E-X[23-28]-P-C-X[2-4]-C(SEQ ID NO: 12).

Aspect 40 is the composition of any preceding aspect, wherein the altered cytidine deaminase is a member of the APOBEC3A subfamily and comprises a ZDD motif HXEX24SW(S/T)PCX[2-4]CX6FX8LX5R(L/I)YX[8-11]LX2LX[10]M (SEQ ID NO: 13).

Aspect 41 is the composition of any preceding aspect, wherein the substitution mutation at the position functionally equivalent to Tyr130 comprises a mutation to alanine, glycine, phenylalanine, histidine, glutamine, methionine, asparagine, lysine, valine, aspartic acid, glutamic acid, serine, cysteine, proline, arginine, or threonine.

Aspect 42 is the composition of any preceding aspect, wherein the substitution mutation at the position functionally equivalent to (Tyr/Phe)130 comprises a mutation to alanine.

Aspect 43 is the composition of any preceding aspect, wherein the cytidine deaminase converts cytosine (C) to uracil (U) by deamination at a greater rate than conversion of 5 methylcytosine (5mC) to thymidine (T) by deamination.

Aspect 44 is the composition of any preceding aspect, wherein the substitution mutation at the position functionally equivalent to Tyr130 comprises a mutation to leucine or tryptophan.

Aspect 45 is the composition of any preceding aspect, wherein the helicase unwinds double-stranded DNA (dsDNA).

Aspect 46 is the composition of any preceding aspect, wherein the helicase is a member of helicase Superfamily 1, Superfamily 2, Superfamily 3, or Family 4.

Aspect 47 is the composition of any preceding aspect, wherein the helicase comprises Walker A motif (SEQ ID NO:68), a Walker B motif (SEQ ID NO:69), or both.

Aspect 48 is the composition of any preceding aspect, wherein the helicase in a member of RecQ family.

Aspect 49 is the composition of any preceding aspect, wherein the helicase comprises a RecQ helicase from Escherichia coli (SEQ ID NO:92).

Aspect 50 is the composition of any preceding aspect, wherein the sample comprises a helicase recruitment motif.

Aspect 51 is the composition of any preceding aspect, wherein helicase recruitment motif comprises a Y-shaped adapter structure, a partial duplex, a single-stranded DNA (ssDNA) bubble, a fork, a blunt duplex, a three-strand junction, or a combination thereof.

Aspect 52 is a method comprising: providing a sample of DNA suspected of comprising dsDNA comprising at least one 5-methyl cytosine (5mC); contacting the dsDNA with the protein complex of any one of aspects 1-20 under conditions suitable for conversion of 5 methylcytosine (5mC) to thymidine (T) to result in converted double-stranded DNA, wherein 5mC is converted to T; and processing the converted double-stranded DNA to produce a sequencing library.

Aspect 53 is the method of any preceding aspect, further comprising: providing a surface comprising a plurality of amplification sites, wherein the amplification sites comprise at least two populations of attached single-stranded capture oligonucleotides having a free 3′ end, and contacting the surface comprising amplification sites with the sequencing library under conditions suitable to produce a plurality of amplification sites that each comprise a clonal population of amplicons from an individual member of the sequencing library.

Aspect 54 is the method of any preceding aspect, wherein the attached single-stranded capture oligonucleotides comprise a helicase recruitment motif.

Aspect 55 is the method of any preceding aspect, wherein helicase recruitment motif comprises a Y-shaped adapter structure, a partial duplex, a single-stranded DNA (ssDNA) bubble, a fork, a blunt duplex, a three-strand junction, or a combination thereof.

Aspect 56 is the method of any preceding aspect, wherein processing the converted double-stranded DNA to produce a sequencing library occurs after contacting the dsDNA with the protein complex.

Aspect 57 is the method of any preceding aspect, wherein processing the converted double-stranded DNA to produce a sequencing library occurs before contacting the dsDNA with the protein complex.

Aspect 58 is the method of any preceding aspect, wherein the processing comprises fragmentation or tagmentation of the double-stranded DNA and addition of a universal sequence to the double-stranded DNA fragments.

Aspect 59 is the method of any preceding aspect, wherein the universal sequence is part of an adapter added to the double-stranded DNA fragments.

Aspect 60 is the method of any preceding aspect, wherein the sample is a biological sample.

Aspect 61 is the method of any preceding aspect, wherein the biological sample comprises cell-free DNA.

Aspect 62 is the method of any preceding aspect, wherein the biological sample comprises a fluid selected from blood or serum.

Aspect 63 is the method of any preceding aspect, wherein the sample comprises single cells or isolated nuclei.

Aspect 64 is the method of any preceding aspect, wherein the biological sample comprises a tissue.

Aspect 65 is the method of any preceding aspect, wherein the tissue comprises tumor tissue.

Aspect 66 is a protein complex comprising: a means for converting a 5 methylcytosine to a thymine; and a means for directionally separating double-stranded nucleic acids.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

### Experimental Assay for Cytidine Deaminase Activity

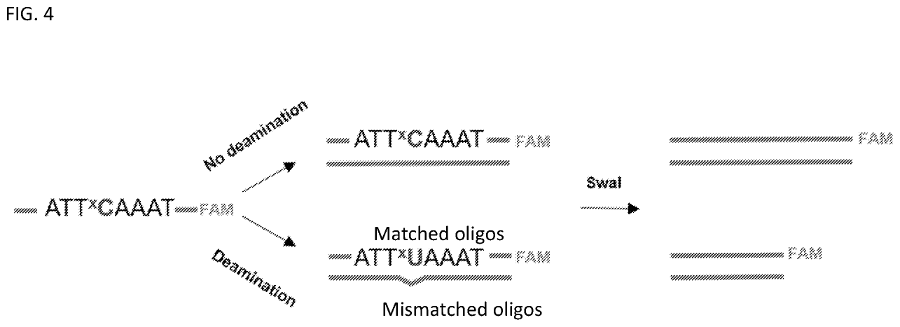

Deamination by cytidine deaminases was monitored using a gel-based assay with the restriction enzyme SwaI (FIG. 4).

Deamination of C to U or 5mC to T results in a mismatched DNA substrate, which can be cleaved by SwaI. These products are visualized as two distinct species on a denaturing polyacrylamide gel.

Upon incubation with APOBEC3A wildtype or its mutants with 5′ fluorescein amidite (FAM) labeled oligonucleotide substrate containing C or 5mC, deamination and no deamination occurs depending on the enzyme substrate preference. Introduction of a complementary oligonucleotide thereafter may result in complete matched or mismatched base pairing. If there is a mismatch, SwaI will cleave the double-stranded oligonucleotide. The original or cleaved 5′-FAM labeled oligonucleotide can be visualized by the extent of migration using 15% Urea-PAGE with FAM filter. Although FAM is used here, essentially any label can be used, and either the 5′ or the 3′ end can be labeled.

This assay was adapted and modified from Schutsky et al., Nucleic Acid Research, 45, 7655-7665, 2017. doi: 10.1093/nar/gkx345. Modifications to Schutsky et al. included the following. Instead of performing DNA precipitation and redissolving the DNA substrate into SwaI compatible buffer, 1 μL of the altered cytidine deaminase APOBEC3A(Y130A) deamination reaction mixture was aliquoted into 9 μL SwaI compatible buffer for restriction enzyme digestion for our gel assay. Appropriate controls were performed to determine the SwaI restriction enzyme digestion efficiency was not compromised by the APOBEC reaction buffer. Instead of introducing 1.5-fold excess complementary strand prior to overnight SwaI restriction enzyme digestion, 3-fold excess complementary strand was introduced. Instead of running the pre-heated 20% acrylamide/Tris-Borate-EDTA (TBE)/urea gel reported by Schutsky et al., the gel run was performed at room temperature with a 15% acrylamide/Tris-Borate-EDTA (TBE)/urea gel and observed good resolution between cut (deaminated) and uncut (unreacted) oligo substrates.

The SwaI assay was first validated using FAM-labeled DNA oligonucleotides that contain a C, 5mC, U, or T residue (FIG. 5). Oligonucleotides purchased from Integrated DNA Technologies (IDT) and visualized by the 15% Urea-PAGE with FAM filter. Synthesized oligonucleotides.oLB1609 contains substrate C, and oLB1610 contains its corresponding deaminated product U. oLB1611 contains substrate 5mC, and oLB1612 contains its corresponding deaminated product T. oLB1679 is the complementary oligonucleotide for oLB1609, oLB1610, oLB1611, and oLB1612 (FIG. 5A). As shown in FIG. 5B, annealing of oLB1609/oLB1679 and oLB1611/oLB1679 resulted in complete base pairing of the oligonucleotide. Addition of SwaI did not result in cleavage of this substrate. However, a single base mismatched substrate formed by annealing of oLB1610/oLB1679 (U/G mismatched) or oLB1612/oLB1679 (T/G mismatched) resulted in a cleavage product upon addition of SwaI. As a specific cleavage product was observed in the presence of U or T, but not C or 5mC, the SwaI assay could serve as a readout of C to U and 5mC to T deamination by APOBEC3A.

As an additional control, C and 5mC-containing DNA oligonucleotides oLB1609 (C oligo) and oLB1612 (5mC oligo) were incubated with APOBEC3A enzyme purchased from New England Biolabs (NEBNext™ Enzymatic Methyl-seq Kit (Catalog #E7120)), which was reported to deaminate both C and 5mC efficiently (FIG. 6). The deamination reaction mixture (5 μL, 2 μL and 1 μL) were then directly added to SwaI assay buffer containing SwaI, resulting in a total volume of 10 μL. 1 μL of deamination reaction was sufficient to observe the cut band, allowing quantification of the extent of deamination. Treatment with NEB APOBEC3A and subsequent digestion by SwaI resulted in formation of a cleavage product with similar mobility to both U and T-containing substrates in FIG. 5A. These data further support the notion that SwaI cleavage can be used to monitor the deaminase activity of APOBEC3A and other cytidine deaminases.

### Example 2

### Purification of APOBEC3A(Y130X) Mutant Proteins

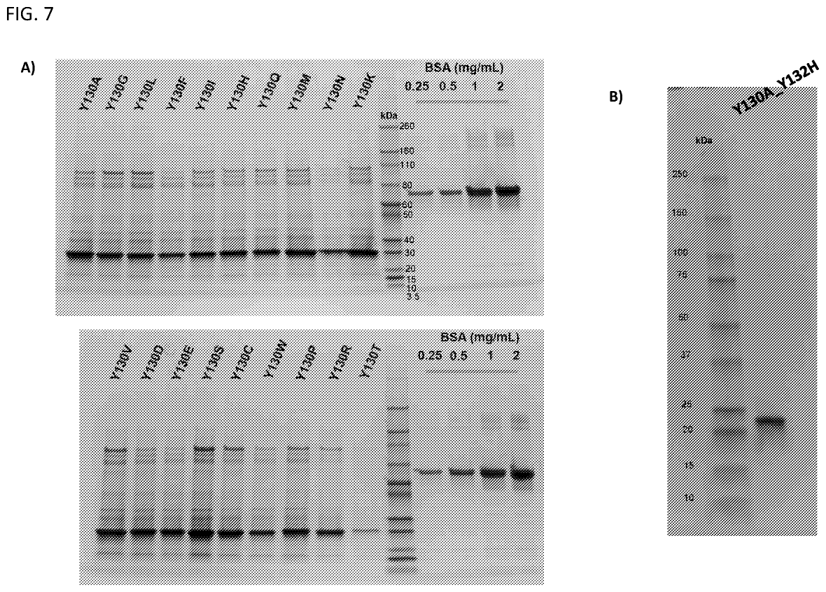

The impact of all possible amino acid substitutions at position 130 of APOBEC3A on the deaminase activity of this enzyme was systematically assessed. To this end, 19 different His-tagged APOBEC3A constructs were cloned, each encoding a different amino acid at position 130 relative to the wild type tyrosine. The corresponding proteins were expressed in BL21(DE3) cells, purified using Ni-NTA agarose beads, and desalted/concentrated using spin columns to storage buffer (50 mM Tris pH 7.5, 200 mM NaCl, 5% (v/v) glycerol, 0.01% (v/v) Tween-20, 0.5 mM DTT). This yielded APOBEC3A(Y130X) mutant protein preparations with 80-85% purity, as judged by SDS-PAGE analysis (FIG. 7A).

### Example 3

### DNA Deaminase Activity of APOBEC3A(Y130X) Mutant Proteins

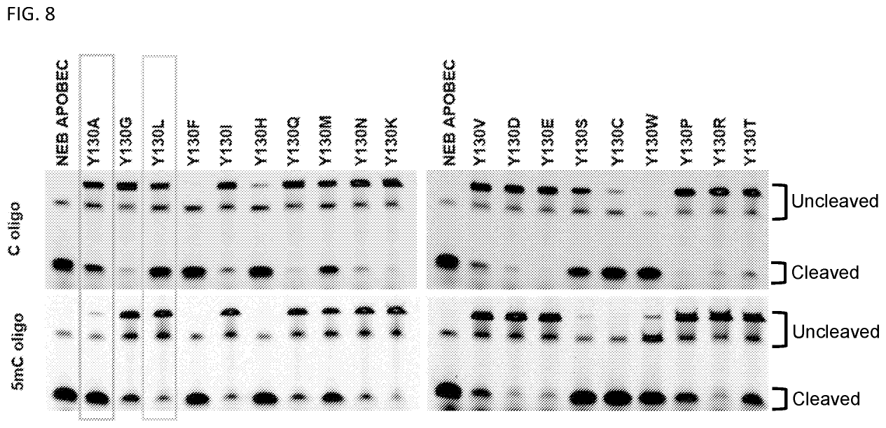

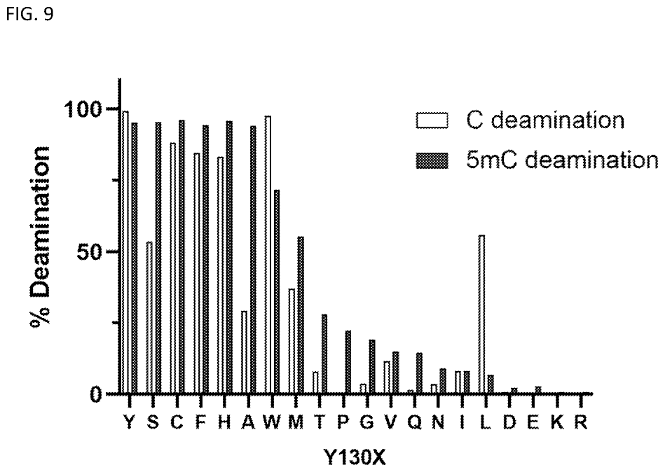

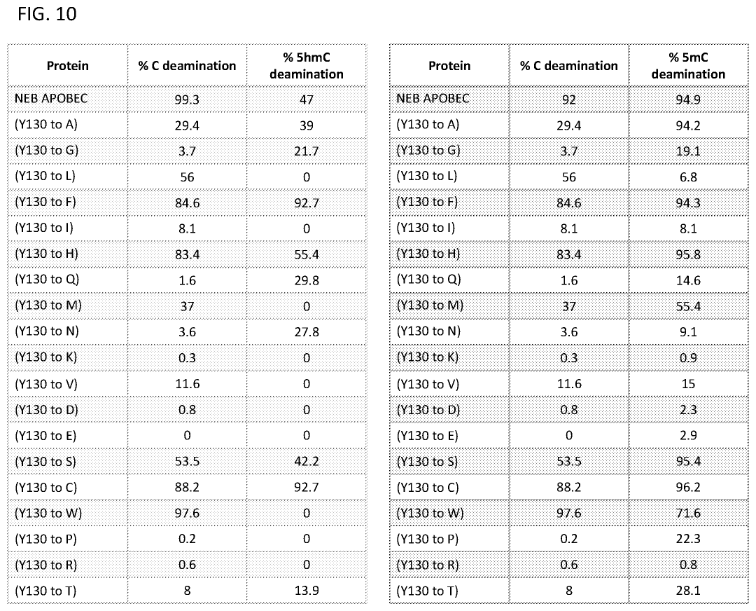

The deaminase activity of all purified APOBEC3A(Y130X) proteins was then analyzed using the SwaI assay, with a 37° C./2 hour reaction time and NEB APOBEC3A as positive control (FIG. 8). 10-20 pM final concentration of Y130X recombinant enzymes were incubated with oLB1609 (C oligo, top panel) and oLB1612 (5mC oligo, bottom panel) at 37° C. for 2 hours. NEB APOBEC3A enzyme was purchased from NEBNext™ Enzymatic Methyl-seq Kit (Catalog #E7120). Wild type APOBEC3A deaminated 5mC and C substrates to completion, consistent with previous literature. Different mutants exhibited a wide range of reactivities towards 5mC and C substrates, with some showing preference towards either substrate. Remarkably, APOBEC3A(Y130A) (first box) deaminated 5mC substrates almost completely (94.2%), while it deaminated the corresponding C substrate to a minor extent (29.4%). Other mutants, such as APOBEC3A(Y130P) and APOBEC3A(Y130T), also exhibited more complete deamination of the 5mC than C substrate, albeit to a lesser extent than APOBEC3A(Y130A). In contrast, APOBEC3A(Y130L) (second box) deaminated approximately half of the C substrate (56%), but almost none of the 5mC substrate (6.8%). The deaminase activity of all APOBEC3A(Y130X) mutants is quantified and summarized in FIG. 8, FIG. 9, and FIG. 10.

Because these SwaI assays were performed as a single endpoint measurement (2 hour), it could be possible that the respective deamination reactions had already saturated. A time course analysis of APOBEC3A(Y130A) deaminase activity was therefore performed. The extent of C and 5mC deamination was monitored at 0, 5, 10, 30, 60 and 120 minutes by incubation of ~10-20 pM of APOBEC(Y130A) with 500 nM C and 5mC oligonucleotide substrate (FIG. 10). A greater difference in the extent of 5mC versus C deamination was observed at t≤30 min.

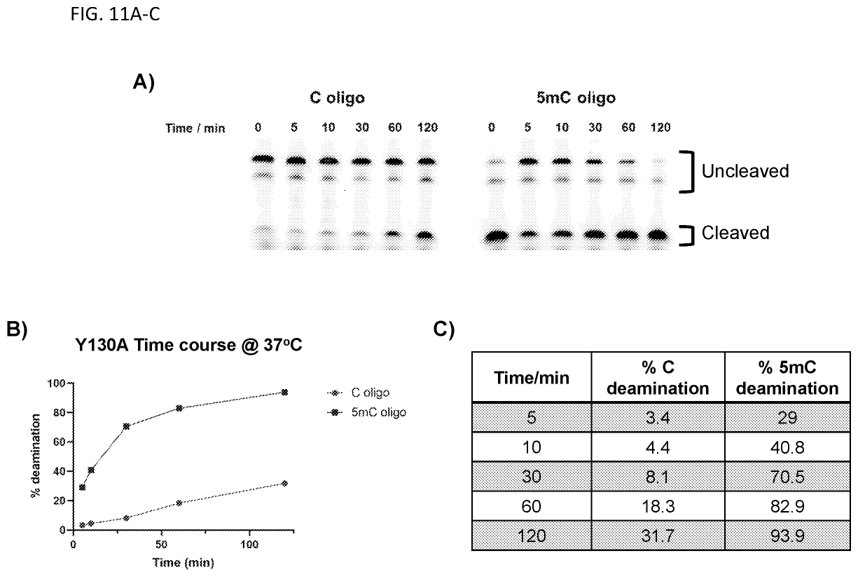

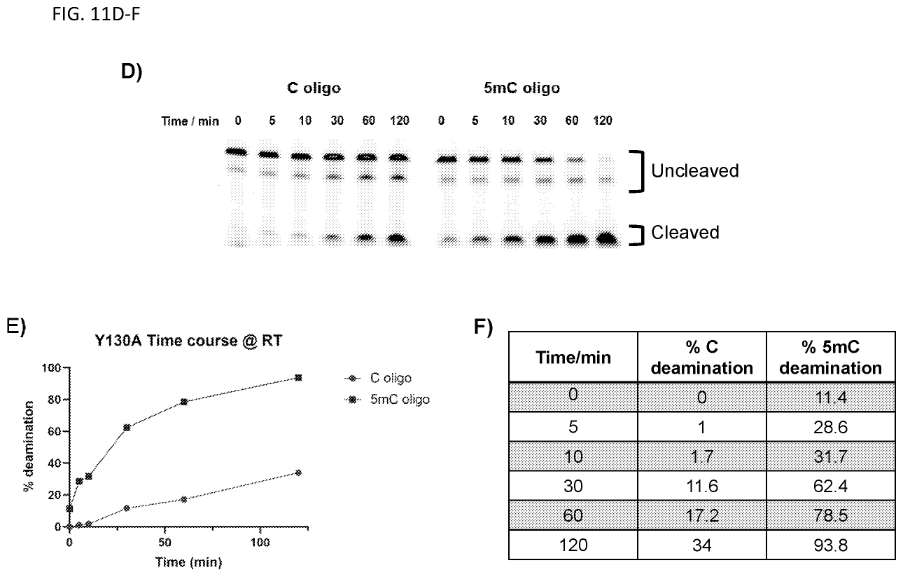

The kinetics of deamination by wild type APOBEC3A and mutant APOBEC3A(Y130A) were quantitively compared. The initial deamination reaction velocity was measured at a range of DNA substrate concentrations and used to construct Michaelis-Menten curves for 5mC and C substrates, respectively. The resulting Km and Kcat values were then derived from these data. The catalytic efficiency of APOBEC3A(Y130A) was ~100-fold higher on 5mC than C substrates (FIG. 11), corroborating the endpoint SwaI assays shown in FIG. 8, FIG. 9, and FIG. 10.

### Example 4

### Purification of APOBEC3A(Y130A-Y132H) Double Mutant Protein

APOBEC3A(Y130A-Y132H) protein was expressed in BL21(DE3) cells, purified using Ni-NTA agarose beads, and desalted/concentrated using spin columns to storage buffer (50 mM Tris pH 7.5, 200 mM NaCl, 5% (v/v) glycerol, 0.01% (v/v) Tween-20, 0.5 mM DTT). This yielded APOBEC3A(Y130A-Y132H) mutant protein preparations with 90-95% purity, as judged by SDS-PAGE analysis (FIG. 7B).

### Example 5

### DNA Deaminase Activity of APOBEC3A(Y130A-Y132H) Double Mutant Protein

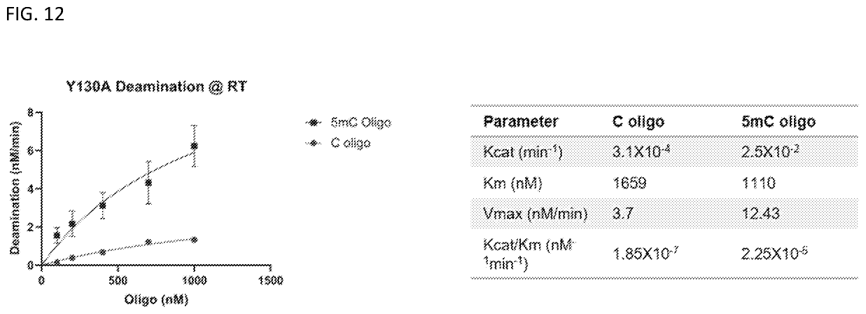

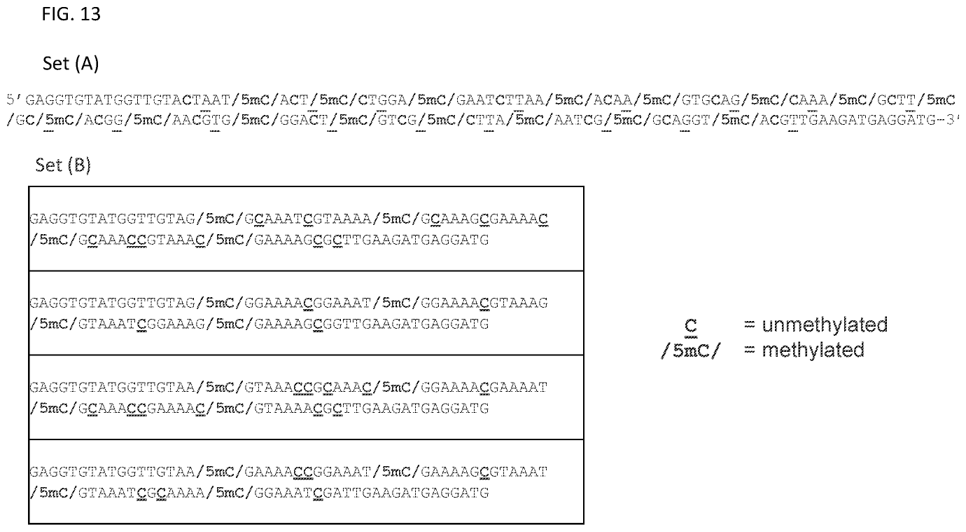

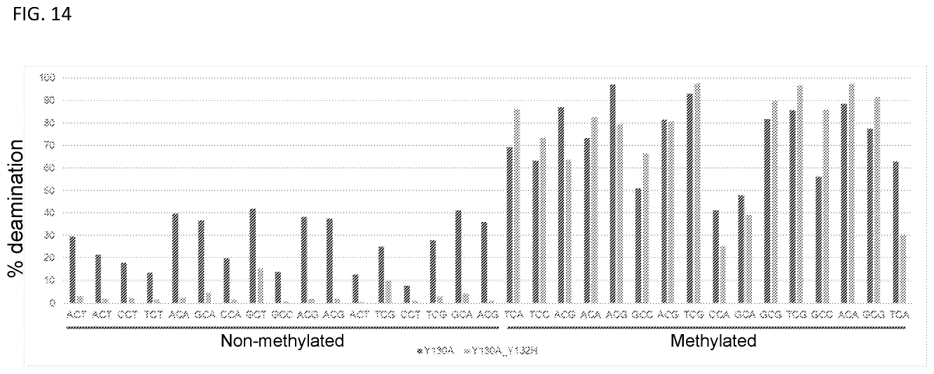

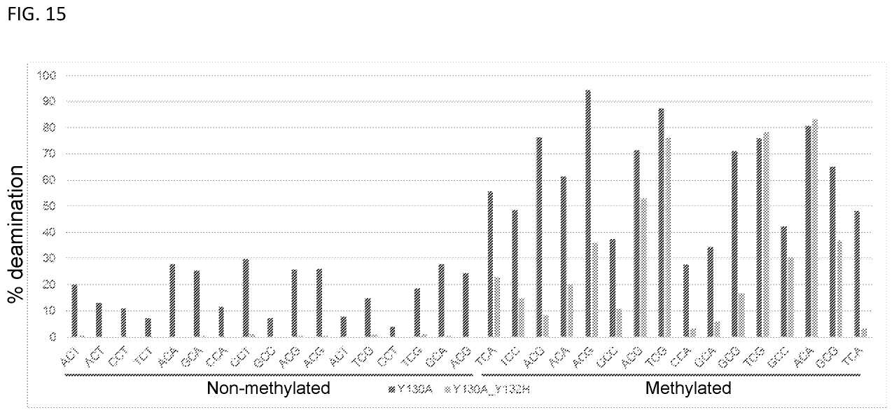

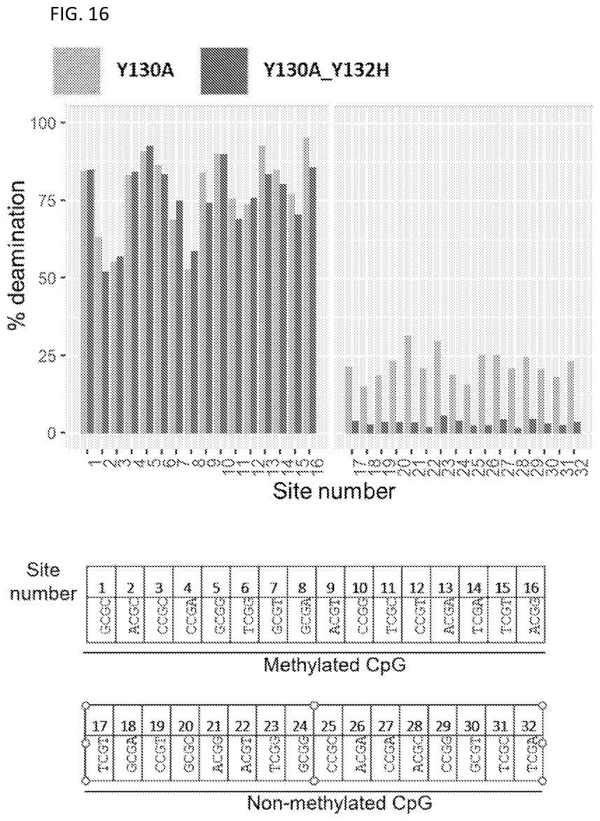

The deaminase activity of purified APOBEC3A(Y130A-Y132H) double mutant protein was then analyzed using the SwaI assay, with a 37° C./2 hour reaction time and NEB APOBEC3A as positive control. The conditions used were the same as described in Example 3 with the exception that the SwaI assay used reaction conditions of 40 mM sodium acetate pH 5.2, 37° C. for 1 hour to 16 hours. The DNA substrates are shown in FIG. 12. After the deaminase reaction the deaminated oligo substrates were PCR-amplified, sequenced, and the number of C and 5mC deamination events per read were counted. The DNA oligonucleotide substrates used for experiments with APOBEC3A(Y130A-Y132H) are shown in FIG. 12. APOBEC3A(Y130A-Y132H) exhibited higher levels of deamination at all methylated sites compared to unmethylated sites. This was consistent across both CpG and non-CpG contexts, and was robust to variation in reaction time (FIG. 13, 14). The difference in deamination level between methylated and unmethylated sites was markedly higher for APOBEC3A(Y130A-Y132H) than APOBEC3A(Y130A), indicating that APOBEC3A(Y130A-Y132H) achieves better discrimination of methylated sites than APOBEC3A(Y130A). In addition, APOBEC3A(Y130A-Y132H) deaminated methylated sites more efficiently than unmethylated sites across all xCpGx motifs (FIG. 15).

### Example 6

### Purification of APOBEC3A(Y130W) Mutant Protein

Recombinant human His-tagged APOBEC3A(Y130W) protein was expressed in Escherichia coli BL21(DE3) cells, purified using Ni-NTA affinity chromatography, and desalted/concentrated using spin columns to storage buffer (50 mM Tris pH 7.5, 200 mM NaCl, 5% (v/v) glycerol, 0.01% (v/v) Tween-20, 0.5 mM DTT).

### Example 7

### DNA Deaminase Activity of APOBEC3A(Y130W) Mutant Protein

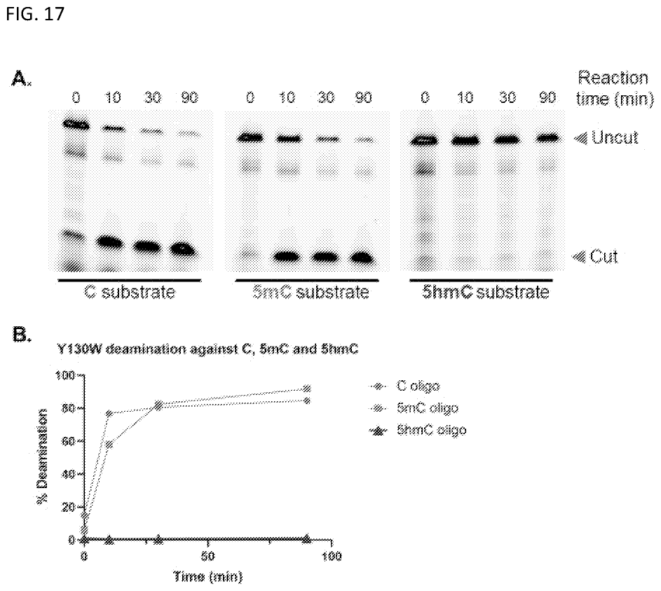

Using the SwaI assay, we measured the activity of APOBEC3A(Y130W) on C, 5mC, and 5hmC substrates across a 90 min time interval. While the majority of C and 5mC was deaminated, no detectable activity was observed for 5hmC. (FIG. 17).

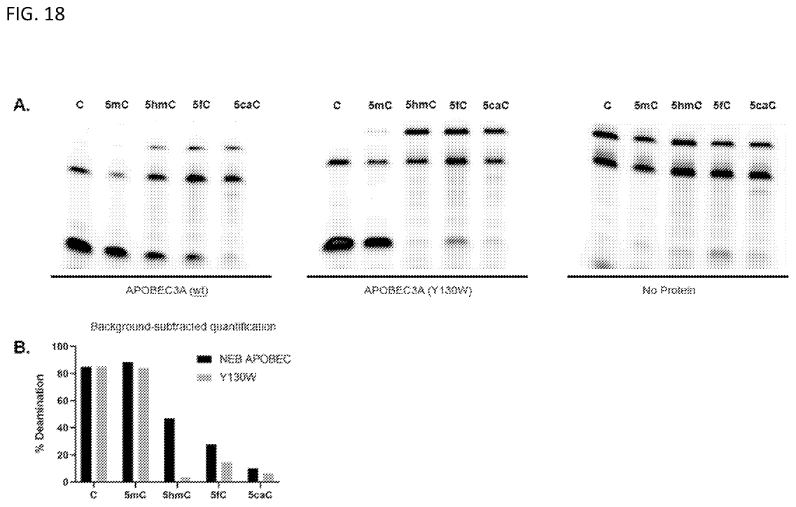

To better understand the substrate preferences of APOBEC3A(Y130W), we assayed deaminase activity on C, 5mC, and all oxidized derivatives of 5mC (FIG. 18). The SwaI restriction enzyme assay was performed with a 90 min reaction time to measure deaminase activities of APOBEC3A wild type and Y130W mutant enzymes. A no-protein control was included to account for potential degradation of oligonucleotide substrates and account for non-specific activity of SwaI during the course of the assay. APOBECY130A(Y130W) showed no detectable deamination of 5hmC, 5fC, and 5caC, while wild type APOBEC retained significant deaminase activity towards 5hmC and showed residual activity on 5fC and 5caC. Both enzymes deaminated C and 5mC efficiently.

### Example 8

### Detection of 5mC

The following example generally follows the methods described in Schutsky et al. Nature biotechnology, 10.1038/nbt.4204. 8 Oct. 2018, doi:10.1038/nbt.4204. Genomic DNA (gDNA) from an organism suspected of having 5hmC is provided. 20 ng of the gDNA mixture is then treated as follows.

In order to provide single-stranded DNA and facilitate deamination activity, 1 μL of DMSO is added and the sample is denatured at 95° C. for 5 min and snap cooled by transfer to a PCR tube rack pre-incubated at −80° C. Before thawing, reaction buffer is overlaid to a final concentration of 20 mM MES pH 6.0+0.1% Tween, and an altered cytidine deaminase described herein is added to the sample to a final concentration of 5 pM in a total volume of 10 μL. The deamination reaction is incubated under linear ramping temperature conditions from 4-50° C. over 2 hrs.

After deamination, the sample is prepared for Illumina sequencing using the Accel Methyl-NGS kit (Swift Biosciences). Specifically, the reactions are purified using Zymo Oligo Clean and Concentrator Kit and eluted in 15 μL elution buffer (10 mM Tris pH 8.0). The Accel-NGS Methyl-Seq kit (Swift Biosciences) is then used for library preparation of single-stranded DNA according to manufacturer's instructions. After purification of the library, 1-5 ng amplified DNA is run on an Agilent Bioanalyzer High Sensitivity DNA Chip to confirm proper library fragment sizes. The resulting ACE-Seq library is sequenced at 1.9 pM with single-end mode on a NextSeq 500 sequencer (Illumina) using the NextSeq 500/500 High Output kit v2 (150 cycles).

Sequencing data from the sample is analyzed to detect 5mC at 500 CpG alleles suspected of 5mC modification. Those CpG sites that sequence as thymidine are characterized as likely containing 5mC modifications in the original sample.

### Example 9

### Detection of 5hmC

The following example generally follows the methods described in Schutsky et al., Nature biotechnology, 10.1038/nbt.4204. 8 Oct. 2018, doi:10.1038/nbt.4204. Genomic DNA (gDNA) from an organism suspected of having 5hmC is provided. Two aliquots of 20 ng each are provided. One of the aliquots of 20 ng of the gDNA mixture is glucosylated using UDP-glucose and T4 β-glucosyltransferase (βGT, NEB). Specifically, 0.5 μL of 10X Cutsmart Buffer (NEB), 0.1 μL of 50×UDP-Glucose, 0.5 μL of T4 βGT (NEB) are combined with 20 ng of gDNA and water is added for a total volume of 5 μL. A control reaction is assembled using the other 20 ng aliquot of gDNA, and the reaction components are mixed as described above, but with 0.5 μL water added in place of T4 βGT. The reactions are incubated at 37° C. for 1 hr.

In order to provide single-stranded DNA and facilitate deamination activity, 1 μL of DMSO is added and the samples are denatured at 95° C. for 5 min and snap cooled by transfer to a PCR tube rack pre-incubated at −80° C. Before thawing, reaction buffer is overlaid to a final concentration of 20 mM MES pH 6.0+0.1% Tween, and an altered cytidine deaminase is added to each sample to a final concentration of 5 pM in a total volume of 10 μL. The deamination reactions are incubated under linear ramping temperature conditions from 4-50° C. over 2 hrs.

After deamination, the samples are prepared for Illumina sequencing using the Accel Methyl-NGS kit (Swift Biosciences). Specifically, the reactions are purified using Zymo Oligo Clean and Concentrator Kit and eluted in 15 μL elution buffer (10 mM Tris pH 8.0). The Accel-NGS Methyl-Seq kit (Swift Biosciences) is used for library preparation according to manufacturer's instructions. After purification of the libraries, 1-5 ng amplified DNA is run on an Agilent Bioanalyzer High Sensitivity DNA Chip to confirm proper library fragment sizes. The resulting ACE-Seq libraries are sequenced at 1.9 pM with single-end mode on a NextSeq 500 sequencer (Illumina) using the NextSeq 500/500 High Output kit v2 (150 cycles).

Sequencing data from the sample and control are analyzed to detect 5hmC and 5mC at 500 CpG alleles suspected of 5hmC modification. Specifically, any CpG sites that are sequenced as cytosine in the control sample are compared to the same CpG sites in the sample treated with βGT. Those sites that sequence as thymidine are characterized as likely containing 5hmC modifications in the original sample.

### Example 10

### Detection of 5mC and 5hmC

The following example generally follows the methods described in Schutsky et al. Nature biotechnology, 10.1038/nbt.4204. 8 Oct. 2018, doi:10.1038/nbt.4204. Genomic DNA (gDNA) from an organism suspected of having 5mC and 5hmC is provided. Two aliquots of 20 ng each are provided. Each 20 ng aliquot of the gDNA mixture is then treated as follows.

In order to provide single-stranded DNA and facilitate deamination activity, 1 μL of DMSO is added and the sample is denatured at 95° C. for 5 min and snap cooled by transfer to a PCR tube rack pre-incubated at −80° C. Before thawing, reaction buffer is overlaid to a final concentration of 20 mM MES pH 6.0+0.1% Tween. To one of the aliquots, and the altered cytidine deaminase (Y130W) described herein is added to the sample to a final concentration of 5 pM in a total volume of 10 μL. To the other aliquot, wild type cytidine deaminase is added to the sample to a final concentration of 5 pM in a total volume of 10 μL. The deamination reactions are incubated under linear ramping temperature conditions from 4-50° C. over 2 hrs.

After deamination, the samples are prepared for Illumina sequencing using the Accel Methyl-NGS kit (Swift Biosciences). Specifically, the reactions are purified using Zymo Oligo Clean and Concentrator Kit and eluted in 15 μL elution buffer (10 mM Tris pH 8.0). The Accel-NGS Methyl-Seq kit (Swift Biosciences) is then used for library preparation of single-stranded DNA according to manufacturer's instructions. After purification of the library, 1-5 ng amplified DNA is run on an Agilent Bioanalyzer High Sensitivity DNA Chip to confirm proper library fragment sizes. The resulting APOBEC-coupled epigenetic sequencing (ACE-seq) library is sequenced at 1.9 pM with single-end mode on a NextSeq 500 sequencer (Illumina) using the NextSeq 500/500 High Output kit v2 (150 cycles).

Sequencing data from the sample is analyzed to detect 5mC at 500 CpG alleles suspected of 5mC and/or 5hmC modification. Those CpG sites that sequence predominantly as thymidine in the wild type sample but that sequence predominantly as cytosine in the Y130W sample are characterized as likely containing 5hmC modifications in the original sample.

### Example 11

### Detection of 5mC and 5hmC

Human genomic DNA is combined with fully unmethylated lambda control DNA (New England Biolabs) and enzymatically CpG methylated pUC19 control DNA and mechanically sheared to give fragments of approximately ~300 bp. This sheared DNA (10-100 ng) is then subjected to end-repair, A-tailing and adapter ligation according to standard Illumina library preparation procedures. The sample is then split into 2 aliquots to enable differential treatment.

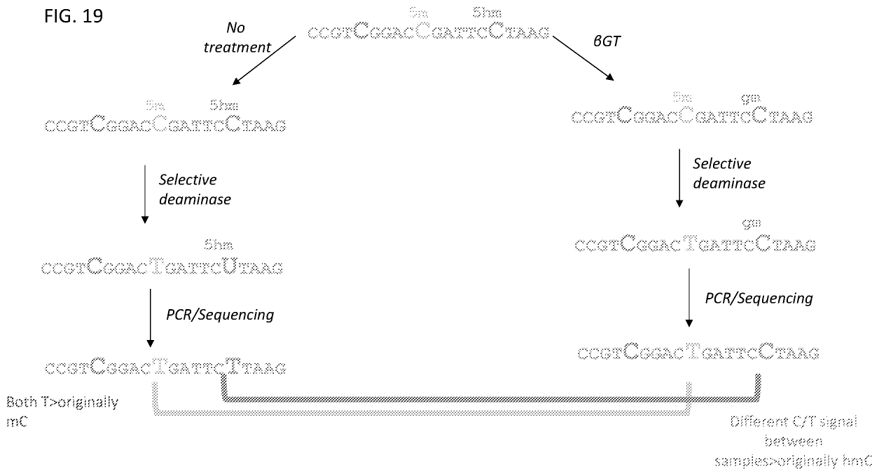

One of the aliquots of the adapter-ligated DNA is glucosylated using UDP-glucose and T4 β-glucosyltransferase (OGT, NEB). Specifically, the sample is treated with 0.5 U/uL of T4 βGT (NEB) in 1× CutSmart Buffer (NEB) with 40 pM UDP-Glucose at 37° C. for 3 hr. A control reaction is assembled using the other aliquot of the DNA sample, omitting the T4 βGT enzyme. Subsequently, the samples are optionally SPRI purified and then denatured via incubation in 0.02 N sodium hydroxide at 50° C. for 10 minutes. Subsequently, ssDNA samples are enzymatically deaminated in 50 mM Bis-Tris (pH 6.5), 10 μg/mL RNAse A with the cytidine deaminase (200 nM) for 25 minutes at 37 C. The libraries are then PCR amplified using unique-dual indexing primers and Q5U (New England Biolabs) using 9 cycles of PCR. Samples are sequenced on a NovaSeq6000 and analysis is performed with DRAGEN Methylation Pipeline. The methylation calls between the two treatment conditions (+/−T4 βGT) are compared in order to determine which sites were hydroxymethylated. Bases that are detected as methylated in both sample treatments are assigned mC, whereas bases that are detected as methylated in the −βGT condition and unmethylated in the +βGT condition are assigned hmC (FIG. 19).

### Example 12

### Generalized Method for Creation of Methylation Sequencing Libraries

Sheared DNA or cfDNA (10-200 ng) was first subjected to end-repair, A-tailing and adapter ligation according to standard library preparation procedures. Suitable adapters for this method can have unmodified C's or pyrrolo-C modifications to disfavor deamination of the adapter sequence. The adapter ligated DNA was then denatured to ssDNA. Subsequently, this ssDNA sample was enzymatically deaminated in a buffered solution with an engineered deaminase (APOBEC3A-Y130A-Y132H, 50 nM-1000 nM) for 5 minutes to 3 hours at incubation temperatures ranging between 20° C. to 55° C. Deaminated libraries were optionally SPRI purified before PCR amplification using unique-dual indexing primers, using either a uracil tolerant polymerase (for instance, Q5U®, KapaU™) or a uracil intolerant polymerase (for instance, Q5 HiFi®, KAPA HiFi™) using 9 to 12 cycles of PCR. Libraries were then sequenced on a NextSeq550 or NovaSeq 6000 and analysis was performed with DRAGEN Methylation Pipeline.

### Methods for Denaturation

A variety of methods for denaturation known to those skilled in the art. These include, but are not limited to: (i) heating in the presence of NaOH or high pH buffer at a moderate temperature (e.g., 0.02 N sodium hydroxide at 50° C. for 10 minutes); (ii) heating to high temperatures (e.g., 95° C. for 10 minutes); (iii) heating in the presence of DMF (e.g., 50% DMF at 95° C. for 10 minutes); (iv) heating in the presence of formamide (e.g., 50% formamide at 95° C. for 10 minutes); and (v) heating in the presence of DMSO (e.g., 50% DMSO at 95° C. for 10 minutes).

### Suitable Buffers for Deamination

Typically, the deamination buffer is added to the denatured DNA sample, and thus any additives to promote denaturation will be present at a lower (diluted) concentration in the deamination reaction.

Several buffer systems have been identified in which deamination can occur. The buffer 50 mM Bis Tris, pH 6.5 can be used, and other pH levels between 5-7.5 may be feasible. Additionally, other buffer strengths (concentrations) may be feasible. The skilled person will recognize that if NaOH is used for denaturation, adequate buffer strength/pH can be used to ensure that the final pH is within the ideal range for the deaminase. Alternative buffer systems (e.g., tcichemicals.com/US/en/c/10367), including MES, may also provide good results.

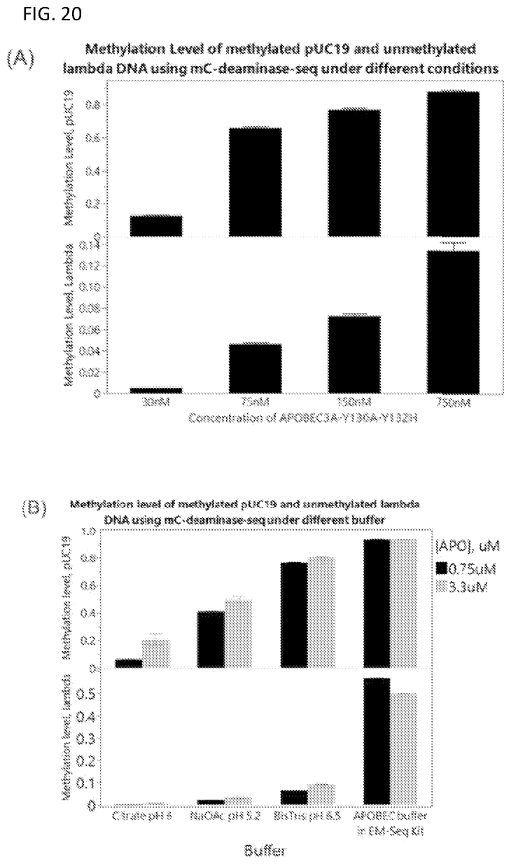

In some embodiments, other buffer systems have been shown to have lower performance when used for the deamination of a genomic DNA (gDNA) sample. Examples include citrate buffer and sodium acetate buffer (FIG. 20). These buffer systems may have lower performance due to sodium content, which is believed to be detrimental to activity of the altered deaminase. APOBEC3A is a zinc-dependent enzyme (Marx et al., Scientific Reports 5, no. December (2015): 1-9. https://doi.org/10.1038/srep18191), and thus the presence of metal cations (e.g., sodium, magnesium) may generally be detrimental. Another explanation is that metal cations can impact the formation of secondary structure in nucleic acids (Einert et al., Biophysical Journal 100, no. 11 (2011): 2745-53, doi.org/10.1016/j.bpj.2011.04.038), which may also impact activity of the deaminase on the substrate, since APOBEC3A cannot deaminate Cs in double stranded DNA such as hairpins. Therefore, it would be expected that any buffer system can be used that would maintain pH in the desired range, and in some embodiments buffers with minimal sodium can be used.

### Selectivity of Altered Cytidine Deaminases on Genomic DNA

The altered cytidine deaminase maintains some activity for unmethylated cytidine deamination, thus it can be useful to tune the activity to maintain the desired selectivity for methylated cytosines. For example, high concentrations of the enzyme, high incubation times, or buffers that promote high activity of the enzyme may lead to undesired levels of unmethylated cytidine deamination. Typically, enzyme concentration may be from 50 nM to 1000 nM), the reaction can occur from 5 minutes to 3 hours at incubation temperatures ranging from 20° C. to 55° C. In particular, it may be helpful to tune enzyme concentration based on the purification method of the deaminase and how much activity a given enzyme preparation contains.

As an example, libraries were prepared, deaminated according to the general methods described above, and sequenced. Variation of the concentration of APOBEC-Y130A-Y132H led to observable differences in the level of activity and level of off-target cytidine deamination (FIG. 20A). Furthermore, although one may expect a commercial buffer for APOBEC activity to work well for this application, testing of the APOBEC buffer included in the EM-Seq™ kit (New England Biolabs) led to an undesirable high level of activity on the cytidine substrate, resulting in elevated conversion of unmethylated C nucleobases in lambda DNA (FIG. 20B).

### Methods for Determining Suitable Conditions for Deamination

To determine whether conditions were suitable for selective deamination on genomic DNA, a DNA mixture containing NA12878 (human) DNA, fully CpG methylated pUC19, and fully unmethylated lambda was prepared. After adapter ligation, the samples were subjected to various deamination conditions and prepared as a sequencing library, using the general methods described above. The methylation level of pUC19 and lambda was used to select conditions with desired 5mC activity and selectivity.

### Example 13

Specific Examples of Conditions used for Preparation and Deamination of Libraries by Altered Cytidine Deaminase

In all methods described below in Examples 13-20, the altered cytidine deaminase refers specifically to APOBEC3A-Y130A-Y132H. A variety of human genomic DNA samples were tested for different application assessments.

Method A: Human genomic DNA was combined with fully unmethylated lambda control DNA and enzymatically CpG methylated pUC19 control DNA and mechanically sheared to give fragments of approximately ~300 bp. This sheared DNA (10-100 ng) was then subjected to end-repair, A-tailing, and adapter ligation according to standard Illumina library preparation procedures. The adapter ligated DNA was denatured via incubation in 0.02 N sodium hydroxide at 50° C. for 10 minutes. Subsequently, ssDNA samples were enzymatically deaminated in 50 mM Bis-Tris (pH 6.5), 10 μg/mL RNAse A with the cytidine deaminase (200 nM) for 25 minutes at 37° C. The libraries were then PCR amplified using unique-dual indexing primers and Q5U (New England Biolabs) using 9 cycles of PCR. Samples were sequenced on a NovaSeq6000 and analysis was performed with DRAGEN Methylation Pipeline.

Method B: Human genomic DNA was combined with fully unmethylated lambda control DNA and enzymatically CpG methylated pUC19 control DNA and mechanically sheared to give fragments of approximately ~300 bp. This sheared DNA (10-100 ng) was then subjected to end-repair, A-tailing, and adapter ligation according to standard Illumina library preparation procedures. The adapter ligated DNA was denatured via incubation in 0.02 N sodium hydroxide at 50° C. for 10 minutes. Subsequently, ssDNA samples were enzymatically deaminated in 50 mM Bis-Tris (pH 6.5), 10 μg/mL RNAse A with the altered cytidine deaminase (200 nM) for 15 minutes at 37° C. The libraries were then PCR amplified using unique-dual indexing primers and Q5U (New England Biolabs) using 9 cycles of PCR. Samples were sequenced on a NovaSeq6000 and analysis was performed with DRAGEN Methylation Pipeline.

Method C: Human genomic DNA was combined with fully unmethylated lambda control DNA and enzymatically CpG methylated pUC19 control DNA and mechanically sheared to give fragments of approximately ~300 bp. This sheared DNA (10-100 ng) was then subjected to end-repair, A-tailing, and adapter ligation according to standard Illumina library preparation procedures. The adapter ligated DNA was denatured via incubation in 0.02 N sodium hydroxide at 50° C. for 10 minutes. Subsequently, ssDNA samples were enzymatically deaminated in 50 mM Bis-Tris (pH 6.5), 10 μg/mL RNAse A with the altered cytidine deaminase (200 nM) for 15 minutes at 37° C. The libraries were then PCR amplified using unique-dual indexing primers and Q5 HiFi (New England Biolabs) using 9 cycles of PCR. Samples were sequenced on a NovaSeq6000 and analysis was performed with DRAGEN Methylation Pipeline.

Method D: Human genomic DNA was combined with fully unmethylated lambda control DNA and enzymatically CpG methylated pUC19 control DNA and mechanically sheared to give fragments of approximately ~300 bp. This sheared DNA (10-100 ng) was then subjected to end-repair, A-tailing and adapter ligation according to standard Illumina library preparation procedures. The adapter ligated DNA was denatured via incubation in 0.02 N sodium hydroxide at 50° C. for 10 minutes. Subsequently, ssDNA samples were enzymatically deaminated in 50 mM Bis-Tris (pH 6.5) with the altered cytidine deaminase (200 nM) for 25 minutes at 37° C. The libraries were then PCR amplified using unique-dual indexing primers and Q5U (New England Biolabs) using 9 cycles of PCR. Samples were sequenced on a NovaSeq6000 and analysis was performed with DRAGEN Methylation Pipeline.

### Use of RNAse A

Literature suggests that RNAses may increase the activity of cytidine deaminases by removing contaminating RNA (Bransteitter et al., Proceedings of the National Academy of Sciences of the United States of America 100, no. 7 (2003): 4102-7. https://doi.org/10.1073/pnas.0730835100).

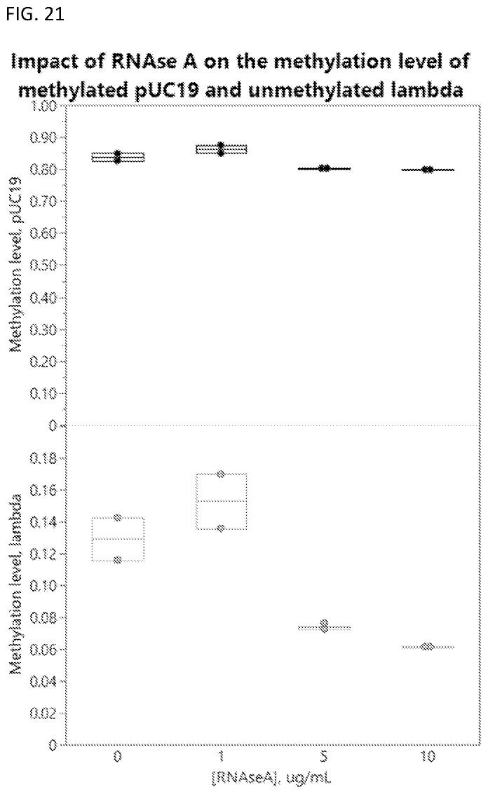

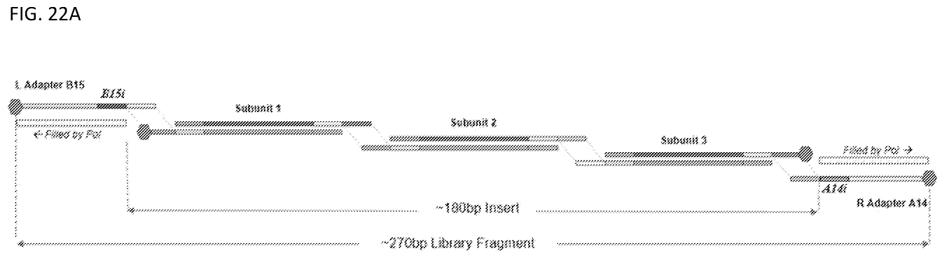

However, testing of RNAse A showed, contrary to this hypothesis, that RNAse A reduced activity of the altered cytidine deaminase, with a more pronounced impact to the reduction of off-target cytosine deamination, thus leading to the result of more 5mC selectivity (FIG. 21).

### Example 14

### Deamination of 5hmC

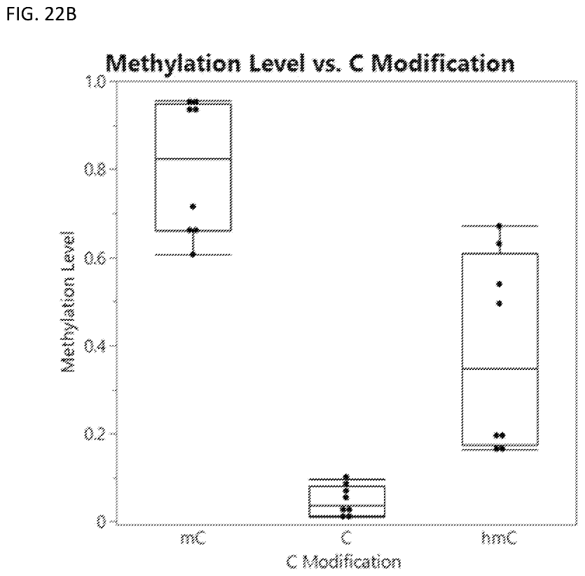

To assess 5hmC deamination, oligos were designed with C, 5mC, or 5hmC modifications in defined contexts. These oligos were assembled together using ligation, and oligos were built such that the resulting ligated fragment would contain handles needed for subsequent amplification and sequencing (FIG. 22A). The assembled control oligo was spiked into an adapter-ligated DNA library, and treated according to Method A (Example 12). Analysis of the reported methylation from this control oligo showed that 5mC was the most preferred substrate for APOBEC Y130A-Y132H, however there was still significant activity on 5hmC (FIG. 22B).

### Example 15

### Determination of Methylation on CpG Islands

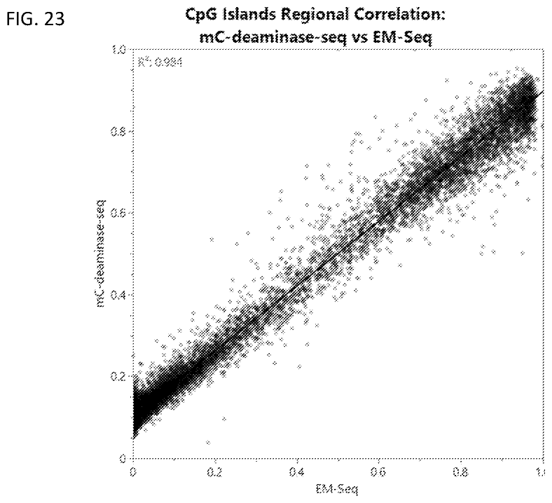

Using NA12878 gDNA, libraries were prepared and deaminated according to Method A (Example 13). A comparative dataset was also generated using EM-Seq™ conversion (New England Biolabs). To assess the methylation performance on the human genome, regional methylation values for CpG islands across the human genome were calculated using methylpy. The per-region methylation values were plotted against the per-region methylation values derived from the EM-Seq™ dataset (FIG. 23). The regional analysis demonstrates high correlation between the use of the Y130A-Y132H deaminase and EM-Seq™, a commercial methylation detection method. This indicates that the 5mC-selective deamination assay described herein can effectively detect and report methylation in CpG islands.

### Example 16

### Variant Calling on Methylated Libraries

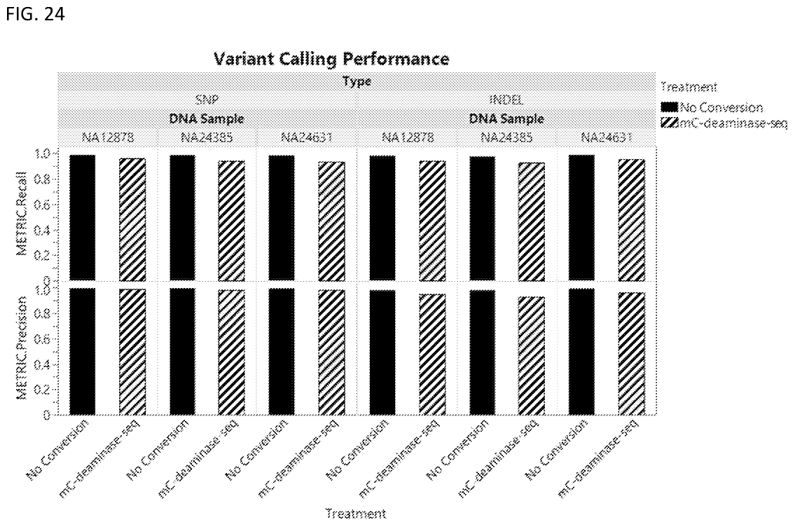

Libraries from human genome samples NA12878, NA24385, and NA24631 were prepared and deaminated according to Method A (Example 13). A comparative dataset was also generated without conversion, by proceeding directly to PCR after adapter ligation. After running variant calling analysis, comparison to truth sets for each genome using hap.py showed that the libraries subjected to the methylation conversion (the altered cytidine deaminase) provided good SNV/indel calling performance, approaching the performance of the no conversion controls (FIG. 24). While SNV/indel calling is discussed here, other types of variant calling, including copy number variation (CNV) and short tandem repeats (STR), and structural variants (SV) are also feasible.

### Example 17

### Differentially Methylated Region (DMR) Calling

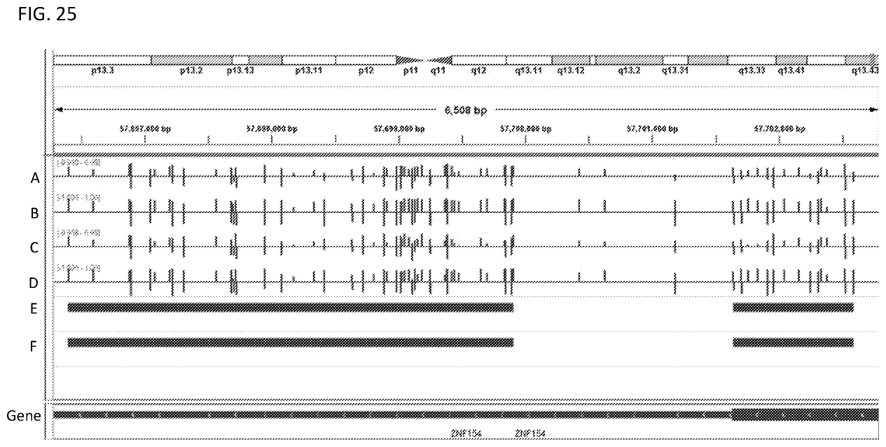

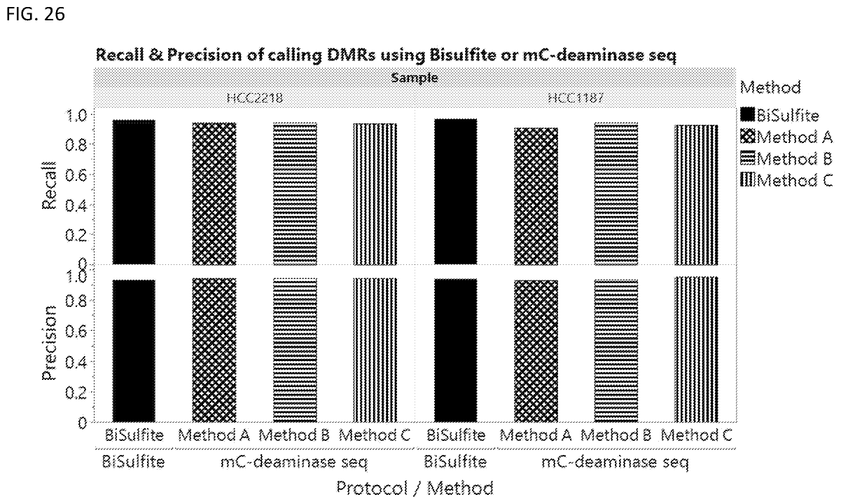

Differential methylation analysis is commonly employed for comparison of methylomes across different diseases, tissues, and cell types (Chen et al., Briefings in Functional Genomics 15, no. 6 (2016): 485-90. https://doi.org/10.1093/bfgp/elw018). Libraries were prepared and deaminated according to Method A (Example 13) using genomic DNA isolated from HCC2218 tumor and normal cell types (CRL-2363D, CRL-2343D, ATCC) and HCC1187 tumor and normal cell types (CRL-2323D, CRL-2322D, ATCC). Comparative dataset was also generated using EM-Seq™ conversion (New England Biolabs) and Bisulfite conversion (EZ DNA Methylation-Gold Kit—Zymo Research). For differential methylation analysis, HOME, a program for identifying DMRs, was used (Srivastava et al., BMC Bioinformatics 20, no. 1 (2019): 1-15, doi.org/10.1186/s12859-019-2845-y). Comparisons of the methylation between tumor and normal samples were performed for each methylation assay. As shown in FIG. 25, the assay was able to detect a relevant differentially methylated region in the ZNF-154 gene (Almeida et al., BMC Cancer 19, no. 1 (2019): 1-12. https://doi.org/10.1186/s12885-019-5403-0). Furthermore, quantitative assessment of DMR calling performance using Methods A, B, and C (Example 13) showed that the altered cytidine deaminase-based methods detected expected DMRs with high precision and recall (FIG. 26).

### Example 18

### Methylation at Promoters

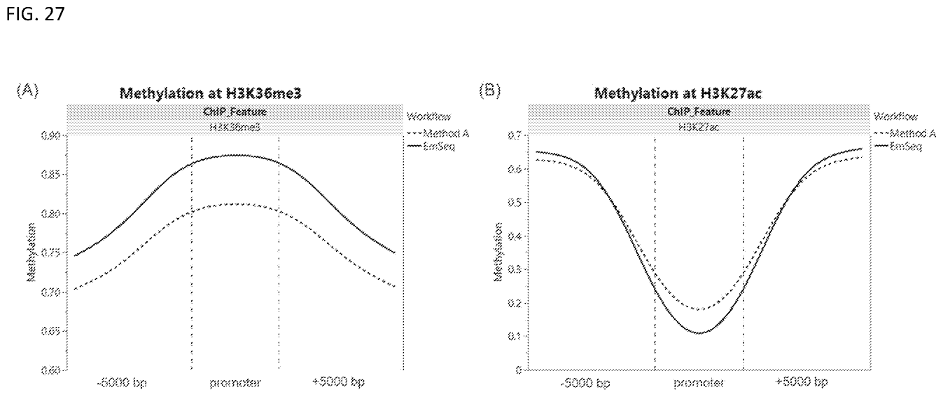

The methylation status of promoter regions can be correlated to both histone status as well as gene expression activity. In order to assess whether the altered cytidine deaminase assay could be used to probe the methylation status of promoters, libraries from human genome samples NA12878 were prepared and deaminated according to Method A (Example 13). Comparative libraries were also generated with EM-Seq™ conversion. The methylation level at known histone marker sites, including H3K36me3 and H3K27ac were quantified from the resulting data. H3K36me3 sites are expected to be inactive promoters with high histone and DNA methylation, while H3K27ac sites are expected to be active promoters with high histone acetylation and low DNA methylation. The altered cytidine deaminase assay was able to report these methylation trends, as expected (FIG. 27).

### Example 19

### Detecting Tumor Sample in a Background of Normal Sample

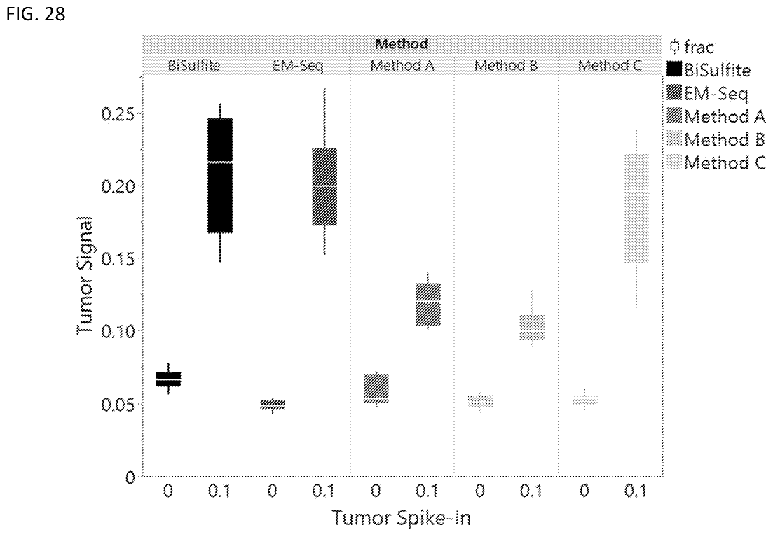

Libraries from human genome samples with either HCC2218 normal DNA or a 10% spike-in of HCC2218 tumor DNA into a background of HCC2218 normal DNA were made with appropriate adapters. Assessment of methylation level for the 10% spike-in samples were made in comparison to established methylation methods on CpGs within regions also targeted by the PanSeer cancer panel (Chen, et al. Nature Communications 11, no. 1 (2020): 1-10, https://doi.org/10.1038/s41467-020-17316-z). Libraries subjected to the altered cytidine deaminase reflected methylation changes between the HCC2218 normal DNA and a 10% spike-in of HCC2218 tumor DNA into HCC2218 normal DNA. Methods A, B, and C (Example 13) produced higher tumor signal in the 10% tumor spike-in sample compared to a sample with no tumor DNA spiked in (FIG. 28). This reflects the ability of the altered cytidine deaminase to detect low levels of tumor DNA, which could be applied to cell free DNA (cfDNA) samples for early cancer detection (Jamshidi et al., Cancer Cell 40, no. 12 (2022): 1537-1549.e12. doi.org/10.1016/j.ccell.2022.10.022.) or minimal residual disease (MRD) testing (Jin et al., Proceedings of the National Academy of Sciences of the United States of America 118, no. 5 (2021): 1-8, doi.org/10.1073/pnas.2017421118.).

### Example 20

### Enrichment

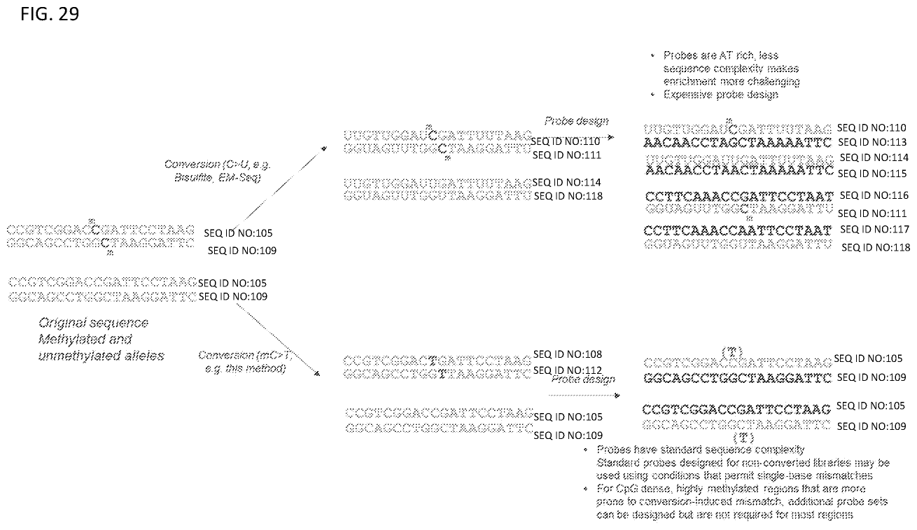

Most on-market methylation assays convert C>T, leading to extensive C>T conversion and low complexity sequences. This makes enrichment very challenging, because probes must be designed for all possible methylation states/strands (www.twistbioscience.com/sites/default/files/resources/2020-02/AppNote_Methylation-singles.pdf). This makes hybrid-enrichment sequencing of methyl-converted libraries expensive and more difficult to optimize. An alternative is amplicon-based targeted sequencing; however, advantages of hybridization-based targeted sequencing include the ability to easily sequence both the un-enriched (whole genome sequence (WGS)) and enriched sample and target sequencing on multiple contiguous regions of interest (Singh et al., Diagnostics (Basel, Switzerland) 12, no. 7 (Jun. 24, 2022): 1539. doi.org/10.3390/diagnostics12071539.). Because of the 5mC>T conversion enabled by the altered cytidine deaminase, enrichment of methyl-converted libraries can be accomplished using less complex panels while still maintaining high quality methylation information (FIG. 29). Standard probe designs prepared for non-converted libraries may be used, as only a small percentage of cytosines are methylated and are expected to be converted. Furthermore, hybridization probes tolerate mismatches (Paskey et al., BMC Genomics, 20(1). doi.org/10.1186/S12864-019-5543-2), and thus some methylation conversion can be tolerated by standard panels. In the case of highly CpG dense, methylated regions of the genome, it may be beneficial to design extra probes that account for mismatches to optimize performance.

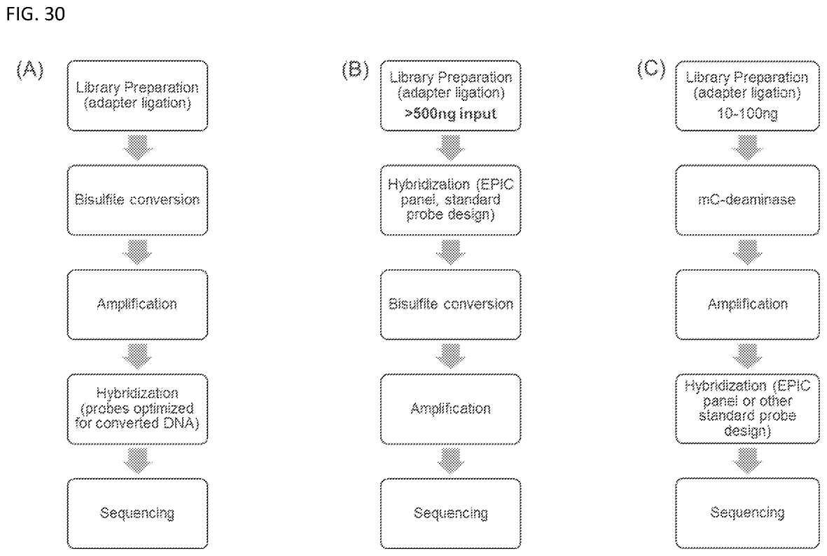

In order to assess enrichment performance, the Illumina Methyl Capture EPIC panel, a probe panel typically employed prior to bisulfite-based conversion, was utilized. This panel is designed to target unconverted DNA due to the challenges with enrichment of converted DNA, as discussed above. However, the Illumina Methyl Capture EPIC kit requires high inputs (500 ng) due to enrichment upstream of the conversion step. In contrast, in the example below, samples were converted, amplified, and then enriched, enabling lower inputs (FIG. 30A-C).

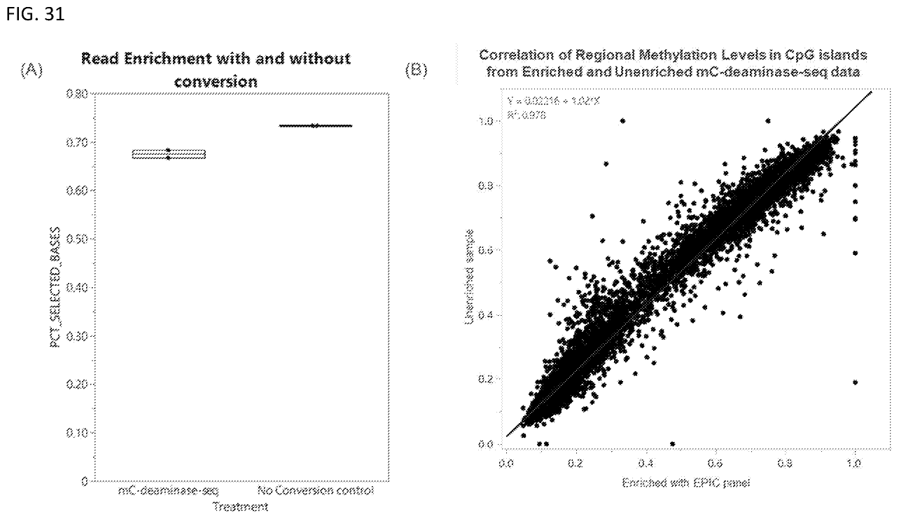

Libraries containing NA12878 genomic DNA were prepared according to Method A (Example 13). As a control, libraries were also prepared that were not deaminated (no conversion control). Following conversion and amplification, libraries were sequenced to generate whole genome methylation control data. Samples were also enriched according to the protocols set forth in the RNA Prep with Enrichment Reference Guide (support.illumina.com/content/dam/illumina-support/documents/documentation/chemistry_documentation/illumina_prep/RNA/illumina-rna-prep-reference-guide-1000000124435-03.pdf) using the Illumina Methyl Capture EPIC panel. Read enrichment was assessed using Picard CollectHsMetrics, showing that the deaminated libraries achieved similar read enrichment compared to non-converted libraries (FIG. 31A). CpG island methylation was assessed using the methods described in Example 15. Comparison of sequencing data from unenriched libraries to the enriched libraries showed that the methylation levels reported with and without enrichment showed good agreement, suggesting enrichment can be used to target and report methylation data (FIG. 31B).

### Example 21

Altered Cytidine Deaminase-Mediated Detection of 5mC Loci Via qPCR

5mC to T conversion can be read out by PCR using mismatch-sensitive primers. Upon 5mC to T conversion, these primers can anneal without mismatches to the substrate DNA. An unmethylated DNA substrate is not converted by an altered cytidine deaminase; accordingly, these substrates exhibit mismatches within primer annealing sequences, resulting in poor PCR amplification. Measuring amplification with respect to standard curves using qPCR would yield a quantitative readout of percent methylation at a target locus.

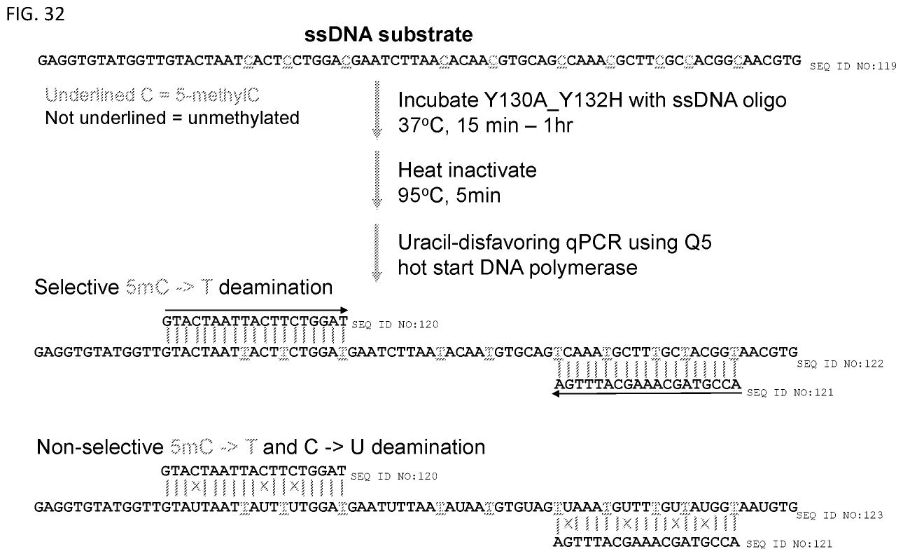

A 78 nt ssDNA oligo containing 5mC and C was incubated with APOBEC3A(Y130A/Y132H) at 37° C. for 15 min, allowing 5mC deamination to occur (FIG. 32). Following this, the deamination reaction was heat-inactivated at 95° C. for 5 min, and a small aliquot was directly added to a qPCR master mix containing PCR buffer, dNTPs, primers, and Q5 hot start DNA polymerase. The resulting mixture was cycled in a standard qPCR instrument. (Note: that Q5 hot start DNA polymerase was used for the qPCR assay as it is selective against uracil-containing templates, thus providing an additional layer of discrimination against spurious C to U conversion by APOBEC3A(Y130A/Y132H).) The Cq value obtained with APOBEC3A(Y130A/Y132H) and a slightly-less selective variant (Y130A) was significantly lower when compared to wild type APOBEC3A [which is non-selective for 5mC]. Importantly, when a catalytically inactive variant, APOBEC3A(Y130A/Y132H E72A), was used for deamination, Cq values match non-enzyme controls (BSA) confirming that the observed qPCR detection of 5mC is dependent on the evolved APOBEC variant.

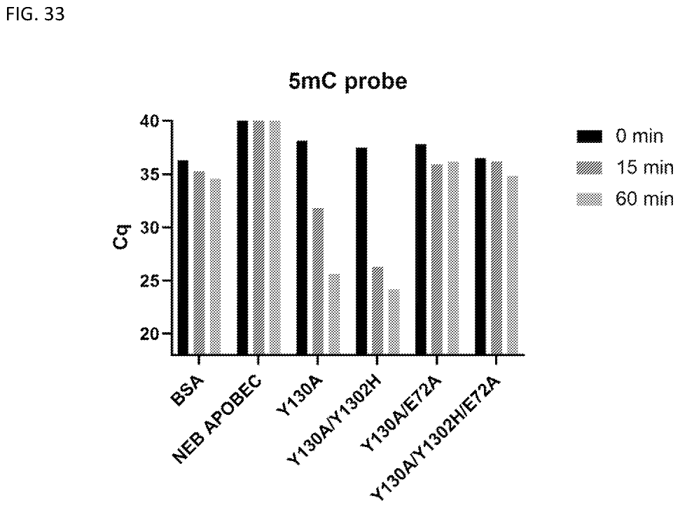

Because the qPCR primers are selective for 5mC-converted DNA, significant qPCR amplification can be detected even though only a small fraction of substrate is deaminated. A 15 min reaction time was sufficient to observe a significant decrease in Cq value for APOBEC3A(Y130A/Y132H) (FIG. 33). Based on this initial success, we expect that alternative iterations of this detection methodology are compatible with any number of DNA amplification-based diagnostic approaches including: (1) LAMP, (2) Recombinase-polymerase amplifications, (3) other isothermal amplification methodologies.

### Example 22

### Detection of 5mC Loci Using an Altered Cytidine Deaminase and CRISPR-Cas12

CRISPR-Cas systems have emerged as promising tools for the rapid and specific quantification of nucleic acid sequences. Techniques such as DETECTR (Chen et al., Science. 2018; 360: 436-439) and CDetection (Teng et al., Genome Biol. 2019; 20:132) use Cas12-family enzymes to detect single-nucleotide polymorphisms in analyte DNA with high sensitivity. Cas12-family enzymes bind and cleave DNA substrates, dictated by complementarity to a guide RNA that is complexed with Cas12. Mismatches between the guide RNA and target DNA inhibit this process. Importantly, Cas12 orthologs exhibit ‘collateral cleavage’ activity: target DNA binding results in the activation of a highly processive, non-specific nuclease activity, leading to the cleavage of ssDNA in trans. This collateral cleavage activity can be visualized using a separate ssDNA reporter substrate containing a fluorophore and quencher. RNA-guided engagement of target DNA by Cas12 results in trans-cleavage of the ssDNA reporter. Subsequent liberation of the fluorophore from quencher can be visualized with a fluorimeter. Because the trans-cleavage activity of Cas12 is highly catalytic, sub-attomolar concentrations of target DNA can be detected (Teng et al., Genome Biol. 2019; 20:132).

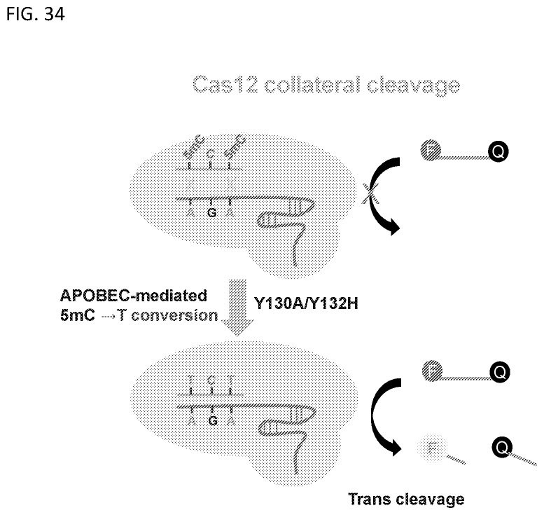

The collateral cleavage activity of Cas12 can be harnessed for the sensitive detection of 5mC (FIG. 34). The workflow involves: 1) analyte DNA denaturation, 2) 5mC to T conversion using an altered cytidine deaminase, and 3) application of Cas12-guide RNA complex to the sample. The guide RNA is fully complementary to 5mC to T converted DNA, therefore only allowing Cas12 to engage analyte DNA if it contained 5mC. The ensuing activation of Cas12-mediated trans-cleavage results in cleavage of reporter ssDNA and increased fluorescence. This effect is measured using standard plate reader instruments.

Cas12-family enzymes exhibit different requirements for target DNA engagement, being specific for either dsDNA or ssDNA. The use of ssDNA-specific Cas12b or Cas12f is advantageous for the above workflow as it would bypass the requirement for target DNA rehybridization prior to Cas12-gRNA application. Cas12b/f-gRNA and APOBEC could then be combined in a one-pot reaction, as Cas12b/f will engage the target ssDNA after APOBEC-mediated 5mC to T conversion has occurred. This will accelerate the development of this workflow as a point-of-care (POC) diagnostic for DNA methylation.

### Example 23

### Spatial Detection of 5mC Using Fluorescence In Situ Hybridization (FISH)

FISH is routinely employed to detect the abundance and spatial localization of DNA sequences of interest in fixed cell and tissue sections using fluorescently labeled probes. It is increasingly used in IVD applications to diagnose cytogenic abnormalities such as chromosome translocations, deletions, and copy number variation. For example, deletion of chromosome 5q is associated with poor prognosis in acute myeloid leukemia, and has thus been assayed as a biomarker using FISH probes (molecular.abbott/us/en/products/oncology/vysis-egr1-fish-probe-kit).

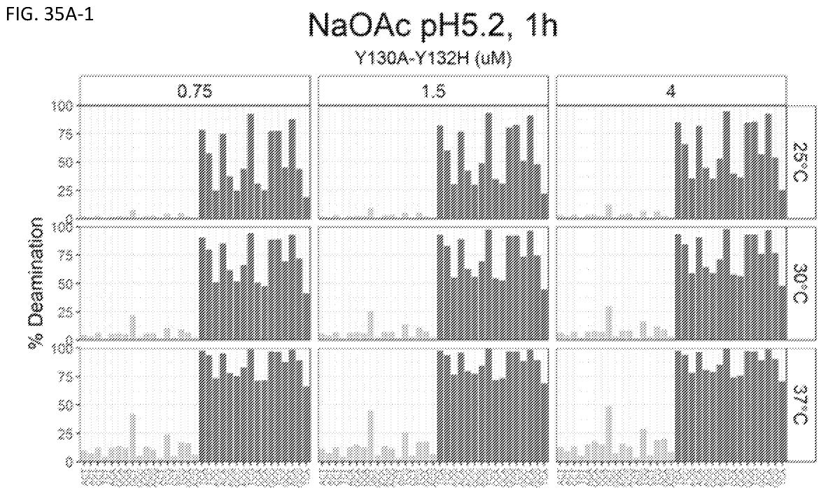

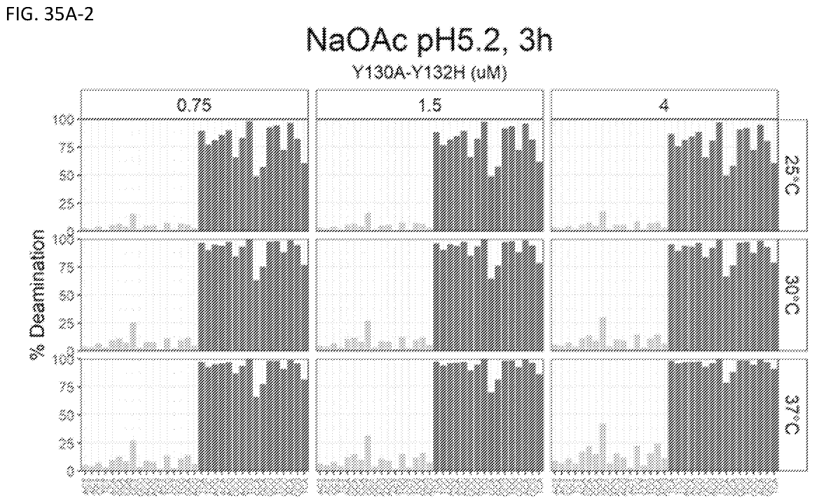

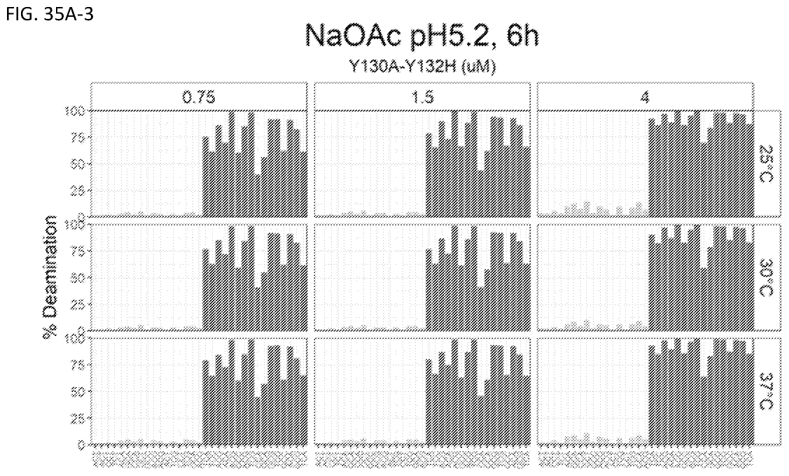

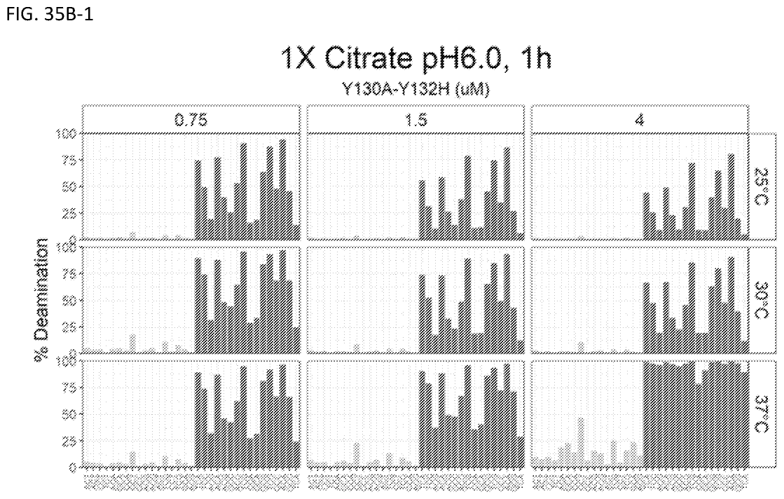

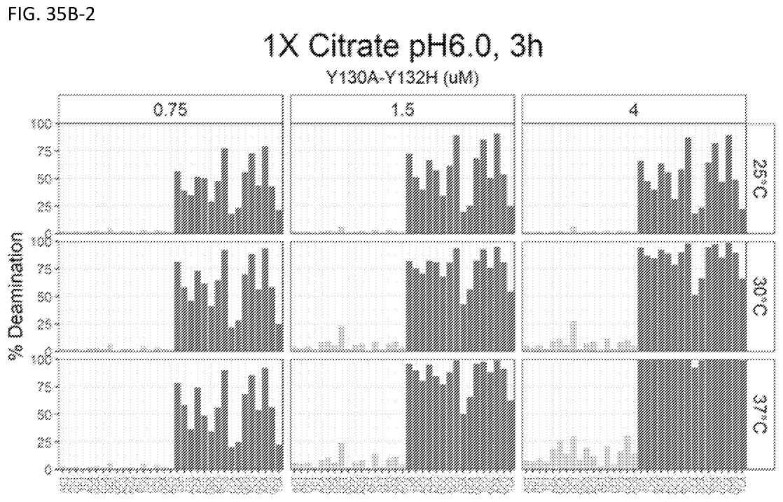

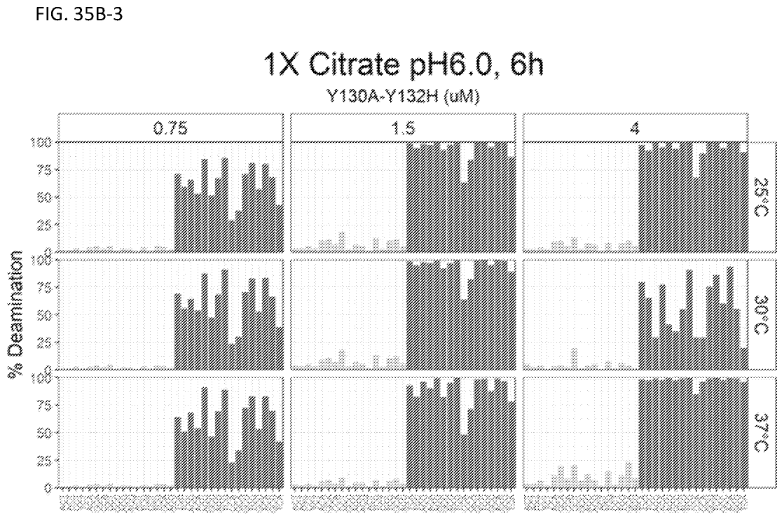

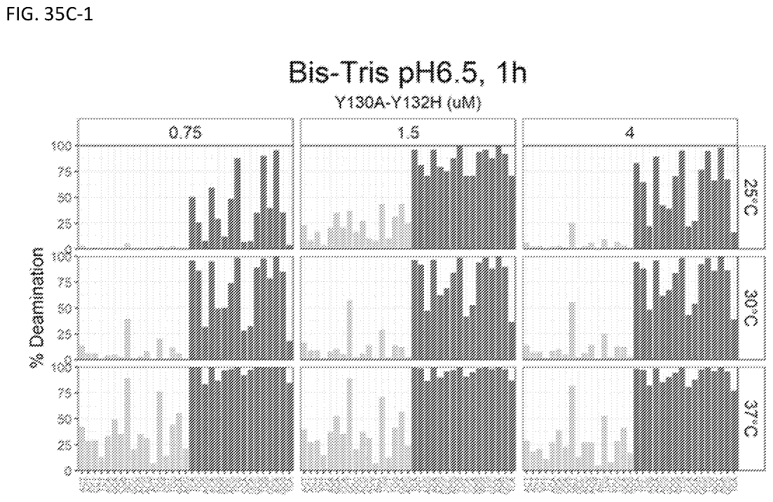

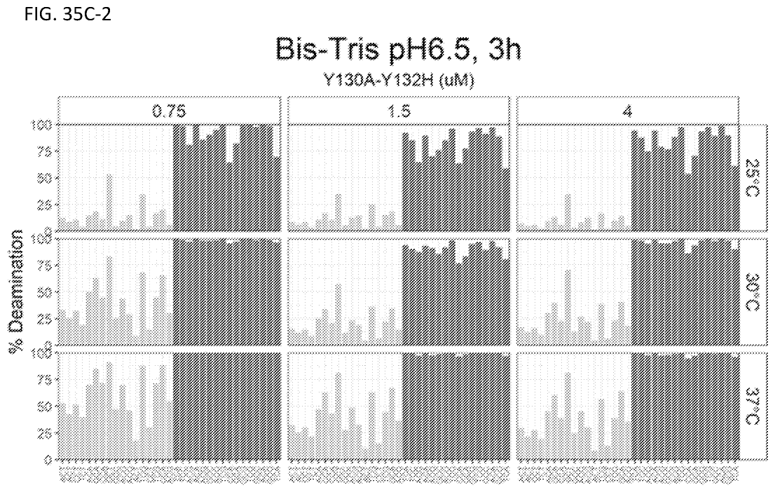

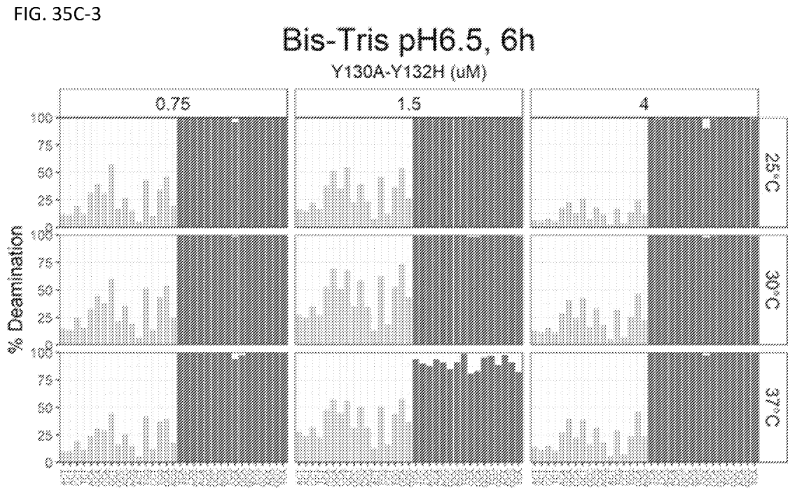

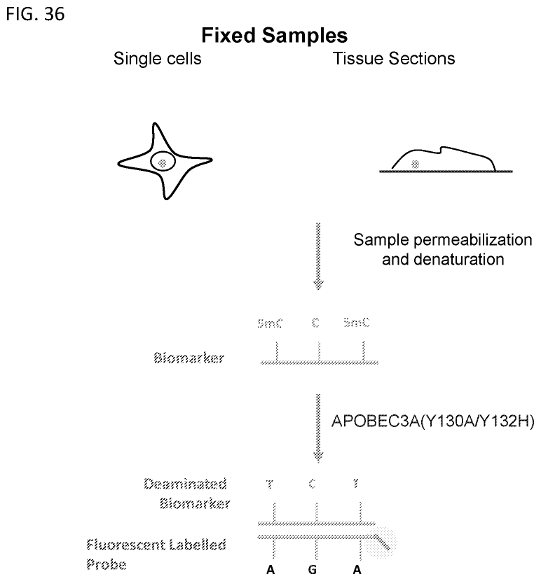

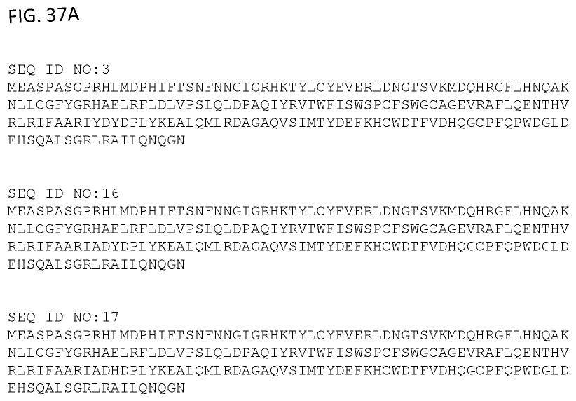

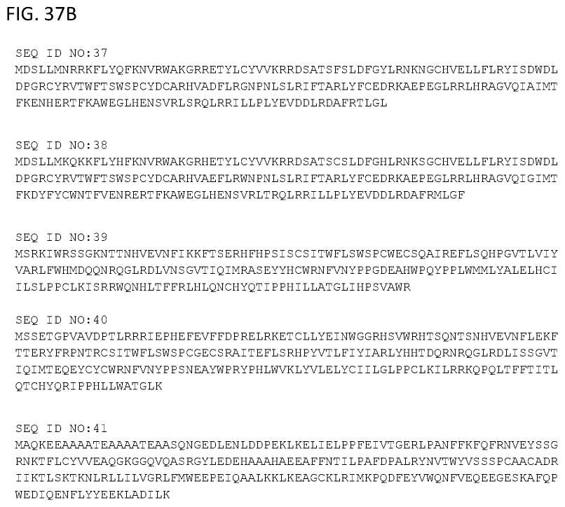

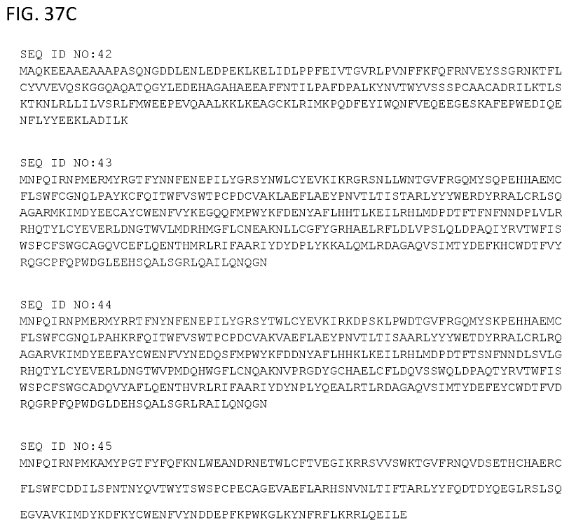

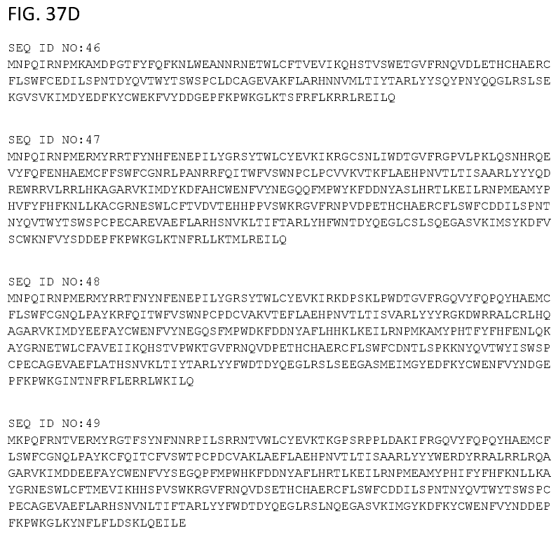

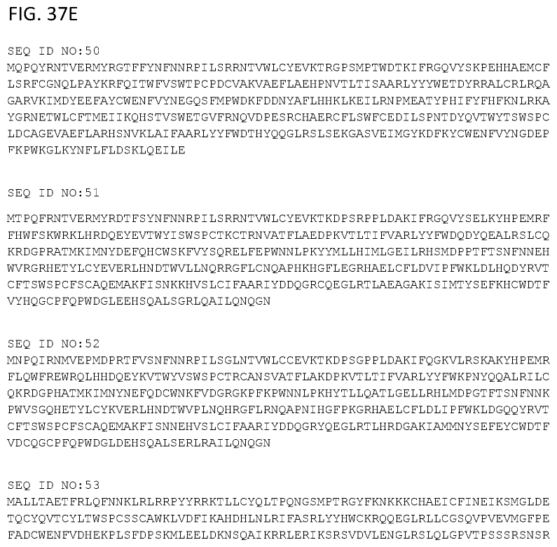

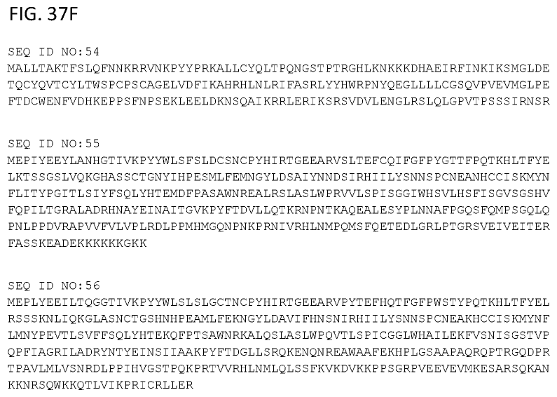

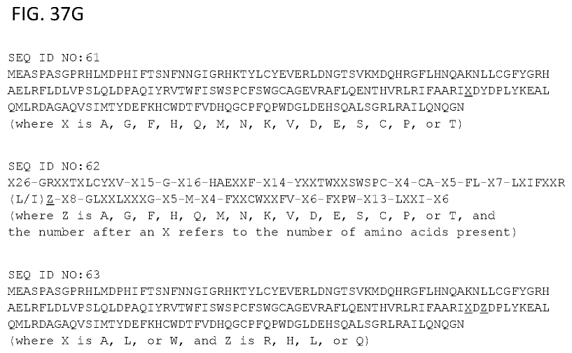

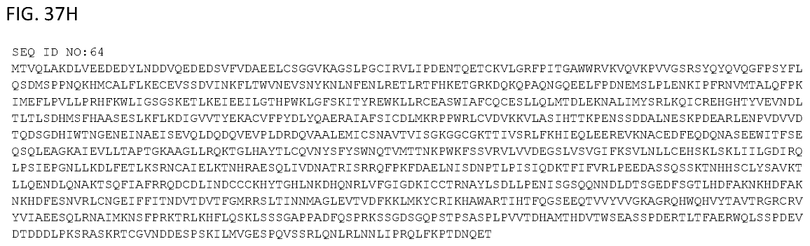

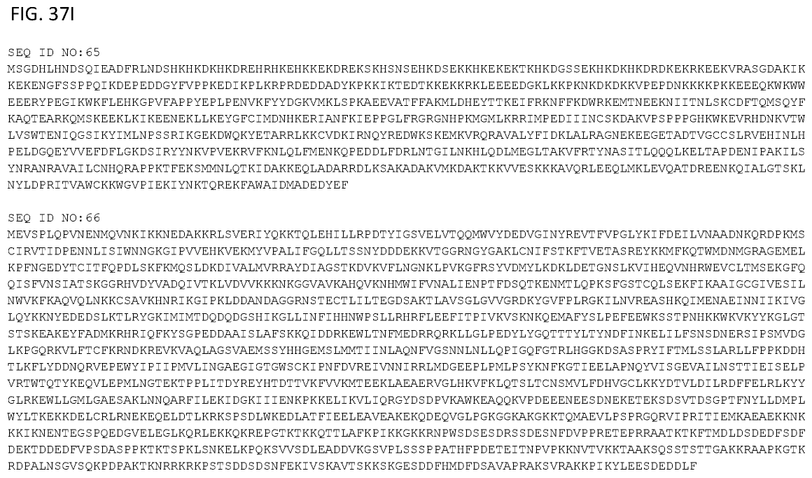

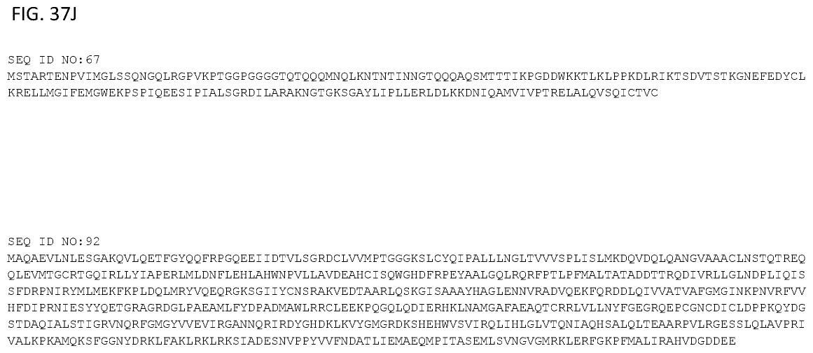

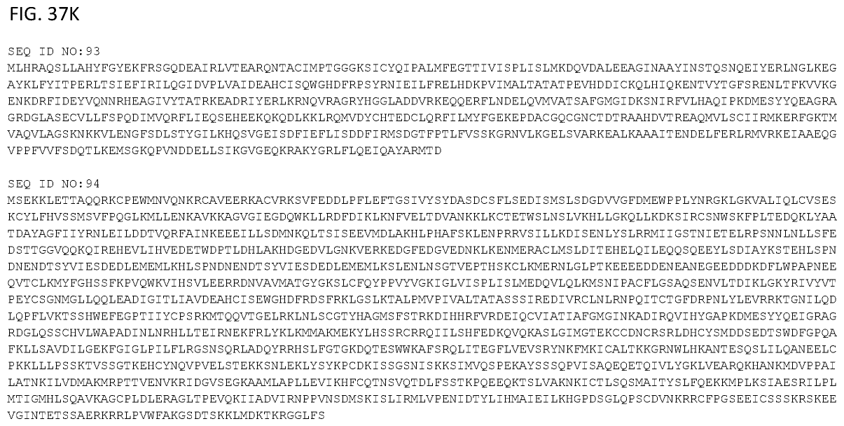

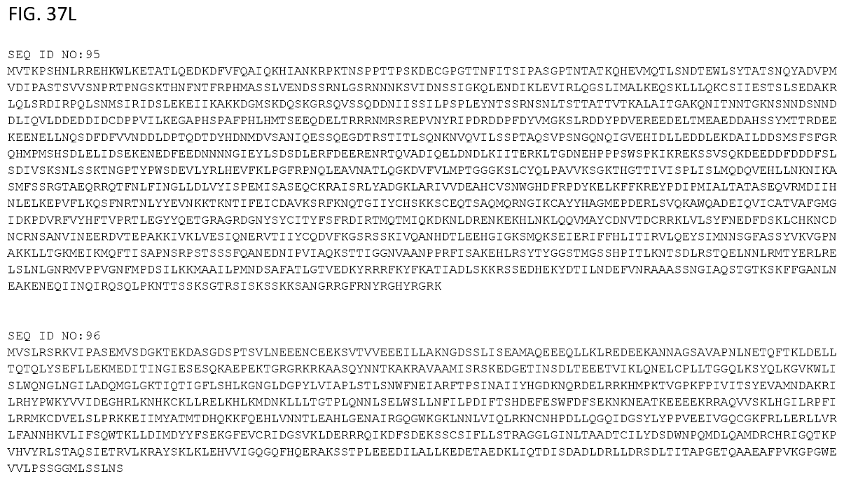

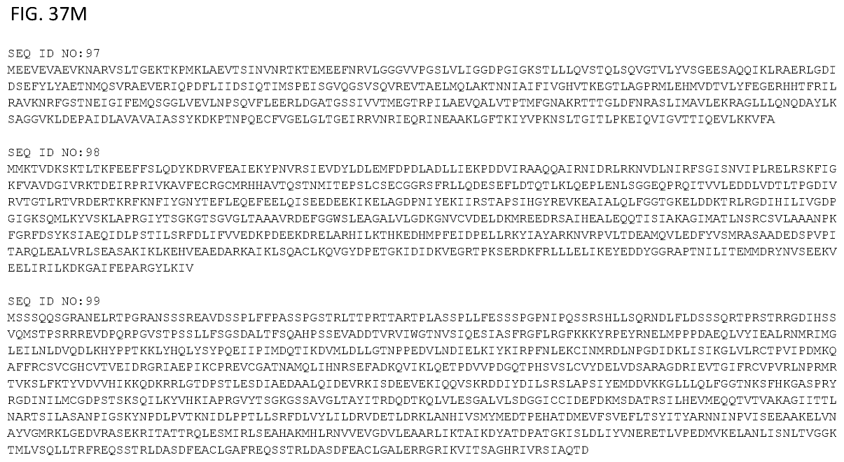

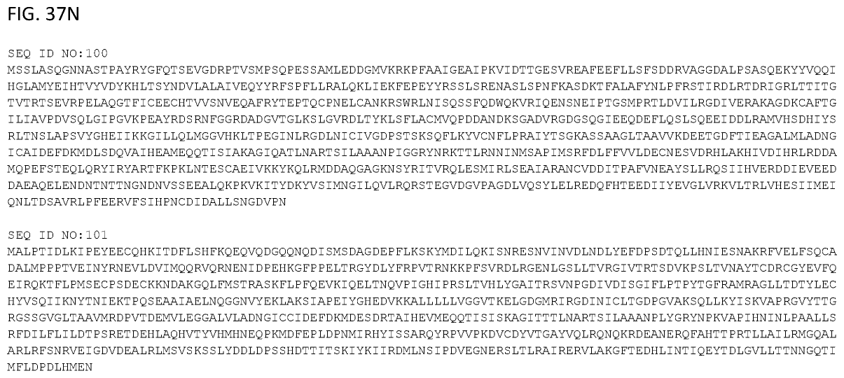

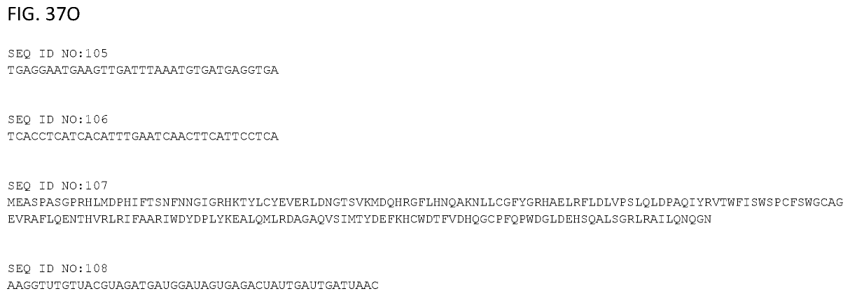

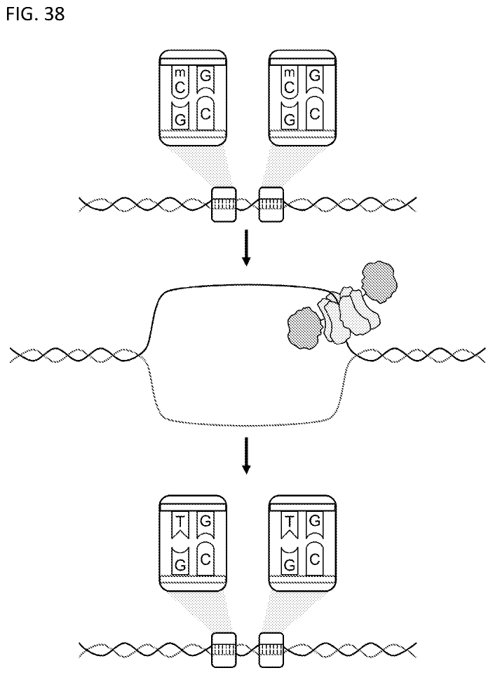

While the detection of DNA sequences by FISH is well-established, extending this technique to detect DNA modifications such as 5mC remains challenging due to technical difficulties with in situ bisulfite treatment of cells or tissue sections. Altered cytidine deaminase-mediated 5mC to T conversion enables such a workflow due to the mild conditions required for APOBEC enzymatic activity: for instance, 37° C. at pH 5.2-6.5. Furthermore, we have discovered that the 5mC-selective APOBEC3A(Y130A/Y132H) enzyme is active in citrate buffer pH 6.0 (FIG. 35) buffer is commonly used in FISH protocols to increase overall probe signal intensity (Yu et al., Exp Ther Med. 2021; 22:1480). The envisaged APOBEC-FISH protocol involves: 1) permeabilization and denaturation of fixed cells or tissue sections, 2) 5mC to T conversion using APOBEC, and 3) hybridization of probes that are specific to the deaminated DNA sequence (FIG. 36).

### Example 24

### Array-Based Detection of 5mC

ILMN methylation array products typically involve the selective hybridization of bisulfite-converted DNA to bead-based probes. Using an altered cytidine deaminase for direct 5mC to T conversion lowers DNA input requirements and simplifies probe design as overall 4-base genome complexity is maintained.

In the case of 4 base genomes, array probes are more specific, so quantification of methylated samples is expected to be accurate than quantification of unmethylated samples. Hybridization efficiency for 4-base genome is better than degenerated low complexity genome due to the preservation of complexity between the DNA and the probe, and can produce better resolutions.

### Example 25

Use of Y-shaped adapter library prep and RecQ in addition to WT cytidine deaminase and altered cytidine deaminase to observe deamination.

In this example, five aliquots of a dsDNA having a known sequence and methylation status carrying Y-shaped adapters are prepared and treated with enzymes. Each enzyme is provided as an independent protein not attached to any other enzyme. The first aliquot is treated with RecQ, a dsDNA helicase. The second aliquot is treated with a wild-type (WT) cytidine deaminase. The third aliquot is treated with RecQ and WT cytidine deaminase. The fourth aliquot is treated with an altered cytidine deaminase. The fifth aliquot is treated with RecQ and an altered cytidine deaminase.

After treatment, each sample is prepared for Illumina sequencing using the Accel Methyl-NGS kit (Swift Biosciences). Specifically, the reactions are purified using Zymo Oligo Clean and Concentrator Kit and eluted in 15 μL elution buffer (10 mM Tris pH 8.0). The Accel-NGS Methyl-Seq kit (Swift Biosciences) is then used for library preparation of single-stranded DNA according to manufacturer's instructions. After purification of the library, 1-5 ng amplified DNA is run on an Agilent Bioanalyzer High Sensitivity DNA Chip to confirm proper library fragment sizes. The resulting ACE-Seq library is sequenced at 1.9 pM with single-end mode on a NextSeq 500 sequencer (Illumina) using the NextSeq 500/500 High Output kit v2 (150 cycles).

Sequencing data from the sample is analyzed to detect known sites of 5mC and unmodified C. Deaminated 5mC and unmodified C sites are read as T during sequencing. Each detected T is compared to the known sequence to determine whether it was a T, 5mC, or unmodified C in the original sample.

For the aliquot treated with RecQ only, no deamination is detected. In the aliquot treated with WT cytidine deaminase without RecQ, no deamination is detected. In the aliquot treated with RecQ and WT cytidine deaminase, moderate levels of deamination of both 5mC and C are detected. This level of deamination is attributed to RecQ unwinding sections of dsDNA for treatment by the WT cytidine deaminase. It is learned that treatment of a sample with RecQ increases deamination of both 5mC and C in a dsDNA sample by WT cytidine deaminase.

In the aliquot treated with an altered cytidine deaminase without RecQ, a low level of deamination of 5mC is detected. In the aliquot treated with an altered cytidine deaminase with RecQ, a high level of 5mC deamination and a low level of C deamination is observed. The high level of 5mC deamination is attributed to RecQ unwinding sections of dsDNA for treatment by the altered cytidine deaminase. It is learned that treatment of a sample with RecQ increases deamination of 5mC, but not C, in a dsDNA sample by an altered cytidine deaminase.

### Example 26

Screen of helicases for recombinant expression and solubility.

While RecQ is commercially available, other helicases may be more suitable for use with the cytidine deaminases and altered cytidine deaminase of this disclosure.

The protein coding sequences of many helicases from different domains of life are identified and obtained. A first panel of helicases that are ATP-dependent are screened. A second panel of ATP-independent helicase are also screened. Each helicase is recombinantly expressed and purified. Helicases that are expressed at greater than 5 mg/L are determined to be abundant enough to be used as candidates for further screening. From this example, it is learned which helicases are potential candidates for use with a cytidine deaminase.

### Example 27

Screen of recruitment motifs for helicases.

Each candidate from Example 26 is screened against a set of libraries of known methylation status including different adapter motifs. Adapter motifs are designed to recruit helicases. Motifs of interest include Y-shaped adapters, partial duplexes, ssDNA bubbles, forks, blunt duplexes, three-strand junctions, and others. Motifs are ligated as adapters at the 5′ or 3′ end of a test dsDNA. Arrays are prepared with each motif as a 5′ or 3′ adapter. Each array is treated with a helicase and a cytidine deaminase. The cytidine deaminase is active on ssDNA, and is only able to deaminate C and 5mC when the dsDNA has been separated by a helicase.

Deamination is measured by digesting the dsDNA into individual nucleosides. After digestion, the nucleosides are measured using LC/MS to determine the rate of deamination. Levels of deamination are compared across different adapter motifs.

It is observed that the efficacy of each motif is approximately the same whether it is attached to the 5′ or 3′ end of a test dsDNA. Combinations of motifs and candidate helicases which result in high levels of deamination of C and 5mC in test dsDNA are identified. These combinations are determined to be the highest performing candidates.

### Example 28

### Expression of Helicase-Cytidine Deaminase Fusion Proteins

While RecQ is commercially available, it may or may not be suitable for expression as a fusion protein with a cytidine deaminase. Many helicases are tested for expression as fusion proteins with a cytidine deaminase.

The protein coding sequences from Example 26 are cloned into expression cassettes as fusion proteins with wild-type cytidine deaminases and altered cytidine deaminases. Each helicase is recombinantly expressed as a fusion protein with a cytidine deaminase. Each helicase is tested as both an N-terminal fusion and a C-terminal fusion to the cytidine deaminase.

Helicase fusion proteins that are expressed at greater than 5 mg/L are determined to be abundant enough to be used as candidates for further screening. From this example, it is learned which helicases are potential candidates for expression as a fusion protein with a cytidine deaminase.

### Example 29

Expression of helicases independently for creation of conjugated helicase-cytidine deaminase protein complexes.

The protein coding sequences of the helicases identified in Example 26 are recombinantly expressed independently or as fusion proteins with different conjugation tags. Multiple conjugation tags are tested, including Sortag, SpyTag, and split intein systems.

Cytidine deaminase and altered cytidine deaminases of interest are recombinantly expressed as fusion proteins to different conjugation tags. The conjugation tags used are compatible with those expressed as fusion proteins with the helicases. Each pair of fusion tagged proteins is combined in suitable conditions to facilitate conjugation. Multiple chemical conjugation methods are also tested with each helicase, including biotin-avidin conjugation and click chemistry.

Percent formation of each conjugated protein complex is measured by gel electrophoresis and immunoblotting showing a change in the molecular weight of the protein. Mass spectrometry will be used to confirm the sequence identity of each protein. Proteins that successfully form covalent complexes are determined to be potential candidates for use with the workflows described herein.

### Example 30

Screen of helicase-altered cytidine deaminase complexes for dsDNA deamination activity.

In this Example, the degree to which attaching the cytidine deaminase and helicase impacts deamination of dsDNA samples is investigated The protein complexes identified as highest performing candidates in Examples 28 and 29 expressed, purified, and conjugated when applicable. Aliquots of dsDNA having a known sequence and methylation status appended to each helicase recruitment motif identified in Example 27 are prepared. Each protein complex and helicase recruitment motif are tested in combination.

After treatment, each sample is prepared for Illumina sequencing using the Accel Methyl-NGS kit (Swift Biosciences). Specifically, the reactions are purified using Zymo Oligo Clean and Concentrator Kit and eluted in 15 μL elution buffer (10 mM Tris pH 8.0). The Accel-NGS Methyl-Seq kit (Swift Biosciences) is then used for library preparation of single-stranded DNA according to manufacturer's instructions. After purification of the library, 1-5 ng amplified DNA is run on an Agilent Bioanalyzer High Sensitivity DNA Chip to confirm proper library fragment sizes. The resulting ACE-Seq library is sequenced at 1.9 pM with single-end mode on a NextSeq 500 sequencer (Illumina) using the NextSeq 500/500 High Output kit v2 (150 cycles).

Sequencing data from the sample is analyzed to detect known sites of 5mC and unmodified C. Deaminated 5mC and unmodified C sites are read as T during sequencing. Each detected T is compared to the known sequence to determine whether it was a T, 5mC, or unmodified C in the original sample.

The level of deamination of 5mC and unmodified C is measured in each sample. For some protein complexes including wild-type cytidine deaminases, a high level of deamination of both 5mC and unmodified C is detected. For some protein complexes including altered cytidine deaminases, a low level of unmodified C deamination and a high level of 5mC deamination is detected. These protein complexes are determined to be the most suitable for use with the workflows described herein.

The complete disclosure of all patents, patent applications, and publications, and electronically available material (including, for instance, nucleotide sequence submissions in, e.g., GenBank and RefSeq, and amino acid sequence submissions in, e.g., SwissProt, UniProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A protein complex comprising a cytidine deaminase and a helicase,
wherein the cytidine deaminase is an altered cytidine deaminase comprising amino acid substitution mutations at positions functionally equivalent to (Tyr/Phe)130 and Tyr132 in a wild-type APOBEC3A protein.

2. The protein complex of claim 1, wherein the cytidine deaminase and the helicase are attached.

3. The protein complex of claim 2, wherein the cytidine deaminase and the helicase comprise a fusion protein.

4.-8. (canceled)

9. The protein complex of claim 1, wherein the substitution mutation at the position functionally equivalent to Tyr130 comprises a mutation to Ala, Val, or Trp, the substitution mutation at the position functionally equivalent to Tyr132 comprises a mutation to Arg, His, Leu, or Gln, or a combination thereof.

10. (canceled)

11. (canceled)

12. The protein complex of claim 1, wherein the altered cytidine deaminase is member of the AID subfamily, the APOBEC1 subfamily, the APOBEC2 subfamily, the APOBEC3A subfamily, the APOBEC3B subfamily, the APOBEC3C subfamily, the APOBEC3D subfamily, the APOBEC3F subfamily, the APOBEC3G subfamily, the APOBEC3G subfamily, the APOBEC3H subfamily, or the APOBEC4 subfamily.

13. (canceled)

14. (canceled)

15. The protein complex of claim 1, wherein the helicase unwinds double-stranded DNA (dsDNA).

16.-21. (canceled)

22. A composition comprising:
the protein complex of claim 1; and
a DNA sample comprising double-stranded DNA comprising at least one 5mC.

23. The composition of claim 22, wherein the sample comprises genomic DNA.

24. (canceled)

25. A composition comprising:
a protein complex comprising:
a cytidine deaminase, and
a helicase; and
a sample comprising double-stranded DNA comprising at least one 5mC.

26.-29. (canceled)

30. The composition of claim 25, wherein the sample comprises a helicase recruitment motif comprising a Y-shaped adapter structure, a partial duplex, a single-stranded DNA (ssDNA) bubble, a fork, a blunt duplex, a three-strand junction, or a combination thereof.

31.-33. (canceled)

34. The composition of claim 25, wherein the cytidine deaminase comprises an altered cytidine deaminase comprising an amino acid substitution mutation at a position functionally equivalent to (Tyr/Phe)130 in a wild-type APOBEC3A protein.

35. (canceled)

36. The composition of claim 34, wherein the substitution mutation at the position functionally equivalent to Tyr130 comprises a mutation to Ala, Val, or Trp.

37. The composition of claim 34, wherein the altered cytidine deaminase converts 5-methyl cytosine (5mC) to thymidine (T) by deamination at a greater rate than conversion of cytosine (C) to uracil (U) by deamination.

38. The composition of claim 37, wherein the rate is at least 100-fold greater.

39.-41. (canceled)

42. The composition of claim 34, wherein the substitution mutation at the position functionally equivalent to Tyr130 comprises a mutation to alanine, glycine, phenylalanine, histidine, glutamine, methionine, asparagine, lysine, valine, aspartic acid, glutamic acid, serine, cysteine, proline, arginine, or threonine.

43.-52. (canceled)

53. A method comprising:
providing a sample of DNA suspected of comprising dsDNA comprising at least one 5-methyl cytosine (5mC);
contacting the sample with the protein complex of claim 1 under conditions suitable for conversion of 5 methylcytosine (5mC) to thymidine (T) to result in converted dsDNA, wherein 5mC is converted to T; and
processing the converted dsDNA to produce a sequencing library.

54. The method of claim 53, further comprising:
providing a surface comprising a plurality of amplification sites,
wherein the amplification sites comprise at least two populations of attached single-stranded capture oligonucleotides having a free 3′ end, and
contacting the surface comprising amplification sites with the sequencing library under conditions suitable to produce a plurality of amplification sites that each comprise a clonal population of amplicons from an individual member of the sequencing library.

55. (canceled)

56. (canceled)

57. The method of claim 53, wherein processing the converted double-stranded DNA to produce a sequencing library occurs after contacting the dsDNA with the protein complex.

58. (canceled)

59. The method of claim 53, wherein the processing comprises fragmentation or tagmentation of the double-stranded DNA and addition of a universal sequence to the double-stranded DNA fragments.

60. (canceled)

61. The method of claim 53, wherein the sample is a biological sample comprising cell-free DNA.

62.-67. (canceled)


SEQUENCE LISTING (129 sequences)

SEQ ID NO: 1 - protein, 198 aa, Homo sapiens [sequence omitted]

SEQ ID NO: 2 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 3 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 4 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 5 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 6 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 7 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 8 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 9 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 10 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKIPGVRAQGRYMDILCDAEV

SEQ ID NO: 11 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 12 - protein, 38 aa, synthetic construct
  VARIANT 2: note = P, A, or V
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 34: note = Any amino acid
  VARIANT 35: note = Any amino acid
  VARIANT 36: note = Any amino acid
  VARIANT 37: note = Any amino acid
  VARIANT 4..31: note = This region may encompass 23-28 residues
  VARIANT 34..37: note = This region may encompass 2-4 residues
HXEXXXXXXXXXXXXXXXXXXXXXXXXXXXXPCXXXXC

SEQ ID NO: 13 - protein, 87 aa, synthetic construct
  VARIANT 2: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 30: note = S or T
  VARIANT 33: note = Any amino acid
  VARIANT 34: note = Any amino acid
  VARIANT 35: note = Any amino acid
  VARIANT 36: note = Any amino acid
  VARIANT 38: note = Any amino acid
  VARIANT 39: note = Any amino acid
  VARIANT 40: note = Any amino acid
  VARIANT 41: note = Any amino acid
  VARIANT 42: note = Any amino acid
  VARIANT 43: note = Any amino acid
  VARIANT 45: note = Any amino acid
  VARIANT 46: note = Any amino acid
  VARIANT 47: note = Any amino acid
  VARIANT 48: note = Any amino acid
  VARIANT 49: note = Any amino acid
  VARIANT 50: note = Any amino acid
  VARIANT 51: note = Any amino acid
  VARIANT 52: note = Any amino acid
  VARIANT 54: note = Any amino acid
  VARIANT 55: note = Any amino acid
  VARIANT 56: note = Any amino acid
  VARIANT 57: note = Any amino acid
  VARIANT 58: note = Any amino acid
  VARIANT 60: note = L or I
  VARIANT 62: note = Any amino acid
  VARIANT 63: note = Any amino acid
  VARIANT 64: note = Any amino acid
  VARIANT 65: note = Any amino acid
  VARIANT 66: note = Any amino acid
  VARIANT 67: note = Any amino acid
  VARIANT 68: note = Any amino acid
  VARIANT 69: note = Any amino acid
  VARIANT 70: note = Any amino acid
  VARIANT 71: note = Any amino acid
  VARIANT 72: note = Any amino acid
  VARIANT 74: note = Any amino acid
  VARIANT 75: note = Any amino acid
  VARIANT 77: note = Any amino acid
  VARIANT 78: note = Any amino acid
  VARIANT 79: note = Any amino acid
  VARIANT 80: note = Any amino acid
  VARIANT 81: note = Any amino acid
  VARIANT 82: note = Any amino acid
  VARIANT 83: note = Any amino acid
  VARIANT 84: note = Any amino acid
  VARIANT 85: note = Any amino acid
  VARIANT 86: note = Any amino acid
  VARIANT 33..36: note = This region may encompass 2-4 residues
  VARIANT 62..72: note = This region may encompass 8-11 residues
HXEXXXXXXXXXXXXXXXXXXXXXXXXSWXPCXXXXCXXXXXXFXXXXXXXXLXXXXXRXYXXXXXXXXXXXLXXLXXXXXXXXXXM

SEQ ID NO: 14 - protein, 199 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 29: note = Any amino acid
  VARIANT 30: note = Any amino acid
  VARIANT 32: note = Any amino acid
  VARIANT 36: note = Any amino acid
  VARIANT 38: note = Any amino acid
  VARIANT 39: note = Any amino acid
  VARIANT 40: note = Any amino acid
  VARIANT 41: note = Any amino acid
  VARIANT 42: note = Any amino acid
  VARIANT 43: note = Any amino acid
  VARIANT 44: note = Any amino acid
  VARIANT 45: note = Any amino acid
  VARIANT 46: note = Any amino acid
  VARIANT 47: note = Any amino acid
  VARIANT 48: note = Any amino acid
  VARIANT 49: note = Any amino acid
  VARIANT 50: note = Any amino acid
  VARIANT 51: note = Any amino acid
  VARIANT 52: note = Any amino acid
  VARIANT 54: note = Any amino acid
  VARIANT 55: note = Any amino acid
  VARIANT 56: note = Any amino acid
  VARIANT 58: note = Any amino acid
  VARIANT 59: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 65: note = Any amino acid
  VARIANT 66: note = Any amino acid
  VARIANT 67: note = Any amino acid
  VARIANT 68: note = Any amino acid
  VARIANT 69: note = Any amino acid
  VARIANT 73: note = Any amino acid
  VARIANT 74: note = Any amino acid
  VARIANT 76: note = Any amino acid
  VARIANT 77: note = Any amino acid
  VARIANT 78: note = Any amino acid
  VARIANT 79: note = Any amino acid
  VARIANT 80: note = Any amino acid
  VARIANT 81: note = Any amino acid
  VARIANT 82: note = Any amino acid
  VARIANT 83: note = Any amino acid
  VARIANT 84: note = Any amino acid
  VARIANT 85: note = Any amino acid
  VARIANT 86: note = Any amino acid
  VARIANT 87: note = Any amino acid
  VARIANT 88: note = Any amino acid
  VARIANT 89: note = Any amino acid
  VARIANT 91: note = Any amino acid
  VARIANT 92: note = Any amino acid
  VARIANT 95: note = Any amino acid
  VARIANT 96: note = Any amino acid
  VARIANT 102: note = Any amino acid
  VARIANT 103: note = Any amino acid
  VARIANT 104: note = Any amino acid
  VARIANT 105: note = Any amino acid
  VARIANT 108: note = Any amino acid
  VARIANT 109: note = Any amino acid
  VARIANT 110: note = Any amino acid
  VARIANT 111: note = Any amino acid
  VARIANT 112: note = Any amino acid
  VARIANT 115: note = Any amino acid
  VARIANT 116: note = Any amino acid
  VARIANT 117: note = Any amino acid
  VARIANT 118: note = Any amino acid
  VARIANT 119: note = Any amino acid
  VARIANT 120: note = Any amino acid
  VARIANT 121: note = Any amino acid
  VARIANT 123: note = Any amino acid
  VARIANT 125: note = Any amino acid
  VARIANT 126: note = Any amino acid
  VARIANT 127: note = Any amino acid
  VARIANT 129: note = L or I
  VARIANT 131: note = Any amino acid
  VARIANT 132: note = Any amino acid
  VARIANT 133: note = Any amino acid
  VARIANT 134: note = Any amino acid
  VARIANT 135: note = Any amino acid
  VARIANT 136: note = Any amino acid
  VARIANT 137: note = Any amino acid
  VARIANT 138: note = Any amino acid
  VARIANT 141: note = Any amino acid
  VARIANT 142: note = Any amino acid
  VARIANT 144: note = Any amino acid
  VARIANT 145: note = Any amino acid
  VARIANT 146: note = Any amino acid
  VARIANT 148: note = Any amino acid
  VARIANT 149: note = Any amino acid
  VARIANT 150: note = Any amino acid
  VARIANT 151: note = Any amino acid
  VARIANT 152: note = Any amino acid
  VARIANT 154: note = Any amino acid
  VARIANT 155: note = Any amino acid
  VARIANT 156: note = Any amino acid
  VARIANT 157: note = Any amino acid
  VARIANT 159: note = Any amino acid
  VARIANT 160: note = Any amino acid
  VARIANT 163: note = Any amino acid
  VARIANT 164: note = Any amino acid
  VARIANT 167: note = Any amino acid
  VARIANT 168: note = Any amino acid
  VARIANT 169: note = Any amino acid
  VARIANT 170: note = Any amino acid
  VARIANT 171: note = Any amino acid
  VARIANT 172: note = Any amino acid
  VARIANT 174: note = Any amino acid
  VARIANT 177: note = Any amino acid
  VARIANT 178: note = Any amino acid
  VARIANT 179: note = Any amino acid
  VARIANT 180: note = Any amino acid
  VARIANT 181: note = Any amino acid
  VARIANT 182: note = Any amino acid
  VARIANT 183: note = Any amino acid
  VARIANT 184: note = Any amino acid
  VARIANT 185: note = Any amino acid
  VARIANT 186: note = Any amino acid
  VARIANT 187: note = Any amino acid
  VARIANT 188: note = Any amino acid
  VARIANT 189: note = Any amino acid
  VARIANT 191: note = Any amino acid
  VARIANT 192: note = Any amino acid
  VARIANT 194: note = Any amino acid
  VARIANT 195: note = Any amino acid
  VARIANT 196: note = Any amino acid
  VARIANT 197: note = Any amino acid
  VARIANT 198: note = Any amino acid
  VARIANT 199: note = Any amino acid
  VARIANT 1..26: note = This region may encompass 16-26 residues
  VARIANT 102..105: note = This region may encompass 2-4 residues
  VARIANT 194..199: note = This region may encompass 2-6 residues
XXXXXXXXXXXXXXXXXXXXXXXXXXGRXXTXLCYXVXXXXXXXXXXXXXXXGXXXNXXXXXXXXXXXXHAEXXFXXXXXXXXXXXXXXYXXTWXXSWSPCXXXXCAXXXXXFLXXXXXXXLXIXXXRXYXXXXXXXXGLXXLXXXGXXXXXMXXXXFXXCWXXFVXXXXXXFXPWXXXXXXXXXXXXXLXXIXXXXXX

SEQ ID NO: 15 - protein, 199 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 29: note = Any amino acid
  VARIANT 30: note = Any amino acid
  VARIANT 32: note = Any amino acid
  VARIANT 36: note = Any amino acid
  VARIANT 38: note = Any amino acid
  VARIANT 39: note = Any amino acid
  VARIANT 40: note = Any amino acid
  VARIANT 41: note = Any amino acid
  VARIANT 42: note = Any amino acid
  VARIANT 43: note = Any amino acid
  VARIANT 44: note = Any amino acid
  VARIANT 45: note = Any amino acid
  VARIANT 46: note = Any amino acid
  VARIANT 47: note = Any amino acid
  VARIANT 48: note = Any amino acid
  VARIANT 49: note = Any amino acid
  VARIANT 50: note = Any amino acid
  VARIANT 51: note = Any amino acid
  VARIANT 52: note = Any amino acid
  VARIANT 54: note = Any amino acid
  VARIANT 55: note = Any amino acid
  VARIANT 56: note = Any amino acid
  VARIANT 57: note = Any amino acid
  VARIANT 58: note = Any amino acid
  VARIANT 59: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 65: note = Any amino acid
  VARIANT 66: note = Any amino acid
  VARIANT 67: note = Any amino acid
  VARIANT 68: note = Any amino acid
  VARIANT 69: note = Any amino acid
  VARIANT 73: note = Any amino acid
  VARIANT 74: note = Any amino acid
  VARIANT 76: note = Any amino acid
  VARIANT 77: note = Any amino acid
  VARIANT 78: note = Any amino acid
  VARIANT 79: note = Any amino acid
  VARIANT 80: note = Any amino acid
  VARIANT 81: note = Any amino acid
  VARIANT 82: note = Any amino acid
  VARIANT 83: note = Any amino acid
  VARIANT 84: note = Any amino acid
  VARIANT 85: note = Any amino acid
  VARIANT 86: note = Any amino acid
  VARIANT 87: note = Any amino acid
  VARIANT 88: note = Any amino acid
  VARIANT 89: note = Any amino acid
  VARIANT 91: note = Any amino acid
  VARIANT 92: note = Any amino acid
  VARIANT 95: note = Any amino acid
  VARIANT 96: note = Any amino acid
  VARIANT 102: note = Any amino acid
  VARIANT 103: note = Any amino acid
  VARIANT 104: note = Any amino acid
  VARIANT 105: note = Any amino acid
  VARIANT 108: note = Any amino acid
  VARIANT 109: note = Any amino acid
  VARIANT 110: note = Any amino acid
  VARIANT 111: note = Any amino acid
  VARIANT 112: note = Any amino acid
  VARIANT 115: note = Any amino acid
  VARIANT 116: note = Any amino acid
  VARIANT 117: note = Any amino acid
  VARIANT 118: note = Any amino acid
  VARIANT 119: note = Any amino acid
  VARIANT 120: note = Any amino acid
  VARIANT 121: note = Any amino acid
  VARIANT 123: note = Any amino acid
  VARIANT 126: note = Any amino acid
  VARIANT 127: note = Any amino acid
  VARIANT 129: note = L or I
  VARIANT 131: note = Any amino acid
  VARIANT 132: note = Any amino acid
  VARIANT 133: note = Any amino acid
  VARIANT 134: note = Any amino acid
  VARIANT 135: note = Any amino acid
  VARIANT 136: note = Any amino acid
  VARIANT 137: note = Any amino acid
  VARIANT 138: note = Any amino acid
  VARIANT 141: note = Any amino acid
  VARIANT 142: note = Any amino acid
  VARIANT 144: note = Any amino acid
  VARIANT 145: note = Any amino acid
  VARIANT 146: note = Any amino acid
  VARIANT 148: note = Any amino acid
  VARIANT 149: note = Any amino acid
  VARIANT 150: note = Any amino acid
  VARIANT 151: note = Any amino acid
  VARIANT 152: note = Any amino acid
  VARIANT 154: note = Any amino acid
  VARIANT 155: note = Any amino acid
  VARIANT 156: note = Any amino acid
  VARIANT 157: note = Any amino acid
  VARIANT 159: note = Any amino acid
  VARIANT 160: note = Any amino acid
  VARIANT 163: note = Any amino acid
  VARIANT 164: note = Any amino acid
  VARIANT 167: note = Any amino acid
  VARIANT 168: note = Any amino acid
  VARIANT 169: note = Any amino acid
  VARIANT 170: note = Any amino acid
  VARIANT 171: note = Any amino acid
  VARIANT 172: note = Any amino acid
  VARIANT 174: note = Any amino acid
  VARIANT 177: note = Any amino acid
  VARIANT 178: note = Any amino acid
  VARIANT 179: note = Any amino acid
  VARIANT 180: note = Any amino acid
  VARIANT 181: note = Any amino acid
  VARIANT 182: note = Any amino acid
  VARIANT 183: note = Any amino acid
  VARIANT 184: note = Any amino acid
  VARIANT 185: note = Any amino acid
  VARIANT 186: note = Any amino acid
  VARIANT 187: note = Any amino acid
  VARIANT 188: note = Any amino acid
  VARIANT 189: note = Any amino acid
  VARIANT 191: note = Any amino acid
  VARIANT 192: note = Any amino acid
  VARIANT 194: note = Any amino acid
  VARIANT 195: note = Any amino acid
  VARIANT 196: note = Any amino acid
  VARIANT 197: note = Any amino acid
  VARIANT 198: note = Any amino acid
  VARIANT 199: note = Any amino acid
XXXXXXXXXXXXXXXXXXXXXXXXXXGRXXTXLCYXVXXXXXXXXXXXXXXXGXXXXXXXXXXXXXXXXHAEXXFXXXXXXXXXXXXXXYXXTWXXSWSPCXXXXCAXXXXXFLXXXXXXXLXIFXXRXYXXXXXXXXGLXXLXXXGXXXXXMXXXXFXXCWXXFVXXXXXXFXPWXXXXXXXXXXXXXLXXIXXXXXX

SEQ ID NO: 16 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIADYDPLYKEALQMLRDAGAQVSIMTYDEFKHCWDTFVDHQGCPFQPWDGLDEHSQALSGRLRAILQNQGN

SEQ ID NO: 17 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIADHDPLYKEALQMLRDAGAQVSIMTYDEFKHCWDTFVDHQGCPFQPWDGLDEHSQALSGRLRAILQNQGN

SEQ ID NO: 18 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARILDYDPLYKEALQMLRDAGAQVSIMTYDEFKHCWDTFVDHQGCPFQPWDGLDEHSQALSGRLRAILQNQGN

SEQ ID NO: 19 - protein, 202 aa, Macaca fascicularis
MDGSPASRPRHLMDPNTFTFNFNNDLSVRGRHQTYLCYEVERLDNGTWVPMDERRGFLHNKAKNVPCGDYGCHVELRFLCEVPSWQLDPAQTYRVTWFISWSPCFRKGCAGQVRAFLQENKHVRLRIFAARIYDYDPRYQEALRTLRDAGAQVSIMTYEEFKHCWDTFVDRQGRPFQPWDGLDEHSQALSGRLRAILQNQGN

SEQ ID NO: 20 - protein, 199 aa, Pongo pygmaeus
MEASPASGPRHLMDPCVFTSNFNNGIRWHKTYLCYEVERLDNGTWVKMDQHRGFLHNQARNPLYGLDGRHAELRFLGLLPYWQLDPAQIYRVTWFISWSPCFSWGCARQVRAFLQENTHVRLRIFAARIYDYDPLYKEALQMLRDAGAQVSIMTYDEFEYCWNTFVDHQGCPFQPWDGLEEHSQALSGKLQAILLNQGN

SEQ ID NO: 21 - protein, 199 aa, Nomascus leucogenys
MEASPASRPGHLMDPQVFTSNFNNGIRWHKTYLCYEVERLDNGTWVKMDQHRGFLHNQAKNLFCGFYGRHAELCFLDLVPSLQLDPAQTYRVTWFISWSPCFSWGCAEQVRAFLQENTHVRLRLFAARIYDYDPLYKEALQMLRGAGAQVSIMTYHEFKHCWDTFVDHQGRPFQPWDGLEEHSQALSGRLQAILQNQGN

SEQ ID NO: 22 - protein, 199 aa, Pan troglodytes
TEASPASGPRHLMDPHIFTSNFNNGIGRRKTYLCYEVERLDNGTSVKMDQHRGFLHNQAKNLLCGFYGRHAELCFLDLVPSLQLDPAQIYRVTWFISWSPCFSWGCAGQVRAFLQENTHVRLRIFAARIYDYDPLYKEALQMLRDAGAQVSIMTYDEFKHCWDTFVDHQGCPFQPWDGLEEHSQALSGRLRAILQNQGN

SEQ ID NO: 23 - protein, 199 aa, Gorilla gorilla
VEASPASGPRHLMDPHIFTSNFNNVIGRHKTYLCYEVERLDNGTWVKMDQHRGFLHNQAKNLLCGFYGRHAELRFLDLVPSLQLDPAQIYRVTWFISWSPCFSWGCAGQVRAFLQENTHVRLHIFAARIYDYDPLYKEALQMLRDAGAQVSIMTYDEFKHCWDTFVDHQGCPFQPWDGLEEHSQALSGRLRAILQNQGN

SEQ ID NO: 24 - DNA, 35 bp, synthetic construct
  source 1..35: mol_type = other DNA
  modified_base 35: mod_base = OTHER; note = 3'-6-carboxyfluorescein modified nucleotide
tgaggaatgaagttgattcaaatgtgatgaggtga

SEQ ID NO: 25 - DNA, 35 bp, synthetic construct
  source 1..35: mol_type = other DNA
  modified_base 19: mod_base = OTHER; note = Uracil
  modified_base 35: mod_base = OTHER; note = 3'-6-carboxyfluorescein modified nucleotide
tgaggaatgaagttgatttaaatgtgatgaggtga

SEQ ID NO: 26 - DNA, 35 bp, synthetic construct; identical to SEQ ID NO: 24
  source 1..35: mol_type = other DNA
  modified_base 19: mod_base = OTHER; note = 5-methyl cytosine
  modified_base 35: mod_base = OTHER; note = 3'-6-carboxyfluorescein modified nucleotide

SEQ ID NO: 27 - DNA, 35 bp, synthetic construct; identical to SEQ ID NO: 25
  source 1..35: mol_type = other DNA
  modified_base 35: mod_base = OTHER; note = 3'-6-carboxyfluorescein modified nucleotide

SEQ ID NO: 28 - DNA, 35 bp, synthetic construct
  source 1..35: mol_type = other DNA
tcacctcatcacatttgaatcaacttcattcctca

SEQ ID NO: 29 - DNA, 126 bp, synthetic construct
  source 1..126: mol_type = other DNA
  modified_base 22: mod_base = OTHER; note = 5-methyl cytosine
  modified_base 26: mod_base = OTHER; note = 5-methyl cytosine
  modified_base 32: mod_base = OTHER; note = 5-methyl cytosine
  modified_base 42: mod_base = OTHER; note = 5-methyl cytosine
  modified_base 47: mod_base = OTHER; note = 5-methyl cytosine
  modified_base 54: mod_base = OTHER; note = 5-methyl cytosine
  modified_base 59: mod_base = OTHER; note = 5-methyl cytosine
  modified_base 64: mod_base = OTHER; note = 5-methyl cytosine
  modified_base 67: mod_base = OTHER; note = 5-methyl cytosine
  modified_base 72: mod_base = OTHER; note = 5-methyl cytosine
  modified_base 79: mod_base = OTHER; note = 5-methyl cytosine
  modified_base 85: mod_base = OTHER; note = 5-methyl cytosine
  modified_base 90: mod_base = OTHER; note = 5-methyl cytosine
  modified_base 95: mod_base = OTHER; note = 5-methyl cytosine
  modified_base 101: mod_base = OTHER; note = 5-methyl cytosine
  modified_base 108: mod_base = OTHER; note = 5-methyl cytosine
gaggtgtatggttgtactaatcactcctggacgaatcttaacacaacgtgcagccaaacgcttcgccacggcaacgtgcggactcgtcgccttacaatcgcgcaggtcacgttgaagatgaggatg

SEQ ID NO: 30 - DNA, 84 bp, synthetic construct
  source 1..84: mol_type = other DNA
  modified_base 18: mod_base = OTHER; note = 5-methyl cytosine
  modified_base 32: mod_base = OTHER; note = 5-methyl cytosine
  modified_base 46: mod_base = OTHER; note = 5-methyl cytosine
  modified_base 60: mod_base = OTHER; note = 5-methyl cytosine
gaggtgtatggttgtagcgcaaatcgtaaaacgcaaagcgaaaaccgcaaaccgtaaaccgaaaagcgcttgaagatgaggatg

SEQ ID NO: 31 - DNA, 84 bp, synthetic construct
  source 1..84: mol_type = other DNA
  modified_base 18: mod_base = OTHER; note = 5-methyl cytosine
  modified_base 32: mod_base = OTHER; note = 5-methyl cytosine
  modified_base 46: mod_base = OTHER; note = 5-methyl cytosine
  modified_base 60: mod_base = OTHER; note = 5-methyl cytosine
gaggtgtatggttgtagcggaaaacggaaatcggaaaacgtaaagcgtaaatcggaaagcgaaaagcggttgaagatgaggatg

SEQ ID NO: 32 - DNA, 84 bp, synthetic construct
  source 1..84: mol_type = other DNA
  modified_base 18: mod_base = OTHER; note = 5-methyl cytosine
  modified_base 32: mod_base = OTHER; note = 5-methyl cytosine
  modified_base 46: mod_base = OTHER; note = 5-methyl cytosine
  modified_base 60: mod_base = OTHER; note = 5-methyl cytosine
gaggtgtatggttgtaacgtaaaccgcaaaccggaaaacgaaaatcgcaaaccgaaaaccgtaaaacgcttgaagatgaggatg

SEQ ID NO: 33 - DNA, 48 bp, synthetic construct
  source 1..48: mol_type = other DNA
  modified_base 7: mod_base = OTHER; note = 5-methyl cytosine
  modified_base 18: mod_base = OTHER; note = 5-methyl cytosine
  modified_base 41: mod_base = OTHER; note = 5-methyl cytosine
aaggtccgtcacgcagacgacggacagcgagaccactgaccgatcaac

SEQ ID NO: 34 - DNA, 48 bp, synthetic construct
  source 1..48: mol_type = other DNA
  modified_base 6: mod_base = OTHER; note = Uracil
  modified_base 10: mod_base = OTHER; note = Uracil
  modified_base 12: mod_base = OTHER; note = Uracil
  modified_base 14: mod_base = OTHER; note = Uracil
  modified_base 21: mod_base = OTHER; note = Uracil
  modified_base 25: mod_base = OTHER; note = Uracil
  modified_base 28: mod_base = OTHER; note = Uracil
  modified_base 33: mod_base = OTHER; note = Uracil
  modified_base 34: mod_base = OTHER; note = Uracil
  modified_base 36: mod_base = OTHER; note = Uracil
  modified_base 40: mod_base = OTHER; note = Uracil
  modified_base 45: mod_base = OTHER; note = Uracil
  modified_base 48: mod_base = OTHER; note = Uracil
aaggtttgttatgtagatgatggatagtgagattattgattgattaat

SEQ ID NO: 35 - DNA, 48 bp, synthetic construct
  source 1..48: mol_type = other DNA
aaggtctgtcacgcagatgacggacagcgagaccactgactgatcaac

SEQ ID NO: 36 - DNA, 84 bp, synthetic construct
  source 1..84: mol_type = other DNA
  modified_base 18: mod_base = OTHER; note = 5-methyl cytosine
  modified_base 32: mod_base = OTHER; note = 5-methyl cytosine
  modified_base 46: mod_base = OTHER; note = 5-methyl cytosine
  modified_base 60: mod_base = OTHER; note = 5-methyl cytosine
gaggtgtatggttgtaacgaaaaccggaaatcgaaaagcgtaaatcgtaaatcgcaaaacggaaatcgattgaagatgaggatg

SEQ ID NO: 37 - protein, 188 aa, Gorilla gorilla
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 38 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 39 - protein, 191 aa, Gorilla gorilla
MSRKIWRSSGKNTTNHVEVNFIKKFTSERHFHPSISCSITWFLSWSPCWECSQAIREFLSQHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 40 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 41 - protein, 230 aa, Gorilla gorilla
MAQKEEAAAATEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 42 - protein, 224 aa, Mus musculus
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 43 - protein, 382 aa, Gorilla gorilla
MNPQIRNPMERMYRGTFYNNFENEPILYGRSYNWLCYEVKIKRGRSNLLWNTGVFRGQMYSQPEHHAEMCFLSWFCGNQLPAYKCFQITWFVSWTPCPDCVAKLAEFLAEYPNVTLTISTARLYYYWERDYRRALCRLSQAGARMKIMDYEECAYCWENFVYKEGQQFMPWYKFDENYAFLHHTLKEILRHLMDPDTFTFNFNNDPLVLRRHQTYLCYEVERLDNGTWVLMDRHMGFLCNEAKNLLCGFYGRHAELRFLDLVPSLQLDPAQIYRVTWFISWSPCFSWGCAGQVCEFLQENTHMRLRIFAARIYDYDPLYKKALQMLRDAGAQVSIMTYDEFKHCWDTFVYRQGCPFQPWDGLEEHSQALSGRLQAILQNQGN

SEQ ID NO: 44 - protein, 382 aa, Macaca mulatta
MNPQIRNPMERMYRRTFNYNFENEPILYGRSYTWLCYEVKIRKDPSKLPWDTGVFRGQMYSKPEHHAEMCFLSWFCGNQLPAHKRFQITWFVSWTPCPDCVAKVAEFLAEYPNVTLTISAARLYYYWETDYRRALCRLRQAGARVKIMDYEEFAYCWENFVYNEDQSFMPWYKFDDNYAFLHHKLKEILRHLMDPDTFTSNFNNDLSVLGRHQTYLCYEVERLDNGTWVPMDQHWGFLCNQAKNVPRGDYGCHAELCFLDQVSSWQLDPAQTYRVTWFISWSPCFSWGCADQVYAFLQENTHVRLRIFAARIYDYNPLYQEALRTLRDAGAQVSIMTYDEFEYCWDTFVDRQGRPFQPWDGLDEHSQALSGRLRAILQNQGN

SEQ ID NO: 45 - protein, 190 aa, Gorilla gorilla
MNPQIRNPMKAMYPGTFYFQFKNLWEANDRNETWLCFTVEGIKRRSVVSWKTGVFRNQVDSETHCHAERCFLSWFCDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 46 - protein, 190 aa, Macaca mulatta
MNPQIRNPMKAMDPGTFYFQFKNLWEANNRNETWLCFTVEVIKQHSTVSWETGVFRNQVDLETHCHAERCFLSWFCEDILSPNTDYQVTWYTSWSPCLDCAGEVAKFLARHNNVMLTIYTARLYYSQYPNYQQGLRSLSEKGVSVKIMDYEDFKYCWEKFVYDDGEPFKPWKGLKTSFRFLKRRLREILQ

SEQ ID NO: 47 - protein, 386 aa, Pan troglodytes
MNPQIRNPMERMYRRTFYNHFENEPILYGRSYTWLCYEVKIKRGCSNLIWDTGVFRGPVLPKLQSNHRQEVYFQFENHAEMCFFSWFCGNRLPANRRFQITWFVSWNPCLPCVVKVTKFLAEHPNVTLTISAARLYYYQDREWRRVLRRLHKAGARVKIMDYKDFAHCWENFVYNEGQQFMPWYKFDDNYASLHRTLKEILRNPMEAMYPHVFYFHFKNLLKACGRNESWLCFTVDVTEHHPPVSWKRGVFRNPVDPETHCHAERCFLSWFCDDILSPNTNYQVTWYTSWSPCPECAREVAEFLARHSNVKLTIFTARLYHFWNTDYQEGLCSLSQEGASVKIMSYKDFVSCWKNFVYSDDEPFKPWKGLKTNFRLLKTMLREILQ

SEQ ID NO: 48 - protein, 374 aa, Macaca mulatta
MNPQIRNPMERMYRRTFNYNFENEPILYGRSYTWLCYEVKIRKDPSKLPWDTGVFRGQVYFQPQYHAEMCFLSWFCGNQLPAYKRFQITWFVSWNPCPDCVAKVTEFLAEHPNVTLTISVARLYYYRGKDWRRALCRLHQAGARVKIMDYEEFAYCWENFVYNEGQSFMPWDKFDDNYAFLHHKLKEILRNPMKAMYPHTFYFHFENLQKAYGRNETWLCFAVEIIKQHSTVPWKTGVFRNQVDPETHCHAERCFLSWFCDNTLSPKKNYQVTWYISWSPCPECAGEVAEFLATHSNVKLTIYTARLYYFWDTDYQEGLRSLSEEGASMEIMGYEDFKYCWENFVYNDGEPFKPWKGINTNFRFLERRLWKILQ

SEQ ID NO: 49 - protein, 373 aa, Gorilla gorilla
MKPQFRNTVERMYRGTFSYNFNNRPILSRRNTVWLCYEVKTKGPSRPPLDAKIFRGQVYFQPQYHAEMCFLSWFCGNQLPAYKCFQITCFVSWTPCPDCVAKLAEFLAEHPNVTLTISAARLYYYWERDYRRALRRLRQAGARVKIMDDEEFAYCWENFVYSEGQPFMPWHKFDDNYAFLHRTLKEILRNPMEAMYPHIFYFHFKNLLKAYGRNESWLCFTMEVIKHHSPVSWKRGVFRNQVDSETHCHAERCFLSWFCDDILSPNTNYQVTWYTSWSPCPECAGEVAEFLARHSNVNLTIFTARLYYFWDTDYQEGLRSLNQEGASVKIMGYKDFKYCWENFVYNDDEPFKPWKGLKYNFLFLDSKLQEILE

SEQ ID NO: 50 - protein, 373 aa, Macaca mulatta
MQPQYRNTVERMYRGTFFYNFNNRPILSRRNTVWLCYEVKTRGPSMPTWDTKIFRGQVYSKPEHHAEMCFLSRFCGNQLPAYKRFQITWFVSWTPCPDCVAKVAEFLAEHPNVTLTISAARLYYYWETDYRRALCRLRQAGARVKIMDYEEFAYCWENFVYNEGQSFMPWDKFDDNYAFLHHKLKEILRNPMEATYPHIFYFHFKNLRKAYGRNETWLCFTMEIIKQHSTVSWETGVFRNQVDPESRCHAERCFLSWFCEDILSPNTDYQVTWYTSWSPCLDCAGEVAEFLARHSNVKLAIFAARLYYFWDTHYQQGLRSLSEKGASVEIMGYKDFKYCWENFVYNGDEPFKPWKGLKYNFLFLDSKLQEILE

SEQ ID NO: 51 - protein, 384 aa, Gorilla gorilla
MTPQFRNTVERMYRDTFSYNFNNRPILSRRNTVWLCYEVKTKDPSRPPLDAKIFRGQVYSELKYHPEMRFFHWFSKWRKLHRDQEYEVTWYISWSPCTKCTRNVATFLAEDPKVTLTIFVARLYYFWDQDYQEALRSLCQKRDGPRATMKIMNYDEFQHCWSKFVYSQRELFEPWNNLPKYYMLLHIMLGEILRHSMDPPTFTSNFNNEHWVRGRHETYLCYEVERLHNDTWVLLNQRRGFLCNQAPHKHGFLEGRHAELCFLDVIPFWKLDLHQDYRVTCFTSWSPCFSCAQEMAKFISNKKHVSLCIFAARIYDDQGRCQEGLRTLAEAGAKISIMTYSEFKHCWDTFVYHQGCPFQPWDGLEEHSQALSGRLQAILQNQGN

SEQ ID NO: 52 - protein, 384 aa, Macaca mulatta
MNPQIRNMVEPMDPRTFVSNFNNRPILSGLNTVWLCCEVKTKDPSGPPLDAKIFQGKVLRSKAKYHPEMRFLQWFREWRQLHHDQEYKVTWYVSWSPCTRCANSVATFLAKDPKVTLTIFVARLYYFWKPNYQQALRILCQKRDGPHATMKIMNYNEFQDCWNKFVDGRGKPFKPWNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRTLHRDGAKIAMMNYSEFEYCWDTFVDCQGCPFQPWDGLDEHSQALSERLRAILQNQGN

SEQ ID NO: 53 - protein, 210 aa, Gorilla gorilla
MALLTAETFRLQFNNKLRLRRPYYRRKTLLCYQLTPQNGSMPTRGYFKNKKKCHAEICFINEIKSMGLDETQCYQVTCYLTWSPCSSCAWKLVDFIKAHDHLNLRIFASRLYYHWCKRQQEGLRLLCGSQVPVEVMGFPEFADCWENFVDHEKPLSFDPSKMLEELDKNSQAIKRRLERIKSRSVDVLENGLRSLQLGPVTPSSSRSNSR

SEQ ID NO: 54 - protein, 210 aa, Macaca mulatta
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 55 - protein, 368 aa, Gorilla gorilla
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQSFQMPSGQLQPNLPPDVRAPVVFVLVPLRDLPPMHMGQNPNKPRNIVRHLNMPQMSFQETEDLGRLPTGRSVEIVEITERFASSKEADEKKKKKKGKK

SEQ ID NO: 56 - protein, 374 aa, Mus musculus
MEPLYEEILTQGGTIVKPYYWLSLSLGCTNCPYHIRTGEEARVPYTEFHQTFGFPWSTYPQTKHLTFYELRSSSKNLIQKGLASNCTGSHNHPEAMLFEKNGYLDAVIFHNSNIRHIILYSNNSPCNEAKHCCISKMYNFLMNYPEVTLSVFFSQLYHTEKQFPTSAWNRKALQSLASLWPQVTLSPICGGLWHAILEKFVSNISGSTVPQPFIAGRILADRYNTYEINSIIAAKPYFTDGLLSRQKENQNREAWAAFEKHPLGSAAPAQRQPTRGQDPRTPAVLMLVSNRDLPPIHVGSTPQKPRTVVRHLNMLQLSSFKVKDVKKPPSGRPVEEVEVMKESARSQKANKKNRSQWKKQTLVIKPRICRLLER

SEQ ID NO: 57 - protein, 1042 aa, Homo sapiens [sequence omitted]

SEQ ID NO: 58 - protein, 1087 aa, Homo sapiens
MARSSPYLRQLQGPLLPPRDLVEEDDDYLNDDVEEDEESVFIDAEELCSGGVKAGSLPGCLRVSICDENTQETCKVFGRFPITGAWWRVKVQVKPVVGSRSYQYQVQGFPSYFLQSDMSPPNQKHICALFLKECEVSSDDVNKFLTWVKEVSNYKNLNFENLRETLRTFHKETGRKDQKQPTQNGQEELFLDNEMSLPLENTIPFRNVMTALQFPKIMEFLPVLLPRHFKWIIGSGSKEMLKEIEEILGTHPWKLGFSKITYREWKLLRCEASWIAFCQCESLLQLMTDLEKNALIMYSRLKQICREDGHTYVEVNDLTLTLSNHMSFHAASESLKFLKDIGVVTYEKSCVFPYDLYHAERAIAFSICDLMKKPPWHLCVDVEKVLASIHTTKPENSSDDALNESKPDEVRLENPVDVVDTQDNGDHIWTNGENEINAEISEVQLDQDQVEVPLDRDQVAALEMICSNPVTVISGKGGCGKTTIVSRLFKHIEQLEEREVKKACEDFEQDQNASEEWITFTEQSQLEADKAIEVLLTAPTGKAAGLLRQKTGLHAYTLCQVNYSFYSWTQTMMTTNKPWKFSSVRVLVVDEGSLVSVGIFKSVLNLLCEHSKLSKLIILGDIRQLPSIEPGNLLKDLFETLKSRNCAIELKTNHRAESQLIVDNATRISRRQFPKFDAELNISDNPTLPISIQDKTFIFVRLPEEDASSQSSKTNHHSCLYSAVKTLLQENNLQNAKTSQFIAFRRQDCDLINDCCCKHYTGHLTKDHQSRLVFGIGDKICCTRNAYLSDLLPENISGSQQNNDLDASSEDFSGTLPDFAKNKRDFESNVRLCNGEIFFITNDVTDVTFGKRRSLTINNMAGLEVTVDFKKLMKYCRIKHAWARTIHTFQGSEEQTVVYVVGKAGRQHWQHVYTAVTRGRCRVYVIAEESQLRNAIMKNSFPRKTRLKHFLQSKLSSSGAPPADFPSPRKSSGDSGGPSTPSASPLPVVTDHAMTNDVTWSEASSPDERTLTFAERWQLSSPDGVDTDDDLPKSRASKRTCGVNDDESPSKIFMVGESPQVSSRLQNLRLNNLIPRQLFKPTDNQET

SEQ ID NO: 59 - protein, 1087 aa, Pan paniscus
MARSSPYLRQLQGPLLPPRDLVEEDDDYLNDDVEEDEESVFIDAEELCSGGVKAGSLPGCLRVSICDENTQETCKVFGRFPITGAWWRVKVQVKPVVGSRSYQYQVQGFPSYFLQSDMSPPNQKHICALFLKECEVSSDDVNKFLTWVKEVSNYKNLNFENLRETLRTFHKETGRKDQKQPTQNGQEELFPDNEMSLPLENTIPFRNVMTALQFPKIMEFLPVLLPRHFKWIIGSGSKEMLKEIEEILGTHPWKLGFSKITYREWKLLRCEASWIAFCQCESLLQLMTDLEKNALIMYSRLKQICREDGHTYVEVNDLTLTLSNHMSFHAASESLKFLKDIGVVTYEKSCVFPYDLYHAERAIAFSICDLMKKPPWHLCVDVEKVLASIHTTKPENSSDDALNESKPDEVRLENPVDVVDTQDNGDHIWTNGENEINAEISEVQLDQDQVEVPLDRDQVAALEMICSNPVTVISGKGGCGKTTIVSRLFKHIEQLEEREVKKACEDFEQDQNASEEWITFTEQSQLEAGKAIEVLLTAPTGKAAGLLRQKTGLHAYTLCQVNYSFYSWTQTMMTTNKPWKFSSVRVLVVDEGSLVSVGIFKSVLNLLCEHSKLSKLIILGDIRQLPSIEPGNLLKDLFETLKSRNCAIELKTNHRAESQLIVDNATRISRRQFPKFDAELNISDNPTLPISIQDKTFIFVRLPEEDASSQSSKTNHHSCLYSAVKTLLQENNLQNAKTSQFIAFRRQDCDLINDCCCKHYTGHLTKDHQSRLVFGIGDKICCTRNAYLSDLLPENISGSQQNNDLDASGEDFSGTLPDFAKNKRDFESNVRLCNGEIFFITNDVTDVTFGKRRSLTINNMAGLEVTVDFKKLMKYCRIKHAWARTIHTFQGSEEQTVVYVVGKAGRQHWQHVYTAVTRGRCRVYVIAEESQLRNAIMKNSFPRKTRLKHFLQSKLSSSGAPPADFPSPQKSSGDSGGPSTPSASPLPVVTDHAMTNDVTWSEASSPDERTLTFAERWQLSSPDGVDTDDDLPKSRASKRTCGVNDDESPSKIFMVGESPQVSSRLQNLRLNNLIPRQLFKPTDNQET

SEQ ID NO: 60 - protein, 1087 aa, Pongo abelii
MARSSRYLRQLQGPLLPPRDLVEEDDDYLNDDVEEDEESVFIDAEELCSGGVKAGSLPGCLRVSICDENTQETCKVFGRFPITGAWWRVKVQVKPVVGSRSYQYQVQGFPSYFLQSDMSPPNQKHICALFLKECEVSSDVVNKFLTWVKEVSNYENLNFENLRETLRTFHKETGKKDHKQPAQNGQEELFPDNEMSLPLENTIPFRNVMTALQFPKIMEFLPVLLPRHFKWIIGSGSKEMLKEIEEILGTHPWKLGFSKITYREWKLLRCEASWIAFCQCESLLQLMTDLEKNALIMYSRLKQICREHGHTYVEVNDLTLTLSDHMSFHAASESLKFLKDIGVVTYEKSCVFPYDLYHAERAIAFSICDLMKKPPWHLCVDVEKVLASIHTAKPENSSDDAWNESKPDEVRLENPVDVVDTQDSGDHIWTNGENEINAEISEVQLDQDQVEVPLDQDQVAALEMICTNPVTVISGKGGCGKTTIVSRLFKHMEQLEEREVKKACEDFEQDQNASEEWITFTEQSQLEAGKAIEVLLTAPTGKAAGLLRQKTGLHAYTLCQVNYSFYSWTQTMMTTNKPWKFSSVRVLVVDEGSLVSVGIFKSVLNLLCEHSKLSKLIILGDIRQLPSIEPGNLLKDLFETLKSRNCAIELKTNHRAESQLIVDNATRISRRQFPKFDAELNISDNPTLPISIQDKTFIFVRLPEEDASSQSSKTNHYSCLYSAVKTLLQENDLQNAKTSQFIAFRRQDCDLINDCCCKHYTGHLTKDHQNRLVFGIGDKICCTRNAYLSDLLPENISGSQQHNDLDASGEDFSGTLHDFAKNKHDFESNVRLCNGEIFFITNDVTDVTFGKRRSLTINNMAGLEVTVDFKKLMKYCHIKHAWARTIHTFQGSEEQTVVYVVGKAGRQHWQHVYTAVTRGRCRVYVIAEESQLRNAIMKNSFPRKTRLKHFLQSKLFSSGAPPADFQSPQKSSGDSGGPSTPSASPLPVVTDHTMINDVTWSEACLPDERTLTFPERWQLSSPDGVDADDDLPKSRASKRTCGVNDDESPSKILMVGESPQVSSRLQNLRLNNLIPRQLFKPTDNQES

SEQ ID NO: 61 - protein, 1087 aa, Pan troglodytes
MARSSPYLRQLQGPLLPPRDLVEEDDDYLNDDVEEDEESVFIDAEELCSGGVKAGSLPGCLRVSICDENTQETCKVFGRFPITGAWWRVKVQVKPVVGSRSYQYQVQGFPSYFLQSDMSPPNQKHICALFLKECEVSSDDVNKFLTWVKEVSNYKNLNFENLRETLRTFHKETGRKDQKQPTQNGQEELFPDNEMSLPLENTIPFRNVMTALQFPKIMEFLPVLLPRHFKWIIGSGSIEMLKEIEEILGTHPWKLGFSKITYREWKLLRCEASWIAFCQCESLLQLMTDLEKNALIIYSRLKQICREDGHTYVEVNDLTLTLSNHMSFHAASESLKFLKDIGVVTYEKSCVFPYDLYHAERAIAFSICDLMKKPPWHLCVDVEKVLASIHTTKPENSSDDALNESKPDEVRLENPVDVVDTQDNGDHIWTNGENEINAEISEVQLDQDQVEVPLDRDQVAALEMICSNPVTVISGKGGCGKTTIVSRLFKHIEQLEEREVKKACEDFEQDQNASEEWITFTEQSQLEAGKAIEVLLTAPTGKAAGLLRQKTGLHAYTLCQVNYSFYSWTQTMMTTNKPWKFSSVRVLVVDEGSLVSVGIFKSVLNLLCEHSKLSKLIILGDIRQLPSIEPGNLLKDLFETLKSRNCAIELKTNHRAESQLIVDNATRISRRQFPKFDAELNISDNPTLPISIQDKTFIFVRLPEEDASSQSSKTNHHSCLYSAVKTLLQENNLQNAKTSQFIAFRRQDCDLINDCCCKHYTGHLTKDHQSRLVFGIGDKICCTRNAYLSDLLPENISGSQQNNDLDASGEDFSGTLPDFAKNKRDFESNVRLCNGEIFFITNDVTDVTFGKRRSLTINNMAGLEVTVDFKKLMKYCRIKHAWARTIHTFQGSEEQTVVYVVGKAGRQHWQHVYTAVTRGRCRVYVIAEESQLRNAIMKNSFPRKTRLKHFLQSKLSSSGAPPADFPSPQKSSGDSGGPSTPSASPLPVVTDHAMTNDVTWSEASSPDERTLTFAERWQLSSPDGVDTDDDLPKSRASKRTCGVNDDESPSKIFMVGESPQVSSRLQNLRLNNLIPRQLFKPTDNQET

SEQ ID NO: 62 - protein, 1087 aa, Gorilla gorilla
MARSSRYLRQLQGPLLPPRDLVEEDDDYLNDDVEEDEESVFIDAEELCSGGVKAGSLPGCLRVSICDENTQETYKVFGRFPITGAWWRVKVQVKPVVGSRSYQYQVQGFPSYFLQSDMSPPNQKHICALFLKECEVSSDVVNKFLTWVKEVSNYRNLNFENLRETLRTFHKETGRKDQKQPAQNGQEELFPDNEMSLPLENTIPFRNVMTALQFPKIMEFLPVLLPRHFKWIIGSGSKEMLKEIEEILGTHPWKLGFSKITYREWKLLRCEASWIAFCQCESLLQLMTDLEKNALIMYSRLKQICREDGHTYVEVNDLTLTLSNHMSFHAASESLKFLKDIGVVTYEKSCIFPYDLYHAERAIAFSICDLMKKPPWHLCVDVEKVLASIHTTKPENSSDDALNESKPDEVRLENPVDVVDTQDNGDHIWTNGENEINADVSEVQLDQDQVEVPLDRDQVAALEMICSNPVTVISGKGGCGKTTIVSRLFKHIEQLEEREVKKACEDFEQDQNASEEWITFTEQSQLEAGKAIEVLLTAPTGKAAGLLRQKTGLHAYTLCQVNYSFYSWTQTMMTTNKPWKFSSVRVLVVDEGSLVSVGIFKSVLNLLCEHSKLSKLIILGDIRQLPSIEPGNLLKDLFETLKSRNCAIELKTNHRAESQLIVDNATRISRRQFPKFDAELNISDNPTLPISIQDKTFIFVRLPEEDASSQSSKTNHHSCLYSAVKTLLQENNLQNAKTSQFIAFRRQDCDLINDCCCKHYTGHLTKDHQSRLVFGIGDKICCTRNAYLSDLLPENISGSQQNNDLDASGEDFSGTLPDFAKNKRDFESNIRLCNGEIFFITNDVTDVTFGKRRSLTINNMAGLEVTVDFKKLMKYCRIKHAWARTIHTFQGSEEQTVVYVVGKAGRQHWQHVYTAVTRGRCRVYVIAEESQLRNAIMKNSFPRKTRLKHFLQSKLSSSGAPPADFQSPQKSSGDSGGPSTPSASPLPVVTDHAMTNDVTWSEASSPDERTLTFAERWQLSSPDRVDTDDDLPKSRASKRTCGVNDDESPSKIFMVGESPQVSSRLQNLRLNNLIPRQLFKPTDNQET

SEQ ID NO: 63 - protein, 1069 aa, Macaca fascicularis
MARSSRYLRQLQGPLLPPKDLVEEDEDYLNDDVQEDEDSVFVDAEELCSGGVKAGSLPGCIRVLIPDENTQETCKVLGRFPITGAWWRVKVQVKPVVGSRSYQYQVQGFPSYFLQSDMSPPNQKHICALFLKECEVSSDVINKFLAWVNEVSNYKNLNFENLRETLRTFHKETGRKDQKQPAQNGQEELFPDNEMSLPLENKIPYRNVMTALQFPKIMEFLPVLLPRHFKWLIESGSKEMLKEIEEILGTHPWKLGFSKITYRKWKLLRCEASWIAFCQCESLLQLMTDLEKNALIMYSKLKQICREHGHTYVEVNDLTLTLSDHMSFHAASESLKFLKDIGVVTYEKACVFPYDLYQAERAIAFSICDLMQRPPWRLCVDVKKVLASIHTTKPENSSDDALNESKPDEARLENPVDVVDTQDSGDHIWTNGENEINAEISEVQLDQDQVEVPLDRDQVAALEMICSNPVTVISGKGGCGKTTIVSRLFKHIEQLEEREVKNACEDFEQDQNASEEWNTFTEQSQLEAGKAIEVLLTAPTGKAAGLLRQKTGLHAYTLCQVNYSFYSWNQTMMTTNKPWKFSSVRVLVVDEGSLVSVGIFKSVLNLLCEHSKLSKLIILGDIRQLPSIEPGNLLKDLFETLKSRNCAIELKTNHRAESQLIVDNATRISRRQFPKFDAELNISDNPTLPISIQDKTFIFVRLPEEDASSQSSKTNHHSCLYSTVKTLLQENDLQNAKTSQFIAFRRQDCDLINDCCCKHYTGHLNKDHQNRLVFGVGDKICCTRNAYLSDLLPENISGSQQNNDLDTSGEDFSGTLHDFAKNKHDFESNVRLCNGEIFFITNDVTDVTFGMRRSLTINNMAGLEVTVDFKKLMKYCRIKHAWARTIHTFQGSEEQTVVYVVGKAGRQHWQHVYTAVTRGRCRVYVIAEESQLRNAIMKNSFPRKTRLKHFLQSKLSSSGAPPADFQTPRKSSGDSGGPSTPSASPLPVVTDHAMTNDVTWSEASSPDERTLTFAERWQLSSPDGVDTDDDLPKSRASKRTCGVNDDESPSKILMVGVMFACSQVLFKYC

SEQ ID NO: 64 - protein, 1082 aa, Piliocolobus tephrosceles
MTVQLAKDLVEEDEDYLNDDVQEDEDSVFVDAEELCSGGVKAGSLPGCIRVLIPDENTQETCKVLGRFPITGAWWRVKVQVKPVVGSRSYQYQVQGFPSYFLQSDMSPPNQKHMCALFLKECEVSSDVINKFLTWVNEVSNYKNLNFENLRETLRTFHKETGRKDQKQPAQNGQEELFPDNEMSLPLENKIPFRNVMTALQFPKIMEFLPVLLPRHFKWLIGSGSKETLKEIEEILGTHPWKLGFSKITYREWKLLRCEASWIAFCQCESLLQLMTDLEKNALIMYSRLKQICREHGHTYVEVNDLTLTLSDHMSFHAASESLKFLKDIGVVTYEKACVFPYDLYQAERAIAFSICDLMKRPPWRLCVDVKKVLASIHTTKPENSSDDALNESKPDEARLENPVDVVDTQDSGDHIWTNGENEINAEISEVQLDQDQVEVPLDRDQVAALEMICSNAVTVISGKGGCGKTTIVSRLFKHIEQLEEREVKNACEDFEQDQNASEEWITFSEQSQLEAGKAIEVLLTAPTGKAAGLLRQKTGLHAYTLCQVNYSFYSWNQTVMTTNKPWKFSSVRVLVVDEGSLVSVGIFKSVLNLLCEHSKLSKLIILGDIRQLPSIEPGNLLKDLFETLKSRNCAIELKTNHRAESQLIVDNATRISRRQFPKFDAELNISDNPTLPISIQDKTFIFVRLPEEDASSQSSKTNHHSCLYSAVKTLLQENDLQNAKTSQFIAFRRQDCDLINDCCCKHYTGHLNKDHQNRLVFGIGDKICCTRNAYLSDLLPENISGSQQNNDLDTSGEDFSGTLHDFAKNKHDFAKNKHDFESNVRLCNGEIFFITNDVTDVTFGMRRSLTINNMAGLEVTVDFKKLMKYCRIKHAWARTIHTFQGSEEQTVVYVVGKAGRQHWQHVYTAVTRGRCRVYVIAEESQLRNAIMKNSFPRKTRLKHFLQSKLSSSGAPPADFQSPRKSSGDSGQPSTPSASPLPVVTDHAMTHDVTWSEASSPDERTLTFAERWQLSSPDEVDTDDDLPKSRASKRTCGVNDDESPSKILMVGESPQVSSRLQNLRLNNLIPRQLFKPTDNQET

SEQ ID NO: 65 - protein, 765 aa, Homo sapiens
MSGDHLHNDSQIEADFRLNDSHKHKDKHKDREHRHKEHKKEKDREKSKHSNSEHKDSEKKHKEKEKTKHKDGSSEKHKDKHKDRDKEKRKEEKVRASGDAKIKKEKENGFSSPPQIKDEPEDDGYFVPPKEDIKPLKRPRDEDDADYKPKKIKTEDTKKEKKRKLEEEEDGKLKKPKNKDKDKKVPEPDNKKKKPKKEEEQKWKWWEEERYPEGIKWKFLEHKGPVFAPPYEPLPENVKFYYDGKVMKLSPKAEEVATFFAKMLDHEYTTKEIFRKNFFKDWRKEMTNEEKNIITNLSKCDFTQMSQYFKAQTEARKQMSKEEKLKIKEENEKLLKEYGFCIMDNHKERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYNASITLQQQLKELTAPDENIPAKILSYNRANRAVAILCNHQRAPPKTFEKSMMNLQTKIDAKKEQLADARRDLKSAKADAKVMKDAKTKKVVESKKKAVQRLEEQLMKLEVQATDREENKQIALGTSKLNYLDPRITVAWCKKWGVPIEKIYNKTQREKFAWAIDMADEDYEF

SEQ ID NO: 66 - protein, 1531 aa, Homo sapiens
MEVSPLQPVNENMQVNKIKKNEDAKKRLSVERIYQKKTQLEHILLRPDTYIGSVELVTQQMWVYDEDVGINYREVTFVPGLYKIFDEILVNAADNKQRDPKMSCIRVTIDPENNLISIWNNGKGIPVVEHKVEKMYVPALIFGQLLTSSNYDDDEKKVTGGRNGYGAKLCNIFSTKFTVETASREYKKMFKQTWMDNMGRAGEMELKPFNGEDYTCITFQPDLSKFKMQSLDKDIVALMVRRAYDIAGSTKDVKVFLNGNKLPVKGFRSYVDMYLKDKLDETGNSLKVIHEQVNHRWEVCLTMSEKGFQQISFVNSIATSKGGRHVDYVADQIVTKLVDVVKKKNKGGVAVKAHQVKNHMWIFVNALIENPTFDSQTKENMTLQPKSFGSTCQLSEKFIKAAIGCGIVESILNWVKFKAQVQLNKKCSAVKHNRIKGIPKLDDANDAGGRNSTECTLILTEGDSAKTLAVSGLGVVGRDKYGVFPLRGKILNVREASHKQIMENAEINNIIKIVGLQYKKNYEDEDSLKTLRYGKIMIMTDQDQDGSHIKGLLINFIHHNWPSLLRHRFLEEFITPIVKVSKNKQEMAFYSLPEFEEWKSSTPNHKKWKVKYYKGLGTSTSKEAKEYFADMKRHRIQFKYSGPEDDAAISLAFSKKQIDDRKEWLTNFMEDRRQRKLLGLPEDYLYGQTTTYLTYNDFINKELILFSNSDNERSIPSMVDGLKPGQRKVLFTCFKRNDKREVKVAQLAGSVAEMSSYHHGEMSLMMTIINLAQNFVGSNNLNLLQPIGQFGTRLHGGKDSASPRYIFTMLSSLARLLFPPKDDHTLKFLYDDNQRVEPEWYIPIIPMVLINGAEGIGTGWSCKIPNFDVREIVNNIRRLMDGEEPLPMLPSYKNFKGTIEELAPNQYVISGEVAILNSTTIEISELPVRTWTQTYKEQVLEPMLNGTEKTPPLITDYREYHTDTTVKFVVKMTEEKLAEAERVGLHKVFKLQTSLTCNSMVLFDHVGCLKKYDTVLDILRDFFELRLKYYGLRKEWLLGMLGAESAKLNNQARFILEKIDGKIIIENKPKKELIKVLIQRGYDSDPVKAWKEAQQKVPDEEENEESDNEKETEKSDSVTDSGPTFNYLLDMPLWYLTKEKKDELCRLRNEKEQELDTLKRKSPSDLWKEDLATFIEELEAVEAKEKQDEQVGLPGKGGKAKGKKTQMAEVLPSPRGQRVIPRITIEMKAEAEKKNKKKIKNENTEGSPQEDGVELEGLKQRLEKKQKREPGTKTKKQTTLAFKPIKKGKKRNPWSDSESDRSSDESNFDVPPRETEPRRAATKTKFTMDLDSDEDFSDFDEKTDDEDFVPSDASPPKTKTSPKLSNKELKPQKSVVSDLEADDVKGSVPLSSSPPATHFPDETEITNPVPKKNVTVKKTAAKSQSSTSTTGAKKRAAPKGTKRDPALNSGVSQKPDPAKTKNRRKRKPSTSDDSDSNFEKIVSKAVTSKKSKGESDDFHMDFDSAVAPRAKSVRAKKPIKYLEESDEDDLF

SEQ ID NO: 67 - protein, 187 aa, Homo sapiens [sequence omitted]

SEQ ID NO: 68 - protein, 9 aa, synthetic construct
  VARIANT 2: note = Any amino acid
  VARIANT 3: note = Any amino acid
  VARIANT 4: note = Any amino acid
  VARIANT 5: note = Any amino acid
GXXXXGKTS

SEQ ID NO: 69 - protein, 15 aa, synthetic construct
  VARIANT 3: note = Any amino acid
  VARIANT 4: note = Any amino acid
  VARIANT 5: note = Any amino acid
  VARIANT 7: note = Any amino acid
  VARIANT 8: note = Any amino acid
  VARIANT 9: note = Any amino acid
  VARIANT 11: note = Any hydrophobic amino acid
  VARIANT 12: note = Any hydrophobic amino acid
  VARIANT 13: note = Any hydrophobic amino acid
RKXXXGXXXLXXXDE

SEQ ID NO: 70 - protein, 11 aa, synthetic construct
  VARIANT 1: note = Any hydrophobic amino acid
  VARIANT 2: note = Any hydrophobic amino acid
  VARIANT 3: note = Any amino acid
  VARIANT 5: note = Any amino acid
  VARIANT 8: note = Any hydrophilic amino acid
XXXGXAGXGKS

SEQ ID NO: 71-74 - skipped (4 entries)

SEQ ID NO: 75 - protein, 15 aa, synthetic construct
  VARIANT 1: note = Any amino acid
  VARIANT 2: note = Any amino acid
  VARIANT 4: note = Any hydrophobic amino acid
  VARIANT 5: note = Any amino acid
  VARIANT 6: note = Any amino acid
  VARIANT 7: note = Any amino acid
  VARIANT 10: note = Any hydrophobic amino acid
  VARIANT 11: note = Any hydrophilic amino acid
  VARIANT 12: note = Any hydrophobic amino acid
  VARIANT 13: note = Any hydrophilic amino acid
  VARIANT 14: note = Any hydrophilic amino acid
XXTXXXXQGXXXXXV

SEQ ID NO: 76 - protein, 8 aa, synthetic construct
  VARIANT 3: note = Any hydrophobic amino acid
  VARIANT 6: note = Any amino acid
  VARIANT 7: note = Any hydrophilic amino acid
  VARIANT 8: note = Any hydrophilic amino acid
VAXTRXXX

SEQ ID NO: 77 - protein, 11 aa, synthetic construct
  VARIANT 1: note = Any hydrophobic amino acid
  VARIANT 2: note = Any hydrophobic amino acid
  VARIANT 3: note = Any amino acid
  VARIANT 4: note = Any amino acid
  VARIANT 5: note = Any amino acid
  VARIANT 6: note = Any hydrophilic amino acid
  VARIANT 8: note = Any amino acid
XXXXXXGXGKT

SEQ ID NO: 78-82 - skipped (5 entries)

SEQ ID NO: 83 - protein, 8 aa, synthetic construct
  VARIANT 2: note = Any amino acid
  VARIANT 3: note = Any amino acid
  VARIANT 6: note = Any amino acid
  VARIANT 7: note = Any amino acid
QXXGRXXR

SEQ ID NO: 84 - protein, 12 aa, synthetic construct
  VARIANT 1: note = Any hydrophobic amino acid
  VARIANT 2: note = Any hydrophobic amino acid
  VARIANT 3: note = Any hydrophobic amino acid
  VARIANT 4: note = Any amino acid
  VARIANT 7: note = Any amino acid
XXXXGPXGTGKS

SEQ ID NO: 85-86 - skipped (2 entries)

SEQ ID NO: 87 - protein, 14 aa, synthetic construct
  VARIANT 1: note = Any amino acid
  VARIANT 2: note = Any hydrophobic amino acid
  VARIANT 3: note = Any amino acid
  VARIANT 4: note = Any hydrophobic amino acid
  VARIANT 5: note = Any hydrophobic amino acid
  VARIANT 6: note = Any amino acid
  VARIANT 9: note = Any amino acid
  VARIANT 10: note = Any amino acid
  VARIANT 11: note = Any hydrophobic amino acid
XXXXXXARXXXGKT

SEQ ID NO: 88 - protein, 8 aa, synthetic construct
  VARIANT 3: note = Any amino acid
  VARIANT 4: note = Any hydrophobic amino acid
VLXXSLEM

SEQ ID NO: 89 - protein, 7 aa, synthetic construct
  VARIANT 1: note = Any hydrophobic amino acid
  VARIANT 3: note = Any hydrophobic amino acid
  VARIANT 4: note = Any hydrophobic amino acid
XIXXDYL

SEQ ID NO: 90 - protein, 23 aa, synthetic construct
  VARIANT 2: note = Any amino acid
  VARIANT 3: note = Any amino acid
  VARIANT 5: note = Any amino acid
  VARIANT 6: note = Any amino acid
  VARIANT 7: note = Any hydrophilic amino acid
  VARIANT 11: note = Any hydrophobic amino acid
  VARIANT 13: note = Any amino acid
  VARIANT 14: note = Any hydrophilic amino acid
  VARIANT 16: note = Any amino acid
  VARIANT 18: note = Any hydrophobic amino acid
  VARIANT 19: note = Any amino acid
  VARIANT 20: note = Any amino acid
  VARIANT 21: note = Any hydrophobic amino acid
  VARIANT 22: note = Any amino acid
IXXIXXXLKAXAXXLXPXXXXXQ

SEQ ID NO: 91 - protein, 20 aa, synthetic construct
  VARIANT 2: note = Any amino acid
  VARIANT 3: note = Any amino acid
  VARIANT 4: note = Any amino acid
  VARIANT 8: note = Any amino acid
  VARIANT 11: note = Any amino acid
  VARIANT 13: note = Any amino acid
  VARIANT 15: note = Any amino acid
  VARIANT 18: note = Any amino acid
  VARIANT 20: note = Any hydrophobic amino acid
PXXXDLRXSGXIXQXADXIX

SEQ ID NO: 92 - protein, 609 aa, Escherichia coli
MAQAEVLNLESGAKQVLQETFGYQQFRPGQEEIIDTVLSGRDCLVVMPTGGGKSLCYQIPALLLNGLTVVVSPLISLMKDQVDQLQANGVAAACLNSTQTREQQLEVMTGCRTGQIRLLYIAPERLMLDNFLEHLAHWNPVLLAVDEAHCISQWGHDFRPEYAALGQLRQRFPTLPFMALTATADDTTRQDIVRLLGLNDPLIQISSFDRPNIRYMLMEKFKPLDQLMRYVQEQRGKSGIIYCNSRAKVEDTAARLQSKGISAAAYHAGLENNVRADVQEKFQRDDLQIVVATVAFGMGINKPNVRFVVHFDIPRNIESYYQETGRAGRDGLPAEAMLFYDPADMAWLRRCLEEKPQGQLQDIERHKLNAMGAFAEAQTCRRLVLLNYFGEGRQEPCGNCDICLDPPKQYDGSTDAQIALSTIGRVNQRFGMGYVVEVIRGANNQRIRDYGHDKLKVYGMGRDKSHEHWVSVIRQLIHLGLVTQNIAQHSALQLTEAARPVLRGESSLQLAVPRIVALKPKAMQKSFGGNYDRKLFAKLRKLRKSIADESNVPPYVVFNDATLIEMAEQMPITASEMLSVNGVGMRKLERFGKPFMALIRAHVDGDDEE

SEQ ID NO: 93 - protein, 591 aa, Bacillus subtilis
MLHRAQSLLAHYFGYEKFRSGQDEAIRLVTEARQNTACIMPTGGGKSICYQIPALMFEGTTIVISPLISLMKDQVDALEEAGINAAYINSTQSNQEIYERLNGLKEGAYKLFYITPERLTSIEFIRILQGIDVPLVAIDEAHCISQWGHDFRPSYRNIEILFRELHDKPVIMALTATATPEVHDDICKQLHIQKENTVYTGFSRENLTFKVVKGENKDRFIDEYVQNNRHEAGIVYTATRKEADRIYERLKRNQVRAGRYHGGLADDVRKEQQERFLNDELQVMVATSAFGMGIDKSNIRFVLHAQIPKDMESYYQEAGRAGRDGLASECVLLFSPQDIMVQRFLIEQSEHEEKQKQDLKKLRQMVDYCHTEDCLQRFILMYFGEKEPDACGQCGNCTDTRAAHDVTREAQMVLSCIIRMKERFGKTMVAQVLAGSKNKKVLENGFSDLSTYGILKHQSVGEISDFIEFLISDDFIRMSDGTFPTLFVSSKGRNVLKGELSVARKEALKAAAITENDELFERLRMVRKEIAAEQGVPPFVVFSDQTLKEMSGKQPVNDDELLSIKGVGEQKRAKYGRLFLQEIQAYARMTD

SEQ ID NO: 94 - protein, 1432 aa, Homo sapiens
MSEKKLETTAQQRKCPEWMNVQNKRCAVEERKACVRKSVFEDDLPFLEFTGSIVYSYDASDCSFLSEDISMSLSDGDVVGFDMEWPPLYNRGKLGKVALIQLCVSESKCYLFHVSSMSVFPQGLKMLLENKAVKKAGVGIEGDQWKLLRDFDIKLKNFVELTDVANKKLKCTETWSLNSLVKHLLGKQLLKDKSIRCSNWSKFPLTEDQKLYAATDAYAGFIIYRNLEILDDTVQRFAINKEEEILLSDMNKQLTSISEEVMDLAKHLPHAFSKLENPRRVSILLKDISENLYSLRRMIIGSTNIETELRPSNNLNLLSFEDSTTGGVQQKQIREHEVLIHVEDETWDPTLDHLAKHDGEDVLGNKVERKEDGFEDGVEDNKLKENMERACLMSLDITEHELQILEQQSQEEYLSDIAYKSTEHLSPNDNENDTSYVIESDEDLEMEMLKHLSPNDNENDTSYVIESDEDLEMEMLKSLENLNSGTVEPTHSKCLKMERNLGLPTKEEEEDDENEANEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKLLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSTKPQEEQKTSLVAKNKICTLSQSMAITYSLFQEKKMPLKSIAESRILPLMTIGMHLSQAVKAGCPLDLERAGLTPEVQKIIADVIRNPPVNSDMSKISLIRMLVPENIDTYLIHMAIEILKHGPDSGLQPSCDVNKRRCFPGSEEICSSSKRSKEEVGINTETSSAERKRRLPVWFAKGSDTSKKLMDKTKRGGLFS

SEQ ID NO: 95 - protein, 1447 aa, Saccharomyces cerevisiae
MVTKPSHNLRREHKWLKETATLQEDKDFVFQAIQKHIANKRPKTNSPPTTPSKDECGPGTTNFITSIPASGPTNTATKQHEVMQTLSNDTEWLSYTATSNQYADVPMVDIPASTSVVSNPRTPNGSKTHNFNTFRPHMASSLVENDSSRNLGSRNNNKSVIDNSSIGKQLENDIKLEVIRLQGSLIMALKEQSKLLLQKCSIIESTSLSEDAKRLQLSRDIRPQLSNMSIRIDSLEKEIIKAKKDGMSKDQSKGRSQVSSQDDNIISSILPSPLEYNTSSRNSNLTSTTATTVTKALAITGAKQNITNNTGKNSNNDSNNDDLIQVLDDEDDIDCDPPVILKEGAPHSPAFPHLHMTSEEQDELTRRRNMRSREPVNYRIPDRDDPFDYVMGKSLRDDYPDVEREEDELTMEAEDDAHSSYMTTRDEEKEENELLNQSDFDFVVNDDLDPTQDTDYHDNMDVSANIQESSQEGDTRSTITLSQNKNVQVILSSPTAQSVPSNGQNQIGVEHIDLLEDDLEKDAILDDSMSFSFGRQHMPMSHSDLELIDSEKENEDFEEDNNNNGIEYLSDSDLERFDEERENRTQVADIQELDNDLKIITERKLTGDNEHPPPSWSPKIKREKSSVSQKDEEDDFDDDFSLSDIVSKSNLSSKTNGPTYPWSDEVLYRLHEVFKLPGFRPNQLEAVNATLQGKDVFVLMPTGGGKSLCYQLPAVVKSGKTHGTTIVISPLISLMQDQVEHLLNKNIKASMFSSRGTAEQRRQTFNLFINGLLDLVYISPEMISASEQCKRAISRLYADGKLARIVVDEAHCVSNWGHDFRPDYKELKFFKREYPDIPMIALTATASEQVRMDIIHNLELKEPVFLKQSFNRTNLYYEVNKKTKNTIFEICDAVKSRFKNQTGIIYCHSKKSCEQTSAQMQRNGIKCAYYHAGMEPDERLSVQKAWQADEIQVICATVAFGMGIDKPDVRFVYHFTVPRTLEGYYQETGRAGRDGNYSYCITYFSFRDIRTMQTMIQKDKNLDRENKEKHLNKLQQVMAYCDNVTDCRRKLVLSYFNEDFDSKLCHKNCDNCRNSANVINEERDVTEPAKKIVKLVESIQNERVTIIYCQDVFKGSRSSKIVQANHDTLEEHGIGKSMQKSEIERIFFHLITIRVLQEYSIMNNSGFASSYVKVGPNAKKLLTGKMEIKMQFTISAPNSRPSTSSSFQANEDNIPVIAQKSTTIGGNVAANPPRFISAKEHLRSYTYGGSTMGSSHPITLKNTSDLRSTQELNNLRMTYERLRELSLNLGNRMVPPVGNFMPDSILKKMAAILPMNDSAFATLGTVEDKYRRRFKYFKATIADLSKKRSSEDHEKYDTILNDEFVNRAAASSNGIAQSTGTKSKFFGANLNEAKENEQIINQIRQSQLPKNTTSSKSGTRSISKSSKKSANGRRGFRNYRGHYRGRK

SEQ ID NO: 96 - protein, 764 aa, Arabidopsis thaliana
MVSLRSRKVIPASEMVSDGKTEKDASGDSPTSVLNEEENCEEKSVTVVEEEILLAKNGDSSLISEAMAQEEEQLLKLREDEEKANNAGSAVAPNLNETQFTKLDELLTQTQLYSEFLLEKMEDITINGIESESQKAEPEKTGRGRKRKAASQYNNTKAKRAVAAMISRSKEDGETINSDLTEEETVIKLQNELCPLLTGGQLKSYQLKGVKWLISLWQNGLNGILADQMGLGKTIQTIGFLSHLKGNGLDGPYLVIAPLSTLSNWFNEIARFTPSINAIIYHGDKNQRDELRRKHMPKTVGPKFPIVITSYEVAMNDAKRILRHYPWKYVVIDEGHRLKNHKCKLLRELKHLKMDNKLLLTGTPLQNNLSELWSLLNFILPDIFTSHDEFESWFDFSEKNKNEATKEEEEKRRAQVVSKLHGILRPFILRRMKCDVELSLPRKKEIIMYATMTDHQKKFQEHLVNNTLEAHLGENAIRGQGWKGKLNNLVIQLRKNCNHPDLLQGQIDGSYLYPPVEEIVGQCGKFRLLERLLVRLFANNHKVLIFSQWTKLLDIMDYYFSEKGFEVCRIDGSVKLDERRRQIKDFSDEKSSCSIFLLSTRAGGLGINLTAADTCILYDSDWNPQMDLQAMDRCHRIGQTKPVHVYRLSTAQSIETRVLKRAYSKLKLEHVVIGQGQFHQERAKSSTPLEEEDILALLKEDETAEDKLIQTDISDADLDRLLDRSDLTITAPGETQAAEAFPVKGPGWEVVLPSSGGMLSSLNS

SEQ ID NO: 97 - protein, 420 aa, Streptococcus pneumoniae
MEEVEVAEVKNARVSLTGEKTKPMKLAEVTSINVNRTKTEMEEFNRVLGGGVVPGSLVLIGGDPGIGKSTLLLQVSTQLSQVGTVLYVSGEESAQQIKLRAERLGDIDSEFYLYAETNMQSVRAEVERIQPDFLIIDSIQTIMSPEISGVQGSVSQVREVTAELMQLAKTNNIAIFIVGHVTKEGTLAGPRMLEHMVDTVLYFEGERHHTFRILRAVKNRFGSTNEIGIFEMQSGGLVEVLNPSQVFLEERLDGATGSSIVVTMEGTRPILAEVQALVTPTMFGNAKRTTTGLDFNRASLIMAVLEKRAGLLLQNQDAYLKSAGGVKLDEPAIDLAVAVAIASSYKDKPTNPQECFVGELGLTGEIRRVNRIEQRINEAAKLGFTKIYVPKNSLTGITLPKEIQVIGVTTIQEVLKKVFA

SEQ ID NO: 98 - protein, 666 aa, Methanothermobacter thermautotrophicus
MMKTVDKSKTLTKFEEFFSLQDYKDRVFEAIEKYPNVRSIEVDYLDLEMFDPDLADLLIEKPDDVIRAAQQAIRNIDRLRKNVDLNIRFSGISNVIPLRELRSKFIGKFVAVDGIVRKTDEIRPRIVKAVFECRGCMRHHAVTQSTNMITEPSLCSECGGRSFRLLQDESEFLDTQTLKLQEPLENLSGGEQPRQITVVLEDDLVDTLTPGDIVRVTGTLRTVRDERTKRFKNFIYGNYTEFLEQEFEELQISEEDEEKIKELAGDPNIYEKIIRSTAPSIHGYREVKEAIALQLFGGTGKELDDKTRLRGDIHILIVGDPGIGKSQMLKYVSKLAPRGIYTSGKGTSGVGLTAAAVRDEFGGWSLEAGALVLGDKGNVCVDELDKMREEDRSAIHEALEQQTISIAKAGIMATLNSRCSVLAAANPKFGRFDSYKSIAEQIDLPSTILSRFDLIFVVEDKPDEEKDRELARHILKTHKEDHMPFEIDPELLRKYIAYARKNVRPVLTDEAMQVLEDFYVSMRASAADEDSPVPITARQLEALVRLSEASAKIKLKEHVEAEDARKAIKLSQACLKQVGYDPETGKIDIDKVEGRTPKSERDKFRLLLELIKEYEDDYGGRAPTNILITEMMDRYNVSEEKVEELIRILKDKGAIFEPARGYLKIV

SEQ ID NO: 99 - protein, 931 aa, unidentified
MSSSQQSGRANELRTPGRANSSSREAVDSSPLFFPASSPGSTRLTTPRTTARTPLASSPLLFESSSPGPNIPQSSRSHLLSQRNDLFLDSSSQRTPRSTRRGDIHSSVQMSTPSRRREVDPQRPGVSTPSSLLFSGSDALTFSQAHPSSEVADDTVRVIWGTNVSIQESIASFRGFLRGFKKKYRPEYRNELMPPPDAEQLVYIEALRNMRIMGLEILNLDVQDLKHYPPTKKLYHQLYSYPQEIIPIMDQTIKDVMLDLLGTNPPEDVLNDIELKIYKIRPFNLEKCINMRDLNPGDIDKLISIKGLVLRCTPVIPDMKQAFFRCSVCGHCVTVEIDRGRIAEPIKCPREVCGATNAMQLIHNRSEFADKQVIKLQETPDVVPDGQTPHSVSLCVYDELVDSARAGDRIEVTGIFRCVPVRLNPRMRTVKSLFKTYVDVVHIKKQDKRRLGTDPSTLESDIAEDAALQIDEVRKISDEEVEKIQQVSKRDDIYDILSRSLAPSIYEMDDVKKGLLLQLFGGTNKSFHKGASPRYRGDINILMCGDPSTSKSQILKYVHKIAPRGVYTSGKGSSAVGLTAYITRDQDTKQLVLESGALVLSDGGICCIDEFDKMSDATRSILHEVMEQQTVTVAKAGIITTLNARTSILASANPIGSKYNPDLPVTKNIDLPPTLLSRFDLVYLILDRVDETLDRKLANHIVSMYMEDTPEHATDMEVFSVEFLTSYITYARNNINPVISEEAAKELVNAYVGMRKLGEDVRASEKRITATTRQLESMIRLSEAHAKMHLRNVVEVGDVLEAARLIKTAIKDYATDPATGKISLDLIYVNERETLVPEDMVKELANLISNLTVGGKTMLVSQLLTRFREQSSTRLDASDFEACLGAFREQSSTRLDASDFEACLGALERRGRIKVITSAGHRIVRSIAQTD

SEQ ID NO: 100 - protein, 892 aa, unidentified
MSSLASQGNNASTPAYRYGFQTSEVGDRPTVSMPSQPESSAMLEDDGMVKRKPFAAIGEAIPKVIDTTGESVREAFEEFLLSFSDDRVAGGDALPSASQEKYYVQQIHGLAMYEIHTVYVDYKHLTSYNDVLALAIVEQYYRFSPFLLRALQKLIEKFEPEYYRSSLSRENASLSPNFKASDKTFALAFYNLPFRSTIRDLRTDRIGRLTTITGTVTRTSEVRPELAQGTFICEECHTVVSNVEQAFRYTEPTQCPNELCANKRSWRLNISQSSFQDWQKVRIQENSNEIPTGSMPRTLDVILRGDIVERAKAGDKCAFTGILIAVPDVSQLGIPGVKPEAYRDSRNFGGRDADGVTGLKSLGVRDLTYKLSFLACMVQPDDANDKSGADVRGDGSQGIEEQDEFLQSLSQEEIDDLRAMVHSDHIYSRLTNSLAPSVYGHEIIKKGILLQLMGGVHKLTPEGINLRGDLNICIVGDPSTSKSQFLKYVCNFLPRAIYTSGKASSAAGLTAAVVKDEETGDFTIEAGALMLADNGICAIDEFDKMDLSDQVAIHEAMEQQTISIAKAGIQATLNARTSILAAANPIGGRYNRKTTLRNNINMSAPIMSRFDLFFVVLDECNESVDRHLAKHIVDIHRLRDDAMQPEFSTEQLQRYIRYARTFKPKLNTESCAEIVKKYKQLRMDDAQGAGKNSYRITVRQLESMIRLSEAIARANCVDDITPAFVNEAYSLLRQSIIHVERDDIEVEEDDAEAQELENDNTNTTNGNDNVSSEEALQKPKVKITYDKYVSIMNGILQVLRQRSTEGVDGVPAGDLVQSYLELREDQFHTEEDIIYEVGLVRKVLTRLVHESIIMEIQNLTDSAVRLPFEERVFSIHPNCDIDALLSNGDVPN

SEQ ID NO: 101 - protein, 760 aa, unidentified
MALPTIDLKIPEYEECQHKITDFLSHFKQEQVQDGQQNQDISMSDAGDEPFLKSKYMDILQKISNRESNVINVDLNDLYEFDPSDTQLLHNIESNAKRFVELFSQCADALMPPPTVEINYRNEVLDVIMQQRVQRNENIDPEHKGFPPELTRGYDLYFRPVTRNKKPFSVRDLRGENLGSLLTVRGIVTRTSDVKPSLTVNAYTCDRCGYEVFQEIRQKTFLPMSECPSDECKKNDAKGQLFMSTRASKFLPFQEVKIQELTNQVPIGHIPRSLTVHLYGAITRSVNPGDIVDISGIFLPTPYTGFRAMRAGLLTDTYLECHYVSQIIKNYTNIEKTPQSEAAIAELNQGGNVYEKLAKSIAPEIYGHEDVKKALLLLLVGGVTKELGDGMRIRGDINICLTGDPGVAKSQLLKYISKVAPRGVYTTGRGSSGVGLTAAVMRDPVTDEMVLEGGALVLADNGICCIDEFDKMDESDRTAIHEVMEQQTISISKAGITTTLNARTSILAAANPLYGRYNPKVAPIHNINLPAALLSRFDILFLILDTPSRETDEHLAQHVTYVHMHNEQPKMDFEPLDPNMIRHYISSARQYRPVVPKDVCDYVTGAYVQLRQNQKRDEANERQFAHTTPRTLLAILRMGQALARLRFSNRVEIGDVDEALRLMSVSKSSLYDDLDPSSHDTTITSKIYKIIRDMLNSIPDVEGNERSLTLRAIRERVLAKGFTEDHLINTIQEYTDLGVLLTTNNGQTIMFLDPDLHMEN

SEQ ID NO: 102 - protein, 199 aa, synthetic construct
  VARIANT 130: note = A, G, F, H, Q, M, N, K, V, D, E, S, C, P, or T
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIXDYDPLYKEALQMLRDAGAQVSIMTYDEFKHCWDTFVDHQGCPFQPWDGLDEHSQALSGRLRAILQNQGN

SEQ ID NO: 103 - protein, 199 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 29: note = Any amino acid
  VARIANT 30: note = Any amino acid
  VARIANT 32: note = Any amino acid
  VARIANT 36: note = Any amino acid
  VARIANT 38: note = Any amino acid
  VARIANT 39: note = Any amino acid
  VARIANT 40: note = Any amino acid
  VARIANT 41: note = Any amino acid
  VARIANT 42: note = Any amino acid
  VARIANT 43: note = Any amino acid
  VARIANT 44: note = Any amino acid
  VARIANT 45: note = Any amino acid
  VARIANT 46: note = Any amino acid
  VARIANT 47: note = Any amino acid
  VARIANT 48: note = Any amino acid
  VARIANT 49: note = Any amino acid
  VARIANT 50: note = Any amino acid
  VARIANT 51: note = Any amino acid
  VARIANT 52: note = Any amino acid
  VARIANT 54: note = Any amino acid
  VARIANT 55: note = Any amino acid
  VARIANT 56: note = Any amino acid
  VARIANT 57: note = Any amino acid
  VARIANT 58: note = Any amino acid
  VARIANT 59: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 65: note = Any amino acid
  VARIANT 66: note = Any amino acid
  VARIANT 67: note = Any amino acid
  VARIANT 68: note = Any amino acid
  VARIANT 69: note = Any amino acid
  VARIANT 73: note = Any amino acid
  VARIANT 74: note = Any amino acid
  VARIANT 76: note = Any amino acid
  VARIANT 77: note = Any amino acid
  VARIANT 78: note = Any amino acid
  VARIANT 79: note = Any amino acid
  VARIANT 80: note = Any amino acid
  VARIANT 81: note = Any amino acid
  VARIANT 82: note = Any amino acid
  VARIANT 83: note = Any amino acid
  VARIANT 84: note = Any amino acid
  VARIANT 85: note = Any amino acid
  VARIANT 86: note = Any amino acid
  VARIANT 87: note = Any amino acid
  VARIANT 88: note = Any amino acid
  VARIANT 89: note = Any amino acid
  VARIANT 91: note = Any amino acid
  VARIANT 92: note = Any amino acid
  VARIANT 95: note = Any amino acid
  VARIANT 96: note = Any amino acid
  VARIANT 102: note = Any amino acid
  VARIANT 103: note = Any amino acid
  VARIANT 104: note = Any amino acid
  VARIANT 105: note = Any amino acid
  VARIANT 108: note = Any amino acid
  VARIANT 109: note = Any amino acid
  VARIANT 110: note = Any amino acid
  VARIANT 111: note = Any amino acid
  VARIANT 112: note = Any amino acid
  VARIANT 115: note = Any amino acid
  VARIANT 116: note = Any amino acid
  VARIANT 117: note = Any amino acid
  VARIANT 118: note = Any amino acid
  VARIANT 119: note = Any amino acid
  VARIANT 120: note = Any amino acid
  VARIANT 121: note = Any amino acid
  VARIANT 123: note = Any amino acid
  VARIANT 126: note = Any amino acid
  VARIANT 127: note = Any amino acid
  VARIANT 129: note = L or I
  VARIANT 130: note = A, G, F, H, Q, M ,N, K, V, D, E, S, P, or T
  VARIANT 131: note = Any amino acid
  VARIANT 132: note = Any amino acid
  VARIANT 133: note = Any amino acid
  VARIANT 134: note = Any amino acid
  VARIANT 135: note = Any amino acid
  VARIANT 136: note = Any amino acid
  VARIANT 137: note = Any amino acid
  VARIANT 138: note = Any amino acid
  VARIANT 141: note = Any amino acid
  VARIANT 142: note = Any amino acid
  VARIANT 144: note = Any amino acid
  VARIANT 145: note = Any amino acid
  VARIANT 146: note = Any amino acid
  VARIANT 148: note = Any amino acid
  VARIANT 149: note = Any amino acid
  VARIANT 150: note = Any amino acid
  VARIANT 151: note = Any amino acid
  VARIANT 152: note = Any amino acid
  VARIANT 154: note = Any amino acid
  VARIANT 155: note = Any amino acid
  VARIANT 156: note = Any amino acid
  VARIANT 157: note = Any amino acid
  VARIANT 159: note = Any amino acid
  VARIANT 160: note = Any amino acid
  VARIANT 163: note = Any amino acid
  VARIANT 164: note = Any amino acid
  VARIANT 167: note = Any amino acid
  VARIANT 168: note = Any amino acid
  VARIANT 169: note = Any amino acid
  VARIANT 170: note = Any amino acid
  VARIANT 171: note = Any amino acid
  VARIANT 172: note = Any amino acid
  VARIANT 174: note = Any amino acid
  VARIANT 177: note = Any amino acid
  VARIANT 178: note = Any amino acid
  VARIANT 179: note = Any amino acid
  VARIANT 180: note = Any amino acid
  VARIANT 181: note = Any amino acid
  VARIANT 182: note = Any amino acid
  VARIANT 183: note = Any amino acid
  VARIANT 184: note = Any amino acid
  VARIANT 185: note = Any amino acid
  VARIANT 186: note = Any amino acid
  VARIANT 187: note = Any amino acid
  VARIANT 188: note = Any amino acid
  VARIANT 189: note = Any amino acid
  VARIANT 191: note = Any amino acid
  VARIANT 192: note = Any amino acid
  VARIANT 194: note = Any amino acid
  VARIANT 195: note = Any amino acid
  VARIANT 196: note = Any amino acid
  VARIANT 197: note = Any amino acid
  VARIANT 198: note = Any amino acid
  VARIANT 199: note = Any amino acid
XXXXXXXXXXXXXXXXXXXXXXXXXXGRXXTXLCYXVXXXXXXXXXXXXXXXGXXXXXXXXXXXXXXXXHAEXXFXXXXXXXXXXXXXXYXXTWXXSWSPCXXXXCAXXXXXFLXXXXXXXLXIFXXRXXXXXXXXXXGLXXLXXXGXXXXXMXXXXFXXCWXXFVXXXXXXFXPWXXXXXXXXXXXXXLXXIXXXXXX

SEQ ID NO: 104 - protein, 199 aa, synthetic construct
  VARIANT 130: note = A, L, or W
  VARIANT 132: note = R, H, L, or G
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIXDXDPLYKEALQMLRDAGAQVSIMTYDEFKHCWDTFVDHQGCPFQPWDGLDEHSQALSGRLRAILQNQGN

SEQ ID NO: 105 - protein, 1045 aa, Pan troglodytes
MRRFKRKHLTAIDCQHLARSHLAVTQPFGQRWTNRDANHGLYPKPRTKRGSRGQGSQRCIPEFFLAGKRPCTNDMAKSNSVGQDSCQDSEGDMIFPAESSCALPQEGSGEAGLGSSGSAPPSRKRSWSSEEESNQATGTSWWDGVSKKAPRHHLSVPCTRPREARQEAEDSTSRLSAESGETDQDAGDMGPDPIPDSYYGLLGTLPCQEALSHICSLPSEVLRHVFAFLPVEDLYWNLSLVCHLWREIISDPLFIPWKKLYHRYLMNEEQAVSKVDGILSNCGIEKESDLCVLNLIRYTATTKCSSSVDPERVLWSLRDHPLLPEAEACVRQHLPDLYAAAGGVNIWALVAAVVLLSSSVNDIQRLLFCLRRPSSTVTMPDVTETLYCIAVLLYAMREKGINISNRIHYNIFYCLYLQENSCTQATKVKEEPSVWPGKKTIQLTHEQQLILNHKMEPLQVVKIMAFAGTGKTSTLVKYAEKWSQSRFLYVTFNKSIAKQAERVFPSNVICKTFHSMAYGHIGRKYQSKKKLNLFKLTPFMVNSVLAEGKGGFIRAKLVCKTLENFFASADEELTIDHVPIWCKNSQGQRVMVEQSEKLNGVLEASRLWDNMRKLGECTEEAYQMTHDGYLKLWQLSKPLLASFDAIFVDEAQDCTPAIMNIVLSQPCGKIFVGDPHQQIYTFRGAVNALFTVPHTHVFYLTQSFRFGVEIAYVGATILDVCKRVRKKTLVGGNHQSGIRGDAKGQVALLSRTNANVFDEAVRVTEGEFPSRIHLIGGIKSFGLDRIIDIWILLQPEEERRKQNLVIKDKFIRRWVHKEGFSGFKRYVTAAEDKELEAKIAVVEKYNIRIPELVQRIEKCHIEDLDFAEYILGTVHKAKGLEFDTVHVLDDFVKVPCARHNLPQLPHFRVESFSEDEWNLLYVAVTRAKKRLIMTKSLENILTLAGEYFLQAELTSNVLKTGVVRCCVGQCNNAIPVDTVLTMKKLPITYSNRKENKGGYLCHSCAEQRIGPLAFLTASPEQVRAMERTVENIVLPRHEALLFLVF

SEQ ID NO: 106 - DNA, 35 bp, synthetic construct; identical to SEQ ID NO: 25
  source 1..35: mol_type = other DNA

SEQ ID NO: 107 - DNA, 35 bp, synthetic construct; identical to SEQ ID NO: 28
  source 1..35: mol_type = other DNA

SEQ ID NO: 108 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIWDYDPLYKEALQMLRDAGAQVSIMTYDEFKHCWDTFVDHQGCPFQPWDGLDEHSQALSGRLRAILQNQGN

SEQ ID NO: 109 - DNA, 48 bp, synthetic construct
  source 1..48: mol_type = other DNA
  modified_base 6: mod_base = OTHER; note = Uracil
  modified_base 10: mod_base = OTHER; note = Uracil
  modified_base 14: mod_base = OTHER; note = Uracil
  modified_base 21: mod_base = OTHER; note = Uracil
  modified_base 25: mod_base = OTHER; note = Uracil
  modified_base 28: mod_base = OTHER; note = Uracil
  modified_base 34: mod_base = OTHER; note = Uracil
  modified_base 36: mod_base = OTHER; note = Uracil
  modified_base 40: mod_base = OTHER; note = Uracil
  modified_base 45: mod_base = OTHER; note = Uracil
aaggtttgttacgtagatgatggatagtgagactattgattgattaac

SEQ ID NO: 110 - skipped

SEQ ID NO: 111 - DNA, 20 bp, synthetic construct
  source 1..20: mol_type = other DNA
ccgtcggaccgattcctaag

SEQ ID NO: 112 - DNA, 20 bp, synthetic construct
  source 1..20: mol_type = other DNA
  modified_base 16: mod_base = OTHER; note = Uracil
ccgtcggactgattcttaag

SEQ ID NO: 113 - DNA, 20 bp, synthetic construct
  source 1..20: mol_type = other DNA
ccgtcggactgattcttaag

SEQ ID NO: 114 - DNA, 20 bp, synthetic construct
  source 1..20: mol_type = other DNA
ccgtcggactgattcctaag

SEQ ID NO: 115 - DNA, 20 bp, synthetic construct
  source 1..20: mol_type = other DNA
cttaggaatcggtccgacgg

SEQ ID NO: 116 - DNA, 20 bp, synthetic construct
  source 1..20: mol_type = other DNA
  modified_base 1: mod_base = OTHER; note = Uracil
  modified_base 2: mod_base = OTHER; note = Uracil
  modified_base 5: mod_base = OTHER; note = Uracil
  modified_base 9: mod_base = OTHER; note = Uracil
  modified_base 15: mod_base = OTHER; note = Uracil
  modified_base 16: mod_base = OTHER; note = Uracil
ttgttggatcgatttttaag

SEQ ID NO: 117 - DNA, 20 bp, synthetic construct
  source 1..20: mol_type = other DNA
  modified_base 1: mod_base = OTHER; note = Uracil
  modified_base 14: mod_base = OTHER; note = Uracil
  modified_base 15: mod_base = OTHER; note = Uracil
  modified_base 18: mod_base = OTHER; note = Uracil
tttaggaatcggtttgatgg

SEQ ID NO: 118 - DNA, 20 bp, synthetic construct
  source 1..20: mol_type = other DNA
cttaggaattggtccgacgg

SEQ ID NO: 119 - DNA, 20 bp, synthetic construct
  source 1..20: mol_type = other DNA
cttaaaaatcgatccaacaa

SEQ ID NO: 120 - DNA, 20 bp, synthetic construct
  source 1..20: mol_type = other DNA
  modified_base 1: mod_base = OTHER; note = Uracil
  modified_base 2: mod_base = OTHER; note = Uracil
  modified_base 5: mod_base = OTHER; note = Uracil
  modified_base 9: mod_base = OTHER; note = Uracil
  modified_base 10: mod_base = OTHER; note = Uracil
  modified_base 15: mod_base = OTHER; note = Uracil
  modified_base 16: mod_base = OTHER; note = Uracil
ttgttggattgatttttaag

SEQ ID NO: 121 - DNA, 20 bp, synthetic construct
  source 1..20: mol_type = other DNA
cttaaaaatcaatccaacaa

SEQ ID NO: 122 - DNA, 20 bp, synthetic construct
  source 1..20: mol_type = other DNA
ccttcaaaccgattcctaat

SEQ ID NO: 123 - DNA, 20 bp, synthetic construct
  source 1..20: mol_type = other DNA
ccttcaaaccaattcctaat

SEQ ID NO: 124 - DNA, 20 bp, synthetic construct
  source 1..20: mol_type = other DNA
  modified_base 1: mod_base = OTHER; note = Uracil
  modified_base 10: mod_base = OTHER; note = Uracil
  modified_base 14: mod_base = OTHER; note = Uracil
  modified_base 15: mod_base = OTHER; note = Uracil
  modified_base 18: mod_base = OTHER; note = Uracil
tttaggaattggtttgatgg

SEQ ID NO: 125 - DNA, 78 bp, synthetic construct
  source 1..78: mol_type = other DNA
gaggtgtatggttgtactaatcactcctggacgaatcttaacacaacgtgcagccaaacgcttcgccacggcaacgtg

SEQ ID NO: 126 - DNA, 19 bp, synthetic construct
  source 1..19: mol_type = other DNA
gtactaattacttctggat

SEQ ID NO: 127 - DNA, 19 bp, synthetic construct
  source 1..19: mol_type = other DNA
accgtagcaaagcatttga

SEQ ID NO: 128 - DNA, 78 bp, synthetic construct
  source 1..78: mol_type = other DNA
gaggtgtatggttgtactaattacttctggatgaatcttaatacaatgtgcagtcaaatgctttgctacggtaacgtg

SEQ ID NO: 129 - DNA, 78 bp, synthetic construct
  source 1..78: mol_type = other DNA
  modified_base 17: mod_base = OTHER; note = Uracil
  modified_base 24: mod_base = OTHER; note = Uracil
  modified_base 27: mod_base = OTHER; note = Uracil
  modified_base 37: mod_base = OTHER; note = Uracil
  modified_base 44: mod_base = OTHER; note = Uracil
  modified_base 51: mod_base = OTHER; note = Uracil
  modified_base 55: mod_base = OTHER; note = Uracil
  modified_base 61: mod_base = OTHER; note = Uracil
  modified_base 66: mod_base = OTHER; note = Uracil
  modified_base 69: mod_base = OTHER; note = Uracil
  modified_base 75: mod_base = OTHER; note = Uracil
gaggtgtatggttgtattaattatttttggatgaattttaatataatgtgtagttaaatgttttgttatggtaatgtg